(19) United States
(12) Patent Application Publication
(10) Pub. No.: US 2026/0275423 A1
(43) Pub. Date: Sep. 17, 2026

(54) METHODS OF DIAGNOSING AND TREATING PATIENTS WITH CUTANEOUS SQUAMOUS CELL CARCINOMA

(71) Applicant: CASTLE BIOSCIENCES, INC., Friendswood, TX (US)

(72) Inventors: Robert Willis COOK, Houston, TX (US); Kyle R. COVINGTON, Missouri City, TX (US); Derek MAETZOLD, Friendswood, TX (US); Sarah J KURLEY, Friendswood, TX (US); Jennifer J. Siegel, Friendswood, TX (US); Alexander Renwick, Friendswood, TX (US); Brian MARTIN, Friendswood, TX (US); Lenka HURTON, Friendswood, TX (US)

(21) Appl. No.: 19/676,777

(22) Filed: May 13, 2026

Related U.S. Application Data
(63) Continuation-in-part of application No. 19/412,836, filed on Dec. 8, 2025.
Application No. 19/412,836 is a continuation-in-part of application No. 18/627,054, filed on Apr. 4, 2024.
(63) Continuation of application No. 16/993,401, filed on Aug. 14, 2020, now Pat. No. 11,976,333.

(51) Int. Cl. C12Q 1/6886

(52) U.S. Cl. CPC C12Q 1/6886; C12Q 2600/106; C12Q 2600/118; C12Q 2600/158

(57) ABSTRACT

The present disclosure relates to methods for predicting the risk of local recurrence and/or metastasis, or both in primary cutaneous squamous cell carcinoma (cSCC).

### CROSS-REFERENCE TO RELATED APPLICATIONS

This application is a continuation-in-part of U.S. application Ser. No. 19/412,836, filed Dec. 8, 2025, which is a continuation-in-part of U.S. application Ser. No. 18/627,054, filed Apr. 4, 2024, which is a continuing application of U.S. application Ser. No. 16/993,401, filed Aug. 14, 2020 (now U.S. Pat. No. 11,976,333), which is a continuation-in-part of U.S. application Ser. No. 16/779,515, filed Jan. 31, 2020 (now U.S. Pat. No. 11,976,331). This application is also a continuation-in-part of U.S. application Ser. No. 19/086,718, filed Mar. 21, 2025, which is a continuation-in-part of U.S. application Ser. No. 18/627,054, filed Apr. 4, 2024, which is a continuing application of U.S. application Ser. No. 16/993,401, filed Aug. 14, 2020 (now U.S. Pat. No. 11,976,333), which is a continuation-in-part of U.S. application Ser. No. 16/779,515, filed Jan. 31, 2020 (now U.S. Pat. No. 11,976,331). The disclosures of each of which are explicitly incorporated by reference herein in their entirety.

### FIELD OF THE DISCLOSURE

The present disclosure relates to methods for predicting the risk of recurrence and/or metastasis in primary cutaneous squamous cell carcinoma (cSCC).

### BACKGROUND

Cutaneous squamous cell carcinoma (cSCC) is rivaled only by basal cell carcinoma as the most common cancer in the U.S. Though most cases are cured by excision, a subset recur and become incurable with the number of deaths approximating melanoma (Karia et al., J. Am. Acad. Dermatol. 68(6): 957-66 (2013)). Despite overall good prognosis for patients with cSCC, a subset will develop local, regional, or distant recurrences/metastases following complete excision of the primary tumor. Those at high risk of recurrence are eligible for adjuvant treatment options. While specific clinical and pathologic features are associated with recurrence, they collectively fail to identify 30-40% of all cSCC recurrences and many tumors that possess high risk features will not recur. Furthermore, the rates of metastasis in high-risk patients (e.g., immunocompromised) and those diagnosed with tumors with high-risk features can exceed 20%. Once metastasis is detected, survival rates are poor. Prediction models with increased positive predictive values while maintaining high negative predictive values are needed to accurately identify patients with high-risk features who are at a much higher risk of developing metastasis and dying from cSCC than the high-risk features alone suggest. Prediction models with increased positive predictive values while maintaining high negative predictive values are critical and may allow for early intervention with adjuvant therapies. Similarly, many patients with high-risk features do not have recurrences and thus maintaining a high negative predictive value is important to avoid overtreatment and prevent unnecessary procedures in patients with low risk cSCC that are mis-categorized as high risk cSCC when using clinical and pathologic features alone. Patients with high-risk features but who are at an actual low risk of disease progression can avoid overtreatment of low risk tumors.

To address the need for more accurate predictive factors and to facilitate appropriate intervention strategies, gene expression analysis was used to determine a signature associated with recurrence and/or metastasis in patients with cSCC, and the combination of the novel signature with clinicopathologic risk factors demonstrated improved risk stratification, which can facilitate risk-appropriate management decisions for high-risk cSCC patients. Furthermore, the clinical need for more accurate risk prediction is addressed by incorporating the tumor biology captured by the 40-GEP test with critical clinical pathologic factors, largely available at the time of biopsy, to provide early and accurate predictions of cSCC disease progression risk to guide clinical decision-making, including surveillance and indications for adjuvant therapies. Starting with the solid foundation provided by the 40-GEP, we extended predictions with the integration of clinical pathologic factors and event-specific predictions to further refine prognostic accuracy in the integrated 40-GEP test (i40-GEP). The i40-GEP test provides more accurate identification of patients whose individual risk places them above or below actionable risk thresholds to optimize care and identifies a larger proportion of patients at high risk of progression, as well as identifying those at-risk who are most likely to benefit from ART.

### SUMMARY

There is a need in the art for a more objective method of predicting which tumors display aggressive local recurrence and/or regional and/or distant metastatic activity. Development of an accurate molecular footprint, such as the gene expression profile assay disclosed herein, would be a significant advance forward for the field. In particular, the integrated 40-gene expression profile (i40-GEP) demonstrated herein integrates tumor biology with clinicopathologic factors for patients with high-risk cutaneous squamous cell carcinoma, and the i40-GEP identifies low-risk patients suitable for de-escalation, high-risk patients who may benefit from escalated care (e.g., multidisciplinary consultation, imaging, and/or adjuvant treatment), and those most likely to benefit from adjuvant radiation therapy (ART).

In one aspect, this disclosure provides a method for treating a patient with a cutaneous squamous cell carcinoma (cSCC) tumor, the method comprising:
(a) obtaining a first risk classification in a cSCC tumor sample from the patient, wherein the first risk classification is obtained by:
   determining the expression levels in a first gene set comprising: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839; and comparing the expression levels of the first gene set to a predictive training set to generate the first risk classification;
   wherein the first risk classification provides an indication as to whether the cSCC tumor has a low risk (Class 1), a moderate risk (Class 2A), or a high risk (Class 2B), and
   wherein when the cSCC tumor is identified as having a low risk (Class 1) or a moderate risk (Class 2A) the method further comprises:
(b) obtaining a second risk classification in the cSCC tumor sample from the patient, wherein the second risk classification is obtained by:
   determining the expression levels in a second gene set comprising: ALOX12, LIME1, MMP10, PI3, RUNX3, TFAP2B, SPP1, HDDC3, LOC100287896, TAF6L, MRC1, RNF135, PLS3, and ZNF839; and comparing the expression levels of the second gene set to a predictive training set to generate the second risk classification;
   wherein the second risk classification provides an indication as to whether the cSCC tumor has a low risk (Class 1), a moderate risk (Class 2A), or a high risk (Class 2B), and;
(c) administering to the patient an aggressive treatment when the cSCC tumor is identified as having a moderate risk (Class 2A) or a high risk (Class 2B) based on the second risk classification.

In another aspect, this disclosure provides a method for determining benefit from adjuvant radiotherapy in a subject having a cutaneous squamous cell carcinoma (cSCC) tumor, the method comprising:
a) determining the expression level of at least 10 genes in a gene set; wherein the at least 10 genes in the gene set are selected from: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839;
b) identifying a risk of metastasis of the cSCC tumor and providing an indication as to whether the cSCC tumor has a low risk (Class 1), a moderate risk (Class 2A), or a high risk (Class 2B) of metastasis; and
c) determining that the subject benefit from adjuvant radiotherapy when the determination is made that the subject has a cSCC tumor with a high risk (Class 2B) of metastasis, wherein the risk is determined in combination with at least one risk factor selected from depth of tumor invasion and tumor differentiation.

In yet another aspect, this disclosure provides a method for treating a patient with a cutaneous squamous cell carcinoma (cSCC) tumor, the method comprising:
(a) obtaining a first risk classification in a cSCC tumor sample from the patient, wherein the first risk classification is obtained by:
   determining the expression levels in a first gene set comprising: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839; and comparing the expression levels of the first gene set to a predictive training set to generate the first risk classification;
   wherein the first risk classification provides an indication as to whether the cSCC tumor has a low risk (Class 1), a moderate risk (Class 2A), or a high risk (Class 2B), and
   wherein when the cSCC tumor is identified as having a low risk (Class 1) or a moderate risk (Class 2A) the method further comprises:
(b) obtaining a second risk classification in the cSCC tumor sample from the patient, wherein the second risk classification is obtained by:
   determining the expression levels in a second gene set comprising: ALOX12, LIME1, MMP10, PI3, RUNX3, TFAP2B, SPP1, HDDC3, LOC100287896, TAF6L, MRC1, RNF135, PLS3, and ZNF839; and comparing the expression levels of the second gene set to a predictive training set to generate the second risk classification;
   wherein the second risk classification provides an indication as to whether the cSCC tumor has a lowest risk (Class 1A), a low risk (Class 1B), a high risk (Class 2A), or the highest risk (Class 2B), and;
(c) administering to the patient an aggressive treatment when the cSCC tumor is identified as having the high risk (Class 2A) or the highest risk (Class 2B) based on the second risk classification.

In another aspect, this disclosure provides a method for determining benefit from adjuvant radiotherapy in a subject having a cutaneous squamous cell carcinoma (cSCC) tumor, the method comprising:
a) determining the expression level of at least 10 genes in a gene set; wherein the at least 10 genes in the gene set are selected from: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839;
b) identifying a risk of metastasis of the cSCC tumor and providing an indication as to whether the cSCC tumor has a lowest risk (Class 1A), a low risk (Class 1B). a high risk (Class 2A), or the highest risk (Class 2B) of metastasis; and
c) determining that the subject will benefit from adjuvant radiotherapy when the determination is made that the subject has a cSCC tumor with the highest risk (Class 2B) of metastasis.

In some embodiments, the first risk classification and/or the second risk classification indicates risk of local recurrence and/or risk of metastasis.

In some embodiments, the method further comprises generating the second risk classification based on the combination with at least one risk factor. In some embodiments, the at least one risk factor is selected from immune status (competent vs suppressed), histological differentiation (well/moderate vs poor), tumor diameter (less than 2 cm or greater than or equal to 2 cm), perineural invasion (PNI), tumor location, immunosuppression, depth of invasion, differentiation, histological subtype, and lymphovascular invasion. In some embodiments, the at least one risk factor is immune status (competent vs suppressed), histological differentiation (well/moderate vs poor) tumor diameter (less than 2 cm or greater than 2 cm), and perineural invasion (PNI).

In some embodiments, the method further comprises determining that the subject benefit from adjuvant radiotherapy (ART) when the patient has a cSCC tumor with a high risk of local recurrence and/or risk of metastasis.

In some embodiments, the method further comprises administering the aggressive treatment selected from one or more of:
a) performing a resection of the cSCC tumor;
b) clinical follow-up of four to twelve times per year for about 3 years;
c) performing baseline and annual nodal imaging at least twice a year for about 2 years;
d) performing a nodal biopsy or a neck dissection;
e) administering an adjuvant treatment; and
f) enrolling in a clinical trial.

In some embodiments, the adjuvant treatment is one or more of chemoradiation, chemotherapy, regional limb therapy, surgery, systemic therapy, or immunotherapy.

In some embodiments, the expression level of each gene in the first gene set and/or the second gene set is determined by reverse transcribing the isolated mRNA and measuring a level of fluorescence for each gene in the gene set by a nucleic acid sequence detection system following RT-PCR.

In some embodiments, the cSCC tumor sample is obtained from a formalin-fixed, paraffin embedded sample.

In some embodiments, the first gene set and/or the second gene set further comprise at least one control gene, and wherein the at least one control gene is selected from the group consisting of BAG6, KMT2D/MLL2, MDM2, FXR1, KMT2C, MDM4, VIM, and NF1B, optionally wherein the control genes are MDM2, KMT2D, BAG6, FXR1, MDM4, and KMT2C.

In some embodiments, the expression level of ACSBG1 is decreased, ALOX12 is decreased, APOBEC3G is increased, ATP6V0E2 is increased, BBC3 is increased, BHLHB9 is decreased, CEP76 is decreased, DUXAP8 is increased, GTPBP2 is decreased, HDDC3 is increased, ID2 is decreased, LCE2B is decreased, LIME1 (ZGPAT) is increased, LOC100287896 is increased, LOC101927502 is increased, MMP10 is decreased, MRC1 is decreased, MSANTD4 is decreased, NFASC is decreased, NFIC is decreased, PDPN is increased, PI3 is decreased, PLS3 is decreased, RCHY1 is increased, RNF135 is increased, RPP38 is decreased, RUNX3 is increased, SLC1A3 is increased, SPP1 is increased, TAF6L is increased, TFAP2B is decreased, ZNF48 is increased, ZNF496 is increased, and ZNF839 is increased, when comparing a recurrent tumor to a non-recurrent sample.

In some embodiments, the expression level of ALOX12 is decreased, HDDC3 is increased, LIME1 (ZGPAT) is increased, LOC100287896 is increased, MMP10 is decreased, MRC1 is decreased, PI3 is decreased, PLS3 is decreased, RNF135 is increased, RUNX3 is increased, SPP1 is increased, TAF6L is increased, TFAP2B is decreased, and ZNF839 is increased, when comparing a recurrent tumor to a non-recurrent sample.

Other aspects, embodiments, and implementations will become apparent from the following detailed description and claims, with reference, where appropriate, to the accompanying drawings.

### BRIEF DESCRIPTION OF THE DRAWINGS

FIG. 1 shows the study design workflow.

FIG. 2 shows the differential expression of 18 genes found to be significantly differentially expressed between recurrent (Rec) and non-recurrent (NR) cSCC cases

FIG. 3 shows another exemplary study design workflow.

FIG. 4 shows a metastasis-free survival curve (regional and distant metastasis) for low risk, Class 1, and high-risk, Class 2, tumors using the 20-1 gene set.

FIG. 5 shows the study cohorts: tissue samples and associated data acquisition. Abbreviations: CRF, case report form; f/u, follow up; event, regional or distant metastasis; QC, quality control.

FIG. 6 shows the Kaplan-Meier analysis of the 40-GEP prognostic test and outcomes from independent validation of cutaneous SCC cases (n=321).

FIG. 7 shows the demographics and clinical characteristics of validation cohort (n=321). Data analyzed using Chi-square test or Kruskal-Wallis F test. Abbreviations: Hx, history; SCC, squamous cell carcinoma; H&N, head and neck; StDev, standard deviation; PNI, perineural invasion; MMS, Mohs micrographic surgery; AJCC8, American Joint Committee on Cancer, Cancer Staging Manual, Eighth Edition; BWH, Brigham and Women's Hospital; NCCN, National Comprehensive Cancer Network. *One (n=1) patient did not report ethnicity. **Tumor diameter reported (n=295). #Tumor thickness reported (n=115). ##Mohs or wide local excision (n=319) with 2 cases not having additional surgery beyond biopsy.

FIG. 8 shows Multivariate Cox regression analyses of risk for metastasis in 40-GEP validation cases (n=321) with binary AJCC and BWH T stage. An event was regional or distant metastasis. Abbreviations: HR, hazard ratio; CI, confidence interval; GEP, gene expression profile; AJCC8, American Joint Committee on Cancer, Cancer Staging Manual, Eighth Edition; BWH, Brigham and Women's Hospital.

FIG. 9 shows classification of cases by 40-GEP Class and clinicopathologic risk group (n=321).

FIG. 10 shows the accuracy of risk prediction of the 40-GEP and risk assessment methods (n=321).

FIG. 11 shows Multivariate Cox regression analyses of risk for metastasis in 40-GEP validation cases (n=321) with AJCC or BWH T stage.

FIG. 12 shows the demographics of the training cohort.

FIG. 13 shows Multivariate Cox regression analyses for risk of metastasis in validation cases with individual pre-operative and post-operative features.

FIG. 14A-14B show the application of 40-GEP test results and T stage to NCCN-defined levels of risk for improving risk-appropriate management of cSCC. FIG. 14A-Using a cohort (n=300) of clinicopathologically defined cSCC patients meeting study criteria and who were NCCN-defined high risk, the 40-GEP test stratified the patients into three groups depending on risk for metastasis at 3 years post-diagnosis: low (Class 1, n=189), high (Class 2A, n=87), or highest (Class 2B, n=24). Patients stratified as Class 1, 2A, and 2B had a 9%, 21%, and 63% risk for metastasis, respectively, per the 40-GEP test alone. Corresponding AJCC and BWH T stages and metastasis rates were analyzed. FIG. 14B-Incorporation of 40-GEP Class plus AJCC and BWH T stages into three metastasis risk bins (<10%, 10-50%, and >50% risk) resulted in low, moderate, and high intensity management strategies. The 40-GEP integration demonstrates low management intensity for 53.0% (AJCC) or 57.7% (BWH), high intensity management for 8.0%, and moderate intensity management for the remainder (39.0%, AJCC; 34.3%, BWH) of the 300-patient cohort.

FIG. 15 shows an exemplary recommended risk-aligned cSCC patient management for prognostic groups based on 40-GEP and T stage. *Risk for metastasis is reported for 40-GEP Class and AJCC T stage.

FIG. 16 shows the characteristics of the NCCN high-risk cSCC cohort (n=300).

FIG. 17 shows the study design and cohort (n=420). Clinicopathologic and outcomes data were collected from 33 institutions from Sep. 3, 2016 to Apr. 1, 2020.

FIG. 18A shows that the 40-GEP test accurately stratified patients based on risk for regional or distant metastasis.

FIG. 18B shows that incorporating the 40-GEP test results identified Class 1 subsets with metastasis rates of 4.0% for 1 risk factor (>50% lower than pre-40-GEP testing).

FIG. 18C shows that incorporating the 40-GEP test results identified Class 1 subsets with metastasis rates of 9.0% for ≥2 risk factors (>50% lower than pre-40-GEP testing).

FIG. 19A shows that when only including cases classified as NCCN high risk (n=407, 62 metastatic cases), stratification of risk by the 40-GEP in line with that of the full cohort was observed.

FIG. 19B shows that when only including cases classified as NCCN high risk (n=407, 62 metastatic cases), stratification of risk by the 40-GEP in line with that of the full cohort was observed and identified Class 1 subsets with metastasis rates of 3.5% for 1 risk factor.

FIG. 19C shows that when only including cases classified as NCCN high risk (n=407, 62 metastatic cases), stratification of risk by the 40-GEP in line with that of the full cohort was observed and identified Class 1 subsets with metastasis rates of 10.9% for ≥2 risk factors.

FIG. 20 shows a consort diagram showing the validation cohort (n=739) that participated in the comprehensive gene expression profile (20-GEP) study.

FIG. 21 shows a 20-GEP composite class result matrix. A combined matrix of two signatures including 20 gene profiles and four clinicopathologic risk factors (immunosuppression, poor histologic differentiation, tumor diameter ≥2 cm, and perineural invasion) was used to predict risk of nonlocal metastasis (regional/distant) and local recurrence. The patients in the validation cohort were classified under Class 1 (low), Class 2A (higher), and Class 2B (highest) based on these risks.

FIG. 22A shows that incorporating the 20-GEP test results significantly stratified patient risk of poor outcomes (local recurrence or metastasis) (log-rank, p<0.001) with a negative predictive value (NPV) of 95.1% based on the event-free survival (EFS) rate of patients in Class 1, Class 2A, and Class 2B.

FIG. 22B shows that incorporating the 20-GEP test results significantly stratified patient risk of metastasis (log-rank, p<0.001) with an NPV of 97.9% based on the metastasis free survival (MFS) rate of patients in Class 1, Class 2A, and Class 2B.

FIG. 22C shows that incorporating the 20-GEP test results significantly stratified patient risk of local recurrence (log-rank, p<0.001) with an NPV of 97.2% based on the local recurrence free survival (LRFS) rate of patients in Class 1, Class 2A, and Class 2B.

FIG. 23A shows MFS rate of patients in class 1, class 2A, and class 2B with an NPV of 98.1% using 20-GEP method on NCCN high-risk (HR) (log-rank, n=473; p<0.001) patient group (based on clinicopathologic data). Incorporating 20-GEP test results significantly differentiated patient risks within NCCN high-risk group.

FIG. 23B shows MFS rate of patients in Class 1, Class 2A, and Class 2B with an NPV of 92.1% using 20-GEP method on NCCN very-high-risk (VHR) (log-rank; n=168; p=0.004) patient group (based on clinicopathologic data). Incorporating 20-GEP test results significantly differentiated patient risk within the NCCN very-high-risk (VHR) group.

FIG. 24A shows LRFS rate of patients in Class 1, Class 2A, and Class 2B with an NPV of 96.7% using 20-GEP method on NCCN high risk (HR) (log-rank; n=473; p<0.001) patient group (based on clinicopathologic data). Incorporating 20-GEP test results significantly differentiated patient risk within the NCCN (HR) group.

FIG. 24B shows LRFS rate of patients in Class 1, Class 2A, and Class 2B with an NPV of 97.4% using 20-GEP method on NCCN very high risk (VHR) (log-rank; n=168; p=0.02) patient group (based on clinicopathologic data). Incorporating 20-GEP test results significantly differentiated patient risk within the NCCN very-high-risk (VHR) group.

FIG. 25 shows a cohort diagram and analysis procedure. Initial eligible patients consisted of a merge of 2 validation cohorts for the 40-gene expression profile (GEP) test for which patients were confirmed as eligible for testing and had a successful 40-GEP test result. After patient exclusions specific to this study were applied, all 920 qualifying patients were matched on clinical risk factors, stratified by adjuvant radiation therapy (ART) status. Random sampling of ART status pairs and bootstrapping were used to avoid excluding any qualified patients and allow results to be generalizable to the cutaneous squamous cell carcinoma high-risk population. Each sampled and resampled cohort was analyzed using survival methods and stratified by GEP result and ART status. Abbreviation: RT=radiation therapy.

FIG. 26A-26C show the sampling distributions for the x10,000 matched resampled cohorts reveal a benefit of adjuvant radiation therapy (ART) for disease progression rates at 5 years and predicted time to metastatic events R (defined as the time until a given cohort is dominated by metastatic events). (FIG. 26A) Median disease progression rate for the 6 40-gene expression profile×ART status groups from ×10,000 sampled cohorts, sampled at 3-month intervals and gently smoothed with a 5-point box filter to visualize the disease progression functions. The red arrow indicates the ~50% increase in 5-year metastasis-free survival specifically for patients with class 2B cutaneous squamous cell carcinoma who received ART. (FIG. 26B) Within the resampled cohort, median difference in disease progression rates at 5 years after diagnosis (±IQR) as noART-ART-treated groups for each 40-gene expression profile class call (positive difference reflects ART benefit). (FIG. 26C) Density plots of the expected time to metastatic events for each sampled cohort. The red arrow indicates the 5-fold increase in time to disease progression for patients with class 2B cutaneous squamous cell carcinoma who received ART relative to those who did not.

FIG. 27A-27D show that patients with class 2B cSCC who received adjuvant radiation therapy (ART) showed significant delay in disease progression and decrease in acceleration of events. (FIG. 27A) Density plots for ART-treated and untreated patients for each 40-gene expression profile class result (see FIG. 26B). (FIG. 27B) Cumulative distribution plots (Kolmogorov-Smirnov test, see Methods) show the underlying function of disease progression. (FIG. 27C) Insets from FIG. 26B show the first 5 years of disease progression. (FIG. 27D) Within-cohort differences in predicted disease progression. *P<0.01; not significant for class 1 and class 2A. Abbreviation: eCDF=empirical cumulative density function.

FIG. 28 shows the control analyses results.

FIG. 29 schematic of cohort and methodological procedures used in the current study. Initial eligible patients for analysis after excluding 48 tumors that did not qualify for study inclusion. Ninety-five percent of eligible tumors were successfully tested with the 40-GEP and included for final analysis. Qualifying tumors were matched on a number of clinical risk factors based on whether the tumor was treated with ART or not. Random sampling of ART-treated and untreated tumor pairs and bootstrapping were used to include all qualified tumors for analyses and extend the results to the high-risk cSCC population. Survival analysis was performed on each sampled and resampled cohort, stratified by 40-GEP Class result and ART treatment status. 40-GEP: 40-gene expression profile; ART: Adjuvant radiation therapy; cSCC: Cutaneous squamous cell carcinoma.

FIG. 30A-30B show sampling distributions for the x10,000 matched resampled cohorts reveal a benefit of adjuvant radiation therapy for disease progression rates at 5 years. (FIG. 30A) The median disease progression rates observed annually for the 40-GEP xART treatment groups are plotted as observed from the x10,000 resampled cohorts for 5 years. Arrow highlights an ~50% decrease in 5-year disease progression rate for Class 2B tumors that received ART. (FIG. 30B) Median differences in disease progression rates at 5 years after cSCC diagnosis are plotted here for within-cohort comparisons (as no ART-ART for each group; +/−interquartile range, IQR) such that a positive difference reflects ART benefit. Note modest benefit observed here for 40-GEP Class 2A tumors, not previously reported. 40-GEP: 40-gene expression profile; ART: Adjuvant radiation therapy; cSCC: Cutaneous squamous cell carcinoma; Met: Lymph node or distant metastasis.

FIG. 31A-31B show a significant delay in disease progression and decreased acceleration of metastatic events was observed for Class 2B adjuvant radiation therapy treated patients. (FIG. 31A) Density plots of the expected time to metastatic events for each of the x10,000 sampled cohorts are shown stratified by ART treatment status, for each 40-GEP class result. Black lines indicate median of each distribution. (FIG. 31B) The underlying function of disease progression is revealed by the associated cumulative distribution plots (Kolmogoro v-Smirno v test, see Methods). *p<0.01 and p<0.05, respectively; not significant for Class 1. 40-GEP: 40-gene expression profile; ART: Adjuvant radiation therapy; eCDF: eEmpirical cumulative density function; yrs: years.

FIG. 32A-32C show the modest ART benefit observed for 40-GEP Class 2A tumors is accounted for by inclusion of the nodal basin in treatment in over 35% of Class 2A patients. (FIG. 32A) Plot showing the difference in median times to metastatic event density plot values between ART-treated and untreated tumors for each 40-GEP Class before and after excluding node-treated patients from analysis. Asterisks indicate significant Kolmogorov-Smirnov test (see Methods). (FIG. 32B) Annual median disease progression rates for the six 40-GEP×ART status groups from ×10,000 sampled cohorts for 5 years after excluding node-treated patients. Note the similar rates observed for Class 2A tumors between ART-treated and untreated tumors. (FIG. 32C) The medians of the difference in disease progression rates within cohorts (noART-ART) at year 5 following diagnosis (+/−interquartile range, IQR) for each 40-GEP class call (positive difference reflects ART benefit). Note the modest benefit shown in FIG. 30 B for 40-GEP Class 2A tumors is no longer observed after exclusion of node-treated patients. 40-GEP: 40-gene expression profile; ART: Adjuvant radiation therapy; Met: Lymph node or distant metastasis; yrs: Years.

FIG. 33 shows a consort diagram. The derivation of the training cohort used in the development of models predicting local recurrence and metastasis and an independent cohort for validation of the i40-GEP test (with local recurrence and metastasis models combined) is shown. Abbreviations: ART, adjuvant radiation therapy; LR, low-risk; Met, metastasis; CI, confidence interval; NCCN, National Comprehensive Cancer Network.

FIG. 34 shows a consort diagram for patient removal from ART benefit analysis.

FIG. 35A-35B show that the i40-GEP significantly stratifies for risk of metastasis and local recurrence. Kaplan-Meier survival curves for the full validation cohort (n=577) stratified by the i40-GEP risk classes for 35A) metastasis-free survival and 35B) local recurrence-free survival. P-values shown are from a global log-rank test. All analyses were capped to exclude recurrence events happening more than 5 years after diagnosis date. Abbreviations: i40-GEP, integrated 40-gene expression profile; LRFS, local recurrence-free survival; MFS, metastasis-free survival CI, confidence interval.

FIG. 36A-36B show that the i40-GEP significantly risk stratifies within NCCN risk subgroups. Kaplan-Meier survival curves for metastasis-free survival in 36A) NCCN high-risk (n=405) and 36B) NCCN very-high-risk (n=161) subgroups, stratified by the i40-GEP risk classes. P-values shown are from a global log-rank test. All analyses were capped to exclude metastatic events happening more than five years after diagnosis date. Abbreviations: CI, confidence interval; HR, high-risk; MFS, metastasis-free survival; NCCN, National Comprehensive Cancer Network; VHR, very-high-risk.

FIG. 37 shows that the i40-GEP identifies patients who will benefit from adjuvant radiation therapy of ART at 5 years after diagnosis. There was a 50% increase in 5-year metastasis-free survival for patients with an i40-GEP Class 2B cSCC who received ART (solid dark curve) versus patients who did not receive ART (dotted dark curve). There was no significant difference for Class 2A patients based on ART treatment in the modeled predictions (lighter curves).

FIG. 38 shows recommendations for risk-aligned patient management guided by the i40-GEP. Abbreviations: ART, adjuvant radiation therapy; i40-GEP, integrated 40-gene expression profile; 40-GEP, foundational 40-gene expression profile; LVI, lymphovascular invasion; MFS, metastasis-free survival; PNI, perineural invasion.

### DETAILED DESCRIPTION

Despite overall good prognosis for patients with cSCC, a subset will develop metastasis (i.e., local, regional, or distant recurrences, or any combination) following complete excision of the primary tumor. Those at high risk of metastasis/recurrence are eligible for adjuvant treatment options. While specific clinical features are associated with metastasis/recurrence, they collectively fail to identify 30-40% of all cSCC recurrences and many tumors that express high risk features will not recur. To address the need for more accurate predictive factors and facilitate appropriate intervention strategies, a gene expression analysis was used to determine a signature associated with metastasis/recurrence in cSCC. The results demonstrate that gene expression differences can distinguish between metastatic/recurrent and non-recurrent cSCC. Such gene expression differences can help identify those patients who might benefit from additional therapeutic interventions and treatments.

Unless otherwise defined, all technical and scientific terms used herein have the same meaning as would be commonly understood by one of ordinary skill in the art to which the claimed invention belongs. Although methods and materials similar or equivalent to those described herein can be used to practice the methods and kits disclosed or claimed herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting. Other features and advantages of the claimed invention will be apparent from the following detailed description.

As used herein, the singular forms “a,” “an,” and “the” include plural referents unless the context clearly dictates otherwise. For example, reference to “a nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment disclosed or claimed herein.

As used herein, the terms “polynucleotide,” “nucleotide,” “oligonucleotide,” and “nucleic acid” can be used interchangeably to refer to nucleic acid comprising DNA, cDNA, RNA, derivatives thereof, or combinations thereof.

In an embodiment, a method for treating a patient with a cutaneous squamous cell carcinoma (cSCC) tumor is disclosed herein, the method comprising: (a) obtaining a diagnosis identifying a risk of disease progression (e.g. local metastasis, local recurrence, regional metastasis, distant metastasis, or any combination), in a cSCC tumor sample from the patient, wherein the diagnosis was obtained by: (1) determining the expression level of at least 10 genes in a gene set; wherein the at least 10 genes in the gene set are selected from: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839; (2) comparing the expression levels of the at least 10 genes in the gene set from the cSCC tumor sample to the expression levels of the 10 genes in the gene set from a predictive training set to generate a probability score of the risk of disease progression, and; (3) providing an indication as to whether the cSCC tumor has a low risk to a high risk of disease progression, based on the probability score generated in step (2); and (4) identifying that the cSCC tumor has a high risk of disease progression, based on the probability score and diagnosing the cSCC tumor as having a high risk of disease progression; (b) administering to the patient an aggressive treatment when the determination is made in the affirmative that the patient has a cSCC tumor with a high risk of disease progression. In certain embodiments, the method further comprises performing a resection of the cSCC tumor when the determination is made in the affirmative that the patient has a cSCC tumor with a high risk of disease progression. In certain embodiments, the method further comprises identifying that the cSCC tumor has a high risk of disease progression based on the probability score in combination with at least one risk factor, wherein the at least one risk factor is selected from tumor size, tumor location, immune status, perineural invasion (PNI), depth of invasion, differentiation, histological subtype, and lymphovascular invasion.

In certain embodiments, the disease progression is risk of local recurrence and/or risk of metastasis. In certain embodiments, the disease progression is risk of local recurrence based on determining the expression level of ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839.

In some embodiments, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR). In certain embodiments, the cSCC tumor sample is obtained from formalin-fixed, paraffin embedded sample.

In another embodiment, the gene set comprises at least one additional gene selected from the genes AIM2, ANXA9, ARPC2, ATP6AP1, BLOC1S1, C1QL4, C21orf59, C3orf70, CCL27, CD163, CHI3L1, CHMP2B, CXCL10, CXCR4, CYP2D6 (LOC101929829), DARS, DCT, DDAH1, DSS1, EGFR, EphB2, FCHSD1, FDFT1, FLG, FN1, HNRNPL, HOXA10 (HOXA9, MIR196B), HPGD, IL24, IL2RB, IL7R, INHBA, IPO5P1, KIT, KLK5, KRT17, KRT18, KRT19, KRT6B, LAMC2, LOR, LRRC47, MIER2, MIR129-1, MIR3916, MKLN1, MMP1, MMP12, MMP13, MMP3, MMP7, MMP9, MRPL21, MYC, NEB, NEFL, NFIA, NFIB, NOA1, PD1, PDL1, PIG3, PIGBOS1, PIM2, PLAU, PTHLH, PTRHD1, RBM33, RPL26L1, S100A8, S100A9, SEPT3, SERPINB2, SERPINB4, SLC25A11, SNORD124, SPATA41, THYN1, TMEM41B, TNNC1, TUBB3, TUFM (MIR4721), TYRP1, UGP2, USP7, VIM, YKT6, and/or ZSCAN31. In other embodiments, the gene set comprises an additional 1, 2, 3, 4, 5, 6, 7, 8, 9, 10, 15, 20, 25, 30, 35, 40, or more than 40 genes selected from the genes listed above.

In an embodiment, a method of treating a patient with a cutaneous squamous cell carcinoma (cSCC) tumor is disclosed herein, the method comprising administering an aggressive cancer treatment regimen to the patient, wherein the patient has a cSCC tumor with moderate risk (Class 2A), or a high risk (Class 2B) of disease progression as generated by comparing the expression levels of at least 10 genes selected from ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839 from the cSCC tumor with the expression levels of the same 10 genes selected from ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839 from a predictive training set.

In one embodiment, the cSCC tumor is determined to have a low risk (Class 1), a moderate risk (Class 2A), or a high risk (Class 2B), wherein a patient having a low risk (Class 1) cSCC tumor has about a 0-10% risk for metastasis, a patient having a moderate risk (Class 2A) cSCC tumor has about a 10-49% risk for metastasis, and a patient having a high risk (Class 2B) cSCC tumor has about a 50-100% risk for disease progression (metastasis, local recurrence, regional metastasis, distant metastasis, or any combination). In certain embodiments, the cSCC tumor is determined to have a low risk (Class 1), a moderate risk (Class 2A), or a high risk (Class 2B), wherein a patient having a low risk (Class 1) cSCC tumor has about a 5-year survival rate of about 100-95%, a patient having a moderate risk (Class 2A) cSCC tumor has about a 5-year survival rate of about of 95-75%, and a patient having a high risk (Class 2B) cSCC tumor has about a 5-year survival rate of about 95-50%. In certain embodiments, the cSCC tumor is determined to have a low risk (Class 1), a high risk (Class 2A), or a highest risk (Class 2B), wherein a patient having a low risk (Class 1) cSCC tumor has about a 5-year survival rate of about 100-95%, a patient having a high risk (Class 2A) cSCC tumor has about a 5-year survival rate of about of 95-75%, and a patient having a highest risk (Class 2B) cSCC tumor has about a 5-year survival rate of about 95-50%.

In certain embodiments, the method further comprises determining that the cSCC tumor has a low risk (Class 1), a moderate risk (Class 2A), or a high risk (Class 2B) based on the expression levels of the at least 10 genes in combination with at least one risk factor, wherein the at least one risk factor is selected from tumor size, tumor location, immune status, perineural invasion (PNI), depth of invasion, differentiation, histological subtype, and lymphovascular invasion.

In another embodiment, the gene set comprises at least one additional gene selected from the genes AIM2, ANXA9, ARPC2, ATP6AP1, BLOC1S1, C1QL4, C21orf59, C3orf70, CCL27, CD163, CHI3L1, CHMP2B, CXCL10, CXCR4, CYP2D6 (LOC101929829), DARS, DCT, DDAH1, DSS1, EGFR, EphB2, FCHSD1, FDFT1, FLG, FN1, HNRNPL, HOXA10 (HOXA9, MIR196B), HPGD, IL24, IL2RB, IL7R, INHBA, IPO5P1, KIT, KLK5, KRT17, KRT18, KRT19, KRT6B, LAMC2, LOR, LRRC47, MIER2, MIR129-1, MIR3916, MKLN1, MMP1, MMP12, MMP13, MMP3, MMP7, MMP9, MRPL21, MYC, NEB, NEFL, NFIA, NFIB, NOA1, PD1, PDL1, PIG3, PIGBOS1, PIM2, PLAU, PTHLH, PTRHD1, RBM33, RPL26L1, S100A8, S100A9, SEPT3, SERPINB2, SERPINB4, SLC25A11, SNORD124, SPATA41, THYN1, TMEM41B, TNNC1, TUBB3, TUFM (MIR4721), TYRP1, UGP2, USP7, VIM, YKT6, and/or ZSCAN31. In other embodiments, the gene set comprises an additional 1, 2, 3, 4, 5, 6, 7, 8, 9, 10, 15, 20, 25, 30, 35, 40, or more than 40 genes selected from the genes listed above.

As used herein, the terms “metastasis” and “recurrence” are used interchangeably, and refer to the recurrence or disease progression that may occur locally (such as local recurrence and in transit disease), regionally (such as regional metastasis, nodal micrometastasis or macrometastasis), or distally (such as distal metastasis to brain, lung and/or other tissues). In certain embodiments, regional metastasis refers to a metastatic lesion within the regional nodal basin, including satellite or in-transit metastasis, but excluding local recurrence, and distant metastasis refers to metastasis beyond the regional lymph node basin. Risk, as used herein, includes low-risk, moderate-risk, or high-risk of disease progression according to any of the statistical methods disclosed herein. Risk, as used herein, can also include low-risk, high-risk, or highest-risk of disease progression according to any of the statistical methods disclosed herein. In one embodiment, risk of recurrence or metastasis for cSCC can be classified from a low risk to a high risk (for example, the cSCC tumor has a graduated risk from low risk to high risk or high risk to low risk of disease progression (metastasis, local recurrence, regional metastasis, or distant metastasis).

In certain embodiments, the first risk classification and/or the second risk classification may be binned, for example a group with an arbitrary designation Class 1 may be selected based on disease progression (e.g. metastasis, local recurrence, regional metastasis, distant metastasis, or any combination) having risk of less than 25%, 20%, 15%, 10%, 5%, or less than 5%. A group with arbitrary designation Class 2A may be selected based on having a risk of disease progression (e.g. metastasis, local recurrence, regional metastasis, distant metastasis, or any combination) of 75%, 70%, 65%, 60%, 55%, 50%, 45%, 40%, 35%, 30%, 25%, or any number in between. A group with arbitrary designation Class 2B may be selected based on having a risk of disease progression (e.g. metastasis, local recurrence, regional metastasis, distant metastasis, or any combination) of 75%, 80%, 85%, 90%, 95%, or higher than 95%. These Class designations may comprise more than three groups or as few as two groups depending on the separation characteristics of the predictive algorithm. A person familiar with the art will be able to determine the optimal binning strategy depending on the distributions of Class probability scores developed by modeling.

In certain embodiments, the first risk classification and/or the second risk classification may be binned, for example a group with an arbitrary designation Class 1A may be selected based on disease progression (e.g. metastasis, local recurrence, regional metastasis, distant metastasis, or any combination) having risk of less than 20%. A group with arbitrary designation Class 1B may be selected based on having a risk of disease progression (e.g. metastasis, local recurrence, regional metastasis, distant metastasis, or any combination) of 30%, 25%, 20%, 15%, 10%, 5%, 4%, 3% or any number in between. A group with arbitrary designation Class 2A may be selected based on having a risk of disease progression (e.g. metastasis, local recurrence, regional metastasis, distant metastasis, or any combination) of 49%, 45%, 40%, 35%, 30%, 25%, 20%, 15%, 10%, 5%, or any number in between. A group with arbitrary designation Class 2B may be selected based on having a risk of disease progression (e.g. metastasis, local recurrence, regional metastasis, distant metastasis, or any combination) of 15%, 20%, 25%, 30%, 35%, 40%, 45%, 50%, 55%, 60%, 65%, 70%, 75%, 80%, 85%, 90%, 95%, or 100%.

In other embodiments, low risk refers to a 3-year relapse-free survival rate, a 3-year metastasis free survival rate, or a 3-year disease specific survival rate of greater than 50%, 55%, 60%, 65%, 70%, 75%, 80%, 85%, 90%, 95%, or more than 95%, and high risk refers to a 3-year relapse-free survival rate, a 3-year metastasis free survival rate, or a 3-year disease specific survival rate of less than 50%, 45%, 40%, 35%, 30%, 25%, 20%, 15%, 10%, 5%, or less than 5%. Class 1, Class 2A, or Class 2B risk of disease progression (metastasis, local recurrence, regional metastasis, distant metastasis, or any combination), as used herein, includes low-risk (Class 1; for example having a disease progression risk of less than 25%, 20%, 15%, 10%, 5%, or less than 5%), moderate risk (Class 2A; for example having a disease progression risk of 75%, 70%, 65%, 60%, 55%, 50%, 45%, 40%, 35%, 30%, 25%, or any number in between) or high-risk (Class 2B; for example, having a disease progression risk of 50, 75%, 80%, 85%, 90%, 95%, or higher than 95%) of disease progression according to any of the statistical methods disclosed herein.

In other embodiments, low risk refers to a 5-year relapse-free survival rate, a 5-year metastasis free survival rate, or a 5-year disease specific survival rate of greater than 50%, 55%, 60%, 65%, 70%, 75%, 80%, 85%, 90%, 95%, or more than 95%, and high risk refers to a 5-year relapse-free survival rate, a 5-year metastasis free survival rate, or a 3-year disease specific survival rate of less than 50%, 45%, 40%, 35%, 30%, 25%, 20%, 15%, 10%, 5%, or less than 5%. Class 1, Class 2A, or Class 2B risk of disease progression (metastasis, local recurrence, regional metastasis, distant metastasis, or any combination), as used herein, includes low-risk (Class 1; for example having a disease progression risk of less than 25%, 20%, 15%, 10%, 5%, or less than 5%), moderate risk (Class 2A; for example having a disease progression risk of 75%, 70%, 65%, 60%, 55%, 50%, 45%, 40%, 35%, 30%, 25%, or any number in between) or high-risk (Class 2B; for example, having a disease progression risk of 50, 75%, 80%, 85%, 90%, 95%, or higher than 95%) of disease progression according to any of the statistical methods disclosed herein.

In certain embodiments, a low risk (Class 1) cSCC tumor has about a 0-10% risk for disease progression, a patient having a moderate risk (Class 2A) cSCC tumor has about a 10-49% risk for disease progression, and a patient having a high risk (Class 2B) cSCC tumor has about a 50-100% risk for disease progression. In certain embodiments, a patient having a low risk (Class 1) cSCC tumor has about a 5-year survival rate of about 100-95%, a patient having a moderate risk (Class 2A) cSCC tumor has about a 5-year survival rate of about of 95-75%, and a patient having a high risk (Class 2B) cSCC tumor has about a 5-year survival rate of about 95-50%. In certain embodiments, a patient having a low risk (Class 1) cSCC tumor has about a 5-year survival rate of about 100-95%, a patient having a high risk (Class 2A) cSCC tumor has about a 5-year survival rate of about of 95-75%, and a patient having a highest risk (Class 2B) cSCC tumor has about a 5-year survival rate of about 95-50%.

In certain embodiments, a lowest risk (Class 1A) cSCC tumor has about a 0-20% risk for disease progression, a low risk (Class 1B) cSCC tumor has about a 3-30% risk for disease progression, a patient having a high risk (Class 2A) cSCC tumor has about a 5-49% risk for disease progression, and a patient having a highest risk (Class 2B) cSCC tumor has about a 15-100% risk for disease progression. In certain embodiments, a patient having a lowest risk (Class 1A) cSCC tumor has about a 5-year survival rate of about 100-95%, a patient having a low risk (Class 1B) cSCC tumor has about a 5-year survival rate of about 95-85%, a patient having a high risk (Class 2A) cSCC tumor has about a 5-year survival rate of about of 95-70%, and a patient having a highest risk (Class 2B) cSCC tumor has about a 5-year survival rate of about 95-50%.

In certain embodiments, a patient having a low risk (Class 1) cSCC tumor has about a 5-year survival rate of about 100-95%, a patient having a moderate risk (Class 2A) cSCC tumor has about a 5-year survival rate of about of 95-75%, and a patient having a high risk (Class 2B) cSCC tumor has about a 5-year survival rate of about 95-50%. In certain embodiments, a patient having a low risk (Class 1) cSCC tumor has about a 5-year survival rate of about 100-95%, a patient having a high risk (Class 2A) cSCC tumor has about a 5-year survival rate of about of 95-75%, and a patient having a highest risk (Class 2B) cSCC tumor has about a 5-year survival rate of about 95-50%.

The term “distant metastasis” or “distal metastasis” as used herein, refers to metastases from a primary cSCC tumor that are disseminated widely. Patients with distant metastases require aggressive treatments, which can eradicate metastatic cSCC, prolong life, and/or cure some patients. In certain embodiments, a low risk (Class 1) cSCC tumor has about a 0-10% risk for distant metastasis, a patient having a moderate risk (Class 2A) cSCC tumor has about a 10-49% risk for distant metastasis, and a patient having a high risk (Class 2B) cSCC tumor has about a 50-100% risk for distant metastasis. In certain embodiments, a lowest risk (Class 1A) cSCC tumor has about a 0-20% risk for distant metastasis, a low risk (Class 1B) cSCC tumor has about a 3-30% risk for distant metastasis, a patient having a high risk (Class 2A) cSCC tumor has about a 5-49% risk for distant metastasis, and a patient having a highest risk (Class 2B) cSCC tumor has about a 15-100% risk for distant metastasis.

As used herein, the terms “local metastasis” and “local recurrence” can be used interchangeably and refer to cancer cells that have spread to tissue immediately surrounding the primary cSCC tumor or were not completely ablated or removed by previous treatment or surgical resection. Local recurrences are typically resistant to chemotherapy and radiation therapy. Local recurrence can be difficult to control and/or treat if: (1) the primary cSCC tumor is located or involves a vital organ or structure that limits the potential for treatment; (2) recurrence after surgery or other therapy occurs, because while likely not a result from metastasis, high rates of recurrence indicate an advanced cSCC tumor; and (3) presence of lymph node metastases, while rare in cSCC, indicate advanced disease.

In some embodiments, the methods described herein can comprise determining that the cSCC tumor has an increased risk of disease progression (metastasis, local recurrence, regional metastasis, distant metastasis, or any combination) or decreased overall survival by combining with clinical staging factors (i.e., risk factors) recommended by, for example, the American Joint Committee on Cancer (AJCC), the Brigham Women's Hospital (BWH), the National Comprehensive Cancer Network (NCCN), the American Academy of Dermatology (AAD), or the American College of Mohs Surgeons (ACMS), to stage the primary cSCC tumor, or other histological features associated with risk of cSCC tumor metastasis or disease-related death.

As used herein, the terms “risk factor” or “clinical staging factors” or “clinicopathologic factor” or “CP” refer to any staging factor (i.e., risk factor) recommended by, for example, the American Joint Committee on Cancer (AJCC), the Brigham Women's Hospital (BWH), the National Comprehensive Cancer Network (NCCN), the American Academy of Dermatology (AAD), or the American College of Mohs Surgeons (ACMS), to stage the primary cSCC tumor, or other histological features associated with risk of cSCC tumor metastasis or disease-related death. For example, a risk factors can include, but are not limited to tumor size (any size on the head, neck, genitalia, hands, feet or pretibial surface (Areas H or M), or ≥2 cm size (or ≥1 cm if keratoacanthoma type) on any other area of the body (Area L)), tumor diameter (less than 2 cm or greater than or equal to 2 cm), tumor location, immune status (competent vs suppressed), perineural invasion (PNI; large (>0.1 mm), named nerve involvement, <0.1 mm in caliber, or unknown), depth of invasion (for example, any one or combination of: invasion beyond subcutaneous fat; depth ≥2 mm; and/or Clark level ≥IV), differentiation (i.e., poorly differentiated tumor histology), histological differentiation (well/moderate vs poor), histological subtype (for example aggressive histological subtypes, which can be for example, any of acantholytic, adenosquamous, desmoplastic, sclerosing, basosquamous, small cell, spindle cell, infiltrating, clear cell, lymphoepithelial, sarcomatoid, or metaplastic subtypes), immunosuppression, and lymphovascular invasion (see also Table 16). Tumor location definitions can be assigned according to the National Comprehensive Cancer Network (NCCN) Guidelines. For example, Area H, ‘mask areas’ of face (central face, eyelids, eyebrows, periorbital, nose, lips [cutaneous and vermillion], chin, mandible, preauricular and postauricular skin/sulci, temple, and ear), genitalia, hands, and feet; Area M, cheeks, forehead, scalp, neck, and pretibia; and Area L, trunk and extremities (excluding hands, nail units, pretibial, ankles, and feet). Immune status can refer to immunosuppressed, and types of immunosuppression can include patients that had an organ transplant, or have leukemia, lymphoma, or HIV.

As used herein, the terms “cutaneous squamous cell carcinoma” or “cSCC” or “SCC” refer to any cutaneous squamous cell carcinoma, regardless of tumor size, in patients without clinical or histologic evidence of regional or distant metastatic disease. A cutaneous squamous cell carcinoma sample may be obtained through a variety of sampling methods such as punch biopsy, shave biopsy, surgical excision (including Mohs micrographic surgery and wide local excision, or similar technique), core needle biopsy, incisional biopsy, endoscope ultrasound (EUS) guided-fine needle aspirate (FNA) biopsy, percutaneous biopsy, and other means of extracting RNA from the primary cSCC tumor. A carcinoma is a type of cancer that develops from epithelial cells. Specifically, a carcinoma is a cancer that begins in a tissue that lines the inner or outer surfaces of the body, and that arises from cells originating in the endodermal, mesodermal, and ectodermal germ layer during embryogenesis. Squamous cell carcinomas have observable features and characteristics indicative of squamous differentiation (e.g., intercellular bridges, keratinization, squamous pearls). The most recognized risk factor for cSCC is exposure to sunlight; thus, most cSCC tumors develop on sun-exposed skin sites, for example, the head or neck area. They can also be found on the face, ears, lips, trunk, arms, legs, hands, or feet. Squamous cell carcinoma is the second most common skin cancer.

As used herein, “overall survival” (OS) refers to the percentage of people in a study or treatment group who are still alive for a certain period of time after they were diagnosed with or started treatment for a disease, such as cancer. The overall survival rate for cSCC is often stated as a three-year survival rate, which is the percentage of people in a study or treatment group who are alive three years after their diagnosis or the start of treatment.

The phrase “measuring the gene-expression levels” or “determining the gene-expression levels,” as used herein, refers to determining or quantifying RNA or proteins expressed by the gene or genes. The term “RNA” includes mRNA transcripts, and/or specific spliced variants of mRNA. The term “RNA product of the gene,” as used herein, refers to RNA transcripts transcribed from the gene and/or specific spliced variants. In some embodiments, mRNA is converted to cDNA before the gene expression levels are measured. With respect to proteins, gene expression refers to proteins translated from the RNA transcripts transcribed from the gene. The term “protein product of the gene” refers to proteins translated from RNA products of the gene. A number of methods can be used to detect or quantify the level of RNA products of the gene or genes within a sample, including microarrays, Real-Time PCR (RT-PCR; including quantitative RT-PCR), nuclease protection assays, RNA-sequencing (RNA-seq), and Northern blot analyses. In one embodiment, the assay uses the APPLIED BIOSYSTEMS™ HT7900 fast Real-Time PCR system. In addition, a person skilled in the art will appreciate that a number of methods can be used to determine the amount of a protein product of a gene of the methods disclosed herein, including immunoassays such as Western blots, ELISA, and immunoprecipitation followed by SDS-PAGE and immunocytochemistry. In certain embodiments, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR).

A person skilled in the art will appreciate that a number of detection agents can be used to determine gene expression. For example, to detect RNA products of the biomarkers, probes, primers, complementary nucleotide sequences, or nucleotide sequences that hybridize to the RNA products can be used. In another example, to detect cDNA products of the biomarkers, probes, primers, complementary nucleotide sequences, or nucleotide sequences that hybridize to the cDNA products can be used. To detect protein products of the biomarkers, ligands or antibodies that specifically bind to the protein products can be used.

As used herein, the term “hybridize” refers to the sequence specific non-covalent binding interaction with a complementary nucleic acid. In one embodiment, the hybridization is under high stringency conditions. Appropriate stringency conditions that promote hybridization are known to those skilled in the art.

As used herein, the terms “probe” and “primer” refer to a nucleic acid sequence that will hybridize to a nucleic acid target sequence. In one example, the probe and/or primer hybridizes to an RNA product of the gene or a complementary nucleic acid sequence. In another example, the probe and/or primer hybridizes to a cDNA product. The length of probe or primer depends on the hybridizing conditions and the sequences of the probe or primer and nucleic acid target sequence. In one embodiment, the probe or primer is at least 8, 10, 15, 20, 25, 50, 75, 100, 150, 200, 250, 400, 500, or more than 500 nucleotides in length. Probes and/or primers may include one or more label. Probes and/or primers may be commercially sourced from various providers (e.g., ThermoFisher Scientific). In certain embodiments, a label may be any substance capable of aiding a machine, detector, sensor, device, or enhanced or unenhanced human eye from differentiating a labeled composition from an unlabeled composition. Examples of labels include, but are not limited to, a radioactive isotope or chelate thereof, dye (fluorescent or non-fluorescent), stain, enzyme, or nonradioactive metal. Specific examples include, but are not limited to, fluorescein, biotin, digoxigenin, alkaline phosphates, biotin, streptavidin, ³H, ¹⁴C, ³²P, ³⁵S, or any other compound capable of emitting radiation, rhodamine, 4-(4′-dimethylamino-phenylazo) benzoic acid; 4-(4′-dimethylamino-phenylazo) sulfonic acid (sulfonyl chloride); 5-((2-aminoethyl)-amino)-naphtalene-1-sulfonic acid; Psoralene derivatives, haptens, cyanines, acridines, fluorescent rhodol derivatives, cholesterol derivatives; ethylene-diamine-tetra-acetic acid and derivatives thereof, or any other compound that may be differentially detected. The label may also include one or more fluorescent dyes. Examples of dyes include, but are not limited to, CAL-Fluor Red 610, CAL-Fluor Orange 560, dR110, 5-FAM, 6FAM, dR6G, JOE, HEX, VIC, TET, dTAMRA, TAMRA, NED, dROX, PET, BHQ+, Gold540, and LIZ.

As used herein, a “sequence detection system” is any computational method in the art that can be used to analyze the results of a PCR reaction. One example is the APPLIED BIOSYSTEMS™ HT7900 fast Real-Time PCR system. In certain embodiments, gene expression can be analyzed using, e.g., direct DNA expression in microarray, Sanger sequencing analysis, Northern blot, the NANOSTRING® technology, serial analysis of gene expression (SAGE), RNA-seq, tissue microarray, or protein expression with immunohistochemistry or western blot technique. PCR generally involves the mixing of a nucleic acid sample, two or more primers that are designed to recognize the template DNA, a DNA polymerase, which may be a thermostable DNA polymerase such as Taq or Pfu, and deoxyribose nucleoside triphosphates (dNTP's). Reverse transcription PCR, quantitative reverse transcription PCR, and quantitative real time reverse transcription PCR are other specific examples of PCR. In real-time PCR analysis, additional reagents, methods, optical detection systems, and devices known in the art are used that allow a measurement of the magnitude of fluorescence in proportion to concentration of amplified DNA. In such analyses, incorporation of fluorescent dye into the amplified strands may be detected or measured. In one embodiment, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR).

As used herein, the terms “differentially expressed” or “differential expression” refer to a difference in the level of expression of the genes that can be assayed by measuring the level of expression of the products of the genes, such as the difference in level of messenger RNA transcript expressed (or converted cDNA) or proteins expressed of the genes. In one embodiment, the difference can be statistically significant. The term “difference in the level of expression” refers to an increase or decrease in the measurable expression level of a given gene as measured by the amount of messenger RNA transcript (or converted cDNA) and/or the amount of protein in a sample as compared with the measurable expression level of a given gene in a control, or control gene or genes in the same sample (for example, a non-recurrence sample). In another embodiment, the differential expression can be compared using the ratio of the level of expression of a given gene or genes as compared with the expression level of the given gene or genes of a control, wherein the ratio is not equal to 1.0. For example, an RNA, cDNA, or protein is differentially expressed if the ratio of the level of expression in a first sample as compared with a second sample is greater than or less than 1.0. For example, a ratio of greater than 1, 1.2, 1.5, 1.7, 2, 3, 3, 5, 10, 15, 20, or more than 20, or a ratio less than 1, 0.8, 0.6, 0.4, 0.2, 0.1, 0.05, 0.001, or less than 0.0001. In yet another embodiment, the differential expression is measured using p-value. For instance, when using p-value, a biomarker is identified as being differentially expressed as between a first sample and a second sample when the p-value is less than 0.1, less than 0.05, less than 0.01, less than 0.005, or less than 0.001.

The terms “increased expression” or “decreased expression,” as used herein, refer to an expression level of one or more genes, or prognostic RNA transcripts, or their corresponding cDNAs, or their expression products that has been found to be differentially expressed in recurrent versus non-recurrent cSCC tumors. The higher the expression level of a gene that predominantly has increased expression in tumors of patients who had recurrence, the higher is the likelihood that the patient suffering from this tumor is expected to have a poor clinical outcome (i.e., higher risk of recurrence, metastasis, or both). In contrast, the lower the expression level of a gene that predominantly has increased expressed in tumors of patients who have recurrent tumors, the higher is the likelihood that the patient suffering from this tumor is expected to have a promising clinical outcome (i.e., decreased risk of recurrence, metastasis, or both). The lower the expression level of a gene that predominantly has decreased expression in tumors of patients who had recurrence, the higher is the likelihood that the patient suffering from this tumor is expected to have a poor clinical outcome (i.e., higher risk of recurrence, metastasis, or both). In contrast, the higher the expression level of a gene that predominantly has decreased expressed in tumors of patients who have recurrent tumors, the higher is the likelihood that the patient suffering from this tumor is expected to have a promising clinical outcome (i.e., decreased risk of recurrence, metastasis, or both).

References herein to the “same” level of biomarker indicate that the level of biomarker measured in each sample is identical (i.e., when compared to the selected reference). References herein to a “similar” level of biomarker indicate that levels are not identical but the difference between them is not statistically significant (i.e., the levels have comparable quantities).

As used herein, the terms “control” and “standard” refer to a specific value that one can use to determine the value obtained from the sample. In one embodiment, a dataset may be obtained from samples from a group of subjects known to have a cutaneous squamous cell carcinoma or subtype. The expression data of the genes in the dataset can be used to create a control (standard) value that is used in testing samples from new subjects. In such an embodiment, the “control” or “standard” is a predetermined value for each gene or set of genes obtained from subjects with a cutaneous squamous cell carcinoma whose gene expression values and tumor types are known. In certain embodiments of the methods disclosed herein, non-limiting examples of control genes can include, but are not limited to, BAG6 (probe ID: Hs00190383), KMT2D/MLL2 (probe ID: Hs00912419_m1), MDM2 (probe ID: Hs00540450_s1), FXR1 (probe ID: Hs01096876_g1), KMT2C (probe ID: Hs01005521_m1), MDM4 (probe ID: Hs00967238_m1), VIM, and NF1B. In certain embodiments of the methods disclosed herein, the control genes are BAG6 (probe ID: Hs00190383), KMT2D/MLL2 (probe ID: Hs00912419_m1), MDM2 (probe ID: Hs00540450_s1), FXR1 (probe ID: Hs01096876_g1), KMT2C (probe ID: Hs01005521_m1), and MDM4 (probe ID: Hs00967238_m1). In some embodiments, a control population may comprise healthy individuals, individuals with cancer, or a mixed population of individuals with or without cancer. In certain embodiments, a control population may comprise individuals with non-metastatic cancer or cancer that did not recur.

As used herein, the term “normal” when used with respect to a sample population refers to an individual or group of individuals that does/do not have a particular disease or condition (e.g., cSCC or recurrent cSCC) and is also not suspected of having or being at risk for developing the disease or condition. The term “normal” is also used herein to qualify a biological specimen or sample (e.g., a biological fluid) isolated from a normal or healthy individual or subject (or group of such subjects), for example, a “normal control sample.” The “normal” level of expression of a marker is the level of expression of the marker in cells in a similar environment or response situation, in a patient not afflicted with cancer. A normal level of expression of a marker may also refer to the level of expression of a “reference sample” (e.g., a sample from a healthy subject not having the marker associated disease). A reference sample expression may be comprised of an expression level of one or more markers from a reference database. Alternatively, a “normal” level of expression of a marker is the level of expression of the marker in non-tumor cells in a similar environment or response situation from the same patient that the tumor is derived from.

As used herein, the terms “gene-expression profile,” “GEP,” or “gene-expression profile signature” refer to any combination of genes, the measured messenger RNA transcript expression levels, cDNA levels, or direct DNA/RNA expression levels, or immunohistochemistry levels of which can be used to distinguish between two biologically different corporal tissues and/or cells and/or cellular changes. In certain embodiments, a gene-expression profile is comprised of the gene-expression levels of 14 discriminant genes of ALOX12, LIME1, MMP10, PI3, RUNX3, TFAP2B, SPP1, HDDC3, LOC100287896, TAF6L, MRC1, RNF135, PLS3, and ZNF839. In some embodiments, the gene set further comprises 6 control genes or normalization genes selected from: BAG6 (probe ID: Hs00190383), KMT2D/MLL2 (probe ID: Hs00912419_m1), MDM2 (probe ID: Hs00540450_s1), FXR1 (probe ID: Hs01096876_g1), KMT2C (probe ID: Hs01005521_m1), MDM4 (probe ID: Hs00967238_m1), VIM, and NF1B. In certain embodiments of the methods disclosed herein, the 6 control genes are BAG6 (probe ID: Hs00190383), KMT2D/MLL2 (probe ID: Hs00912419_m1), MDM2 (probe ID: Hs00540450_s1), FXR1 (probe ID: Hs01096876_g1), KMT2C (probe ID: Hs01005521_m1), and MDM4 (probe ID: Hs00967238_m1).

In certain embodiments, a gene-expression profile is comprised of the gene-expression levels of at least 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 genes, or less than 10 genes. In one embodiment, the gene-expression profile is comprised of 56 genes. In another embodiment, the gene-expression profile is comprised of 40 genes. In another embodiment, the gene-expression profile is comprised of 30 genes. In another embodiment, the gene-expression profile is comprised of 20 genes. In certain embodiments, the genes selected are: ACSBG1, AIM2, ALOX12, ANXA9, APOBEC3G, ARPC2, ATP6AP1, ATP6V0E2, BBC, BHLHB9, BLOC1S1, C1QL4, C21orf59, C3orf70, CCL27, CD163, CEP76, CHI3L1, CHMP2B, CXCL10, CXCR4, CYP2D6 (LOC101929829), DARS, DCT, DDAH1, DSS1, DUXAP8, EGFR, EphB2, FCHSD1, FDFT1, FLG, FN1, GTPBP2, HDDC3, HNRNPL, HOXA10 (HOXA9, MIR196B), HPGD, ID2, IL24, IL2RB, IL7R, INHBA, IPO5P1, KIT, KLK5, KRT17, KRT18, KRT19, KRT6B, LAMC2, LCE2B, LIME1 (ZGPAT), LOC100287896, LOC101927502, LOR, LRRC47, MIER2, MIR129-1, MIR3916, MKLN1, MMP1, MMP10, MMP12, MMP13, MMP3, MMP7, MMP9, MRC1, MRPL21, MSANTD4, MYC, NEB, NEFL, NFASC, NFIA, NFIB, NFIC, NOA1, PD1, PDL1, PDPN, PI3, PIG3, PIGBOS1, PIM2, PLAU, PLS3, PTHLH, PTRHD1, RBM33, RCHY1, RNF135, RPL26L1, RPP38, RUNX3, S100A8, S100A9, SEPT3, SERPINB2, SERPINB4, SLC1A3, SLC25A11, SNORD124, SPATA41, SPP1, TAF6L, TFAP2B, THYN1, TMEM41B, TNNC1, TUBB3, TUFM (MIR4721), TYRP1, UGP2, USP7, VIM, YKT6, ZNF48, ZNF496, ZNF839, and/or ZSCAN31. In other embodiments, the gene set comprises 20 genes, 30 genes, or 40 genes selected from the genes listed above. In some embodiments, the gene set further comprises control genes or normalization genes selected from: BAG6 (probe ID: Hs00190383), KMT2D/MLL2 (probe ID: Hs00912419_m1), MDM2 (probe ID: Hs00540450_s1), FXR1 (probe ID: Hs01096876_g1), KMT2C (probe ID: Hs01005521_m1), MDM4 (probe ID: Hs00967238_m1), VIM, and NF1B.

As used herein, the term “predictive training set” refers to a cohort of cSCC tumors with known clinical outcome for metastasis (i.e., local recurrence, regional metastasis, distant metastasis, or any combination) and known genetic expression profile, used to define or establish all other cSCC tumors, based upon the genetic expression profile of each, as, for example, a low-risk, Class 1 tumor type or a high-risk, Class 2 tumor type. Additionally, included in the predictive training set is the definition of “threshold points,” which are points at which a classification of metastatic risk is determined, specific to each individual gene expression level.

As used herein, the term “altered in a predictive manner” refers to changes in genetic expression profile that predict disease progression (metastasis, local recurrence, regional metastasis, distant metastasis, or any combination), or predict overall survival, event-free survival, metastasis-free survival. In some embodiments, predictive modeling risk assessment can be measured as: 1) a binary outcome having risk of metastasis or overall survival that is classified as low risk (e.g., termed Class 1 herein; wherein Class 1A is a lowest risk, and Class 1B is the low risk) vs. high risk (e.g., termed Class 2 herein; wherein Class 2A is a high risk/moderate risk, and Class 2B is the highest risk); and/or 2) a linear outcome based upon a probability score from 0 to 1 that reflects the correlation of the genetic expression profile of a cSCC tumor with the genetic expression profile of the samples that comprise the training set used to predict risk outcome. Within the probability score range from 0 to 1, a probability score, for example, of less than 0.5 reflects a tumor sample with a low risk of disease progression (metastasis, local recurrence, regional metastasis, distant metastasis, or any combination), or death from disease, while a probability score, for example, of greater than 0.5 reflects a tumor sample with a high risk of disease progression (metastasis, local recurrence, regional metastasis, distant metastasis, or any combination), or death from disease. The increasing probability score from 0 to 1 reflects incrementally declining metastasis free survival.

In one embodiment, the probability score is a bimodal, two-Class analysis, wherein a patient having a value of between 0 and 0.499 is designated as Class 1 (low risk; for example, having a 3-year relapse-free survival rate, a 3-year metastasis free survival rate, or a 3-year disease specific survival rate of greater than 50%, 55%, 60%, 65%, 70%, 75%, 80%, 85%, 90%, 95%, or more than 95%) and a patient having a value of between 0.500 and 1.00 is designated as Class 2 (high risk; for example, having a 3-year metastasis free survival rate, or a 3-year disease specific survival rate of less than 50%, 45%, 40%, 35%, 30%, 25%, 20%, 15%, 10%, 5%, or less than 5%).

In certain embodiments, the probability score is a tri-modal, three-Class analysis, wherein patients are designated as Class 1 (low risk; for example having a disease progression risk of less than 25%, 20%, 15%, 10%, 5%, or less than 5%), Class 2A (moderate risk; for example having a disease progression risk of 75%, 70%, 65%, 60%, 55%, 50%, 45%, 40%, 35%, 30%, 25%, or any number in between), or Class 2B (high risk; for example, having a disease progression risk of 75%, 80%, 85%, 90%, 95%, or higher than 95%). To develop a ternary, or three-class system of risk assessment, with Class 1 having a low risk of disease progression (metastasis, local recurrence, regional metastasis, distant metastasis, or any combination) or death from disease, Class 2A having an moderate risk, and Class 2B having a high risk, the median probability score value for all low risk or high risk tumor samples in the training set was determined, and one standard deviation from the median was established as a numerical boundary to define low or high risk. For example, low risk cSCC tumors within the ternary classification system can have a 3-year metastasis free survival of 100% (e.g., Class 1; with a probability score of 0-0.337), compared to high risk (e.g., Class 2B; with a probability score of 0.673-1) cSCC tumors which can have a 20% 3-year metastasis free survival. Cases falling outside of one standard deviation from the median low or high risk probability scores have an moderate risk, and moderate risk (Class 2A; with a probability score of 0.338-0.672) cSCC tumors can have a 55% 3-year metastasis free survival rate. In certain embodiments, a patient having a low risk (Class 1) cSCC tumor has about a 5-year survival rate of about 100-95%, a patient having a moderate risk (Class 2A) cSCC tumor has about a 5-year survival rate of about of 95-75%, and a patient having a high risk (Class 2B) cSCC tumor has about a 5-year survival rate of about 95-50%. In certain embodiments, a patient having a low risk (Class 1) cSCC tumor has about a 5-year survival rate of about 100-95%, a patient having a high risk (Class 2A) cSCC tumor has about a 5-year survival rate of about of 95-75%, and a patient having a highest risk (Class 2B) cSCC tumor has about a 5-year survival rate of about 95-50%.

In certain embodiments, the probability score is a four-Class analysis, wherein patients are designated as a lowest risk (Class 1A) cSCC tumor has about a 0-20% risk for disease progression, a low risk (Class 1B) cSCC tumor has about a 3-30% risk for disease progression, a patient having a high risk (Class 2A) cSCC tumor has about a 5-49% risk for disease progression, and a patient having a highest risk (Class 2B) cSCC tumor has about a 15-100% risk for disease progression. In certain embodiments, a patient having a lowest risk (Class 1A) cSCC tumor has about a 5-year survival rate of about 100-95%, a patient having a low risk (Class 1B) cSCC tumor has about a 5-year survival rate of about 95-85%, a patient having a high risk (Class 2A) cSCC tumor has about a 5-year survival rate of about of 95-70%, and a patient having a highest risk (Class 2B) cSCC tumor has about a 5-year survival rate of about 95-50%.

The TNM (Tumor-Node-Metastasis) status system is the most widely used cancer staging system among clinicians and is maintained by the American Joint Committee on Cancer (AJCC) and the International Union for Cancer Control (UICC). Cancer staging systems codify the extent of cancer to provide clinicians and patients with the means to quantify prognosis for individual patients and to compare groups of patients in clinical trials and who receive standard care around the world.

Local recurrence rates for cSCC have been reported to be 1-10%, but can be as high as 47% in patients who have cSCCs with high-risk clinical features. While the overall rate of metastasis is ~5%, this rate increases up to ~45% in patients with high-risk clinical features or who have already experienced a recurrence. After regional or distant metastasis occurs, prognosis is usually poor, with 5-year survival rates ranging from 26-34% and 10-year survival rates of 16%. Although the overall percentages of patients who die from cSCC (~1%) are low, the absolute number of deaths are estimated to be equal to or greater than those attributed to melanoma, due to the large number of yearly cSCC diagnoses (400,000-700,000 patients), and account for the majority of NMSC-related deaths. In effect, local and regional recurrence from primary cSCC tumors remains a significant health burden.

Cutaneous squamous cell carcinoma stems from interfollicular epidermal keratinocytes and can arise from precancerous lesions, the most common of which are actinic keratoses. Once the malignant cells enter the dermis, the cSCC becomes invasive. Squamous cell carcinoma can present as smooth or hyperkeratinized lesions that are pink or skin-colored. They can exhibit ulceration and bleed when traumatized. Risk factors that contribute to the development of cSCC include exposures to ultraviolet radiation, ionizing radiation, and chemicals, as well as increased age and male gender. Immunosuppressed individuals, those with a history of non-Hodgkin lymphoma, including chronic lymphocytic leukemia, those with certain genetic skin conditions, and those who have had organ transplants are at a significantly increased risk for developing cSCC. In fact, the latter group has risk up to 100 times that of the normal population. Some drugs used to treat other types of skin cancer (e.g., basal cell carcinoma (BCC), melanoma), including hedgehog, BRAF, and MET inhibitors, can also increase the propensity for cSCC. Small, low-risk lesions can be treated with cryosurgery, curettage and electrodessication, or surgery, while larger, higher risk lesions are generally treated with surgical excision or Mohs surgery. Radiotherapy can be used in conjunction with surgery if margins are not cleared surgically or if there is perineural invasion. If regional recurrence occurs, the lymph nodes are the primary site of involvement, accounting for ~80-85% of cSCC recurrences, while distant metastasis occurs in ~15-20% of patients.

Because the development of regional or distant metastasis leads to an increase death from cSCC and because there are effective adjuvant interventions, there has been an increased interest in more accurately identifying such lesions beyond clinical and pathologic features alone. As such, the National Comprehensive Cancer Network (NCCN) and American Joint Committee on Cancer (AJCC) have recently proposed parameters to distinguish high risk lesions and follow-up measures for these lesions. These high-risk features include tumor size and location (“mask” areas of the face and/or ear and non-glabrous lip), increased thickness or Clark's level, immunosuppression, recurrent lesions, sites of chronic inflammation or previous radiation, poor differentiation, and perineural invasion. However, high-risk cSCC definitions from different groups are discordant, with the AJCC classifying a majority of lesions as low-risk and NCCN classifying a majority as high-risk. Such discrepancies, especially in the T2a and T2b groups, have led to the proposal of alternative staging criteria that can better elucidate high risk cSCC cases. However, in an attempt to improve the positive predictive values, these alternative approaches have a lower sensitivity and categorize many patients who will metastasize as low risk. In effect, there is a clinically unmet need for better markers to identify high-risk lesions, particularly molecular biomarkers that can be objectively evaluated. The validated prognostic gene expression profiles disclosed herein could inform clinical decision-making on, for example: (1) preoperative surgical staging, based on shave biopsy; (2) adjuvant radiation, nodal staging, adjuvant systemic therapy to reduce regional/distant metastasis; and (3) improving identification of patients with cSCC who can benefit from surgical, radiation and immunotherapy interventions.

Squamous cell carcinoma that is predicted to have an increased risk of recurrence, progression, or metastasis can be treated with an aggressive cancer treatment regimen (see NCCN Guidelines® v1. 2020-October 2019). Advanced cSCC may be defined under two headings: (1) local disease; and/or (2) regional nodal/distant metastases. Local disease can be difficult to control and/or treat if: (1) the primary cSCC has invaded into neuronal or vascular structures; (2) there is presence of lymph node metastases, which indicate advanced disease; or (3) distant metastases have been detected.

In an embodiment, a method for predicting risk of disease progression (metastasis, recurrence, regional metastasis, distant metastasis, or any combination), in a patient with a cutaneous squamous cell carcinoma (cSCC) tumor is disclosed herein, the method comprising: (a) obtaining a cSCC tumor sample from the patient and isolating mRNA from the sample; (b) determining the expression level of at least 10 genes in a gene set; wherein the 10 genes in the gene set are selected from: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839; (c) comparing the expression levels of the 10 genes in the gene set from the cSCC tumor sample to the expression levels of the 10 genes in the gene set from a predictive training set to generate a probability score of the risk of disease progression (metastasis, local recurrence, regional metastasis, distant metastasis, or any combination); and (d) providing an indication as to whether the cSCC tumor has a low risk to a high risk of local metastasis (recurrence, regional metastasis, distant metastasis, or any combination), based on the probability score generated in step (c).

In some embodiments, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR). In certain embodiments, the cSCC tumor sample is obtained from formalin-fixed, paraffin embedded sample. In one embodiment, the method further comprises identifying the cSCC tumor as having a high risk of disease progression (metastasis, local recurrence, regional metastasis, distant metastasis, or any combination), based on the probability score, and administering to the patient an aggressive tumor treatment.

In another embodiment, the gene set comprises at least one additional gene selected from the genes AIM2, ANXA9, ARPC2, ATP6AP1, BLOC1S1, C1QL4, C21orf59, C3orf70, CCL27, CD163, CHI3L1, CHMP2B, CXCL10, CXCR4, CYP2D6 (LOC101929829), DARS, DCT, DDAH1, DSS1, EGFR, EphB2, FCHSD1, FDFT1, FLG, FN1, HNRNPL, HOXA10 (HOXA9, MIR196B), HPGD, IL24, IL2RB, IL7R, INHBA, IPO5P1, KIT, KLK5, KRT17, KRT18, KRT19, KRT6B, LAMC2, LOR, LRRC47, MIER2, MIR129-1, MIR3916, MKLN1, MMP1, MMP12, MMP13, MMP3, MMP7, MMP9, MRPL21, MYC, NEB, NEFL, NFIA, NFIB, NOA1, PD1, PDL1, PIG3, PIGBOS1, PIM2, PLAU, PTHLH, PTRHD1, RBM33, RPL26L1, S100A8, S100A9, SEPT3, SERPINB2, SERPINB4, SLC25A11, SNORD124, SPATA41, THYN1, TMEM41B, TNNC1, TUBB3, TUFM (MIR4721), TYRP1, UGP2, USP7, VIM, YKT6, and/or ZSCAN31. In other embodiments, the gene set comprises an additional 1, 2, 3, 4, 5, 6, 7, 8, 9, 10, 15, 20, 25, 30, 35, 40, or more than 40 genes selected from the genes listed above.

In an embodiment, a method for predicting risk of disease progression (metastasis, recurrence, metastasis, or both), in a patient with a cutaneous squamous cell carcinoma (cSCC) tumor is disclosed herein, the method comprising: (a) obtaining a cSCC tumor sample from the patient and isolating mRNA from the sample; (b) determining the expression level of at least 10 genes in a gene set; wherein the 10 genes in the gene set are selected from: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839; and (c) providing an indication as to whether the cSCC tumor has a low risk to a high risk of disease progression (metastasis, local recurrence, regional metastasis, distant metastasis, or any combination), based on the expression level of the at least 10 genes generated in step (b).

In some embodiments, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR). In certain embodiments, the cSCC tumor sample is obtained from formalin-fixed, paraffin embedded sample.

In another embodiment, the gene set comprises at least one additional gene selected from the genes AIM2, ANXA9, ARPC2, ATP6AP1, BLOC1S1, C1QL4, C21orf59, C3orf70, CCL27, CD163, CHI3L1, CHMP2B, CXCL10, CXCR4, CYP2D6 (LOC101929829), DARS, DCT, DDAH1, DSS1, EGFR, EphB2, FCHSD1, FDFT1, FLG, FN1, HNRNPL, HOXA10 (HOXA9, MIR196B), HPGD, IL24, IL2RB, IL7R, INHBA, IPO5P1, KIT, KLK5, KRT17, KRT18, KRT19, KRT6B, LAMC2, LOR, LRRC47, MIER2, MIR129-1, MIR3916, MKLN1, MMP1, MMP12, MMP13, MMP3, MMP7, MMP9, MRPL21, MYC, NEB, NEFL, NFIA, NFIB, NOA1, PD1, PDL1, PIG3, PIGBOS1, PIM2, PLAU, PTHLH, PTRHD1, RBM33, RPL26L1, S100A8, S100A9, SEPT3, SERPINB2, SERPINB4, SLC25A11, SNORD124, SPATA41, THYN1, TMEM41B, TNNC1, TUBB3, TUFM (MIR4721), TYRP1, UGP2, USP7, VIM, YKT6, and/or ZSCAN31. In other embodiments, the gene set comprises an additional 1, 2, 3, 4, 5, 6, 7, 8, 9, 10, 15, 20, 25, 30, 35, 40, or more than 40 genes selected from the genes listed above.

In certain embodiments, the expression level of: ACSBG1 is decreased, AIM2 is increased, ALOX12 is decreased, ANXA9 is decreased, APOBEC3G is increased, ARPC2 is decreased, ATP6AP1 is decreased, ATP6V0E2 is increased, BBC is increased, BHLHB9 is decreased, BLOC1S1 is decreased, C1QL4 is increased, C21orf59 is increased, C3orf70 is increased, CCL27 is decreased, CD163 is increased, CEP76 is decreased, CHI3L1 is increased, CHMP2B is decreased, CXCL10 is decreased, CXCR4 is increased, CYP2D6 (LOC101929829) is decreased, DARS is decreased, DCT is decreased, DDAH1 is decreased, DSS1 is decreased, DUXAP8 is increased, EGFR is increased, EphB2 is increased, FCHSD1 is decreased, FDFT1 is decreased, FLG is decreased, FN1 is increased, GTPBP2 is decreased, HDDC3 is increased, HNRNPL is decreased, HOXA10 (HOXA9, MIR196B) is decreased, HPGD is decreased, ID2 is decreased, IL24 is increased, IL2RB is decreased, IL7R is increased, INHBA is increased, IPO5P1 is increased, KIT is increased, KLK5 is decreased, KRT17 is decreased, KRT18 is increased, KRT19 is decreased, KRT6B is decreased, LAMC2 is decreased, LCE2B is decreased, LIME1 (ZGPAT) is increased, LOC100287896 is increased, LOC101927502 is decreased, LOR is decreased, LRRC47 is increased, MIER2 is increased, MIR129-1 is increased, MIR3916 is increased, MKLN1 is increased, MMP1 is increased, MMP10 is decreased, MMP12 is increased, MMP13 is increased, MMP3 is increased, MMP7 is increased, MMP9 is decreased, MRC1 is increased, MRPL21 is increased, MSANTD4 is decreased, MYC is decreased, NEB is decreased, NEFL is decreased, NFASC is decreased, NFIA is decreased, NFIB is decreased, NFIC is decreased, NOA1 is increased, PD1 is decreased, PDL1 is increased, PDPN is increased, PI3 is decreased, PIG3 is decreased, PIGBOS1 is increased, PIM2 is increased, PLAU is increased, PLS3 is decreased, PTHLH is decreased, PTRHD1 is decreased, RBM33 is increased, RCHY1 is increased, RNF135 is increased, RPL26L1 is increased, RPP38 is decreased, RUNX3 is increased, S100A8 is decreased, S100A9 is decreased, SEPT3 is decreased, SERPINB2 is decreased, SERPINB4 is decreased, SLC1A3 is increased, SLC25A11 is increased, SNORD124 is increased, SPATA41 is increased, SPP1 is increased, TAF6L is increased, TFAP2B is decreased, THYN1 is increased, TMEM41B is decreased, TNNC1 is decreased, TUBB3 is decreased, TUFM (MIR4721) is increased, TYRP1 is decreased, UGP2 is decreased, USP7 is decreased, VIM is increased, YKT6 is increased, ZNF48 is increased, ZNF496 is increased, ZNF839 is increased, and/or ZSCAN31 is decreased. In certain embodiments, the increase or decrease in the expression level is the gene level from a recurrent tumor sample versus a non-recurrent tumor sample.

In an embodiment, a method for treating a patient with cutaneous squamous cell carcinoma (cSCC) tumor is disclosed herein, the method comprising: (a) obtaining a cSCC tumor sample from the patient and isolating mRNA from the sample; (b) determining the expression level of at least 10 genes in a gene set; wherein the at least 10 genes in the gene set are selected from: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839; (c) providing an indication as to whether the cSCC tumor has a low risk to a high risk of disease progression (metastasis, recurrence, regional metastasis, distant metastasis, or any combination), based on the expression level of at least 10 genes generated in step (b); and (d) administering to the patient an aggressive treatment when the determination is made in the affirmative that the patient has a cSCC tumor with a high risk of disease progression (metastasis, local recurrence, regional metastasis, distant metastasis, or any combination).

In some embodiments, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR). In certain embodiments, the cSCC tumor sample is obtained from formalin-fixed, paraffin embedded sample.

In another embodiment, the gene set comprises at least one additional gene selected from the genes AIM2, ANXA9, ARPC2, ATP6AP1, BLOC1S1, C1QL4, C21orf59, C3orf70, CCL27, CD163, CHI3L1, CHMP2B, CXCL10, CXCR4, CYP2D6 (LOC101929829), DARS, DCT, DDAH1, DSS1, EGFR, EphB2, FCHSD1, FDFT1, FLG, FN1, HNRNPL, HOXA10 (HOXA9, MIR196B), HPGD, IL24, IL2RB, IL7R, INHBA, IPO5P1, KIT, KLK5, KRT17, KRT18, KRT19, KRT6B, LAMC2, LOR, LRRC47, MIER2, MIR129-1, MIR3916, MKLN1, MMP1, MMP12, MMP13, MMP3, MMP7, MMP9, MRPL21, MYC, NEB, NEFL, NFIA, NFIB, NOA1, PD1, PDL1, PIG3, PIGBOS1, PIM2, PLAU, PTHLH, PTRHD1, RBM33, RPL26L1, S100A8, S100A9, SEPT3, SERPINB2, SERPINB4, SLC25A11, SNORD124, SPATA41, THYN1, TMEM41B, TNNC1, TUBB3, TUFM (MIR4721), TYRP1, UGP2, USP7, VIM, YKT6, and/or ZSCAN31. In other embodiments, the gene set comprises an additional 1, 2, 3, 4, 5, 6, 7, 8, 9, 10, 15, 20, 25, 30, 35, 40, or more than 40 genes selected from the genes listed above.

As used herein, the terms “treatment,” “treat,” or “treating” refer to a method of reducing the effects of a disease or condition or symptom of the disease or condition. Thus, in the methods disclosed herein, treatment can refer to a 5%, 10%, 20%, 30%, 40%, 50%, 60%, 70%, 80%, 90%, or 100% reduction in the severity of an established disease or condition or symptom of the disease or condition. For example, a method of treating a disease is considered to be a treatment if there is a 5% reduction in one or more symptoms of the disease in a subject as compared to a control. Thus, the reduction can be a 5%, 10%, 20%, 30%, 40%, 50%, 60%, 70%, 80%, 90%, 100% or any percent reduction between 5% and 100% as compared to native or control levels. It is understood that treatment does not necessarily refer to a cure or complete ablation of the disease, condition, or symptoms of the disease or condition. After a cSCC is found and staged, a medical professional or team of medical professionals will recommend one or several treatment options. In determining a treatment plan, factors to consider include the type, location, and stage of the cancer, as well as the patient's overall physical health. Patients with cSCC typically are managed by a health care team made up of doctors from different specialties, such as: a dermatologist (in particular, a dermatologist who specializes in Mohs micrographic surgery), an orthopedic surgeon (in particular, a surgeon who specializes in diseases of the bones, muscles, and joints), a surgical oncologist, a thoracic surgeon, a medical oncologist, a radiation oncologist, and/or a physiatrist (or rehabilitation doctor). After a cSCC is found and staged, a medical professional or team of medical professionals will typically recommend one or several treatment options including one or more of surgery, radiation, chemotherapy, and targeted therapy.

The NCCN Guidelines® define low risk cSCC tumors as tumors that involve: (1) an area of less than 20 mm (for truck and extremities) or less than 10 mm for the cheeks, forehead, scalp, neck and pretibial; (2) well defined borders; (3) primary cSCC tumor; (4) not rapidly growing; (5) from a patient who has no neurologic symptoms and is not considered immunosuppressed; (6) from a site free of chronic inflammation; (7) well or moderately differentiated; (8) free of acantholytic, adenosquamous, desmoplastic, or metaplastic subtypes; (9) depths of less than 2 mm; and (10) free of perineural, lymphatic, or vascular involvement.

The NCCN Guidelines® define high risk cSCC tumors as tumors that involve: (1) an area of greater than 20 mm (for trunk and extremities), greater than 10 mm for the cheeks, forehead, scalp, neck and pretibial, or any cSCC involving the “mask areas” (such as central face, eyelids, eyebrows, periorbital, nose, lips, chin, mandible, temple or ear), genitalia, hands and feet; (2) poorly defined borders; (3) recurrent cSCC tumor; (4) rapidly growing; (5) from a patient who has neurologic symptoms or is considered immunosuppressed; (6) from a site with chronic inflammation; (7) poorly differentiated; (8) presence of acantholytic, adenosquamous, desmoplastic, or metaplastic subtypes; (9) depths of greater than or equal 2 mm; and (10) presence of perineural, lymphatic, or vascular involvement.

As used herein, the term “aggressive cancer treatment regimen” refers to a treatment regimen that is determined by a medical professional or team of medical professionals and can be specific to each patient. In certain embodiments, a cSCC tumor predicted to have a high-risk of recurrence or a high-risk of metastasis, or a decreased chance of survival using the methods and kits disclosed herein, would be treated using an aggressive cancer treatment regimen. Whether a treatment is considered to be aggressive will generally depend on the cancer-type, the age of the patient, and other factors known to those of skill in the art. For example, in breast cancer, adjuvant chemotherapy is a common aggressive treatment given to complement the less aggressive standards of surgery and hormonal therapy. Those skilled in the art are familiar with various other aggressive and less aggressive treatments for each type of cancer. An aggressive cancer treatment regimen is defined by the National Comprehensive Cancer Network (NCCN), and has been defined in the NCCN Guidelines® as including one or more of: 1) imaging (CT scan, PET/CT, MRI, chest X-ray), 2) discussion and/or offering of tumor resection if a tumor is determined to be resectable (e.g., by Mohs micrographic surgery or resection with complete circumferential margin assessment), 3) radiation therapy (RT), 4) chemoradiation, 5) chemotherapy, 6) regional limb therapy, 7) palliative surgery, 8) systemic therapy, 9) immunotherapy, and 10) inclusion in ongoing clinical trials. Guidelines for clinical practice are published in the National Comprehensive Cancer Network (NCCN Guidelines® Squamous Cell Skin Cancer Version 2.2018, updated Oct. 5, 2017, available on the World Wide Web at NCCN.org).

Additional therapeutic options may include, but are not limited to: 1) combination regimens such as: AD (doxorubicin, dacarbazine); AIM (doxorubicin, ifosfamide, mesna); MAID (mesna, doxorubicin, ifosfamide, dacarbazine); ifosfamide, epirubicin, mesna; gemcitabine and docetaxel; gemcitabine and vinorelbine; gemcitabine and dacarbazine; doxorubicin and olaratumab; methotrexate and vinblastine; tamoxifen and sulindac; vincristine, dactinomycin, cylclophosphamide; vincristine, doxorubicin, cyclophosphamide; vincristine, doxorubicin, cyclophosphamide with ifosfamide and etoposide; vincristine, doxorubicin, ifosfamide; cyclophosphamide topotecan; or ifosfamide, doxorubicin; and/or 2) single agents, such as: cisplatin or other metallic compounds, 5-FU/capecitabine (Xeloda®), cetuximab (Erbitux®), cemiplimab (Libtayo®), pembrolizumab (MK-3475), panitumumab (Vectibix®), dacomitinib (PF-00299804), gefitinib (ZD1839, Iressa), doxorubicin, ifosfamide, epirubicin, gemcitabine, dacarbazine, temozolomide, vinorelbine, eribulin, trabectedin, pazopanib, imatinib, sunitinib, regorafenib, sorafenib, nilotinib, dasatinib, interferon, toremifene, methotrexate, irinotecan, topotecan, paclitaxel, nab-paclitaxel (abraxane), docetaxel, bevacizumab, temozolomide, sirolimus (Rapamune®), everolimus, temsirolimus, crizotinib, ceritinib, or palbociclib.

While surgical excision remains the mainstay for treating operable (Stage I-III) cSCC patients, for Stage I patients, en bloc resection with negative margins is generally considered sufficient for long-term local control. For those with incomplete excision margins and/or other unfavorable pathologic features, pre- or post-operative chemotherapy and/or radiation treatment can be recommended. No therapy has shown consistent efficacy for the treatment of excised cSCC, and treatment options for unresectable or advanced cSCC are limited.

Immunotherapy using an anti-PD1 inhibitor has shown promising results in early phase studies with cSCC patients. Examples of immunotherapies (that can be used alone or in combination with any one or more of tumor resection if a tumor is determined to be resectable, radiation therapy, chemoradiation, chemotherapy, regional limb therapy, palliative surgery, systemic therapy, additional immunotherapeutic, or inclusion in ongoing clinical trials), can include, for example, pembrolizumab (Keytruda®) and nivolumab (Opdivo®), cemiplimab (Libtayo®; a fully human monoclonal antibody to Programmed Death-1). PD-1 is a protein on T-cells that normally help keep T-cells from attacking other cells in the body. By blocking PD-1, these drugs can boost the immune response against cancer cells. CTLA-4 inhibitors (for example, ipilimumab (Yervoy®)) are another class of drugs that can boost the immune response. In some instances, cytokine therapy (such as, interferon-alpha and interleukin-2) can be used to boost the immune system. Examples of interferon and interleukin-based treatments can include, but are not limited to, aldesleukin (Proleukin®), interferon alpha-2b (INTRON®), and pegylated interferon alpha-2b (Sylvatron®; PEG-INTRON®, PEGASYS). In another embodiment, oncolytic virus therapy can be used. Along with killing the cells directly, the oncolytic viruses can also alert the immune system to attack the cancer cells. For example, talimogene laherparepvec (Imlygic®), also known as T-VEC, is an oncolytic virus that can be used to treat melanomas. Additional immunotherapies may include CV8102.

Additionally, targeted therapies may be used to treat patients with cSCC. For example, targeted therapies can include, but are not limited to, vemurafenib (Zelboraf®), dabrafenib (Tafinlar®), trametinib (Mekinist®), CLL442, and cobimetinib (Cotellic®). These drugs target common genetic mutations, such as the BRAFV600 mutation, that may be found in a subset of cSCC patients.

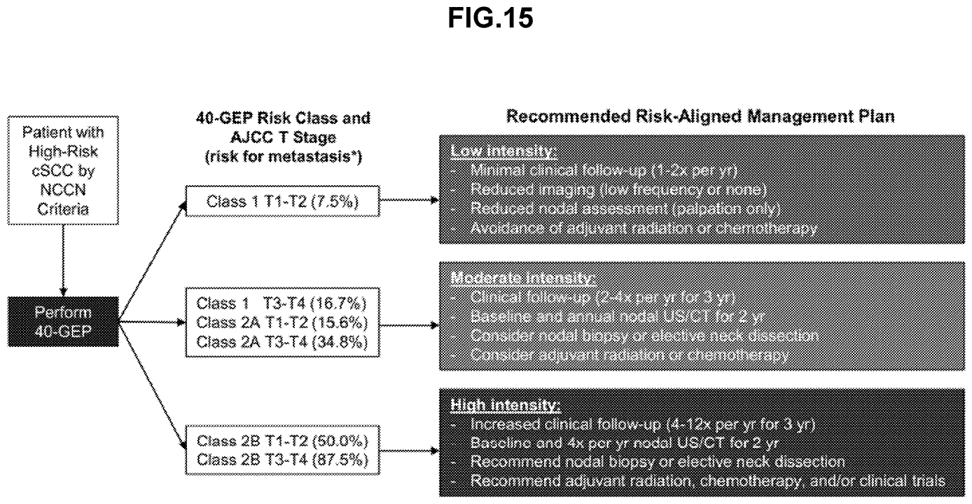

In certain embodiments, the methods as disclosed herein can be used to determine a recommended risk-aligned management plan. For example, patients determined to have a low risk (Class 1) tumor can be managed under a low intensity management plan. A low intensity management plan can comprise minimal clinical follow-up (e.g., 1-2× per year), a reduced imaging (low frequency or no imaging performed), a reduced nodal assessment (palpation only), and/or an avoidance of adjuvant radiation or chemotherapy. For example, patients determined to have a moderate risk (Class 2A) tumor can be managed under a moderate intensity management plan. A moderate intensity management plan can comprise a high frequency of clinical follow-up (e.g., 2-4× per year for about 3 years), imaging (e.g., baseline and annual nodal US/CT for 2 years), consideration of nodal biopsy or elective neck dissection, and/or a consideration of adjuvant radiation or chemotherapy. For example, patients determined to have a high risk (Class 2B) tumor can be managed under a high intensity management plan. A high intensity management plan can comprise the highest frequency of clinical follow-up (e.g., 4-12× per year for about 3 years), imaging (e.g., baseline and 4× per year nodal US/CT for 2 years), recommendation of nodal biopsy or elective neck dissection, and/or a recommendation of adjuvant radiation, chemotherapy, and/or clinical trials. Importantly, these risk-stratified management plans fall within the current NCCN Guidelines® for patients identified as having a high risk cSCC tumor as defined by clinical and pathologic features only (see also FIG. 15).

Adjuvant radiation therapy (ART) is challenging given the large number and extent of recommended clinicopathologic factors that inform risk assessment and indications for ART. Adjuvant radiation therapy for cutaneous squamous cell carcinoma is recommended based on a number of wide-ranging clinicopathologic features, which encompass a broad array of patients. The gene expression profiles (GEP) disclosed herein classify cutaneous squamous cell carcinoma tumors into low (class1), moderate (class2A), or high (class2B) risk of nodal and/or distant metastasis, and demonstrate that: (1) local recurrence is associated with metastatic disease progression; and (2) the GEP disclosed herein also predicts an increase (improvement) in metastasis-free survival after receiving ART.

As used herein, the terms “adjuvant radiation therapy” or “adjuvant radiotherapy” or “ART” refer to radiotherapy or radiation therapy cancer treatment given after primary treatment of the tumor, (for example, typically surgical resection), to lower the risk that the cancer will recur. For example, ART is often used after (or before) a primary surgical treatment (resection) in order to decrease the chance of the primary cancer recurring. In surgery, where all detectable disease has been removed, there remains a statistical risk of relapse or recurrence due to the presence of undetected disease. Adjuvant therapy given before the primary treatment is called neoadjuvant therapy.

Radiotherapy or radiation therapy, uses high-energy radiation to shrink tumors and kill cancer cells. X-rays, gamma rays and charged particles (e.g. electrons, protons, positrons, alpha particles) are examples of types of radiation used for cancer treatment. The radiation may be delivered by a machine outside the body (external radiotherapy), or it may come from radioactive material placed in the body, within or near cancer cells (internal radiotherapy). Internal radiation therapy may be brachytherapy. Alternatively, internal radiation therapy may be performed using a radiopharmaceutical, i.e. a radioactive drug, which is typically swallowed or administered parenterally. Another method is to use radioactive embolization particles.

In some embodiments, radiation therapy typically uses a source of energy of at least 50 keV, 60 keV, 70 keV, 80 keV, 90 keV, or more than 100 keV. Radiation therapy may for instance use a source of energy equal to or greater than 200 keV, for instance equal to or greater than 400 keV.

In some embodiments, radiation therapy can comprise supplying X-ray or gamma ray photons with an incident energy of more than 50 keV, for instance with an incident energy equal to or greater than 60 keV, for example equal to or greater than 70 keV, or equal to or greater than 80 keV.

In some embodiments, radiation therapy can comprise supplying X-ray or gamma ray photons with an incident energy equal to or greater than 100 keV, for instance with an incident energy equal to or greater than 200 keV, or equal to or greater than 400 keV. The photons may for instance have an incident energy of from 0.05 MeV (50 keV) to 10 MeV, for instance from 0.06 MeV (60 keV) to 10 MeV, or for instance from 0.08 MeV (80 keV) to 10 MeV, for example from 0.1 MeV (100 keV) to 1 MeV. The photons may for instance have an incident energy of from 0.2 MeV (200 keV) to 10 MeV, for instance from 0.4 MeV (400 keV) to 10 MeV.

In some embodiments, radiation therapy can comprise supplying electrons, positrons or protons with an incident energy that is equal to or greater than 10 MeV, for instance with an incident energy that is equal to or greater than 50 MeV. The incident energy may for instance be from 60 MeV to 300 MeV, for example from 70 MeV to 250 MeV. The treatment may for instance use, proton beam radiation (proton beam therapy) wherein the incident energy is thus defined, for example is from 70 MeV to 250 MeV.

In some embodiments, adjuvant radiation therapy (ART) can be used to treat the cSCC. In some embodiments, ART is used to treat the local cSCC tumor. In some embodiments, ART is used to treat both the local cSCC tumor and the nodal basin or regional node (i.e. nearest the cSCC tumor). Additional therapeutic options may include, but are not limited to: 1) combination regimens such as: AD (doxorubicin, dacarbazine); AIM (doxorubicin, ifosfamide, mesna); MAID (mesna, doxorubicin, ifosfamide, dacarbazine); ifosfamide, epirubicin, mesna; gemcitabine and docetaxel; gemcitabine and vinorelbine; gemcitabine and dacarbazine; doxorubicin and olaratumab; methotrexate and vinblastine; tamoxifen and sulindac; vincristine, dactinomycin, cylclophosphamide; vincristine, doxorubicin, cyclophosphamide; vincristine, doxorubicin, cyclophosphamide with ifosfamide and etoposide; vincristine, doxorubicin, ifosfamide; cyclophosphamide topotecan; or ifosfamide, doxorubicin; and/or 2) single agents, such as: cisplatin or other metallic compounds, 5-FU/capecitabine (Xeloda®), cetuximab (Erbitux®), cemiplimab (Libtayo®), pembrolizumab (MK-3475), panitumumab (Vectibix®), dacomitinib (PF-00299804), gefitinib (ZD1839, Iressa), doxorubicin, ifosfamide, epirubicin, gemcitabine, dacarbazine, temozolomide, vinorelbine, eribulin, trabectedin, pazopanib, imatinib, sunitinib, regorafenib, sorafenib, nilotinib, dasatinib, interferon, toremifene, methotrexate, irinotecan, topotecan, paclitaxel, nab-paclitaxel (abraxane), docetaxel, bevacizumab, temozolomide, sirolimus (Rapamune®), everolimus, temsirolimus, crizotinib, ceritinib, or palbociclib.

As used herein, the term “adjuvant therapy” refers to additional cancer treatment given after a primary treatment to lower the risk that the cancer will recur. For example, adjuvant therapy is often used before and/or after a primary surgical treatment in order to decrease the chance of the primary cancer recurring. In surgery, where all detectable disease has been removed, there remains a statistical risk of relapse or recurrence due to the presence of undetected disease. Adjuvant therapy given before the primary treatment is called neoadjuvant therapy. Neoadjuvant therapy can also decrease the chance of the cancer recurring, and it's often used to make the primary treatment, such as an operation or radiation treatment more effective. Adjuvant therapy can include radiation, chemotherapy, radiation therapy, hormone therapy, targeted therapy, immunotherapies, or biological therapy.

In some embodiments, the cSCC tumor is a frozen sample. In another embodiment, the cSCC sample is formalin-fixed and paraffin embedded. In certain embodiments, the cSCC sample is taken from a formalin-fixed, paraffin embedded wide local excision sample. In another embodiment, the cSCC tumor is taken from a formalin-fixed, paraffin embedded primary biopsy sample. In some embodiments, the cSCC sample can be from image guided surgical biopsy, shave biopsy, wide excision, or a lymph node dissection.

In certain embodiments, analysis of genetic expression and determination of outcome is carried out using radial basis machine and/or partial least squares analysis (PLS), partition tree analysis, logistic regression analysis (LRA), K-nearest neighbor, neural networks, ensemble learners, voting algorithms, or other algorithmic approach. These analysis techniques take into account the large number of samples required to generate a training set that will enable accurate prediction of outcomes as a result of cut-points established with an in-process training set or cut-points defined for non-algorithmic analysis, but that any number of linear and nonlinear approaches can produce a statistically significant and clinically significant result. As used herein, the term “Kaplan-Meier survival analysis” is understood in the art to be also known as the product limit estimator, which is used to estimate the survival function from lifetime data. In medical research, it is often used to measure the fraction of patients living for a certain amount of time after treatment. JMP GENOMICS®, R, Python libraries including SciPy, SciKit, and numpy software or systems such as TensorFlow provides an interface for utilizing each of the predictive modeling methods disclosed herein, and should not limit the claims to methods performed only with JMP GENOMICS®, R, Python, or TensorFlow software.

### EXAMPLES

The Examples that follow are illustrative of specific embodiments of the claimed invention, and various uses thereof. They are set forth for explanatory purposes only, and should not be construed as limiting the scope of the claimed invention in any way.

### Example 1: CSSC Tumor Sample Preparation and Expression Analysis

a. cSCC Tumor Sample Preparation and RNA Isolation

Formalin-fixed paraffin embedded (FFPE) primary squamous cell carcinoma tumor specimens arranged in 5 μm sections on microscope slides were acquired from multiple institutions under Institutional Review Board (IRB) approved protocols. All tissue was reviewed by a pathologist. Tissue was marked and tumor tissue was dissected from the slide using a sterile disposable scalpel, collected into a microcentrifuge tube, and deparaffinized using xylene. RNA was isolated from each specimen using the QIAGEN QIAsymphony RNA kit (Hilden, Germany) on the QIAGEN QIAsymphony SP sample preparation automated extractor. RNA quantity was assessed using the NanoDrop™ 8000 system.

b. cDNA Generation and RT-PCR Analysis

RNA isolated from FFPE samples was converted to cDNA using the Applied Biosystems High Capacity cDNA Reverse Transcription Kit (Life Technologies Corporation, Grand Island, NY). Prior to performing the RT-PCR assay, each cDNA sample underwent a 14-cycle preamplification step. Pre-amplified cDNA samples were diluted 20-fold in TE buffer. 7.5 μL of each diluted sample was mixed with 7.5 μL of TaqMan OpenArray Real-Time Mastermix, and the solution was loaded to a custom high throughput microfluidics OpenArray card containing primers specific for the genes. Each sample was run in triplicate. The gene expression profile test was performed on a ThermoFisher QuantStudio12k Flex Real-Time PCR system (Life Technologies Corporation, Grand Island, NY).

c. Expression Analysis and Class Assignment

Mean Cₜ values were calculated for triplicate sample sets, and ΔCₜ values were calculated by subtracting the mean Cₜ of each discriminating gene from the geometric mean of the mean Cₜ values of all endogenous control genes. ΔCₜ values were standardized according to the mean of the expression of all discriminant genes with a scale equivalent to the standard deviation. Various predictive modeling methods, including radial basis machine, k-nearest neighbor, partition tree, logistic regression, discriminant analysis and distance scoring, and neural network analysis were performed using R version 3.3.2.

### Example 2: CSCC Metastatic Risk Genetic Signature and Biomarker Expression

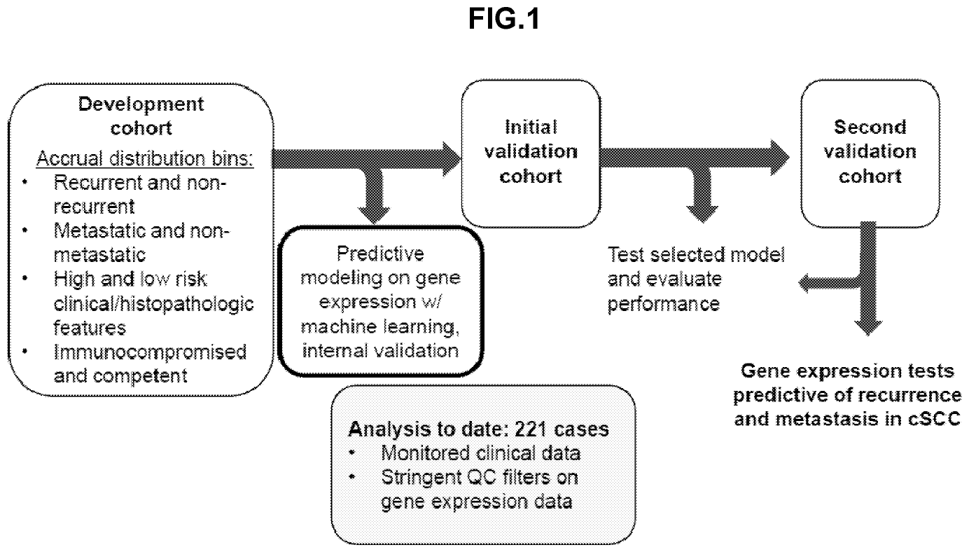

The study design workflow is shown in FIG. 1. First, in order to develop the gene expression profile for cSCC prognostication, cases with annotated clinical data and sufficient follow-up were used as a development set. Pre-specified bins of patients within the recurrent and non-recurrent group were created, including immunocompromised, immunocompetent, those with a certain number of high risk features and low risk cases. The goal was to satisfy the pre-specified number of cases in each bin for development. Predictive modeling was performed on gene expression data from the development cohort. The predictive model was then validated. 221 cases were included in the development set (see Table 1). Table 1 shows the demographics for the cohort of 221 cases used in this study. They are also stratified by non-recurrence or recurrence. Recurrence is defined as any recurrence-local nodal (satellitosis through regional nodes and distant metastasis). Note that cases with R1 or R2 and local recurrence in the scar or contiguous to the scar were embargoed from this analysis. Characteristics that are associated with higher risk tumors (such as male sex, compromised immune system, head and neck primary tumor, poor differentiation or undifferentiated, higher Clark Level, perineural invasion, and invasion into subcutaneous fat) are features included. This is after embargoing cases that have not yet had data monitoring and did not meet very stringent gene expression data requirements.

TABLE 1
Demographics for the cohort of 221 cases used in Examples 2 and 3

| | | Non- | With | |
|---|---|---|---|---|
| | All | Recurrence | Recurrence | |
| Feature | (n = 221) | (n = 196) | (n = 25) | p-value |
| Age: Median years (range) | 74 (43-97) | 74 (45-97) | 69 (43-91) | n.s. |
| Mean +/− SD | 72.8 +/− 11.2 | 73.3 +/− 10.8 | 68.6 +/− 13.2 | |
| Definitive surgery: Mohs | 181 (82%) | 161 (82%) | 20 (80%) | n.s. |
| WLE | 39 (18%) | 34 (17%) | 5 (20%) | |
| Male sex | 164 (74%) | 143 (73%) | 21 (84%) | n.s. |
| Patient immunocompromised | 30 (14%) | 20 (10%) | 10 (40%) | p < 0.001 |
| Located on head or neck | 146 (66%) | 129 (66%) | 17 (68%) | n.s. |
| Tumor diameter: | | | | |
| Median cm (range) | 1.4 (0-28) | 1.15 (0-8.8) | 2.9 (0.25-28) | p < 0.001 |
| Mean +/− SD | 1.88 +/− 2.34 | 1.62 +/− 1.47 | 3.89 +/− 5.27 | |
| Differentiation Status: | | | | |
| Poor/Undifferentiated | 12 (5%) | 9 (5%) | 3 (12%) | p < 0.001 |
| Clark Level IV/V | 73 (33%) | 67 (34%) | 6 (24%) | p < 0.001 |
| Perineural invasion present | 12 (5%) | 9 (5%) | 3 (12%) | p < 0.001 |
| Invasion into subcutaneous fat | 22 (10%) | 19 (10%) | 3 (12%) | P = 0.015 |

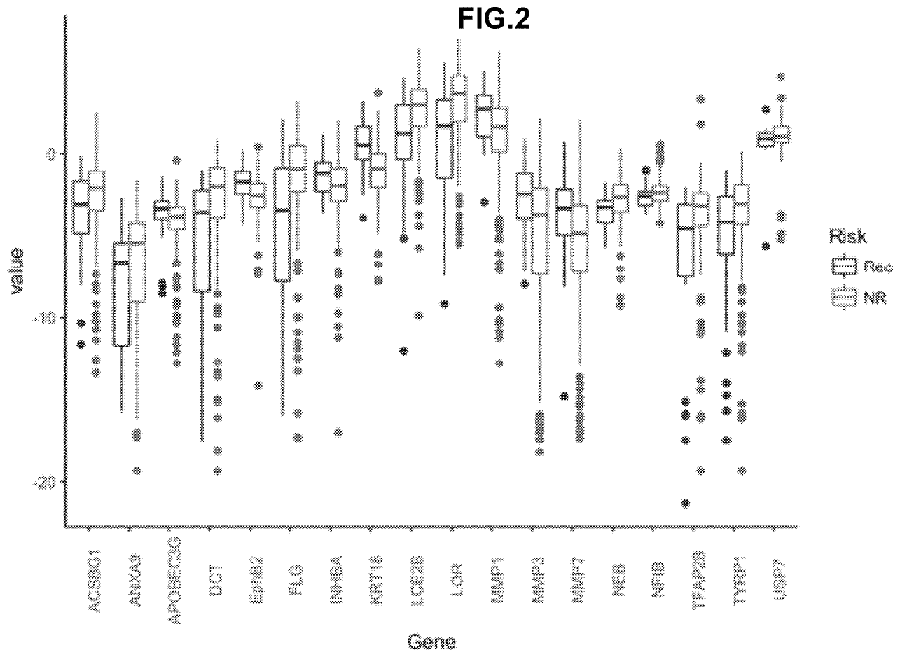

Gene expression differences (RT-PCR data from 73 genes) between recurrent and non-recurrent cSCC cases were evaluated. Using the gene expression data, several control genes were identified that had stable expression across all of the samples. These control genes were then used to normalize the expression of the remaining genes. Gene expression differences between recurrent and non-recurrent cases were investigated to find the genes that are significant. Significant gene expression differences that were associated with local recurrences, regional metastases, and distant metastases were also evaluated. Table 2 below shows genes associated with regional/distant metastases. Genetic expression of the discriminant genes in the signature (Table 2) was assessed in a cohort of 240 cSCC samples using RT-PCR, 18 of these were independently significant to a p-value of p<0.05 (see FIG. 2). As shown in Table 3 below, of the 63 discriminating genes, 18 were altered in metastatic cSCC tumors compared to nonmetastatic tumors with a p-value of p<0.05.

TABLE 2
63 candidate genes for the GEP signature to predict metastatic risk
and/recurrence in cSCC tumors

| Gene symbol | p-value | mean-Recurrence | mean-Non-Recurrence |
|---|---|---|---|
| LOR | 0.000 | 0.310 | 2.935 |
| KRT18 | 0.000 | 0.454 | −1.081 |
| LCE2B | 0.000 | 0.447 | 2.490 |
| EphB2 | 0.001 | −1.846 | −2.648 |
| FLG | 0.001 | −4.104 | −1.594 |
| DCT | 0.001 | −5.696 | −3.003 |
| TFAP2B | 0.001 | −6.571 | −3.836 |
| NEB | 0.002 | −3.495 | −2.807 |
| TYRP1 | 0.006 | −5.869 | −3.709 |
| MMP3 | 0.006 | −2.887 | −5.159 |
| MMP7 | 0.010 | −3.915 | −5.568 |
| MMP1 | 0.014 | 2.290 | 0.970 |
| INHBA | 0.016 | −1.203 | −2.222 |
| ACSBG1 | 0.024 | −3.613 | −2.489 |
| USP7 | 0.029 | 0.689 | 1.076 |
| APOBEC3G | 0.035 | −3.800 | −4.260 |
| NFIB | 0.036 | −2.623 | −2.385 |
| ANXA9 | 0.050 | −8.007 | −7.059 |
| RCHY1 | 0.055 | −2.418 | −2.665 |
| PDPN | 0.056 | 1.672 | 1.251 |
| ALOX12 | 0.066 | −4.112 | −3.688 |
| YKT6 | 0.070 | 1.449 | 1.140 |
| PLAU | 0.091 | 1.873 | 1.913 |
| ID2 | 0.110 | −0.921 | −0.550 |
| MMP10 | 0.119 | −0.067 | −1.164 |
| HPGD | 0.141 | −6.830 | −5.781 |
| FN1 | 0.147 | 1.594 | 1.186 |
| HNRNPL | 0.156 | −0.036 | 0.085 |
| AIM2 | 0.159 | −4.093 | −4.610 |
| MMP13 | 0.178 | −7.096 | −8.365 |
| BBC | 0.179 | −7.464 | −7.738 |
| EGFR | 0.189 | 0.908 | 0.705 |
| SPP1 | 0.200 | −0.144 | −1.332 |
| SERPINB4 | 0.251 | −11.815 | −10.852 |
| NEFL | 0.292 | −2.876 | −1.321 |
| NFASC | 0.301 | −3.832 | −3.738 |
| PI3 | 0.324 | 4.686 | 4.847 |
| PIG3 | 0.333 | −3.420 | −3.698 |
| LAMC2 | 0.350 | 0.480 | 0.196 |
| ARPC2 | 0.353 | −0.053 | −0.006 |
| AADAC | 0.379 | −16.094 | −15.629 |
| IL24 | 0.387 | −3.969 | −4.629 |
| S100A8 | 0.388 | 3.838 | 3.936 |
| CCL27 | 0.398 | −12.922 | −13.230 |
| PTHLH | 0.401 | 0.777 | 0.907 |
| S100A9 | 0.457 | 7.525 | 7.534 |
| DDAH1 | 0.461 | −5.660 | −5.216 |
| PDL1 | 0.471 | −3.067 | −3.124 |
| DSS1 | 0.477 | −5.722 | −5.442 |
| KRT19 | 0.486 | −2.982 | −3.853 |
| KIT | 0.529 | −2.391 | −2.415 |
| TUBB3 | 0.599 | −4.541 | −5.167 |
| MYC | 0.631 | −3.816 | −3.749 |
| CHI3L1 | 0.653 | −0.029 | 0.025 |
| MMP9 | 0.684 | 1.649 | 1.733 |
| CXCR4 | 0.742 | −8.733 | −8.858 |
| ATP6V0E2 | 0.750 | −8.562 | −8.519 |
| CXCL10 | 0.785 | −3.039 | −3.184 |
| PD1 | 0.825 | −1.878 | −1.870 |
| IL7R | 0.872 | −8.524 | −8.094 |
| MMP12 | 0.919 | −2.958 | −3.225 |
| CEP76 | 0.981 | −4.361 | −4.656 |

TABLE 3
18 Genes included in a GEP signature able to predict
recurrence in cSCC

| Gene | | mean- | |
|---|---|---|---|
| symbol | p-value | Recurrence | mean-Non-Recurrence |
| ACSBG1 | 0.024 | −3.613 | −2.489 |
| ANXA9 | 0.050 | −8.007 | −7.059 |
| APOBEC3G | 0.035 | −3.800 | −4.260 |
| DCT | 0.001 | −5.696 | −3.003 |
| EphB2 | 0.001 | −1.846 | −2.648 |
| FLG | 0.001 | −4.104 | −1.594 |
| INHBA | 0.016 | −1.203 | −2.222 |
| KRT18 | 0.000 | 0.454 | −1.081 |
| LCE2B | 0.000 | 0.447 | 2.490 |
| LOR | 0.000 | 0.310 | 2.935 |
| MMP1 | 0.014 | 2.290 | 0.970 |
| MMP3 | 0.006 | −2.887 | −5.159 |
| MMP7 | 0.010 | −3.915 | −5.568 |
| NEB | 0.002 | −3.495 | −2.807 |
| NFIB | 0.036 | −2.623 | −2.385 |
| TFAP2B | 0.001 | −6.571 | −3.836 |
| TYRP1 | 0.006 | −5.869 | −3.709 |
| USP7 | 0.029 | 0.689 | 1.076 |

### Example 3: Initial Training Set Development Studies and Comparison to Validation Cohort

R version 3.3.2 was used to train multiple predictive models (e.g., multiple machine-learning methods such as, neural networks, gradient boosting machine, generalized linear model boost, radial basis function, rule-based classification, decision tree classification, and/or regularized linear discriminant analysis) against the normalized Ct values obtained from RT-PCR analysis in 181 cSCC cases selected at random from the 240 cases in the combined set. The average of the top predictive models was more sensitive than either the Brigham and Women's Hospital (BWH) or American Joint Committee on Cancer (AJCC) models with minimal loss of specificity. These results show that recurrent and non-recurrent cSCC can be identified through gene expression profiling and gene expression can be used to identify cSCC patients with a higher risk of recurrence. A validated prognostic test could inform clinical decision-making on preoperative surgical staging (for example, based on shave biopsy), surgical approach (SLNB) or adjuvant radiation to reduce local recurrence, and adjuvant radiation, nodal staging, adjuvant systemic therapy to reduce regional/distant metastasis. Such a test could improve such intervention decisions and help determine which patients may benefit from additional therapeutic modalities.

TABLE 4
Predictive modeling-local recurrence

| | GEP | | | |
|---|---|---|---|---|
| Local Recurrence | Example 2 | BWH | AJCCv7 | AJCCv8 |
| Sensitivity | 75% | 17% | 0% | 39% |
| Specificity | 92% | 90% | 99.5% | 79% |
| negative predictive value | 98% | 92% | 92% | 94% |
| (NPV) | | | | |
| positive predictive value | 50% | 13% | 94% | 14% |
| (PPV) | | | | |

TABLE 5
Predictive modeling-metastasis

| Regional/Distant | GEP | | | |
|---|---|---|---|---|
| Metastasis | Example 2 | BWH | AJCCv7 | AJCCv8 |
| Sensitivity | 83% | 23% | 0% | 46% |
| Specificity | 95% | 90% | 100% | 79% |
| negative predictive value | 99% | 95% | 94% | 96% |
| (NPV) | | | | |
| positive predictive value | 53% | 13% | 0% | 12% |
| (PPV) | | | | |

### Example 4: Prognostic Gene Expression Profile Test in cSCC in Patients with One or More High-Risk Features

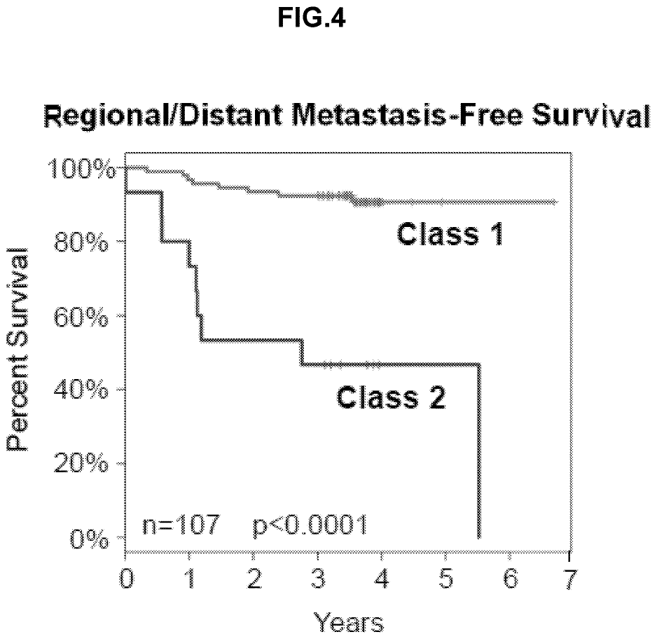

To identify a gene expression profile that accurately predicts: (1) primary cSCC with a high risk of regional nodal/distant metastasis; and (2) primary cSCC with high risk of local recurrence after complete surgical clearance, a multi-center study was performed using archived primary tissue samples with extensive capture of associated clinical data. The approach uses targeted candidate genes from the literature combined with genes from a global approach microarray screen. Samples are from subjects with pathologically confirmed cSCC diagnosed after 2006, minimum 3 years of follow-up or event (see Tables 6 and 7). Two separate outcomes were measured: (1) nodal/distant metastasis; and (2) local recurrence. Accuracy metrics demonstrate that the gene expression signature has prognostic value for in an independent cohort (see Table 8 and FIG. 4). The prognostic test could inform clinical decision-making on: (1) preoperative surgical staging, based on shave biopsy; and (2) adjuvant radiation, nodal staging, adjuvant systemic therapy to reduce regional/distant metastasis.

TABLE 6
Demographics for development stage of Example 4.

| | | | Regional/ |
|---|---|---|---|
| | | Non- | distant |
| | All | Metastatic | metastasis |
| Feature | (n = 122) | (n = 108) | (n = 14) |
| Age: Median years (range) | 74 (49-97) | 74 (50-97) | 74.5 (49-91) |
| Definitive surgery: Mohs | 99 (82%)^{#} | 88 (82%)^{#} | 11 (79%) |
| Male sex | 94 (77%) | 81 (75%) | 13 (93%) |
| Patient | 17 (14%) | 13 (12%) | 4 (29%) |
| immunocompromised | | | |
| Located on head or neck | 87 (71%) | 77 (71%) | 10 (71%) |
| Tumor diameter: | 2.0 +/− 2.9 | 1.5 +/− 1.3 | 5.8 +/− 6.7*** |
| Mean +/− SD | | | |
| Differentiation Status: | | | |
| Poorly differentiated | 5 (4%) | 4 (4%) | 1 (7%) |
| Clark Level IV/V | 45 (37%) | 40 (37%) | 5 (36%) |
| Perineural invasion present | 7 (6%) | 6 (6%) | 1 (7%) |
| Invasion into subcutaneous | 7 (6%) | 4 (4%) | 3 (21%)** |
| fat | | | |

^{#}1 case with unknown surgery type;
Wilcoxon F or Chi-square test p **< 0.01 ***< 0.001

TABLE 7
Demographics for validation stage of Example 4.

| | | | Regional/ |
|---|---|---|---|
| | All | Non-Met | distant met |
| Feature | (n = 107) | (n = 90) | (n = 17) |
| Age: Median years (range) | 72 (30-93) | 72.5 (45-93) | 72 (30-88) |
| Definitive surgery: Mohs | 86 (81%)^{#} | 76 (84%) | 10 (63%)^{#}* |
| Male sex | 78 (73%) | 64 (71%) | 14 (82%) |
| Patient | 12 (11%) | 10 (11%) | 2 (12%) |
| immunocompromised | | | |
| Located on head or neck | 76 (71%) | 62 (69%) | 14 (82%) |
| Tumor diameter: | 1.9 +/− 1.7 | 1.9 +/− 1.2 | 3.3 +/− 2.6** |
| Mean +/− SD | | | |
| Differentiation Status: | | | |
| Poorly differentiated | 13 (12%) | 6 (7%) | 7 (42%)*** |
| Clark Level IV/V | 32 (30%) | 25 (28%) | 7 (41%) |
| Perineural invasion present | 9 (8%) | 3 (3%) | 6 (35%)*** |
| Invasion into subcutaneous | 17 (16%) | 11 (12%) | 6 (35%)* |
| fat | | | |

^{#}1 case with unknown surgery type;
Wilcoxon F or Chi-square test p *< 0.05 **< 0.01 ***< 0.001

TABLE 8
Predictive modeling

| | GEP | | |
|---|---|---|---|
| Metric | Example 4 | AJCC 8 | BWH |
| Sensitivity | 53% | 53% | 41% |
| Specificity | 93% | 87% | 88% |
| negative predictive value | 91% | 91% | 89% |
| (NPV) | | | |
| positive predictive value | 60% | 43% | 39% |
| (PPV) | | | |

### Example 5: Prognostic Gene Expression Signature for Risk Assessment in cSCC with a Subanalysis in the Head and Neck Region

To identify a gene expression profile that accurately predicts: (1) primary cSCC with a high risk of metastasis (regional nodal/distant metastasis); and (2) primary cSCC with high risk of local recurrence after complete surgical clearance, a multi-center study was performed using archived primary tissue samples with extensive capture of associated clinical data. The approach uses targeted candidate genes from the literature combined with genes from a global approach microarray screen. Samples are from subjects with pathologically confirmed cSCC diagnosed after 2006, minimum 3 years of follow-up or event (see Table 9). Two separate outcomes were measured: (1) nodal/distant metastasis; and (2) local recurrence. Accuracy metrics accuracy metrics for all and head and neck cSCC cases suggest that gene expression signature has prognostic value in an independent cohort (see Table 10). The prognostic signature with a robust PPV for high-risk disease will improve identification of patients with cSCC who can benefit from surgical, radiation and immunotherapy interventions.

TABLE 9
Demographics-head and neck subanalysis

| | Non- | |
|---|---|---|
| | Metastatic | Metastasis |
| Feature of head and neck case | (n = 34) | (n = 9) |
| Age: Median years (range) | 75 (49-89) | 77 (49-89) |
| Definitive surgery: Mohs | 33 (97%) | 8 (89%) |
| Male sex | 31 (91%) | 8 (89%) |
| Tumor diameter: Mean cm +/− SD | 2.52 +/− 1.35 | 5.89 +/− 8.36 |
| Differentiation Status: | | |
| Poor/Undifferentiated | 2 (6%) | 2 (22%) |
| Clark Level IV/V | 4 (12%) | 1 (11%) |
| Perineural invasion present | 4 (12%) | 0 (0%) |
| Invasion into subcutaneous fat | 7 (21%) | 1 (11%) |

TABLE 10
Predictive modeling-head and neck subanalysis

| | All (n = 107) | | H&N (n = 76) | |
|---|---|---|---|---|
| | GEP | | GEP | |
| Metric | Example 5 | BWH | this study | BWH |
| Sensitivity | 53% | 41% | 43% | 43% |
| Specificity | 93% | 88% | 94% | 89% |
| negative predictive value | 91% | 89% | 88% | 87% |
| (NPV) | | | | |
| positive predictive value | 60% | 39% | 60% | 46% |
| (PPV) | | | | |

TABLE 11
Genes included in the gene sets that are able to predict risk of recurrence
and/or metastasis

| | | | median | | |
|---|---|---|---|---|---|
| | Probe Identifier | median | Non- | delta | |
| Gene name | (ThermoFisher) | Recurrent | Recurrent | median * | p-value |
| KRT6B | Hs00745492_s1 | 5.522 | 7.091 | −1.569 | 0.000070 |
| LOR | Hs01894962_s1 | 1.970 | 4.492 | −2.522 | 0.000265 |
| FLG | Hs00856927_g1 | −2.724 | 0.303 | −3.027 | 0.000291 |
| LCE2B | Hs04194422_s1 | 1.153 | 3.665 | −2.512 | 0.000809 |
| PLS3 | Hs00543973_m1 | −0.416 | 0.080 | −0.497 | 0.001048 |
| SERPINB2 | Hs01010736_m1 | 0.304 | 1.455 | −1.150 | 0.001277 |
| KLK5 | Hs00202752_m1 | 1.170 | 3.239 | −2.069 | 0.001468 |
| KRT18 | Hs01920599_gH | 0.975 | −0.238 | 1.213 | 0.002094 |
| BBC | Hs00248075_m1 | −4.614 | −5.334 | 0.720 | 0.002663 |
| MIR3916 | Hs04232205_s1 | −0.709 | −1.334 | 0.625 | 0.002734 |
| LOC100287896 | Hs01931732_s1 | −2.224 | −2.796 | 0.572 | 0.003547 |
| TFAP2B | Hs01560931_m1 | −4.288 | −2.456 | −1.832 | 0.004135 |
| HPGD | Hs00960591_m1 | −5.491 | −3.113 | −2.378 | 0.007656 |
| CHMP2B | Hs00387770_m1 | −3.117 | −2.591 | −0.526 | 0.008827 |
| ANXA9 | Hs01070154_m1 | −5.583 | −4.284 | −1.299 | 0.009038 |
| ID2 | Hs00747379_m1 | −0.345 | 0.493 | −0.838 | 0.009695 |
| EphB2 | Hs00362096_m1 | −1.124 | −1.614 | 0.491 | 0.012203 |
| NEB | Hs00189880_m1 | −2.611 | −1.904 | −0.706 | 0.014937 |
| FDFT1 | Hs00926053_m1 | −1.589 | −0.657 | −0.932 | 0.017046 |
| USP7 | Hs00931763_m1 | 1.509 | 1.960 | −0.452 | 0.017046 |
| TAF6L | Hs01008033_m1 | −0.699 | −0.961 | 0.262 | 0.018195 |
| ACSBG1 | Hs01025572_m1 | −2.992 | −1.336 | −1.657 | 0.026077 |
| HNRNPL | Hs00704853_s1 | 0.776 | 0.980 | −0.204 | 0.031337 |
| ARPC2 | Hs01031740_m1 | 0.715 | 1.147 | −0.432 | 0.031337 |
| DUXAP8 | Hs04942686_m1 | −6.816 | −9.507 | 2.691 | 0.039746 |
| PIM2 | Hs01546752_g1 | −1.160 | −1.752 | 0.592 | 0.050944 |
| KRT17 | Hs00356958_m1 | 6.944 | 7.254 | −0.310 | 0.053874 |
| APOBEC3G | Hs00222415_m1 | −2.574 | −3.024 | 0.450 | 0.056942 |
| DSS1 | Hs00428732_m1 | −4.131 | −3.182 | −0.949 | 0.056942 |
| EGFR | Hs01076090_m1 | 1.598 | 1.332 | 0.266 | 0.069464 |
| SERPINB4 | Hs01691258_g1 | −12.838 | −8.116 | −4.722 | 0.070706 |
| UGP2 | Hs00900510_m1 | −1.783 | −1.437 | −0.346 | 0.073246 |
| SPATA41 | Hs03028557_s1 | −12.073 | −13.333 | 1.261 | 0.077195 |
| SNORD124 | Hs03464469_s1 | −2.848 | −2.958 | 0.110 | 0.082729 |
| PI3 | Hs00964384_g1 | 5.550 | 6.140 | −0.589 | 0.085614 |
| LIME1-ZGPAT | Hs00738791_g1 | −4.044 | −4.312 | 0.267 | 0.090094 |
| MMP3 | Hs00968305_m1 | −1.478 | −2.397 | 0.919 | 0.099619 |
| S100A8 | Hs00374264_g1 | 4.237 | 5.014 | −0.777 | 0.104673 |
| PTRHD1 | Hs00415546_m1 | −1.338 | −1.216 | −0.122 | 0.109930 |
| MMP7 | Hs01042796_m1 | −2.399 | −3.937 | 1.538 | 0.115392 |
| TMEM41B | Hs01379134_m1 | −1.979 | −1.562 | −0.417 | 0.119151 |
| SPP1 | Hs00959010_m1 | 1.650 | 0.427 | 1.224 | 0.121066 |
| RBM33 | Hs00997579_m1 | 1.600 | 1.349 | 0.251 | 0.152768 |
| NFIB | Hs01029174_m1 | −1.757 | −1.633 | −0.124 | 0.159806 |
| NEFL | Hs00196245_m1 | −0.069 | 0.561 | −0.631 | 0.162206 |
| NFIC | Hs00232157_m1 | −0.500 | −0.300 | −0.200 | 0.167086 |
| DCT | Hs01098278_m1 | −3.033 | −1.300 | −1.733 | 0.174613 |
| RCHY1 | Hs00996236_m1 | −1.807 | −2.038 | 0.231 | 0.177178 |
| ZSCAN31 | Hs00372831_g1 | −3.639 | −2.926 | −0.713 | 0.179770 |
| IPO5P1 | Hs05052601_s1 | −2.927 | −3.231 | 0.303 | 0.179770 |
| RUNX3 | Hs00231709_m1 | −0.927 | −1.342 | 0.415 | 0.204381 |
| MKLN1 | Hs00992679_m1 | −0.787 | −0.930 | 0.144 | 0.204381 |
| ATP6V0E2 | Hs04189864_m1 | −5.596 | −6.247 | 0.651 | 0.207260 |
| YKT6 | Hs00559914_m1 | 2.007 | 1.788 | 0.220 | 0.210168 |
| FCHSD1 | Hs00703025_s1 | −6.048 | −5.195 | −0.854 | 0.216073 |
| MMP1 | Hs00899658_m1 | 3.156 | 2.381 | 0.774 | 0.225153 |
| CEP76 | Hs00950371_m1 | −3.743 | −3.455 | −0.288 | 0.225153 |
| TUFM-MIR4721 | Hs00944507_g1 | 2.465 | 2.281 | 0.184 | 0.228239 |
| AIM2 | Hs00915710_m1 | −2.525 | −2.720 | 0.195 | 0.244123 |
| PTHLH | Hs00174969_m1 | 0.986 | 1.833 | −0.848 | 0.264188 |
| BHLHB9 | Hs01089557_s1 | −14.090 | −12.657 | −1.433 | 0.264188 |
| CD163 | Hs00174705_m1 | −0.829 | −1.156 | 0.327 | 0.307655 |
| ZNF839 | Hs00901350_g1 | −1.060 | −1.316 | 0.256 | 0.307655 |
| BLOC1S1 | Hs00155241_m1 | −1.061 | −0.787 | −0.273 | 0.311480 |
| HDDC3 | Hs00826827_g1 | −1.299 | −1.567 | 0.267 | 0.319223 |
| TNNC1 | Hs00896999_g1 | −7.015 | −5.911 | −1.105 | 0.323141 |
| S100A9 | Hs00610058_m1 | 8.071 | 8.385 | −0.314 | 0.327091 |
| TUBB3 | Hs00801390_s1 | −3.190 | −2.711 | −0.479 | 0.331071 |
| KIT | Hs00174029_m1 | −1.168 | −1.574 | 0.406 | 0.351443 |
| FN1 | Hs01549976_m1 | 2.302 | 1.859 | 0.443 | 0.364039 |
| INHBA | Hs01081598_m1 | −1.107 | −1.166 | 0.060 | 0.368299 |
| PIGBOS1 | Hs05036222_s1 | −0.970 | −1.132 | 0.162 | 0.372591 |
| THYN1 | Hs01553775_g1 | 0.011 | −0.219 | 0.230 | 0.376913 |
| HOXA10- | Hs00365956_m1 | −3.574 | −2.521 | −1.053 | 0.412594 |
| HOXA9- | | | | | |
| MIR196B | | | | | |
| MYC | Hs00153408_m1 | −2.553 | −2.337 | −0.215 | 0.440624 |
| IL24 | Hs01114274_m1 | −3.039 | −3.394 | 0.355 | 0.455038 |
| NFIA | Hs00379134_m1 | −0.852 | −0.709 | −0.143 | 0.499836 |
| RPL26L1 | Hs01631495_s1 | −6.405 | −6.603 | 0.198 | 0.504954 |
| ZNF48 | Hs00399035_m1 | −3.340 | −3.577 | 0.237 | 0.520473 |
| MIER2 | Hs00380101_m1 | −0.275 | −0.382 | 0.108 | 0.530953 |
| MMP13 | Hs00942584_m1 | −4.547 | −5.058 | 0.511 | 0.536233 |
| TYRP1 | Hs00167051_m1 | −2.547 | −2.510 | −0.037 | 0.546872 |
| VIM | Hs00958111_m1 | 4.763 | 4.373 | 0.390 | 0.552231 |
| LRRC47 | Hs00975850_m1 | 0.130 | 0.070 | 0.060 | 0.552231 |
| ALOX12 | Hs00167524_m1 | −3.032 | −2.563 | −0.469 | 0.590445 |
| PLAU | Hs01547054_m1 | 3.212 | 2.870 | 0.342 | 0.612814 |
| IL7R | Hs00902334_m1 | −4.480 | −4.820 | 0.340 | 0.624137 |
| DARS | Hs00962398_m1 | 2.314 | 2.486 | −0.172 | 0.624137 |
| LOC101927502 | Hs05033260_s1 | −8.529 | −8.227 | −0.302 | 0.624137 |
| MIR129-1 | Hs03302824_pri | −12.122 | −13.033 | 0.910 | 0.647050 |
| PD1 | Hs00240906_m1 | −1.233 | −1.099 | −0.134 | 0.652832 |
| CYP2D6- | Hs03043789_g1 | −5.343 | −5.128 | −0.215 | 0.676166 |
| LOC101929829 | | | | | |
| GTPBP2 | Hs01051445_g1 | −2.289 | −2.127 | −0.163 | 0.687952 |
| CXCL10 | Hs00171042_m1 | −1.850 | −1.595 | −0.255 | 0.693874 |
| SLC1A3 | Hs00904817_m1 | −2.518 | −2.534 | 0.016 | 0.699815 |
| RNF135 | Hs00260480_m1 | −0.694 | −0.725 | 0.030 | 0.711752 |
| NOA1 | Hs00260452_m1 | −2.426 | −2.528 | 0.102 | 0.747977 |
| ZNF496 | Hs00262107_m1 | −1.484 | −1.549 | 0.065 | 0.760181 |
| MMP12 | Hs00159178_m1 | −2.301 | −2.567 | 0.266 | 0.772445 |
| C3orf70 | Hs01395177_m1 | −4.175 | −4.227 | 0.052 | 0.784767 |
| LAMC2 | Hs01043717_m1 | 0.874 | 1.000 | −0.126 | 0.797143 |
| MMP10 | Hs00233987_m1 | −0.255 | 0.441 | −0.696 | 0.803350 |
| C1QL4 | Hs00884853_s1 | −10.397 | −10.511 | 0.113 | 0.822045 |
| C21orf59 | Hs00937509_m1 | 0.903 | 0.829 | 0.074 | 0.822045 |
| KRT19 | Hs01051611_gH | −3.414 | −2.626 | −0.788 | 0.828299 |
| PDL1 | Hs00204257_m1 | −2.051 | −2.218 | 0.166 | 0.847127 |
| SLC25A11 | Hs01087946_g1 | 0.664 | 0.641 | 0.024 | 0.847127 |
| MRC1 | Hs00267207_m1 | −5.005 | −5.020 | 0.015 | 0.853423 |
| PIG3 | Hs00936519_m1 | −3.104 | −2.896 | −0.207 | 0.853423 |
| IL2RB | Hs00386692_m1 | −1.702 | −1.592 | −0.110 | 0.878697 |
| ATP6AP1 | Hs05016463_s1 | 0.173 | 0.183 | −0.011 | 0.878697 |
| MSANTD4 | Hs00411188_g1 | −3.627 | −3.612 | −0.015 | 0.929591 |
| MRPL21 | Hs00698959_m1 | 0.665 | 0.664 | 0.002 | 0.929591 |
| CXCR4 | Hs00607978_s1 | −5.555 | −5.868 | 0.313 | 0.935977 |
| RPP38 | Hs00705626_s1 | −4.839 | −4.719 | −0.120 | 0.935977 |
| SEPT3 | Hs00251883_m1 | −5.165 | −5.094 | −0.071 | 0.942368 |
| PDPN | Hs00366766_m1 | 1.995 | 1.893 | 0.102 | 0.948762 |
| CCL27 | Hs00171157_m1 | −12.962 | −11.407 | −1.555 | 0.967963 |
| CHI3L1 | Hs01072228_m1 | 0.794 | 0.689 | 0.105 | 0.974368 |
| DDAH1 | Hs00201707_m1 | −3.775 | −3.568 | −0.207 | 0.980774 |
| MMP9 | Hs00957562_m1 | 2.233 | 2.368 | −0.135 | 0.987182 |
| NFASC | Hs00978280_m1 | −2.781 | −2.716 | −0.066 | 0.993591 |

*Positive values indicate an INCREASE in gene expression in recurrent cancer when compared to non-recurrent control; and negative values indicate a DECREASE in gene expression in recurrent cancer when compared to non-recurrent control.

TABLE 12
Accuracy of gene sets used to predict risk of recurrence and/or metastasis

| Gene | | | | | | |
|---|---|---|---|---|---|---|
| set | Sensitivity | Specificity | PPV | NPV | AUC | Kappa |
| 20-1 | 0.4958 | 0.9481 | 0.5931 | 0.9370 | 0.8604 | 0.4537 |
| 20-2 | 0.5208 | 0.9333 | 0.5869 | 0.9385 | 0.8101 | 0.4524 |
| 20-3 | 0.4438 | 0.9472 | 0.5829 | 0.9292 | 0.8131 | 0.4147 |
| 20-4 | 0.4708 | 0.9324 | 0.5688 | 0.9318 | 0.8766 | 0.4084 |
| 20-5 | 0.4833 | 0.9324 | 0.4990 | 0.9335 | 0.8242 | 0.4033 |
| 20-6 | 0.4542 | 0.9389 | 0.5722 | 0.9306 | 0.8275 | 0.3991 |
| 20-7 | 0.5396 | 0.9065 | 0.4634 | 0.9395 | 0.8384 | 0.3934 |
| 20-8 | 0.3917 | 0.9537 | 0.5922 | 0.9238 | 0.7275 | 0.3783 |
| 20-9 | 0.4396 | 0.9259 | 0.5132 | 0.9274 | 0.8220 | 0.3673 |
| 20-10 | 0.3708 | 0.9556 | 0.5888 | 0.9228 | 0.7970 | 0.3621 |
| 20-11 | 0.4542 | 0.9241 | 0.4625 | 0.9299 | 0.7701 | 0.3615 |
| 20-12 | 0.4292 | 0.9324 | 0.4984 | 0.9280 | 0.7876 | 0.3613 |
| 20-13 | 0.3896 | 0.9472 | 0.5367 | 0.9234 | 0.8228 | 0.3605 |
| 20-14 | 0.4146 | 0.9343 | 0.5150 | 0.9254 | 0.7698 | 0.3600 |
| 20-15 | 0.4417 | 0.9278 | 0.4798 | 0.9299 | 0.7799 | 0.3553 |
| 20-16 | 0.4271 | 0.9278 | 0.4667 | 0.9262 | 0.7650 | 0.3506 |
| 20-17 | 0.4146 | 0.9287 | 0.4673 | 0.9248 | 0.7613 | 0.3506 |
| 20-18 | 0.4563 | 0.9139 | 0.4518 | 0.9297 | 0.8198 | 0.3491 |
| 20-19 | 0.4188 | 0.9315 | 0.5352 | 0.9270 | 0.8132 | 0.3489 |
| 20-20 | 0.4229 | 0.9296 | 0.4484 | 0.9264 | 0.7674 | 0.3438 |
| 20-21 | 0.4396 | 0.9231 | 0.4449 | 0.9290 | 0.8336 | 0.3420 |
| 20-22 | 0.4354 | 0.9194 | 0.4282 | 0.9268 | 0.8127 | 0.3418 |
| 20-23 | 0.3563 | 0.9537 | 0.5608 | 0.9213 | 0.7605 | 0.3379 |
| 20-24 | 0.3896 | 0.9296 | 0.4846 | 0.9221 | 0.7662 | 0.3357 |
| 20-25 | 0.3896 | 0.9370 | 0.4919 | 0.9218 | 0.8326 | 0.3354 |
| 30-1 | 0.4021 | 0.9648 | 0.6285 | 0.9275 | 0.8091 | 0.3893 |
| 30-2 | 0.4771 | 0.9204 | 0.4672 | 0.9335 | 0.8005 | 0.3739 |
| 30-3 | 0.4438 | 0.9287 | 0.4984 | 0.9287 | 0.8083 | 0.3685 |
| 30-4 | 0.4208 | 0.9306 | 0.5525 | 0.9270 | 0.8064 | 0.3613 |
| 30-5 | 0.4000 | 0.9407 | 0.5432 | 0.9255 | 0.7804 | 0.3513 |
| 30-6 | 0.4542 | 0.9167 | 0.4574 | 0.9288 | 0.7920 | 0.3480 |
| 30-7 | 0.3875 | 0.9407 | 0.5049 | 0.9240 | 0.8209 | 0.3378 |
| 30-8 | 0.3792 | 0.9454 | 0.5218 | 0.9225 | 0.7739 | 0.3375 |
| 30-9 | 0.3542 | 0.9593 | 0.5714 | 0.9207 | 0.6822 | 0.3347 |
| 30-10 | 0.4458 | 0.9157 | 0.4271 | 0.9281 | 0.7544 | 0.3339 |
| 30-11 | 0.4167 | 0.9213 | 0.4288 | 0.9245 | 0.7915 | 0.3329 |
| 30-12 | 0.3813 | 0.9380 | 0.4732 | 0.9216 | 0.7236 | 0.3318 |
| 30-13 | 0.3229 | 0.9565 | 0.6146 | 0.9170 | 0.7093 | 0.3243 |
| 30-14 | 0.3729 | 0.9361 | 0.4768 | 0.9222 | 0.7127 | 0.3187 |
| 30-15 | 0.4042 | 0.9176 | 0.3988 | 0.9225 | 0.7716 | 0.3103 |
| 30-16 | 0.3667 | 0.9306 | 0.4688 | 0.9195 | 0.7127 | 0.3052 |
| 30-17 | 0.3375 | 0.9426 | 0.4744 | 0.9178 | 0.6708 | 0.3025 |
| 30-18 | 0.3813 | 0.9213 | 0.4323 | 0.9205 | 0.8029 | 0.2996 |
| 30-19 | 0.4146 | 0.9102 | 0.3787 | 0.9241 | 0.7652 | 0.2984 |
| 30-20 | 0.3667 | 0.9296 | 0.4427 | 0.9199 | 0.7452 | 0.2954 |
| 30-21 | 0.3583 | 0.9306 | 0.4480 | 0.9197 | 0.7475 | 0.2900 |
| 30-22 | 0.3625 | 0.9241 | 0.4685 | 0.9181 | 0.7671 | 0.2897 |
| 30-23 | 0.3833 | 0.9194 | 0.3956 | 0.9199 | 0.7480 | 0.2891 |
| 30-24 | 0.3417 | 0.9380 | 0.4473 | 0.9187 | 0.7222 | 0.2885 |
| 30-25 | 0.3979 | 0.9120 | 0.3898 | 0.9221 | 0.7419 | 0.2868 |
| 40-1 | 0.4688 | 0.9481 | 0.6105 | 0.9334 | 0.8198 | 0.4340 |
| 40-2 | 0.4021 | 0.9435 | 0.5360 | 0.9242 | 0.7960 | 0.3565 |
| 40-3 | 0.3792 | 0.9426 | 0.5311 | 0.9230 | 0.7486 | 0.3354 |
| 40-4 | 0.3563 | 0.9509 | 0.5030 | 0.9200 | 0.7427 | 0.3325 |
| 40-5 | 0.4125 | 0.9278 | 0.4898 | 0.9257 | 0.8127 | 0.3300 |
| 40-6 | 0.3896 | 0.9361 | 0.4924 | 0.9235 | 0.7824 | 0.3294 |
| 40-7 | 0.3854 | 0.9324 | 0.4662 | 0.9219 | 0.7421 | 0.3248 |
| 40-8 | 0.3646 | 0.9398 | 0.5220 | 0.9212 | 0.7262 | 0.3228 |
| 40-9 | 0.3583 | 0.9380 | 0.5303 | 0.9199 | 0.7621 | 0.3189 |
| 40-10 | 0.3500 | 0.9472 | 0.4906 | 0.9201 | 0.7059 | 0.3161 |
| 40-11 | 0.3938 | 0.9222 | 0.4707 | 0.9225 | 0.7623 | 0.3143 |
| 40-12 | 0.3417 | 0.9500 | 0.5070 | 0.9188 | 0.7769 | 0.3115 |
| 40-13 | 0.3896 | 0.9296 | 0.4195 | 0.9233 | 0.7851 | 0.3047 |
| 40-14 | 0.3479 | 0.9407 | 0.4750 | 0.9178 | 0.8177 | 0.3036 |
| 40-15 | 0.3729 | 0.9269 | 0.4124 | 0.9206 | 0.6769 | 0.3034 |
| 40-16 | 0.3646 | 0.9343 | 0.4224 | 0.9200 | 0.7467 | 0.2989 |
| 40-17 | 0.3792 | 0.9222 | 0.4296 | 0.9204 | 0.7539 | 0.2983 |
| 40-18 | 0.3208 | 0.9435 | 0.5381 | 0.9152 | 0.6774 | 0.2980 |
| 40-19 | 0.3688 | 0.9194 | 0.4660 | 0.9192 | 0.6873 | 0.2940 |
| 40-20 | 0.3854 | 0.9204 | 0.4162 | 0.9224 | 0.8275 | 0.2939 |
| 40-21 | 0.3833 | 0.9167 | 0.3896 | 0.9215 | 0.7007 | 0.2904 |
| 40-22 | 0.3625 | 0.9185 | 0.4270 | 0.9177 | 0.6769 | 0.2904 |
| 40-23 | 0.3313 | 0.9343 | 0.4227 | 0.9160 | 0.6716 | 0.2836 |
| 40-24 | 0.3438 | 0.9407 | 0.4236 | 0.9193 | 0.6736 | 0.2799 |
| 40-25 | 0.3250 | 0.9389 | 0.4582 | 0.9160 | 0.6687 | 0.2774 |

TABLE 13
Exemplary gene sets used to predict risk of recurrence and/or metastasis

| Gene | Probe identifiers used for each gene set (probe |
|---|---|
| set | identifiers from ThermoFisher Scientific). |
| 20-1 | “Hs00705626_s1” “Hs00248075_m1” “Hs01560931_m1” |
| | “Hs00167524_m1” “Hs00366766_m1” “Hs01051445_g1” |
| | “Hs00996236_m1” “Hs01089557_s1” “Hs00262107_m1” |
| | “Hs01931732_s1” “Hs00399035_m1” “Hs00231709_m1” |
| | “Hs00411188_g1” “Hs00978280_m1” “Hs00826827_g1” |
| | “Hs00232157_m1” “Hs00747379_m1” “Hs00233987_m1” |
| | “Hs01008033_m1” “Hs04194422_s1” |
| 20-2 | “Hs00884853_s1” “Hs01920599_gH” “Hs00996236_m1” |
| | “Hs00248075_m1” “Hs00167524_m1” “Hs00747379_m1” |
| | “Hs00942584_m1” “Hs01042796_m1” “Hs00964384_g1” |
| | “Hs05052601_s1” “Hs00356958_m1” “Hs00901350_g1” |
| | “Hs01691258_g1” “Hs00992679_m1” “Hs01051611_gH” |
| | “Hs04194422_s1” “Hs01089557_s1” “Hs01087946_g1” |
| | “Hs05036222_s1” “Hs00856927_g1” |
| 20-3 | “Hs00248075_m1” “Hs00251883_m1” “Hs01089557_s1” |
| | “Hs00356958_m1” “Hs00856927_g1” “Hs00202752_m1” |
| | “Hs00950371_m1” “Hs00899658_m1” “Hs00362096_m1” |
| | “Hs01043717_m1” “Hs01560931_m1” “Hs00826827_g1” |
| | “Hs01010736_m1” “Hs00167524_m1” “Hs01031740_m1” |
| | “Hs01920599_gH” “Hs00201707_m1” “Hs00738791_g1” |
| | “Hs00962398_m1” “Hs00543973_m1” |
| 20-4 | “Hs00962398_m1” “Hs01920599_gH” “Hs01025572_m1” |
| | “Hs00159178_m1” “Hs01089557_s1” “Hs00167524_m1” |
| | “Hs00248075_m1” “Hs00386692_m1” “Hs00856927_g1” |
| | “Hs00996236_m1” “Hs01031740_m1” “Hs01010736_m1” |
| | “Hs00900510_m1” “Hs00826827_g1” “Hs01008033_m1” |
| | “Hs00415546_m1” “Hs04942686_m1” “Hs00801390_s1” |
| | “Hs01072228_m1” “Hs01547054_m1” |
| 20-5 | “Hs00411188_g1” “Hs00248075_m1” “Hs00202752_m1” |
| | “Hs00747379_m1” “Hs01042796_m1” “Hs01920599_gH” |
| | “Hs01114274_m1” “Hs00942584_m1” “Hs00996236_m1” |
| | “Hs00167524_m1” “Hs00978280_m1” “Hs00543973_m1” |
| | “Hs00826827_g1” “Hs01560931_m1” “Hs00931763_m1” |
| | “Hs01089557_s1” “Hs00174029_m1” “Hs01029174_m1” |
| | “Hs00415546_m1” “Hs00964384_g1” |
| 20-6 | “Hs00975850_m1” “Hs04942686_m1” “Hs00202752_m1” |
| | “Hs00233987_m1” “Hs00926053_m1” “Hs00856927_g1” |
| | “Hs00992679_m1” “Hs00251883_m1” “Hs00415546_m1” |
| | “Hs00960591_m1” “Hs00901350_g1” “Hs00747379_m1” |
| | “Hs01089557_s1” “Hs00936519_m1” “Hs03043789_g1” |
| | “Hs01560931_m1” “Hs00232157_m1” “Hs00957562_m1” |
| | “Hs00248075_m1” “Hs01549976_m1” |
| 20-7 | “Hs04194422_s1” “Hs00262107_m1” “Hs01546752_g1” |
| | “Hs01920599_gH” “Hs04189864_m1” “Hs01089557_s1” |
| | “Hs01560931_m1” “Hs00705626_s1” “Hs01043717_m1” |
| | “Hs00747379_m1” “Hs00248075_m1” “Hs00856927_g1” |
| | “Hs01029174_m1” “Hs00543973_m1” “Hs01395177_m1” |
| | “Hs00260480_m1” “Hs00174029_m1” “Hs00387770_m1” |
| | “Hs01894962_s1” “Hs00745492_s1” |
| 20-8 | “Hs01560931_m1” “Hs00738791_g1” “Hs00856927_g1” |
| | “Hs00362096_m1” “Hs00826827_g1” “Hs01098278_m1” |
| | “Hs00975850_m1” “Hs00167524_m1” “Hs00260452_m1” |
| | “Hs04194422_s1” “Hs01043717_m1” “Hs00233987_m1” |
| | “Hs00703025_s1” “Hs00896999_g1” “Hs00167051_m1” |
| | “Hs00942584_m1” “Hs01087946_g1” “Hs00411188_g1” |
| | “Hs00747379_m1” “Hs05016463_s1” |
| 20-9 | “Hs00362096_m1” “Hs00942584_m1” “Hs01560931_m1” |
| | “Hs00167524_m1” “Hs00884853_s1” “Hs00248075_m1” |
| | “Hs01920599_gH” “Hs00996236_m1” “Hs00747379_m1” |
| | “Hs01089557_s1” “Hs00959010_m1” “Hs00372831_g1” |
| | “Hs04194422_s1” “Hs01043717_m1” “Hs00399035_m1” |
| | “Hs01051611_gH” “Hs01042796_m1” “Hs00968305_m1” |
| | “Hs00260452_m1” “Hs01031740_m1” |
| 20-10 | “Hs05033260_s1” “Hs00233987_m1” “Hs04194422_s1” |
| | “Hs00992679_m1” “Hs00926053_m1” “Hs00167524_m1” |
| | “Hs00202752_m1” “Hs01549976_m1” “Hs00415546_m1” |
| | “Hs01072228_m1” “Hs01691258_g1” “Hs00387770_m1” |
| | “Hs00380101_m1” “Hs00231709_m1” “Hs01920599_gH” |
| | “Hs00543973_m1” “Hs00386692_m1” “Hs00705626_s1” |
| | “Hs00196245_m1” “Hs01081598_m1” |
| 20-11 | “Hs01114274_m1” “Hs01560931_m1” “Hs00738791_g1” |
| | “Hs00931763_m1” “Hs00996236_m1” “Hs00362096_m1” |
| | “Hs00747379_m1” “Hs00411188_g1” “Hs00900510_m1” |
| | “Hs01098278_m1” “Hs00233987_m1” “Hs04194422_s1” |
| | “Hs00826827_g1” “Hs00856927_g1” “Hs00232157_m1” |
| | “Hs01010736_m1” “Hs00704853_s1” “Hs00959010_m1” |
| | “Hs00260480_m1” “Hs00915710_m1” |
| 20-12 | “Hs00992679_m1” “Hs00159178_m1” “Hs00167524_m1” |
| | “Hs00958111_m1” “Hs00901350_g1” “Hs00931763_m1” |
| | “Hs00233987_m1” “Hs01549976_m1” “Hs01894962_s1” |
| | “Hs01089557_s1” “Hs00171157_m1” “Hs00153408_m1” |
| | “Hs00248075_m1” “Hs03464469_s1” “Hs04194422_s1” |
| | “Hs00745492_s1” “Hs00366766_m1” “Hs00856927_g1” |
| | “Hs00957562_m1” “Hs01025572_m1” |
| 20-13 | “Hs01379134_m1” “Hs00362096_m1” “Hs05052601_s1” |
| | “Hs00959010_m1” “Hs00251883_m1” “Hs01089557_s1” |
| | “Hs00856927_g1” “Hs00167524_m1” “Hs00159178_m1” |
| | “Hs04942686_m1” “Hs00356958_m1” “Hs01042796_m1” |
| | “Hs03464469_s1” “Hs01029174_m1” “Hs00248075_m1” |
| | “Hs00610058_m1” “Hs01070154_m1” “Hs00703025_s1” |
| | “Hs00964384_g1” “Hs00705626_s1” |
| 20-14 | “Hs00362096_m1” “Hs00996236_m1” “Hs00931763_m1” |
| | “Hs01081598_m1” “Hs01560931_m1” “Hs00167524_m1” |
| | “Hs00543973_m1” “Hs01098278_m1” “Hs00856927_g1” |
| | “Hs03043789_g1” “Hs01089557_s1” “Hs01051445_g1” |
| | “Hs00747379_m1” “Hs01114274_m1” “Hs00826827_g1” |
| | “Hs00936519_m1” “Hs00960591_m1” “Hs00201707_m1” |
| | “Hs00899658_m1” “Hs04189864_m1” |
| 20-15 | “Hs00856927_g1” “Hs04942686_m1” “Hs00248075_m1” |
| | “Hs01029174_m1” “Hs00992679_m1” “Hs00975850_m1” |
| | “Hs03464469_s1” “Hs00167524_m1” “Hs01691258_g1” |
| | “Hs00960591_m1” “Hs01089557_s1” “Hs04194422_s1” |
| | “Hs01114274_m1” “Hs00901350_g1” “Hs00936519_m1” |
| | “Hs00233987_m1” “Hs00411188_g1” “Hs00900510_m1” |
| | “Hs00174969_m1” “Hs01070154_m1” |
| 20-16 | “Hs00826827_g1” “Hs00738791_g1” “Hs00698959_m1” |
| | “Hs00153408_m1” “Hs00167051_m1” “Hs00365956_m1” |
| | “Hs04194422_s1” “Hs00167524_m1” “Hs01560931_m1” |
| | “Hs00610058_m1” “Hs00232157_m1” “Hs00996236_m1” |
| | “Hs00705626_s1” “Hs00362096_m1” “Hs01098278_m1” |
| | “Hs00997579_m1” “Hs00559914_m1” “Hs00856927_g1” |
| | “Hs00944507_g1” “Hs00379134_m1” |
| 20-17 | “Hs00232157_m1” “Hs00543973_m1” “Hs05052601_s1” |
| | “Hs00196245_m1” “Hs01042796_m1” “Hs00411188_g1” |
| | “Hs00899658_m1” “Hs00374264_g1” “Hs01894962_s1” |
| | “Hs04194422_s1” “Hs03464469_s1” “Hs00992679_m1” |
| | “Hs00901350_g1” “Hs00362096_m1” “Hs00856927_g1” |
| | “Hs00167524_m1” “Hs01089557_s1” “Hs01931732_s1” |
| | “Hs01549976_m1” “Hs01395177_m1” |
| 20-18 | “Hs00884853_s1” “Hs00167524_m1” “Hs00978280_m1” |
| | “Hs00747379_m1” “Hs01931732_s1” “Hs00931763_m1” |
| | “Hs04942686_m1” “Hs04194422_s1” “Hs00856927_g1” |
| | “Hs00248075_m1” “Hs00901350_g1” “Hs00415546_m1” |
| | “Hs01089557_s1” “Hs01025572_m1” “Hs00231709_m1” |
| | “Hs00386692_m1” “Hs01920599_gH” “Hs00202752_m1” |
| | “Hs01029174_m1” “Hs00942584_m1” |
| 20-19 | “Hs01547054_m1” “Hs00960591_m1” “Hs03464469_s1” |
| | “Hs01560931_m1” “Hs00153408_m1” “Hs00233987_m1” |
| | “Hs01089557_s1” “Hs00366766_m1” “Hs00248075_m1” |
| | “Hs01010736_m1” “Hs00251883_m1” “Hs00996236_m1” |
| | “Hs00610058_m1” “Hs00900510_m1” “Hs01008033_m1” |
| | “Hs00978280_m1” “Hs00260452_m1” “Hs04194422_s1” |
| | “Hs00196245_m1” “Hs05052601_s1” |
| 20-20 | “Hs01098278_m1” “Hs01072228_m1” “Hs01691258_g1” |
| | “Hs00387770_m1” “Hs00543973_m1” “Hs01920599_gH” |
| | “Hs04189864_m1” “Hs05033260_s1” “Hs00856927_g1” |
| | “Hs00366766_m1” “Hs03043789_g1” “Hs04194422_s1” |
| | “Hs00202752_m1” “Hs00936519_m1” “Hs01560931_m1” |
| | “Hs00958111_m1” “Hs00251883_m1” “Hs00704853_s1” |
| | “Hs00738791_g1” “Hs00962398_m1” |
| 20-21 | “Hs01042796_m1” “Hs01560931_m1” “Hs00884853_s1” |
| | “Hs00958111_m1” “Hs00411188_g1” “Hs05052601_s1” |
| | “Hs01920599_gH” “Hs00248075_m1” “Hs00747379_m1” |
| | “Hs00996236_m1” “Hs00251883_m1” “Hs00202752_m1” |
| | “Hs01008033_m1” “Hs00201707_m1” “Hs01051445_g1” |
| | “Hs00950371_m1” “Hs01029174_m1” “Hs00232157_m1” |
| | “Hs01087946_g1” “Hs00267207_m1” |
| 20-22 | “Hs00559914_m1” “Hs00856927_g1” “Hs00978280_m1” |
| | “Hs01920599_gH” “Hs01560931_m1” “Hs00365956_m1” |
| | “Hs00610058_m1” “Hs01008033_m1” “Hs04194422_s1” |
| | “Hs00975850_m1” “Hs00204257_m1” “Hs00950371_m1” |
| | “Hs00705626_s1” “Hs01089557_s1” “Hs00196245_m1” |
| | “Hs01042796_m1” “Hs00174029_m1” “Hs01546752_g1” |
| | “Hs01098278_m1” “Hs00231709_m1” |
| 20-23 | “Hs04194422_s1” “Hs01089557_s1” “Hs00155241_m1” |
| | “Hs00942584_m1” “Hs05033260_s1” “Hs01546752_g1” |
| | “Hs01920599_gH” “Hs01560931_m1” “Hs00387770_m1” |
| | “Hs00399035_m1” “Hs00232157_m1” “Hs00931763_m1” |
| | “Hs00231709_m1” “Hs00856927_g1” “Hs00233987_m1” |
| | “Hs01098278_m1” “Hs00978280_m1” “Hs04942686_m1” |
| | “Hs00411188_g1” “Hs00201707_m1” |
| 20-24 | “Hs00232157_m1” “Hs01010736_m1” “Hs00704853_s1” |
| | “Hs00167051_m1” “Hs00738791_g1” “Hs00975850_m1” |
| | “Hs00362096_m1” “Hs00826827_g1” “Hs00233987_m1” |
| | “Hs00950371_m1” “Hs00204257_m1” “Hs01560931_m1” |
| | “Hs00196245_m1” “Hs01042796_m1” “Hs00174029_m1” |
| | “Hs00705626_s1” “Hs00856927_g1” “Hs01051445_g1” |
| | “Hs00399035_m1” “Hs05036222_s1” |
| 20-25 | “Hs00233987_m1” “Hs00380101_m1” “Hs00167524_m1” |
| | “Hs04189864_m1” “Hs00543973_m1” “Hs01920599_gH” |
| | “Hs00856927_g1” “Hs00155241_m1” “Hs01098278_m1” |
| | “Hs00703025_s1” “Hs00231709_m1” “Hs00365956_m1” |
| | “Hs00204257_m1” “Hs01395177_m1” “Hs00968305_m1” |
| | “Hs00374264_g1” “Hs00248075_m1” “Hs00202752_m1” |
| | “Hs00801390_s1” “Hs03464469_s1” |
| 30-1 | “Hs00387770_m1” “Hs00155241_m1” “Hs00251883_m1” |
| | “Hs01560931_m1” “Hs00428732_m1” “Hs00992679_m1” |
| | “Hs00705626_s1” “Hs00356958_m1” “Hs00960591_m1” |
| | “Hs00171157_m1” “Hs01072228_m1” “Hs01051445_g1” |
| | “Hs00232157_m1” “Hs00233987_m1” “Hs04232205_s1” |
| | “Hs00559914_m1” “Hs00931763_m1” “Hs00167524_m1” |
| | “Hs00171042_m1” “Hs01089557_s1” “Hs04194422_s1” |
| | “Hs03464469_s1” “Hs00944507_g1” “Hs00196245_m1” |
| | “Hs00950371_m1” “Hs01395177_m1” “Hs01070154_m1” |
| | “Hs01051611_gH” “Hs00399035_m1” “Hs00703025_s1” |
| 30-2 | “Hs00937509_m1” “Hs00379134_m1” “Hs00899658_m1” |
| | “Hs00248075_m1” “Hs01920599_gH” “Hs04194422_s1” |
| | “Hs03043789_g1” “Hs00610058_m1” “Hs01089557_s1” |
| | “Hs00705626_s1” “Hs00362096_m1” “Hs01560931_m1” |
| | “Hs00233987_m1” “Hs00372831_g1” “Hs01072228_m1” |
| | “Hs01379134_m1” “Hs01076090_m1” “Hs04189864_m1” |
| | “Hs00957562_m1” “Hs01931732_s1” “Hs00856927_g1” |
| | “Hs01546752_g1” “Hs00171157_m1” “Hs00992679_m1” |
| | “Hs00931763_m1” “Hs00411188_g1” “Hs01008033_m1” |
| | “Hs00387770_m1” “Hs00996236_m1” “Hs00231709_m1” |
| 30-3 | “Hs00411188_g1” “Hs01395177_m1” “Hs01560931_m1” |
| | “Hs00379134_m1” “Hs00960591_m1” “Hs00745492_s1” |
| | “Hs00232157_m1” “Hs00899658_m1” “Hs00248075_m1” |
| | “Hs01010736_m1” “Hs00167524_m1” “Hs01691258_g1” |
| | “Hs00260452_m1” “Hs01070154_m1” “Hs00196245_m1” |
| | “Hs00747379_m1” “Hs01547054_m1” “Hs00950371_m1” |
| | “Hs00962398_m1” “Hs01087946_g1” “Hs00262107_m1” |
| | “Hs00260480_m1” “Hs00978280_m1” “Hs00428732_m1” |
| | “Hs00944507_g1” “Hs00387770_m1” “Hs00399035_m1” |
| | “Hs00738791_g1” “Hs00698959_m1” “Hs01549976_m1” |
| 30-4 | “Hs00415546_m1” “Hs00705626_s1” “Hs00204257_m1” |
| | “Hs00174969_m1” “Hs00745492_s1” “Hs01920599_gH” |
| | “Hs00387770_m1” “Hs01560931_m1” “Hs00958111_m1” |
| | “Hs01076090_m1” “Hs00379134_m1” “Hs00196245_m1” |
| | “Hs00232157_m1” “Hs01549976_m1” “Hs00801390_s1” |
| | “Hs00399035_m1” “Hs01089557_s1” “Hs00607978_s1” |
| | “Hs00960591_m1” “Hs00171157_m1” “Hs05033260_s1” |
| | “Hs00233987_m1” “Hs00904817_m1” “Hs01031740_m1” |
| | “Hs05016463_s1” “Hs00153408_m1” “Hs00968305_m1” |
| | “Hs00174029_m1” “Hs00944507_g1” “Hs00248075_m1” |
| 30-5 | “Hs05033260_s1” “Hs00362096_m1” “Hs00559914_m1” |
| | “Hs01560931_m1” “Hs00968305_m1” “Hs00931763_m1” |
| | “Hs04942686_m1” “Hs01920599_gH” “Hs00899658_m1” |
| | “Hs00248075_m1” “Hs00958111_m1” “Hs05016463_s1” |
| | “Hs03464469_s1” “Hs01931732_s1” “Hs01076090_m1” |
| | “Hs00992679_m1” “Hs00901350_g1” “Hs00937509_m1” |
| | “Hs00960591_m1” “Hs01114274_m1” “Hs01043717_m1” |
| | “Hs00167524_m1” “Hs01089557_s1” “Hs00204257_m1” |
| | “Hs00703025_s1” “Hs01549976_m1” “Hs01031740_m1” |
| | ““Hs00262107_m1” Hs00957562_m1” “Hs01029174_m1” |
| 30-6 | “Hs00958111_m1” “Hs00411188_g1” “Hs05036222_s1” |
| | “Hs00386692_m1” “Hs00745492_s1” “Hs01010736_m1” |
| | “Hs00997579_m1” “Hs00222415_m1” “Hs03464469_s1” |
| | “Hs00233987_m1” “Hs01547054_m1” “Hs00202752_m1” |
| | “Hs01025572_m1” “Hs00379134_m1” “Hs04194422_s1” |
| | “Hs00926053_m1” “Hs01560931_m1” “Hs00705626_s1” |
| | “Hs01920599_gH” “Hs00171042_m1” “Hs03043789_g1” |
| | “Hs00610058_m1” “Hs01089557_s1” “Hs00362096_m1” |
| | “Hs00380101_m1” “Hs00899658_m1” “Hs00801390_s1” |
| | “Hs00374264_g1” “Hs00964384_g1” “Hs00240906_m1” |
| 30-7 | “Hs00704853_s1” “Hs01560931_m1” “Hs01379134_m1” |
| | “Hs00958111_m1” “Hs00386692_m1” “Hs04942686_m1” |
| | “Hs00747379_m1” “Hs00167524_m1” “Hs03302824_pri” |
| | “Hs00248075_m1” “Hs00387770_m1” “Hs00978280_m1” |
| | “Hs00379134_m1” “Hs00233987_m1” “Hs00992679_m1” |
| | “Hs01931732_s1” “Hs00380101_m1” “Hs00705626_s1” |
| | “Hs00155241_m1” “Hs05033260_s1” “Hs01089557_s1” |
| | “Hs00171157_m1” “Hs00745492_s1” “Hs00937509_m1” |
| | “Hs00374264_g1” “Hs00240906_m1” “Hs01098278_m1” |
| | “Hs00174705_m1” “Hs00260480_m1” “Hs00372831_g1” |
| 30-8 | “Hs00202752_m1” “Hs00231709_m1” “Hs00366766_m1” |
| | “Hs00201707_m1” “Hs00826827_g1” “Hs00856927_g1” |
| | “Hs00174969_m1” “Hs00387770_m1” “Hs00167524_m1” |
| | “Hs01031740_m1” “Hs03464469_s1” “Hs01025572_m1” |
| | “Hs00996236_m1” “Hs00950371_m1” “Hs00610058_m1” |
| | “Hs01920599_gH” “Hs01051445_g1” “Hs00975850_m1” |
| | “Hs00978280_m1” “Hs00167051_m1” “Hs01560931_m1” |
| | “Hs00704853_s1” “Hs00902334_m1” “Hs01553775_g1” |
| | “Hs00962398_m1” “Hs01098278_m1” “Hs00356958_m1” |
| | “Hs01087946_g1” “Hs01029174_m1” “Hs00747379_m1” |
| 30-9 | “Hs00362096_m1” “Hs01560931_m1” “Hs04942686_m1” |
| | “Hs05033260_s1” “Hs00958111_m1” “Hs00232157_m1” |
| | “Hs00899658_m1” “Hs00607978_s1” “Hs01549976_m1” |
| | “Hs00262107_m1” “Hs00968305_m1” “Hs00233987_m1” |
| | “Hs00904817_m1” “Hs00356958_m1” “Hs00900510_m1” |
| | “Hs00174969_m1” “Hs04194422_s1” “Hs01043717_m1” |
| | “Hs00745492_s1” “Hs00171042_m1” “Hs00379134_m1” |
| | “Hs00559914_m1” “Hs04232205_s1” “Hs00957562_m1” |
| | “Hs00167051_m1” “Hs05052601_s1” “Hs00380101_m1” |
| | “Hs01546752_g1” “Hs00801390_s1” “Hs01070154_m1” |
| 30-10 | “Hs00380101_m1” “Hs03464469_s1” “Hs00372831_g1” |
| | “Hs05052601_s1” “Hs04189864_m1” “Hs00248075_m1” |
| | “Hs00968305_m1” “Hs04942686_m1” “Hs00159178_m1” |
| | “Hs00386692_m1” “Hs00937509_m1” “Hs00960591_m1” |
| | “Hs00543973_m1” “Hs01894962_s1” “Hs00233987_m1” |
| | “Hs01051445_g1” “Hs01560931_m1” “Hs01089557_s1” |
| | “Hs00232157_m1” “Hs00387770_m1” “Hs01025572_m1” |
| | “Hs01395177_m1” “Hs00801390_s1” “Hs00884853_s1” |
| | “Hs01072228_m1” “Hs00201707_m1” “Hs00996236_m1” |
| | “Hs01114274_m1” “Hs00747379_m1” “Hs00167051_m1” |
| 30-11 | “Hs00801390_s1” “Hs01920599_gH” “Hs00931763_m1” |
| | “Hs00262107_m1” “Hs04942686_m1” “Hs00233987_m1” |
| | “Hs00399035_m1” “Hs00248075_m1” “Hs00171157_m1” |
| | “Hs00167524_m1” “Hs00899658_m1” “Hs00232157_m1” |
| | “Hs00362096_m1” “Hs00915710_m1” “Hs00174969_m1” |
| | “Hs00900510_m1” “Hs00387770_m1” “Hs00428732_m1” |
| | “Hs00607978_s1” “Hs00380101_m1” “Hs00260480_m1” |
| | “Hs00996236_m1” “Hs00958111_m1” “Hs01070154_m1” |
| | “Hs00543973_m1” “Hs00937509_m1” “Hs01089557_s1” |
| | “Hs00968305_m1” “Hs01072228_m1” “Hs01042796_m1” |
| 30-12 | “Hs00745492_s1” “Hs00950371_m1” “Hs00233987_m1” |
| | “Hs01560931_m1” “Hs00379134_m1” “Hs00167524_m1” |
| | “Hs00826827_g1” “Hs00978280_m1” “Hs00904817_m1” |
| | “Hs00856927_g1” “Hs00899658_m1” “Hs01008033_m1” |
| | “Hs00931763_m1” “Hs00365956_m1” “Hs03302824_pri” |
| | “Hs00428732_m1” “Hs00975850_m1” “Hs01894962_s1” |
| | “Hs00703025_s1” “Hs01072228_m1” “Hs01098278_m1” |
| | “Hs00958111_m1” “Hs00944507_g1” “Hs01010736_m1” |
| | “Hs00996236_m1” “Hs01920599_gH” “Hs00387770_m1” |
| | “Hs01547054_m1” “Hs00610058_m1” “Hs01087946_g1” |
| 30-13 | “Hs00856927_g1” “Hs00915710_m1” “Hs00379134_m1” |
| | “Hs00233987_m1” “Hs01008033_m1” “Hs00167524_m1” |
| | “Hs00899658_m1” “Hs00559914_m1” “Hs00904817_m1” |
| | “Hs04942686_m1” “Hs00386692_m1” “Hs00704853_s1” |
| | “Hs00978280_m1” “Hs00415546_m1” “Hs00399035_m1” |
| | “Hs00374264_g1” “Hs01560931_m1” “Hs01546752_g1” |
| | “Hs00231709_m1” “Hs00387770_m1” “Hs00964384_g1” |
| | “Hs00698959_m1” “Hs00196245_m1” “Hs01395177_m1” |
| | “Hs01076090_m1” “Hs00201707_m1” “Hs03302824_pri” |
| | “Hs00703025_s1” “Hs01114274_m1” “Hs01631495_s1” |
| 30-14 | “Hs00380101_m1” “Hs00232157_m1” “Hs00428732_m1” |
| | “Hs03302824_pri” “Hs01031740_m1” “Hs00171042_m1” |
| | “Hs01549976_m1” “Hs00260452_m1” “Hs00745492_s1” |
| | “Hs00411188_g1” “Hs00167524_m1” “Hs01560931_m1” |
| | “Hs00356958_m1” “Hs00559914_m1” “Hs00262107_m1” |
| | “Hs03464469_s1” “Hs00801390_s1” “Hs00202752_m1” |
| | “Hs00233987_m1” “Hs00196245_m1” “Hs00960591_m1” |
| | “Hs01546752_g1” “Hs01087946_g1” “Hs01547054_m1” |
| | “Hs00260480_m1” “Hs01070154_m1” “Hs00267207_m1” |
| | “Hs00174705_m1” “Hs00747379_m1” “Hs00387770_m1” |
| 30-15 | “Hs00960591_m1” “Hs00231709_m1” “Hs00251883_m1” |
| | “Hs00387770_m1” “Hs00356958_m1” “Hs00196245_m1” |
| | “Hs00174969_m1” “Hs00996236_m1” “Hs00559914_m1” |
| | “Hs00962398_m1” “Hs00856927_g1” “Hs00826827_g1” |
| | “Hs01025572_m1” “Hs00801390_s1” “Hs03043789_g1” |
| | “Hs00950371_m1” “Hs00233987_m1” “Hs00202752_m1” |
| | “Hs00260480_m1” “Hs00944507_g1” “Hs00978280_m1” |
| | “Hs01894962_s1” “Hs00204257_m1” “Hs00174029_m1” |
| | “Hs00703025_s1” “Hs01051611_gH” “Hs00958111_m1” |
| | “Hs00262107_m1” “Hs00167524_m1” “Hs01098278_m1” |
| 30-16 | “Hs00944507_g1” “Hs05016463_s1” “Hs03464469_s1” |
| | “Hs00167051_m1” “Hs00900510_m1” “Hs00904817_m1” |
| | “Hs04232205_s1” “Hs00356958_m1” “Hs00957562_m1” |
| | “Hs00745492_s1” “Hs00931763_m1” “Hs01043717_m1” |
| | “Hs00167524_m1” “Hs00856927_g1” “Hs00992679_m1” |
| | “Hs00380101_m1” “Hs01549976_m1” “Hs00559914_m1” |
| | “Hs01560931_m1” “Hs01098278_m1” “Hs00703025_s1” |
| | “Hs00248075_m1” “Hs01081598_m1” “Hs00428732_m1” |
| | “Hs00968305_m1” “Hs00705626_s1” “Hs00386692_m1” |
| | “Hs00174705_m1” “Hs00251883_m1” “Hs00415546_m1” |
| 30-17 | “Hs00543973_m1” “Hs00159178_m1” “Hs01098278_m1” |
| | “Hs00399035_m1” “Hs01029174_m1” “Hs00705626_s1” |
| | “Hs03302824_pri” “Hs01051611_gH” “Hs05033260_s1” |
| | “Hs01560931_m1” “Hs00167524_m1” “Hs01042796_m1” |
| | “Hs05052601_s1” “Hs00944507_g1” “Hs00978280_m1” |
| | “Hs00747379_m1” “Hs00171042_m1” “Hs00251883_m1” |
| | “Hs00960591_m1” “Hs01072228_m1” “Hs03043789_g1” |
| | “Hs00233987_m1” “Hs04942686_m1” “Hs00801390_s1” |
| | “Hs00992679_m1” “Hs01010736_m1” “Hs00232157_m1” |
| | “Hs00896999_g1” “Hs00826827_g1” “Hs00231709_m1” |
| 30-18 | “Hs01920599_gH” “Hs00248075_m1” “Hs00411188_g1” |
| | “Hs01560931_m1” “Hs00900510_m1” “Hs00996236_m1” |
| | “Hs00745492_s1” “Hs00202752_m1” “Hs00233987_m1” |
| | “Hs00222415_m1” “Hs01089557_s1” “Hs05052601_s1” |
| | “Hs00960591_m1” “Hs00958111_m1” “Hs01379134_m1” |
| | “Hs00380101_m1” “Hs00856927_g1” “Hs04189864_m1” |
| | “Hs01031740_m1” “Hs01042796_m1” “Hs01098278_m1” |
| | “Hs01395177_m1” “Hs00950371_m1” “Hs01691258_g1” |
| | “Hs00899658_m1” “Hs00962398_m1” “Hs00240906_m1” |
| | “Hs00399035_m1” “Hs00428732_m1” “Hs04942686_m1” |
| 30-19 | “Hs00428732_m1” “Hs01560931_m1” “Hs01025572_m1” |
| | “Hs00232157_m1” “Hs01089557_s1” “Hs01043717_m1” |
| | “Hs00705626_s1” “Hs00884853_s1” “Hs00543973_m1” |
| | “Hs00992679_m1” “Hs00899658_m1” “Hs00233987_m1” |
| | “Hs00745492_s1” “Hs01031740_m1” “Hs01076090_m1” |
| | “Hs00260480_m1” “Hs00856927_g1” “Hs00189880_m1” |
| | “Hs00231709_m1” “Hs01920599_gH” “Hs00171042_m1” |
| | “Hs00415546_m1” “Hs01894962_s1” “Hs04189864_m1” |
| | “Hs00174969_m1” “Hs00610058_m1” “Hs00153408_m1” |
| | “Hs01379134_m1” “Hs00915710_m1” “Hs01395177_m1” |
| 30-20 | “Hs00267207_m1” “Hs00366766_m1” “Hs04942686_m1” |
| | “Hs00231709_m1” “Hs01098278_m1” “Hs05016463_s1” |
| | “Hs00155241_m1” “Hs00900510_m1” “Hs00975850_m1” |
| | “Hs04194422_s1” “Hs01114274_m1” “Hs01072228_m1” |
| | “Hs01089557_s1” “Hs00747379_m1” “Hs00944507_g1” |
| | “Hs00705626_s1” “Hs00202752_m1” “Hs00978280_m1” |
| | “Hs01920599_gH” “Hs00196245_m1” “Hs04189864_m1” |
| | “Hs01025572_m1” “Hs05052601_s1” “Hs00380101_m1” |
| | “Hs01081598_m1” “Hs00372831_g1” “Hs00167051_m1” |
| | “Hs00232157_m1” “Hs01553775_g1” “Hs00738791_g1” |
| 30-21 | “Hs00174705_m1” “Hs01042796_m1” “Hs00856927_g1” |
| | “Hs00372831_g1” “Hs03302824_pri” “Hs05033260_s1” |
| | “Hs04189864_m1” “Hs00992679_m1” “Hs01560931_m1” |
| | “Hs00159178_m1” “Hs00196245_m1” “Hs05052601_s1” |
| | “Hs01031740_m1” “Hs00233987_m1” “Hs00975850_m1” |
| | “Hs01098278_m1” “Hs00248075_m1” “Hs00167524_m1” |
| | “Hs00171042_m1” “Hs01920599_gH” “Hs00380101_m1” |
| | “Hs00559914_m1” “Hs04942686_m1” “Hs00202752_m1” |
| | “Hs00937509_m1” “Hs00978280_m1” “Hs00936519_m1” |
| | “Hs00222415_m1” “Hs00365956_m1” “Hs00379134_m1” |
| 30-22 | “Hs00380101_m1” “Hs00233987_m1” “Hs00171042_m1” |
| | “Hs00411188_g1” “Hs00248075_m1” “Hs00559914_m1” |
| | “Hs00826827_g1” “Hs01894962_s1” “Hs01098278_m1” |
| | “Hs00232157_m1” “Hs01920599_gH” “Hs00975850_m1” |
| | “Hs00356958_m1” “Hs00543973_m1” “Hs01029174_m1” |
| | “Hs00159178_m1” “Hs05052601_s1” “Hs04194422_s1” |
| | “Hs01025572_m1” “Hs00950371_m1” “Hs00167524_m1” |
| | “Hs01072228_m1” “Hs00900510_m1” “Hs00705626_s1” |
| | “Hs01114274_m1” “Hs00904817_m1” “Hs00231709_m1” |
| | “Hs00745492_s1” “Hs00155241_m1” “Hs01081598_m1” |
| 30-23 | “Hs00964384_g1” “Hs00899658_m1” “Hs00374264_g1” |
| | “Hs00399035_m1” “Hs00703025_s1” “Hs00415546_m1” |
| | “Hs00232157_m1” “Hs00387770_m1” “Hs00826827_g1” |
| | “Hs01395177_m1” “Hs00202752_m1” “Hs00171042_m1” |
| | “Hs00996236_m1” “Hs00937509_m1” “Hs05016463_s1” |
| | “Hs00233987_m1” “Hs01114274_m1” “Hs01042796_m1” |
| | “Hs00248075_m1” “Hs00957562_m1” “Hs00196245_m1” |
| | “Hs01560931_m1” “Hs01089557_s1” “Hs00222415_m1” |
| | “Hs04194422_s1” “Hs01931732_s1” “Hs00884853_s1” |
| | “Hs00978280_m1” “Hs00959010_m1” “Hs00559914_m1” |
| 30-24 | “Hs01042796_m1” “Hs00801390_s1” “Hs00960591_m1” |
| | “Hs00992679_m1” “Hs00978280_m1” “Hs00937509_m1” |
| | “Hs01931732_s1” “Hs01560931_m1” “Hs05016463_s1” |
| | “Hs00171157_m1” “Hs00167524_m1” “Hs00260452_m1” |
| | “Hs01089557_s1” “Hs00232157_m1” “Hs00899658_m1” |
| | “Hs00904817_m1” “Hs00202752_m1” “Hs00958111_m1” |
| | “Hs00968305_m1” “Hs00248075_m1” “Hs00607978_s1” |
| | “Hs04942686_m1” “Hs01920599_gH” “Hs00365956_m1” |
| | “Hs00944507_g1” “Hs01043717_m1” “Hs00745492_s1” |
| | “Hs00704853_s1” “Hs00610058_m1” “Hs04194422_s1” |
| 30-25 | “Hs00738791_g1” “Hs04232205_s1” “Hs00362096_m1” |
| | “Hs00260452_m1” “Hs01547054_m1” “Hs00196245_m1” |
| | “Hs01560931_m1” “Hs00964384_g1” “Hs00415546_m1” |
| | “Hs00202752_m1” “Hs03043789_g1” “Hs00997579_m1” |
| | “Hs00745492_s1” “Hs00174029_m1” “Hs04194422_s1” |
| | “Hs00899658_m1” “Hs00251883_m1” “Hs00915710_m1” |
| | “Hs01042796_m1” “Hs04942686_m1” “Hs00248075_m1” |
| | “Hs00222415_m1” “Hs01081598_m1” “Hs01089557_s1” |
| | “Hs00705626_s1” “Hs00356958_m1” “Hs00204257_m1” |
| | “Hs00926053_m1” “Hs05052601_s1” “Hs00372831_g1” |
| 40-1 | “Hs00262107_m1” “Hs01560931_m1” “Hs00159178_m1” |
| | “Hs00975850_m1” “Hs00366766_m1” “Hs00543973_m1” |
| | “Hs00231709_m1” “Hs00372831_g1” “Hs00745492_s1” |
| | “Hs01072228_m1” “Hs04194422_s1” “Hs00174029_m1” |
| | “Hs00171157_m1” “Hs01029174_m1” “Hs01089557_s1” |
| | “Hs00801390_s1” “Hs00958111_m1” “Hs01098278_m1” |
| | “Hs01920599_gH” “Hs00233987_m1” “Hs00374264_g1” |
| | “Hs04189864_m1” “Hs00155241_m1” “Hs00610058_m1” |
| | “Hs03464469_s1” “Hs00386692_m1” “Hs00964384_g1” |
| | “Hs01025572_m1” “Hs01546752_g1” “Hs01395177_m1” |
| | “Hs00248075_m1” “Hs00937509_m1” “Hs00704853_s1” |
| | “Hs03028557_s1” “Hs00747379_m1” “Hs00904817_m1” |
| | “Hs00362096_m1” “Hs01087946_g1” “Hs01031740_m1” |
| | “Hs00978280_m1” |
| 40-2 | “Hs00231709_m1” “Hs01098278_m1” “Hs00174969_m1” |
| | “Hs01029174_m1” “Hs05036222_s1” “Hs00262107_m1” |
| | “Hs00856927_g1” “Hs05052601_s1” “Hs00233987_m1” |
| | “Hs00559914_m1” “Hs01931732_s1” “Hs01560931_m1” |
| | “Hs00167524_m1” “Hs00543973_m1” “Hs01010736_m1” |
| | “Hs00174029_m1” “Hs00260480_m1” “Hs00996236_m1” |
| | “Hs00745492_s1” “Hs00204257_m1” “Hs00374264_g1” |
| | “Hs04942686_m1” “Hs00411188_g1” “Hs00380101_m1” |
| | “Hs01051611_gH” “Hs01089557_s1” “Hs00901350_g1” |
| | “Hs01081598_m1” “Hs00738791_g1” “Hs01025572_m1” |
| | “Hs00248075_m1” “Hs01894962_s1” “Hs00957562_m1” |
| | “Hs00189880_m1” “Hs00937509_m1” “Hs00362096_m1” |
| | “Hs00962398_m1” “Hs00171042_m1” “Hs01076090_m1” |
| | “Hs00801390_s1” |
| 40-3 | “Hs00978280_m1” “Hs00900510_m1” “Hs00411188_g1” |
| | “Hs00174969_m1” “Hs00171157_m1” “Hs01546752_g1” |
| | “Hs04194422_s1” “Hs00826827_g1” “Hs01010736_m1” |
| | “Hs01043717_m1” “Hs00931763_m1” “Hs01089557_s1” |
| | “Hs00703025_s1” “Hs00559914_m1” “Hs03302824_pri” |
| | “Hs01087946_g1” “Hs00950371_m1” “Hs01051445_g1” |
| | “Hs00202752_m1” “Hs00380101_m1” “Hs01553775_g1” |
| | “Hs00232157_m1” “Hs00698959_m1” “Hs01098278_m1” |
| | “Hs03464469_s1” “Hs00884853_s1” “Hs00926053_m1” |
| | “Hs00936519_m1” “Hs00745492_s1” “Hs01560931_m1” |
| | “Hs00362096_m1” “Hs00204257_m1” “Hs00240906_m1” |
| | “Hs00366766_m1” “Hs00260480_m1” “Hs01549976_m1” |
| | “Hs00997579_m1” “Hs00738791_g1” “Hs01114274_m1” |
| | “Hs03043789_g1” |
| 40-4 | “Hs00899658_m1” “Hs00543973_m1” “Hs00260452_m1” |
| | “Hs00233987_m1” “Hs03302824_pri” “Hs04189864_m1” |
| | “Hs01089557_s1” “Hs05036222_s1” “Hs00428732_m1” |
| | “Hs00374264_g1” “Hs00958111_m1” “Hs00167524_m1” |
| | “Hs00856927_g1” “Hs00964384_g1” “Hs00900510_m1” |
| | “Hs00747379_m1” “Hs01087946_g1” “Hs05033260_s1” |
| | “Hs00607978_s1” “Hs04942686_m1” “Hs00937509_m1” |
| | “Hs00380101_m1” “Hs00386692_m1” “Hs00155241_m1” |
| | “Hs01114274_m1” “Hs03028557_s1” “Hs00240906_m1” |
| | “Hs01546752_g1” “Hs00975850_m1” “Hs00610058_m1” |
| | “Hs01631495_s1” “Hs00174029_m1” “Hs00411188_g1” |
| | “Hs00559914_m1” “Hs03043789_g1” “Hs00698959_m1” |
| | “Hs00703025_s1” “Hs00745492_s1” “Hs00387770_m1” |
| | “Hs01008033_m1” |
| 40-5 | “Hs00607978_s1” “Hs01031740_m1” “Hs00174969_m1” |
| | “Hs01114274_m1” “Hs04942686_m1” “Hs00931763_m1” |
| | “Hs00996236_m1” “Hs01395177_m1” “Hs01098278_m1” |
| | “Hs00387770_m1” “Hs04194422_s1” “Hs00248075_m1” |
| | “Hs01546752_g1” “Hs00705626_s1” “Hs01920599_gH” |
| | “Hs00801390_s1” “Hs05052601_s1” “Hs00960591_m1” |
| | “Hs01076090_m1” “Hs00232157_m1” “Hs00262107_m1” |
| | “Hs00944507_g1” “Hs00174705_m1” “Hs01089557_s1” |
| | “Hs01029174_m1” “Hs01051445_g1” “Hs00362096_m1” |
| | “Hs00399035_m1” “Hs00745492_s1” “Hs00978280_m1” |
| | “Hs01043717_m1” “Hs03028557_s1” “Hs00233987_m1” |
| | “Hs00704853_s1” “Hs01008033_m1” “Hs00543973_m1” |
| | “Hs00251883_m1” “Hs01691258_g1” “Hs00155241_m1” |
| | “Hs01042796_m1” |
| 40-6 | “Hs01931732_s1” “Hs00174705_m1” “Hs00248075_m1” |
| | “Hs00174969_m1” “Hs00950371_m1” “Hs00374264_g1” |
| | “Hs00232157_m1” “Hs00747379_m1” “Hs00884853_s1” |
| | “Hs00411188_g1” “Hs01089557_s1” “Hs00196245_m1” |
| | “Hs01029174_m1” “Hs01560931_m1” “Hs00379134_m1” |
| | “Hs04942686_m1” “Hs00738791_g1” “Hs00960591_m1” |
| | “Hs00171042_m1” “Hs05016463_s1” “Hs01025572_m1” |
| | “Hs00260480_m1” “Hs00428732_m1” “Hs00705626_s1” |
| | “Hs01114274_m1” “Hs00153408_m1” “Hs00944507_g1” |
| | “Hs00171157_m1” “Hs00962398_m1” “Hs00975850_m1” |
| | “Hs00997579_m1” “Hs00931763_m1” “Hs01379134_m1” |
| | “Hs04194422_s1” “Hs00703025_s1” “Hs01553775_g1” |
| | “Hs00365956_m1” “Hs01098278_m1” “Hs00559914_m1” |
| | “Hs00992679_m1” |
| 40-7 | “Hs00387770_m1” “Hs01031740_m1” “Hs00356958_m1” |
| | “Hs00959010_m1” “Hs01395177_m1” “Hs00705626_s1” |
| | “Hs01010736_m1” “Hs00703025_s1” “Hs00801390_s1” |
| | “Hs01894962_s1” “Hs00233987_m1” “Hs00174029_m1” |
| | “Hs00196245_m1” “Hs01051445_g1” “Hs00153408_m1” |
| | “Hs01081598_m1” “Hs00411188_g1” “Hs01089557_s1” |
| | “Hs01920599_gH” “Hs00901350_g1” “Hs00950371_m1” |
| | “Hs00997579_m1” “Hs00415546_m1” “Hs00884853_s1” |
| | “Hs00155241_m1” “Hs00915710_m1” “Hs03302824_pri” |
| | “Hs01025572_m1” “Hs00698959_m1” “Hs00167524_m1” |
| | “Hs00936519_m1” “Hs00992679_m1” “Hs00704853_s1” |
| | “Hs00960591_m1” “Hs00968305_m1” “Hs00380101_m1” |
| | “Hs00745492_s1” “Hs00201707_m1” “Hs00996236_m1” |
| | “Hs00366766_m1” |
| 40-8 | “Hs00155241_m1” “Hs01025572_m1” “Hs01087946_g1” |
| | “Hs00204257_m1” “Hs01395177_m1” “Hs05052601_s1” |
| | “Hs00962398_m1” “Hs03028557_s1” “Hs05033260_s1” |
| | “Hs00610058_m1” “Hs00174029_m1” “Hs05036222_s1” |
| | “Hs04189864_m1” “Hs00543973_m1” “Hs01081598_m1” |
| | “Hs01894962_s1” “Hs00738791_g1” “Hs00171042_m1” |
| | “Hs00957562_m1” “Hs01560931_m1” “Hs00231709_m1” |
| | “Hs01098278_m1” “Hs00703025_s1” “Hs01089557_s1” |
| | “Hs00374264_g1” “Hs01010736_m1” “Hs00901350_g1” |
| | “Hs00174969_m1” “Hs00260480_m1” “Hs00996236_m1” |
| | “Hs05016463_s1” “Hs00415546_m1” “Hs00362096_m1” |
| | “Hs00704853_s1” “Hs00975850_m1” “Hs00174705_m1” |
| | “Hs00251883_m1” “Hs00196245_m1” “Hs00222415_m1” |
| | “Hs01931732_s1” |
| 40-9 | “Hs00267207_m1” “Hs00904817_m1” “Hs01087946_g1” |
| | “Hs00233987_m1” “Hs04194422_s1” “Hs00196245_m1” |
| | “Hs03302824_pri” “Hs01089557_s1” “Hs00936519_m1” |
| | “Hs01560931_m1” “Hs00559914_m1” “Hs00171157_m1” |
| | “Hs00900510_m1” “Hs00174705_m1” “Hs00856927_g1” |
| | “Hs00950371_m1” “Hs01043717_m1” “Hs00960591_m1” |
| | “Hs01546752_g1” “Hs00957562_m1” “Hs00372831_g1” |
| | “Hs01029174_m1” “Hs00937509_m1” “Hs00411188_g1” |
| | “Hs05016463_s1” “Hs00705626_s1” “Hs00232157_m1” |
| | “Hs00174029_m1” “Hs00978280_m1” “Hs05052601_s1” |
| | “Hs05033260_s1” “Hs01920599_gH” “Hs00231709_m1” |
| | “Hs01098278_m1” “Hs01081598_m1” “Hs00997579_m1” |
| | “Hs01051445_g1” “Hs04232205_s1” “Hs00884853_s1” |
| | “Hs00738791_g1” |
| 40-10 | “Hs01076090_m1” “Hs00387770_m1” “Hs01031740_m1” |
| | “Hs00196245_m1” “Hs03464469_s1” “Hs04232205_s1” |
| | “Hs00884853_s1” “Hs00174969_m1” “Hs00607978_s1” |
| | “Hs04942686_m1” “Hs00931763_m1” “Hs01098278_m1” |
| | “Hs00996236_m1” “Hs00960591_m1” “Hs01920599_gH” |
| | “Hs01546752_g1” “Hs00997579_m1” “Hs01087946_g1” |
| | “Hs01560931_m1” “Hs00950371_m1” “Hs01549976_m1” |
| | “Hs00747379_m1” “Hs01081598_m1” “Hs01691258_g1” |
| | “Hs00231709_m1” “Hs00399035_m1” “Hs00428732_m1” |
| | “Hs00543973_m1” “Hs00240906_m1” “Hs00372831_g1” |
| | “Hs00703025_s1” “Hs00171042_m1” “Hs01043717_m1” |
| | “Hs01025572_m1” “Hs00222415_m1” “Hs00937509_m1” |
| | “Hs00267207_m1” “Hs00915710_m1” “Hs00366766_m1” |
| | “Hs00610058_m1” |
| 40-11 | “Hs00610058_m1” “Hs00745492_s1” “Hs00901350_g1” |
| | “Hs01051611_gH” “Hs01087946_g1” “Hs05052601_s1” |
| | “Hs03028557_s1” “Hs00248075_m1” “Hs01089557_s1” |
| | “Hs00738791_g1” “Hs00374264_g1” “Hs00543973_m1” |
| | “Hs04189864_m1” “Hs00698959_m1” “Hs05036222_s1” |
| | “Hs00962398_m1” “Hs00362096_m1” “Hs03043789_g1” |
| | “Hs03302824_pri” “Hs00380101_m1” “Hs00232157_m1” |
| | “Hs01553775_g1” “Hs01547054_m1” “Hs00174705_m1” |
| | “Hs00936519_m1” “Hs00386692_m1” “Hs00926053_m1” |
| | “Hs00996236_m1” “Hs00960591_m1” “Hs01098278_m1” |
| | “Hs01560931_m1” “Hs00202752_m1” “Hs04942686_m1” |
| | “Hs00428732_m1” “Hs01072228_m1” “Hs00884853_s1” |
| | “Hs00931763_m1” “Hs00964384_g1” “Hs00826827_g1” |
| | “Hs00155241_m1” |
| 40-12 | “Hs00174705_m1” “Hs00698959_m1” “Hs01546752_g1” |
| | “Hs00262107_m1” “Hs03464469_s1” “Hs00996236_m1” |
| | “Hs00960591_m1” “Hs00387770_m1” “Hs00204257_m1” |
| | “Hs00801390_s1” “Hs00610058_m1” “Hs01631495_s1” |
| | “Hs01081598_m1” “Hs00240906_m1” “Hs01553775_g1” |
| | “Hs00362096_m1” “Hs00899658_m1” “Hs00248075_m1” |
| | “Hs01894962_s1” “Hs01089557_s1” “Hs00747379_m1” |
| | “Hs01560931_m1” “Hs00267207_m1” “Hs01010736_m1” |
| | “Hs00901350_g1” “Hs03302824_pri” “Hs01547054_m1” |
| | “Hs00386692_m1” “Hs01098278_m1” “Hs00399035_m1” |
| | “Hs00189880_m1” “Hs00232157_m1” “Hs00356958_m1” |
| | “Hs00167524_m1” “Hs00380101_m1” “Hs04942686_m1” |
| | “Hs00174969_m1” “Hs00968305_m1” “Hs00745492_s1” |
| | “Hs00366766_m1” |
| 40-13 | “Hs01546752_g1” “Hs01920599_gH” “Hs00801390_s1” |
| | “Hs01560931_m1” “Hs04194422_s1” “Hs01081598_m1” |
| | “Hs00251883_m1” “Hs00559914_m1” “Hs00262107_m1” |
| | “Hs00374264_g1” “Hs00543973_m1” “Hs00171157_m1” |
| | “Hs00937509_m1” “Hs01029174_m1” “Hs00174969_m1” |
| | “Hs00232157_m1” “Hs00705626_s1” “Hs05016463_s1” |
| | “Hs01553775_g1” “Hs00704853_s1” “Hs01691258_g1” |
| | “Hs00167524_m1” “Hs00962398_m1” “Hs00978280_m1” |
| | “Hs00248075_m1” “Hs00386692_m1” “Hs00959010_m1” |
| | “Hs01098278_m1” “Hs00884853_s1” “Hs00174705_m1” |
| | “Hs00996236_m1” “Hs00379134_m1” “Hs04942686_m1” |
| | “Hs00202752_m1” “Hs00189880_m1” “Hs00365956_m1” |
| | “Hs03302824_pri” “Hs00926053_m1” “Hs01395177_m1” |
| | “Hs04189864_m1” |
| 40-14 | “Hs00747379_m1” “Hs00362096_m1” “Hs00386692_m1” |
| | “Hs01089557_s1” “Hs01029174_m1” “Hs00960591_m1” |
| | “Hs00248075_m1” “Hs01547054_m1” “Hs01631495_s1” |
| | “Hs00189880_m1” “Hs00167524_m1” “Hs01098278_m1” |
| | “Hs00996236_m1” “Hs00240906_m1” “Hs01087946_g1” |
| | “Hs00884853_s1” “Hs00899658_m1” “Hs00380101_m1” |
| | “Hs04942686_m1” “Hs00936519_m1” “Hs00856927_g1” |
| | “Hs01043717_m1” “Hs00915710_m1” “Hs00231709_m1” |
| | “Hs00964384_g1” “Hs00387770_m1” “Hs00155241_m1” |
| | “Hs01081598_m1” “Hs00926053_m1” “Hs00366766_m1” |
| | “Hs00171042_m1” “Hs01031740_m1” “Hs00959010_m1” |
| | “Hs01546752_g1” “Hs00233987_m1” “Hs01042796_m1” |
| | “Hs00896999_g1” “Hs00374264_g1” “Hs04232205_s1” |
| | “Hs00201707_m1” |
| 40-15 | “Hs00703025_s1” “Hs01087946_g1” “Hs05016463_s1” |
| | “Hs00167524_m1” “Hs00543973_m1” “Hs01010736_m1” |
| | “Hs05052601_s1” “Hs00957562_m1” “Hs00856927_g1” |
| | “Hs04232205_s1” “Hs01029174_m1” “Hs00251883_m1” |
| | “Hs01931732_s1” “Hs00262107_m1” “Hs00374264_g1” |
| | “Hs01560931_m1” “Hs00174969_m1” “Hs00937509_m1” |
| | “Hs00222415_m1” “Hs00801390_s1” “Hs00959010_m1” |
| | “Hs00231709_m1” “Hs00747379_m1” “Hs00411188_g1” |
| | “Hs01051445_g1” “Hs00174029_m1” “Hs01553775_g1” |
| | “Hs01072228_m1” “Hs01089557_s1” “Hs00415546_m1” |
| | “Hs00705626_s1” “Hs00387770_m1” “Hs00745492_s1” |
| | “Hs00153408_m1” “Hs00196245_m1” “Hs00884853_s1” |
| | “Hs04189864_m1” “Hs00936519_m1” “Hs00958111_m1” |
| | “Hs00607978_s1” |
| 40-16 | “Hs01920599_gH” “Hs04189864_m1” “Hs01114274_m1” |
| | “Hs01395177_m1” “Hs00374264_g1” “Hs00174029_m1” |
| | “Hs00958111_m1” “Hs03028557_s1” “Hs00856927_g1” |
| | “Hs00153408_m1” “Hs01089557_s1” “Hs00607978_s1” |
| | “Hs00201707_m1” “Hs00155241_m1” “Hs00233987_m1” |
| | “Hs00745492_s1” “Hs00610058_m1” “Hs00964384_g1” |
| | “Hs04942686_m1” “Hs00996236_m1” “Hs00174969_m1” |
| | “Hs00944507_g1” “Hs00248075_m1” “Hs00362096_m1” |
| | “Hs00174705_m1” “Hs01043717_m1” “Hs00260452_m1” |
| | “Hs00167524_m1” “Hs00380101_m1” “Hs00559914_m1” |
| | “Hs00159178_m1” “Hs04194422_s1” “Hs00399035_m1” |
| | “Hs01931732_s1” “Hs00703025_s1” “Hs00738791_g1” |
| | “Hs00826827_g1” “Hs00997579_m1” “Hs00204257_m1” |
| | “Hs01070154_m1” |
| 40-17 | “Hs00978280_m1” “Hs01553775_g1” “Hs01029174_m1” |
| | “Hs00996236_m1” “Hs00386692_m1” “Hs00738791_g1” |
| | “Hs01089557_s1” “Hs00365956_m1” “Hs00607978_s1” |
| | “Hs00248075_m1” “Hs01098278_m1” “Hs00962398_m1” |
| | “Hs00196245_m1” “Hs00232157_m1” “Hs00167524_m1” |
| | “Hs04942686_m1” “Hs00201707_m1” “Hs00915710_m1” |
| | “Hs01549976_m1” “Hs00901350_g1” “Hs04194422_s1” |
| | “Hs00997579_m1” “Hs01031740_m1” “Hs00904817_m1” |
| | “Hs01631495_s1” “Hs00153408_m1” “Hs00975850_m1” |
| | “Hs00428732_m1” “Hs00366766_m1” “Hs04232205_s1” |
| | “Hs01547054_m1” “Hs00900510_m1” “Hs01560931_m1” |
| | “Hs00747379_m1” “Hs00356958_m1” “Hs00899658_m1” |
| | “Hs00415546_m1” “Hs00362096_m1” “Hs00937509_m1” |
| | “Hs00958111_m1” |
| 40-18 | “Hs00201707_m1” “Hs04942686_m1” “Hs04194422_s1” |
| | “Hs00899658_m1” “Hs00260452_m1” “Hs01043717_m1” |
| | “Hs00262107_m1” “Hs01070154_m1” “Hs01029174_m1” |
| | “Hs00856927_g1” “Hs01098278_m1” “Hs00996236_m1” |
| | “Hs00944507_g1” “Hs00372831_g1” “Hs00356958_m1” |
| | “Hs01051611_gH” “Hs00380101_m1” “Hs01076090_m1” |
| | “Hs00703025_s1” “Hs00251883_m1” “Hs01560931_m1” |
| | “Hs00171157_m1” “Hs00386692_m1” “Hs05016463_s1” |
| | “Hs00167524_m1” “Hs01089557_s1” “Hs00171042_m1” |
| | “Hs00745492_s1” “Hs00610058_m1” “Hs00884853_s1” |
| | “Hs00958111_m1” “Hs04189864_m1” “Hs00704853_s1” |
| | “Hs00543973_m1” “Hs00374264_g1” “Hs01087946_g1” |
| | “Hs00415546_m1” “Hs05036222_s1” “Hs00904817_m1” |
| | “Hs01072228_m1” |
| 40-19 | “Hs01072228_m1” “Hs00915710_m1” “Hs00174705_m1” |
| | “Hs00356958_m1” “Hs00174029_m1” “Hs00958111_m1” |
| | “Hs00248075_m1” “Hs01553775_g1” “Hs01098278_m1” |
| | “Hs00957562_m1” “Hs00937509_m1” “Hs00960591_m1” |
| | “Hs00747379_m1” “Hs00387770_m1” “Hs01547054_m1” |
| | “Hs00698959_m1” “Hs00399035_m1” “Hs00607978_s1” |
| | “Hs04942686_m1” “Hs00240906_m1” “Hs00962398_m1” |
| | “Hs00232157_m1” “Hs00267207_m1” “Hs00996236_m1” |
| | “Hs00704853_s1” “Hs00365956_m1” “Hs00196245_m1” |
| | “Hs00959010_m1” “Hs00884853_s1” “Hs01691258_g1” |
| | “Hs00950371_m1” “Hs00260480_m1” “Hs00174969_m1” |
| | “Hs01560931_m1” “Hs00964384_g1” “Hs00738791_g1” |
| | “Hs00543973_m1” “Hs00171042_m1” “Hs04189864_m1” |
| | “Hs00415546_m1” |
| 40-20 | “Hs00559914_m1” “Hs00856927_g1” “Hs00232157_m1” |
| | “Hs01029174_m1” “Hs00978280_m1” “Hs01051445_g1” |
| | “Hs01031740_m1” “Hs00899658_m1” “Hs00174705_m1” |
| | “Hs00153408_m1” “Hs00362096_m1” “Hs00944507_g1” |
| | “Hs00260452_m1” “Hs00937509_m1” “Hs00826827_g1” |
| | “Hs00248075_m1” “Hs01043717_m1” “Hs01070154_m1” |
| | “Hs01076090_m1” “Hs01560931_m1” “Hs01920599_gH” |
| | “Hs00543973_m1” “Hs00380101_m1” “Hs00997579_m1” |
| | “Hs03043789_g1” “Hs00901350_g1” “Hs03302824_pri” |
| | “Hs00399035_m1” “Hs00747379_m1” “Hs01114274_m1” |
| | “Hs04232205_s1” “Hs05016463_s1” “Hs00387770_m1” |
| | “Hs00366766_m1” “Hs04194422_s1” “Hs01008033_m1” |
| | “Hs01395177_m1” “Hs00904817_m1” “Hs03464469_s1” |
| | “Hs01894962_s1” |
| 40-21 | “Hs00826827_g1” “Hs00899658_m1” “Hs01076090_m1” |
| | “Hs00411188_g1” “Hs05036222_s1” “Hs00171042_m1” |
| | “Hs00926053_m1” “Hs00233987_m1” “Hs00174029_m1” |
| | “Hs00902334_m1” “Hs00958111_m1” “Hs00153408_m1” |
| | “Hs01081598_m1” “Hs01051445_g1” “Hs04189864_m1” |
| | “Hs00962398_m1” “Hs00155241_m1” “Hs00380101_m1” |
| | “Hs01560931_m1” “Hs00167524_m1” “Hs00171157_m1” |
| | “Hs00260480_m1” “Hs00174969_m1” “Hs01114274_m1” |
| | “Hs00705626_s1” “Hs01920599_gH” “Hs00428732_m1” |
| | “Hs01031740_m1” “Hs05016463_s1” “Hs01549976_m1” |
| | “Hs00931763_m1” “Hs00703025_s1” “Hs00960591_m1” |
| | “Hs05052601_s1” “Hs00937509_m1” “Hs01098278_m1” |
| | “Hs00968305_m1” “Hs01087946_g1” “Hs00959010_m1” |
| | “Hs01089557_s1” |
| 40-22 | “Hs00904817_m1” “Hs00260480_m1” “Hs00856927_g1” |
| | “Hs01894962_s1” “Hs00171042_m1” “Hs00171157_m1” |
| | “Hs01098278_m1” “Hs00202752_m1” “Hs01076090_m1” |
| | “Hs00964384_g1” “Hs00155241_m1” “Hs00950371_m1” |
| | “Hs01042796_m1” “Hs00826827_g1” “Hs00958111_m1” |
| | “Hs00899658_m1” “Hs00233987_m1” “Hs00926053_m1” |
| | “Hs01029174_m1” “Hs00174029_m1” “Hs01081598_m1” |
| | “Hs00159178_m1” “Hs00975850_m1” “Hs00232157_m1” |
| | “Hs00251883_m1” “Hs00372831_g1” “Hs00957562_m1” |
| | “Hs00610058_m1” “Hs01043717_m1” “Hs05033260_s1” |
| | “Hs01920599_gH” “Hs00543973_m1” “Hs01560931_m1” |
| | “Hs00901350_g1” “Hs00705626_s1” “Hs05016463_s1” |
| | “Hs00260452_m1” “Hs01553775_g1” “Hs00153408_m1” |
| | “Hs00962398_m1” |
| 40-23 | “Hs00801390_s1” “Hs00174969_m1” “Hs00959010_m1” |
| | “Hs00968305_m1” “Hs01547054_m1” “Hs01072228_m1” |
| | “Hs00262107_m1” “Hs01114274_m1” “Hs00937509_m1” |
| | “Hs01051611_gH” “Hs01920599_gH” “Hs01031740_m1” |
| | “Hs00399035_m1” “Hs00962398_m1” “Hs00233987_m1” |
| | “Hs00167524_m1” “Hs00826827_g1” “Hs01010736_m1” |
| | “Hs03302824_pri” “Hs01549976_m1” “Hs00901350_g1” |
| | “Hs00975850_m1” “Hs00904817_m1” “Hs00380101_m1” |
| | “Hs00387770_m1” “Hs04232205_s1” “Hs00240906_m1” |
| | “Hs00997579_m1” “Hs01560931_m1” “Hs01631495_s1” |
| | “Hs05052601_s1” “Hs00738791_g1” “Hs00171157_m1” |
| | “Hs00232157_m1” “Hs00978280_m1” “Hs00372831_g1” |
| | “Hs00704853_s1” “Hs00745492_s1” “Hs00936519_m1” |
| | “Hs00204257_m1” |
| 40-24 | “Hs01549976_m1” “Hs00260480_m1” “Hs01031740_m1” |
| | “Hs05052601_s1” “Hs05016463_s1” “Hs00196245_m1” |
| | “Hs01025572_m1” “Hs01042796_m1” “Hs00944507_g1” |
| | “Hs00171157_m1” “Hs00167524_m1” “Hs00962398_m1” |
| | “Hs00428732_m1” “Hs00543973_m1” “Hs01560931_m1” |
| | “Hs01087946_g1” “Hs00960591_m1” “Hs04189864_m1” |
| | “Hs00826827_g1” “Hs00380101_m1” “Hs01691258_g1” |
| | “Hs00386692_m1” “Hs00374264_g1” “Hs00745492_s1” |
| | “Hs00884853_s1” “Hs00411188_g1” “Hs00936519_m1” |
| | “Hs00240906_m1” “Hs00232157_m1” “Hs01098278_m1” |
| | “Hs03302824_pri” “Hs00248075_m1” “Hs00801390_s1” |
| | “Hs01553775_g1” “Hs03464469_s1” “Hs00738791_g1” |
| | “Hs01631495_s1” “Hs00703025_s1” “Hs00896999_g1” |
| | “Hs01010736_m1” |
| 40-25 | “Hs04189864_m1” “Hs00386692_m1” “Hs00926053_m1” |
| | “Hs00260452_m1” “Hs00610058_m1” “Hs00958111_m1” |
| | “Hs00704853_s1” “Hs00380101_m1” “Hs00543973_m1” |
| | “Hs00167524_m1” “Hs00747379_m1” “Hs00428732_m1” |
| | “Hs00559914_m1” “Hs00738791_g1” “Hs00260480_m1” |
| | “Hs00703025_s1” “Hs00975850_m1” “Hs01025572_m1” |
| | “Hs01098278_m1” “Hs01560931_m1” “Hs00745492_s1” |
| | “Hs00233987_m1” “Hs00232157_m1” “Hs05016463_s1” |
| | “Hs00607978_s1” “Hs00171042_m1” “Hs00884853_s1” |
| | “Hs01691258_g1” “Hs00196245_m1” “Hs01081598_m1” |
| | “Hs00251883_m1” “Hs00904817_m1” “Hs01114274_m1” |
| | “Hs00222415_m1” “Hs01051445_g1” “Hs01042796_m1” |
| | “Hs00356958_m1” “Hs00944507_g1” “Hs00379134_m1” |
| | “Hs00705626_s1” |

See Table 11 for gene name associated with each probe ID.

### Example 6:40-GEP to Predict Metastatic Risk in Cutaneous SCC

### Study Design-Development and Validation

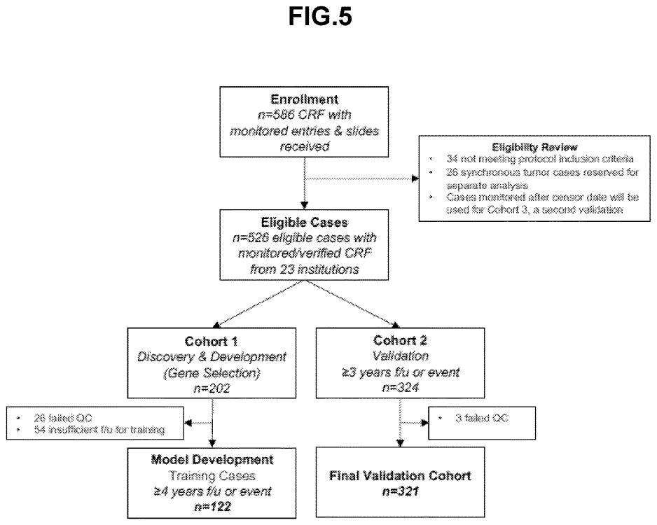

To develop and validate a gene expression signature capable of stratifying patient risk of regional or distant metastasis, a prospectively-designed biomarker study was conducted on 5 archival primary SCC formalin-fixed paraffin-embedded (FFPE) tissue (FIG. 5). The primary end point for this study was metastasis-free survival (MFS), including both regional and distant metastatic events. Regional metastasis was defined as a metastatic lesion within the regional nodal basin, including satellite or in-transit metastasis, but excluding local recurrence. Distant metastasis was defined as metastasis beyond the regional lymph node basin. Disease-specific death, a secondary end point, was defined as death from SCC documented in patient medical records.

Expression of 140 candidate genes was determined for all samples in the discovery and development cases (cohort 1, n=202). Deep learning was applied to expression data from 122 genes passing initial expression thresholds to select genes for further signature training. The prognostic algorithm encompassing the 40-GEP assay was selected based on performance in training and gene coefficients were locked prior to validation. Power calculations indicated that the validation cohort (cohort 2, n=321) could detect a hazard ratio of 2.1 for metastasis with 90% power, alpha=0.05. After validation of the algorithm using cohort 2, clinically-actionable cut-points for probability scores from the models were set to optimize negative predictive value (NPV), positive predictive value (PPV), and sensitivity for metastasis-risk groups (Class 1: low risk; Class 2A: high/moderate risk; Class 2B: highest risk).

### Detailed Study Design—Discovery and Development

For model training (cohort 1, training set), probes were filtered based on the consistency of expression across preliminary runs across 140 probes. The initial set of probes was filtered for amplification and stability of gene expression, resulting in 122 discriminant probes and 6 control probes (MDM2 (Hs00540450_s1), KMT2D (Hs00912419_m1), BAG6 (Hs00190383_m1), FXR1 (Hs01096876_g1), MDM4 (Hs00967238_m1), and KMT2C (Hs01005521_m1). Cases were filtered based on detectable expression of at least 90% of the candidate discriminant probes. Deep learning techniques were applied to gene expression data from cohort 1 for gene selection and model identification. To ensure proper classification, the training set was restricted to cases with a documented metastatic event or at least 4 years of follow up. Gene expression using 140 candidate probes identified by literature review or through preliminary discovery efforts was determined for all samples in cohort 1. Triplicate gene expression data were aggregated and normalized using the control probes identified from the larger case set. Genetic algorithms combined with neural network models were used to generate two independent prediction algorithms from the 122 cases and 122 predictive probes passing initial expression thresholds. Genetic algorithms optimized neural network predictive algorithms across a range of target gene set sizes. Initial models were generated by training neural network models to a set of 100 randomly generated gene lists from the set of 122 without replacement. At each iteration of the genetic algorithm, the top 25% of models were retained and their gene lists mated by randomly selecting approximately 45% of the genes from each list, removing duplicates, and then filling the list to the target size by selecting genes from the remaining 122 gene set. This process provided a minimum 10% mutation rate at each iteration. Genetic improvement continued until the mean kappa value for the population improved by less than 0.01. Neural network hyperparameters were optimized using a training control of 10 times 4-fold repeated cross validation with hyperparameter selection based on the maximum kappa value. The final model was trained against all training data using the optimal hyperparameter set. Two models were developed, one using no weighting, and another weighting metastasis as twice that of nonmetastasis, which together generated the locked algorithm for the 40-GEP test.

### Patient Enrollment, Specimen Acquisition, and Cohort Definitions

Archived FFPE primary cutaneous SCC tissue and associated de-identified clinical data were obtained from 23 independent centers following Institutional Review Board (IRB) approval. Associated clinical, pathological, and outcomes data were entered onto a secure case report form (CRF) and on-site data monitoring was performed for all cases. As part of the ongoing study protocol, 586 archival SCC cases with complete CRFs and FFPE tissue were received. The workflow diagram in FIG. 5 summarizes protocol inclusion/exclusion criteria.

Briefly, inclusion criteria specified pathologically confirmed cutaneous SCC with available FFPE tissue from either the original biopsy or the definitive surgical excision. Subjects had a documented regional or distant metastasis, or a minimum of three years of clinical follow-up without evidence of metastasis. The protocol targeted enrollment of cases for the intent-to-treat patient population. The protocol targeted enrollment of with at least one high-risk feature as defined by guidelines or staging systems (features considered high-risk for targeted enrollment include, but are not limited to, any single clinicopathological feature by which a patient could be deemed NCCN-high risk or increase a patient's T-stage above T1), either at the patient or tumor level, to best model the intent-to-treat patient population. Centralized pathology review of a representative hematoxylin and eosin (H&E) stained tissue section was performed by a board-certified dermatopathologist to confirm diagnosis of SCC and assess for high-risk features. Per study design, the first ~200 cases received and monitored were selected for discovery and development (cohort 1 n=202) and the remaining cases for validation (cohort 2 n=324).

All CRFs were monitored, which included review of all available pathology reports and medical records associated with received lesions. For cohort 2, monitors reviewed 98.4% (314/319) of all pathology reports from definitive surgeries. Two cases did not receive definitive surgery. All cases were categorized as NCCN low or high risk and were restaged by AJCC 8th Edition and BWH criteria per features listed in the original pathology reports, available medical records, and independent dermatopathologist review. Consistent with College of American Pathology (CAP) reporting protocols, histopathological features not reported or not identified were considered negative for staging and analysis.

### Assay Methods

FFPE tissue sections were freshly cut to 5 μm sections at the contributing institution and collected at a central CAP-accredited laboratory. Tumor tissue was macrodissected from slides, including tumor stroma and infiltrating immune cells, and processed to generate RNA and cDNA.

Each cDNA sample underwent a 14-cycle preamplification step prior to dilution, and then was mixed 1:1 with 2× TaqMan Gene Expression Master Mix. Quantitative polymerase chain reaction (qPCR) was then performed using high-throughput microfluidics gene cards containing primers specific to the genes of interest and the QuantStudio 12K Flex Real-Time PCR System (Life Technologies). Each sample was run in triplicate with samples randomized onto plates to distribute metastatic and nonmetastatic cases. Laboratory personnel and clinical monitoring staff were blinded to GEP results during data capture.

### Statistical Analysis

Survival analyses using the Kaplan-Meier method were performed in R (version 2.44) with survival statistics calculated using either the log-rank test or multivariate Cox regression analysis when appropriate. For Cox regression, analysis assumptions of proportional hazards were confirmed using the zph test of the fitted model. In cases where proportional hazards assumptions were violated in Cox regression models, additional multivariate survival regression models were used to confirm the results. Binned 40-GEP results and risk according to established staging methods were included in regression models. Accuracy metrics were assessed for GEP Classes, both Class 2A and 2B as the high-risk group for completeness, and clinical risk staging parameters using functions in the caret package (version 6.0) in R (version 3.6.1).

### Results

### Development of the Prognostic Signature

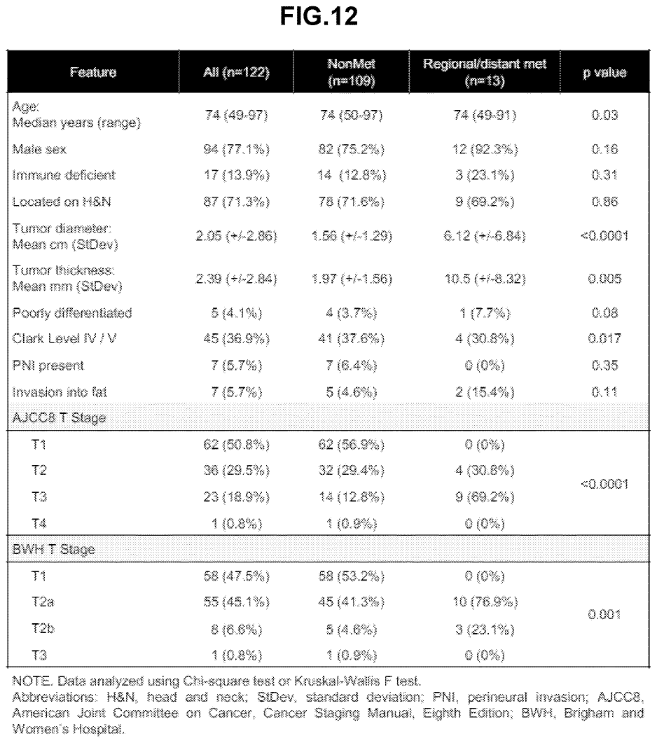

To identify a prognostic signature capable of patient stratification by risk of regional or distant metastasis from primary SCC tumors, deep learning was applied to gene expression data from training cohort (n=122, 13 metastatic cases). Demographics of the training cohort are shown in FIG. 12. The algorithm selected for validation was comprised of two gene expression signatures, inclusive of 6 control and 34 discriminant genes in total, with risk modeling performed using neural networks. This 40-GEP algorithm generated linear scores for probability of metastasis from each predictive signature.

### Independent Validation Cohort Demographics

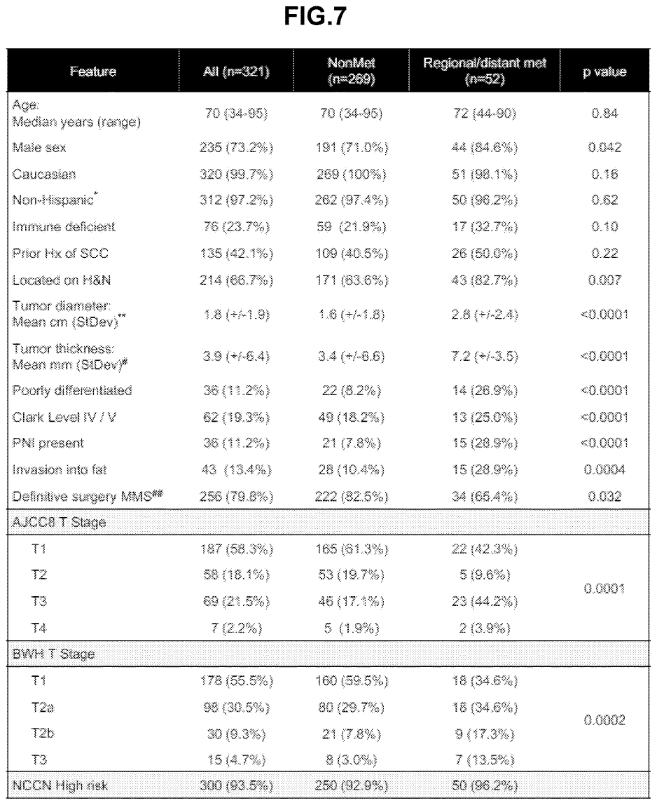

The validation cohort of 321 primary SCC cases was comprised of 52 cases (16%) with documented metastasis, and 269 cases without a metastatic event. Baseline cohort characteristics are summarized in FIG. 7. Most of the patients were Caucasian (99.7%), non-Hispanic (97.2%), male (73.2%), and immunocompetent (76.3%) with tumors located on the head and neck (66.7%), consistent with typical SCC presentation. According to NCCN Guidelines® criteria, 93% were high risk. The surgical treatment modalities were Mohs surgery (79.8%) and wide local excision (19.6%). The following clinicopathologic features were statistically different between metastatic and nonmetastatic cases in univariate analysis: tumor differentiation, perineural invasion, invasion into subcutaneous fat, tumor thickness, tumor diameter, lymphovascular invasion, tumor located on head/neck, definitive surgery as Mohs micrographic surgery, Clark level, and patient sex.

### Independent Validation of the 40-GEP Prognostic Signature

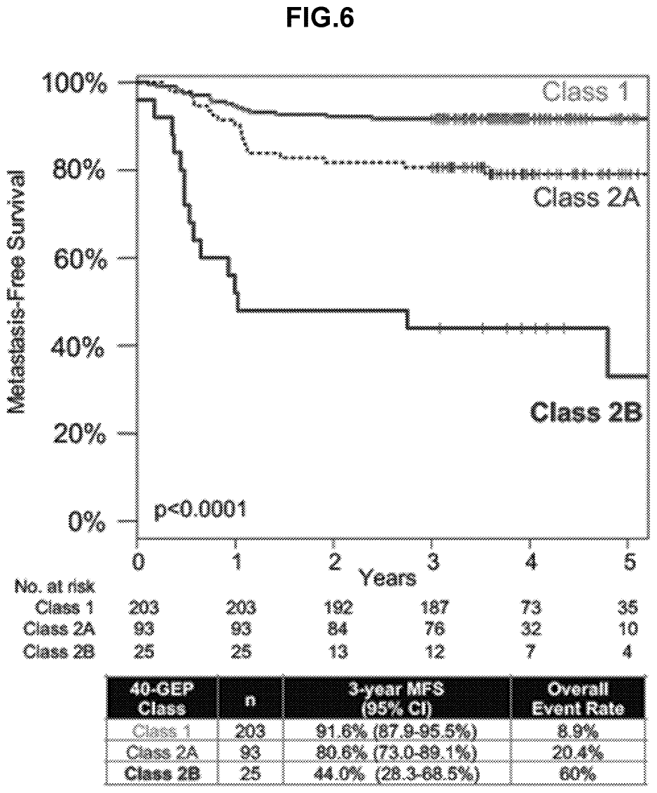

To validate the prognostic capability of the 40-GEP, the algorithm was applied to independent cohort 2. The algorithm demonstrated a statistically significant ability to stratify metastatic risk. The validated 40-GEP was then used to define risk groups with increasing metastasis risk: Class 1 (low risk, n=203), Class 2A (high risk, n=93), and Class 2B (highest risk, n=25). Significantly different 3-year MFS rates were observed for Class 1 (91.6%), Class 2A (80.6%), and Class 2B (44.0%) groups following Kaplan-Meier survival analysis (FIG. 6, log-rank test, p<0.0001). The overall rates of metastasis in each Class were 8.9%, 20.4%, and 60.0%, respectively. The final gene signature identified 64% (34 of 52) of the cases having metastasis as Class 2, with 15 cases identified as Class 2B. The 40-GEP Class was associated with disease-specific death resulting in a hazard ratio of 5.4 and 8.8 for Class 2A and Class 2B, respectively (univariate model; p<0.05, p<0.01). Of the 13 reported deaths due to SCC, 10 were classified as Class 2 (7 Class 2A and 3 Class 2B).

### Prognostic Accuracy of the 40-GEP Test Compared to Staging Systems

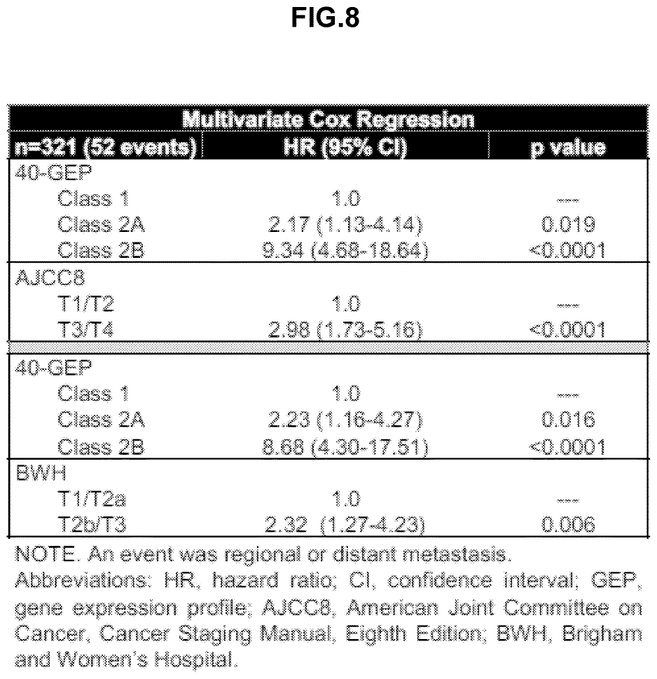

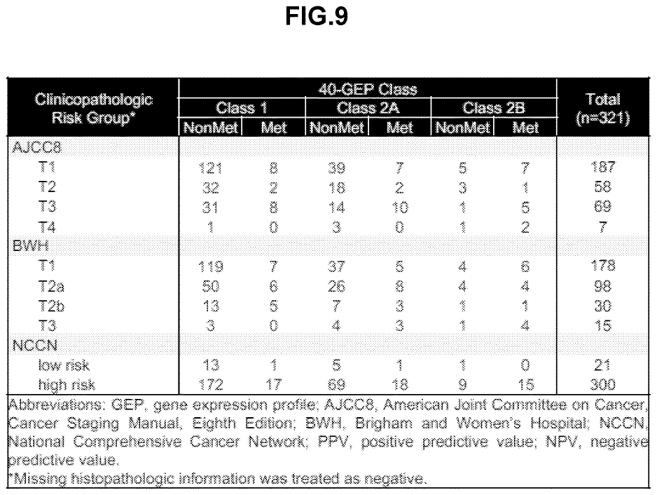

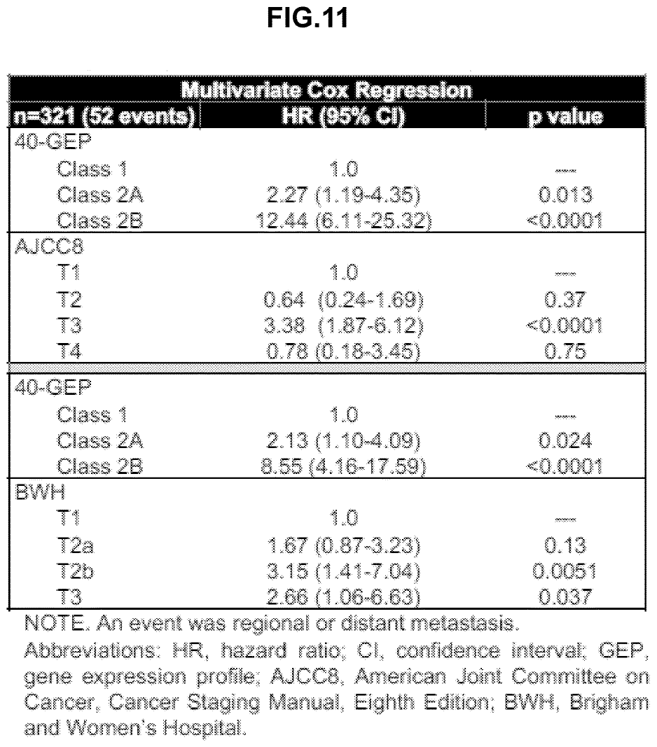

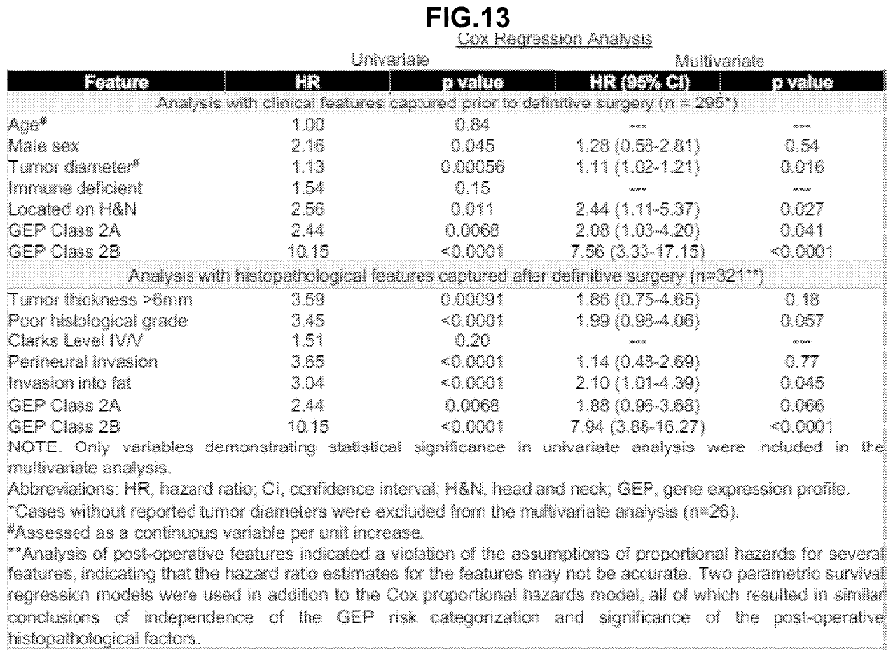

The 40-GEP signature was an independent predictor of risk when analyzed in a multivariate model with AJCC (Class 2A HR=2.17, p=0.019; Class 2B HR=9.34, p<0.0001) or BWH (Class 2A HR=2.23, p=0.016; Class 2B HR-8.68, p<0.0001) staging systems (see FIG. 8 and FIG. 11). Multivariate analysis with individual clinicopathological features also demonstrates that the 40-GEP signature demonstrates independent prognostic value over these features (see FIG. 13). FIG. 9 reports the number of cases with or without metastasis in the validation cohort according to 40-GEP Class and with respect to NCCN risk group or T-stage.

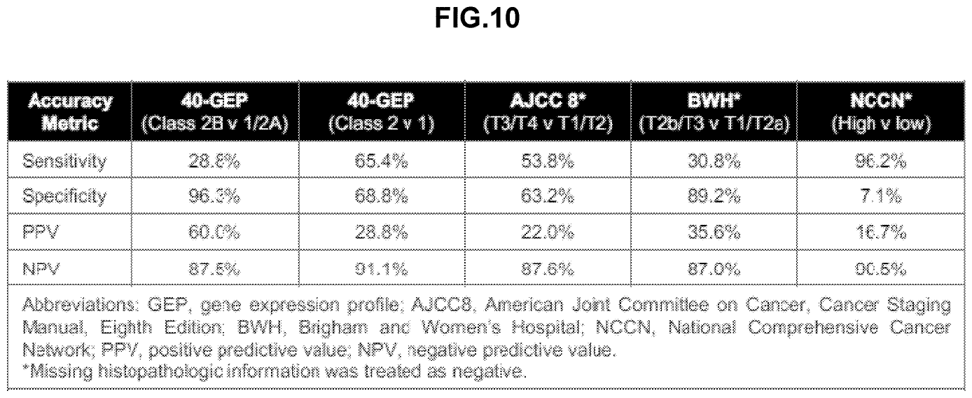

Overall, accuracy metrics for AJCC (T1/T2 vs. T3/T4) and BWH staging (T1/T2a vs. T2b/T3) align with previously published data (FIG. 10; see Ruiz et al., JAMA Dermatol. 155:819 (2019); Karia et al., JAMA Dermatol. 154:175 (2018); Jambusaria-Pahlajani et al., JAMA Dermatol. 149:402 (2013); and Karia et al., JCO 32:327-334 (2014)). The 40-GEP Class 2B group demonstrated a PPV of 60% compared to 16.7%, 22.0%, and 35.6% for NCCN, AJCC, and BWH high-risk groups, respectively (see FIG. 10). The Class 1 group was associated with a 91.1% NPV, exceeding the 87.6% and 87.0% NPV for AJCC and BWH staging, respectively, and matching the 90.5% NPV of NCCN. Importantly, 63% of the validation cohort overall and 67% of the high-risk NCCN cases were identified as low risk Class 1 by the 40-GEP with the highest NPV relative to NCCN, AJCC, and BWH.

TABLE 14
Discriminant genes (n = 34) included in the prognostic signature.

| GENE ID | GENE NAME |
|---|---|
| ACSBG1 | Long-chain-fatty-acid-CoA ligase ACSBG1 |
| ALOX12 | Arachidonate 12-Lipoxygenase, 12S Type |
| APOBEC3G | Apolipoprotein B MRNA Editing Enzyme Catalytic |
| | Subunit 3G |
| ATP6V0E2 | ATPase H+ Transporting V0 Subunit E2 |
| BBC3 | Bcl-2-binding component 3 |
| BHLHB9 | Basic Helix-Loop-Helix Family Member B9 |
| CEP76 | Centrosomal protein of 76 kDa |
| DUXAP8 | Double Homeobox A Pseudogene 8 |
| GTPBP2 | GTP Binding Protein 2 |
| HDDC3 | Guanosine-3′,5′-bis(diphosphate) 3′- |
| | pyrophosphohydrolase MESH1 |
| ID2 | Inhibitor Of DNA Binding 2 |
| LCE2B | Late Cornified Envelope 2B |
| LIME1 (ZGPAT) | Lck Interacting Transmembrane Adaptor 1 |
| LOC100287896 | Uncharacterized LOC100287896 |
| LOC101927502 | Uncharacterized LOC101927502 |
| MMP10 | Matrix Metalloproteinase 10 (Stromelysin 2) |
| MRC1 | Mannose Receptor C-Type 1 |
| MSANTD4 | Myb/SANT DNA Binding Domain Containing 4 With |
| | Coiled-Coils |
| NFASC | Neurofascin |
| NFIC | Nuclear Factor I C |
| PDPN | Podoplanin |
| PI3 | Peptidase Inhibitor 3 |
| PLS3 | Plastin 3 |
| RCHY1 | Ring Finger And CHY Zinc Finger Domain |
| | Containing 1 |
| RNF135 | Ring Finger Protein 135 |
| RPP38 | Ribonuclease P/MRP Subunit P38 |
| RUNX3 | Runt-Related Transcription Factor 3 |
| SLC1A3 | Solute Carrier Family 1 Member 3 |
| SPP1 | Osteopontin |
| TAF6L | TATA-Box Binding Protein Associated Factor 6 Like |
| TFAP2B | Transcription Factor AP-2 Beta |
| ZNF48 | Zinc Finger Protein 48 |
| ZNF496 | Zinc Finger Protein 496 |
| ZNF839 | Zinc Finger Protein 839 |

TABLE 15
34 discriminant genes included in GEP gene set able
to predict risk ofrecurrence and/or metastasis

| | | Change in gene |
|---|---|---|
| | | expression |
| | | in recurrent |
| | | cancer when |
| | Probe Identifier | compared to non- |
| Gene name | (ThermoFisher) | recurrent cancer. |
| ACSBG1 | Hs01025572_m1 | decrease |
| ALOX12 | Hs00167524_m1 | decrease |
| APOBEC3G | Hs00222415_m1 | increase |
| ATP6V0E2 | Hs04189864_m1 | increase |
| BBC3 | Hs00248075_m1 | increase |
| BHLHB9 | Hs01089557_s1 | decrease |
| CEP76 | Hs00950371_m1 | decrease |
| DUXAP8 | Hs04942686_m1 | increase |
| GTPBP2 | Hs01051445_g1 | decrease |
| HDDC3 | Hs00826827_g1 | increase |
| ID2 | Hs00747379_m1 | decrease |
| LCE2B | Hs04194422_s1 | decrease |
| LIME1 (ZGPAT) | Hs00738791_g1 | increase |
| LOC101927502 | Hs05033260_s1 | increase |
| LOC100287896 | Hs01931732_s1 | increase |
| MMP10 | Hs00233987_m1 | decrease |
| MRC1 | Hs00267207_m1 | decrease |
| MSANTD4 | Hs00411188_g1 | decrease |
| NFASC | Hs00978280_m1 | decrease |
| NFIC | Hs00232157_m1 | decrease |
| PDPN | Hs00366766_m1 | decrease |
| PI3 | Hs00964384_g1 | decrease |
| PLS3 | Hs00543973_m1 | decrease |
| RCHY1 | Hs00996236_m1 | increase |
| RNF135 | Hs00260480_m1 | increase |
| RPP38 | Hs00705626_s1 | decrease |
| RUNX3 | Hs00231709_m1 | increase |
| SLC1A3 | Hs00904817_m1 | increase |
| SPP1 | Hs00959010_m1 | increase |
| TAF6L | Hs01008033_m1 | increase |
| TFAP2B | Hs01560931_m1 | decrease |
| ZNF48 | Hs00399035_m1 | increase |
| ZNF496 | Hs00262107_m1 | increase |
| ZNF839 | Hs00901350_g1 | increase |

Control genes: MDM2 (Hs00540450_s1), KMT2D (Hs00912419_m1), BAG6 (Hs00190383_m1), FXR1 (Hs01096876_g1), MDM4 (Hs00967238_m1), and KMT2C (Hs01005521_m1).

### Example 7: Integrating Gene Expression Profiling into NCCN High-Risk Cutaneous Squamous Cell Carcinoma Management Recommendations: Impact on Patient Management and Outcomes

Cutaneous squamous cell carcinoma (cSCC) is the second most common form of skin cancer after basal cell carcinoma. It occurs in approximately one million people in the U.S. and the incidence is rising, partly due to enhanced detection methods and an aging population. Overall, approximately 6% of cSCC patients develop regional or distant metastatic lesions and survival rates are low for those who do develop metastasis. The number of deaths from cSCC, a large proportion of which are preceded by metastasis, has been estimated to rival that from melanoma. Therefore, accurate prediction of risk for metastasis is essential for optimal patient management and improving outcomes.

National Comprehensive Cancer Network (NCCN) Guidelines® outline broad approaches for management of cSCC patients considered high risk for developing recurrence and/or metastasis. Risk stratification and staging systems for cSCC include NCCN Guidelines Criteria®, the American Joint Committee on Cancer (AJCC) Cancer Staging Manual (8th Edition), and the Brigham and Women's Hospital (BWH) tumor classification system. These systems are based on clinical and pathological features; however, they are specifically limited in their ability to predict adverse outcomes (i.e., have low positive predictive value (PPV) for metastasis) and pose a challenge to implementing risk-directed patient management. Patients with cSCC would benefit from improved prognostic tools for determining which patients currently considered clinicopathologically “high risk” are truly at low risk, which patients should consider procedures to detect nodal/distant disease (e.g., node biopsy versus imaging versus clinical examination only), and which should consider therapeutic intervention to reduce risk for recurrence/metastasis (e.g., adjuvant radiation, chemotherapy, additional surgery, and clinical trial enrollment). Given that risk classifications guide treatment plans, improved prognostic tools would enhance shared decision-making between physicians and their patients. Ultimately, the goal is early intervention for individuals who are likely to develop metastasis and avoidance of unnecessary invasive or costly procedures for those who are at lower risk for developing metastasis.

The 40-gene expression profile (40-GEP) test using archival, formalin-fixed paraffin-embedded (FFPE), primary cSCC tissue as disclosed herein stratifies clinicopathologically identified high-risk cSCC tumors into three risk groups based on low (Class 1), high (Class 2A), and highest (Class 2B) risk for regional or distant metastasis at 3 years after diagnosis. A substantially higher PPV (60.0%) was found for the 40-GEP test for Class 2B relative to that found for the AJCC (22.0%) and BWH (35.6%) staging systems, while maintaining a negative predictive value (NPV) of approximately 90.0% (which is similar to that of the AJCC and BWH systems). The primary goal of developing and validating the 40-GEP test was to improve metastasis risk prediction.

Applying the 40-GEP test to risk-directed management recommendations from the NCCN Guidelines® for 300 NCCN-defined high-risk cSCC cases of the 40-GEP clinical validation cohort demonstrated that integration of a molecular prognostic tool with higher PPV, and similar NPV, relative to current staging systems can identify a subgroup (40-GEP Class 1 and low-risk T stage) of NCCN-defined high-risk patients with rates of metastasis similar to those in the clinicopathologic low-risk group, suggesting this subgroup could be managed less aggressively. By comparison, integration of the 40-GEP test also suggested that a patient with a Class 2B tumor with a high risk for metastasis would warrant intensified intervention, thereby achieving risk-appropriate allocation of surgical, imaging, and therapeutic resources. In all, integrating the 40-GEP test into risk-directed guidelines for patient management resulted in more personalized treatment recommendations and potential improvement of net health outcomes. This was accomplished by identifying both a low-risk subgroup (more than 50% of the cohort) that could be managed conservatively (low intensity management) and a smaller subgroup (8%) of patients who were at higher risk for metastasis and would require more aggressive intervention (high intensity management).

### Materials and Methods

Integration of 40-GEP within High-Risk NCCN Patient Management Guidelines

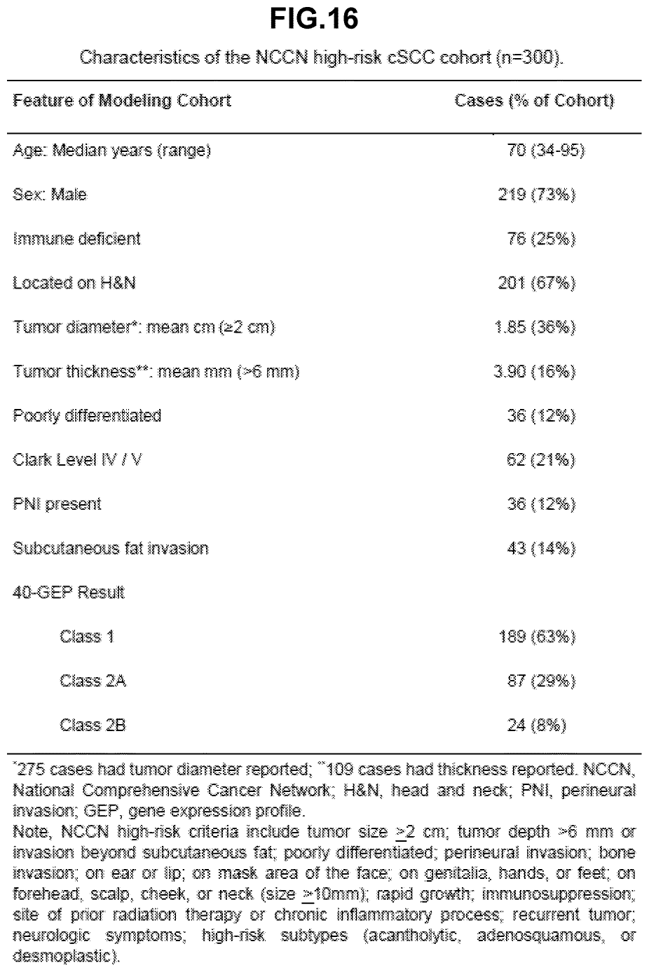

For NCCN-defined high-risk patients from the 40-GEP clinical validation cohort, metastasis risk Class (40-GEP results), T stage, and known patient outcomes were extracted. This high-risk cohort (n=300) included only cases meeting study criteria and having one or more NCCN-defined high-risk feature, as noted in FIG. 16. Briefly, criteria for study inclusion were pathologically-confirmed cSCC diagnosed after Jan. 1, 2006; available archival, FFPE primary cSCC tumor tissue; complete case report forms; and documented metastasis or minimum follow-up period of 3 years without metastasis. Study cohort demographics and clinical characteristics were monitored and underwent centralized pathology review (FIG. 16).

### Data Analysis and Risk-Aligned Management Recommendations

For the NCCN high-risk cohort (n=300), the cases stratified in each 40-GEP Class, along with corresponding metastasis rates and T stage, were analyzed to align each patient group (40-GEP Class/T stage) with risk-appropriate management recommendations. Within the framework of NCCN Guidelines® for management of high-risk cSCC patients with localized disease, risk-aligned management recommendations based on 40-GEP results and T stage were developed for low, moderate, and high intensity management to correspond with metastasis risk bins of <10%, 10-50%, and >50% risk, respectively. Risk-aligned management recommendations addressed follow-up, imaging, nodal assessment, adjuvant therapy, and clinical trials.

### Results

### Cohort Characteristics, 40-GEP Risk Classification, and Outcomes

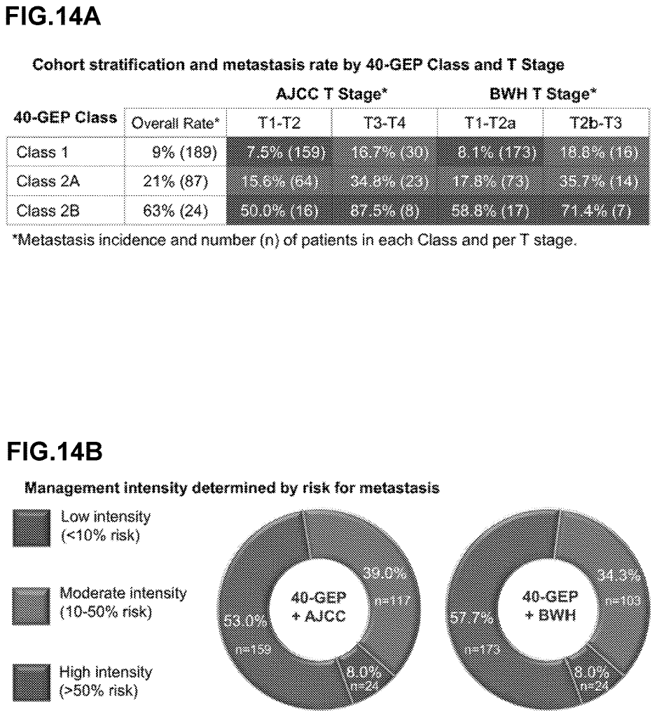

A 300-case cohort of NCCN high-risk cSCC patients (FIG. 16) was used to integrate a recently validated 40-GEP test into NCCN Guidelines® and T stage criteria for patient management to develop risk-aligned management recommendations. The 40-GEP test classifies patients into three risk groups: Class 1, Class 2A, and Class 2B, having low, high, and highest risk for metastasis at 3 years post-diagnosis, respectively. Of the 300 cases, 189 (63.0%) were Class 1, 87 (29.0%) were Class 2A, and 24 (8.0%) were Class 2B with overall metastasis rates of 9%, 21%, and 63%, respectively (see FIG. 14A). More than 50% of the cases were Class 1 and AJCC T1-T2 (n=159, 53.0%) or BWH T1-T2a (n=173, 57.7%) with metastasis rates below 10% (AJCC, 7.5%; BWH, 8.1%) (see FIGS. 14A and 14B). Whereas, Class 1 cases that were also AJCC T3-T4 or BWH T2b-T3, as well as all Class 2A cases, had metastasis rates above 10%, but lower than 50%. All Class 2B cases (8.0% of the cohort) had metastasis rates that were greater than 50%.

### Clinical Utility of Integrating the 40-GEP Test

By combining the low-risk Class 1 result with AJCC T1-T2 stage, a 3-year metastasis rate of 7.5% (NPV, 92.5%) was identified for this subgroup (see FIGS. 14 and 15). This metastasis rate for this subgroup within the NCCN high-risk cohort is approaching the rate reported for the general cSCC patient population (<6% metastasis). Of the Class 1 cases, 159 and 173 were AJCC T1-T2 and BWH T1-T2a, respectively, and were risk-aligned for receiving low intensity management (see FIGS. 14A and 14B). The 40-GEP identified a highest-risk (Class 2B) subpopulation (n=24, 8.0%) which was risk-aligned for receiving high intensity management, consisting of 16 and 8 patients who were AJCC T1-T2 and T3-T4, respectively, and 17 and 7 who were BWH T1-2a and T2b-T3, respectively. Of the remainder of the cohort, 64 were Class 2A/AJCC T1-T2 and 73 were Class 2A/BWH T1-T2a, with a risk for metastasis of 15.6% and 17.8%, respectively (FIG. 14A). These rates are lower than that for the overall cohort, but still more than twice that of the general cSCC patient population. Moderate intensity management was suggested for this group, as well as those patients who were Class 1 or 2A and AJCC T3-T4 or BWH T2b-T3 (see FIGS. 14A and 14B).

The 40-GEP test results, when adjusted for AJCC or BWH T stage in this study, suggest low management intensity for 53.0% or 57.7% of the 300-patient cohort, respectively (FIG. 14). As shown in FIG. 15, low intensity management for these types of Class 1 patients could involve low frequency follow-up visits (1-2 visits/year), low frequency or no imaging, and less intense or no nodal assessments (ultrasound (US) scans versus computed tomography (CT) or nodal palpation in lieu of US or CT). Integration of the 40-GEP test suggests moderate intensity for 39.0% (40-GEP+AJCC) or 34.3% (40-GEP+BWH) of the cohort, and high intensity patient management for 8.0% (FIG. 14). Moderate intensity management could allow for fewer follow-up visits relative to high intensity management (2-4 versus 4-12 visits/year for 3 years), fewer invasive procedures (fewer biopsies and lymph node dissections), and more sparing use of systemic and adjuvant therapy (immunotherapy, chemotherapy, or adjuvant radiation therapy) (FIG. 15). For those patients for whom these risk-aligned recommendations suggest high intensity management, more intensified surveillance and treatment modalities as shown in FIG. would be risk-appropriate.

The 40-GEP test results for a cohort of 300 NCCN-defined high-risk patients were combined with T stage, and risk-aligned recommendations for patient management intensity were developed within the NCCN Guidelines® framework. This integration demonstrates the validated 40-GEP prognostic test has clinical utility for complementing current staging systems and national patient guidelines to refine management pathways for cSCC patients deemed high risk by clinicopathologic methods. The 40-GEP test provides more accurate prediction of risk for metastasis in NCCN-defined high-risk cSCC patients, enabling improved risk-directed management decisions for therapy and surveillance. The current study reports the value of the test to identify within an NCCN high-risk cSCC patient population: 1) low-risk patients, having metastasis rates similar to rates of the general cSCC patient population, and who could benefit from low intensity management; and 2) truly high-risk patients who may benefit from high intensity management. The value of more accurate prognosis would be an improvement in health outcomes through the delivery of risk-appropriate management. Collectively, the 40-GEP test provides independent probability for risk of metastasis that, in combination with AJCC or BWH T stage, could improve risk-directed management in patients diagnosed with NCCN-defined high-risk cSCC. In summary, integration of the 40-GEP test into management of high-risk cSCC could enable net health outcome improvements for the majority of patients tested. The 40-GEP test can be integrated within NCCN guideline recommendations and, in combination with T stage, may have clinical utility for impacting patient management decisions and outcomes.

### Example 8: Incorporation of the 40-Gene Expression Profile Test into Clinicopathological Risk Factor Assessment for Metastasis Prediction in High-Risk Cutaneous Squamous Cell Carcinoma

An estimated 2-6% of cutaneous squamous cell carcinoma (cSCC) patients develop regional or distant metastasis, and approximately 2% die from the disease annually in the U.S. Although the fatality rate is low, the incidence of cSCC is high and continues to grow (estimated at 1-2.5 million cases/year), resulting in a substantial number of patients with poor outcomes. As the distribution of nonmelanoma skin cancer is shifting from a historical 1:4 towards a 1:1 ratio for cSCC to basal cell carcinoma, the estimated mortality rate for cSCC is similar to and will likely surpass that for melanoma.

Development of metastatic disease has a profound impact on cSCC patient survival, underscoring the need for effective identification of patients at risk for metastasis. While the 5-year disease-specific survival rate is >90% for localized disease, those rates drop to 50-83% and below 40% for patients with regional and distant metastases, respectively. Thus, accurate identification of which cSCC tumors have higher metastatic potential is essential for optimizing management decisions, particularly given the effective interventions that are available for cSCC treatment.

Patients with cSCC are broadly classified as having high-risk disease based on clinicopathologic factors associated with increased risk for recurrence and/or metastasis. For example, tumors with diameter >2 cm have been reported to have 2- and 3-fold greater risk for recurrence and metastasis, respectively, relative to smaller tumors. Likewise, tumors invading beyond subcutaneous fat and those with perineural invasion (PNI) of large caliber nerves or poor histologic differentiation have been linked to a 2- to 23-fold increased risk for recurrence and metastasis in univariate analyses. At the patient level, immunosuppressed individuals are at greater risk for developing cSCC and often present with more aggressive cSCC tumors. While these and other factors are used to stratify patient risk, low accuracy, histopathologic discordance, and lack of reporting standardization limit clinical utility of this approach. Standardized methods for cSCC risk assessment are not universally adopted and continue to be refined. The National Comprehensive Cancer Network (NCCN) categorizes a patient as high risk for recurrence and/or metastasis by the presence of a single NCCN-defined high-risk factor, and provides a broad range of management guidelines based on this assessment. Current tumor staging systems, such as the American Joint Committee on Cancer (AJCC) Cancer Staging Manual, 8th Edition (AJCC8), and Brigham and Women's Hospital (BWH) system, help determine recurrence and metastatic risk by incorporation of high-risk factors into tumor (T) stages. While all systems utilize clinicopathologic factors of the primary tumor to categorize risk, their clinical utility is limited, primarily by low positive predictive values (PPV), leading to overestimation of the number of patients at risk for metastasis. Due to these clinical limitations, physicians often rely on professional experience and institution-specific approaches to drive treatment decisions. Despite attempts to improve and implement risk assessment, a standardized and accurate stratification system remains a clinically unmet need in the care of cSCC patients.

The above Examples demonstrate validation of a gene expression profiling-based algorithm (40-GEP; see Table 15) that accurately identifies cSCC tumor risk for metastasis by classifying patients into three groups: Class 1 (low risk), Class 2A (moderate risk), and Class 2B (high risk). In an archival cohort of 321 cSCC patients, the Class 2B group demonstrated a PPV of 60% compared to 33%, 35%, and 17% for AJCC8, BWH, and NCCN systems, respectively. Observed 3-year metastasis-free survival (MFS) rates were 92%, 81%, and 44% for Class 1, Class 2A, and Class 2B patients, respectively; and those having a Class 2B or Class 2A result had a significantly higher hazard ratios (HR) relative to Class 1 patients in multivariate analysis with either AJCC8 or BWH binary T stages, indicating independent prognostic value. The demonstrated accuracy of the 40-GEP test for predicting risk for metastasis in patients with high-risk cSCC is based on tumor-intrinsic factors alone, and improved prognostic value relative to current staging systems.

This Example demonstrates the clinical validity of the 40-GEP test when incorporated into routine clinicopathologic factor-based cSCC risk assessment. In an expanded cohort of cSCC patients (n=420) with high-risk factors, this Example shows independent prognostic value of the 40-GEP test performed using clinical laboratory-developed standard operating procedures (SOPs). Combining novel molecular prognostication with clinicopathologic risk assessment demonstrated improved risk stratification, which can facilitate risk-appropriate management decisions for high-risk cSCC patients.

### Methods

### Study Cohort

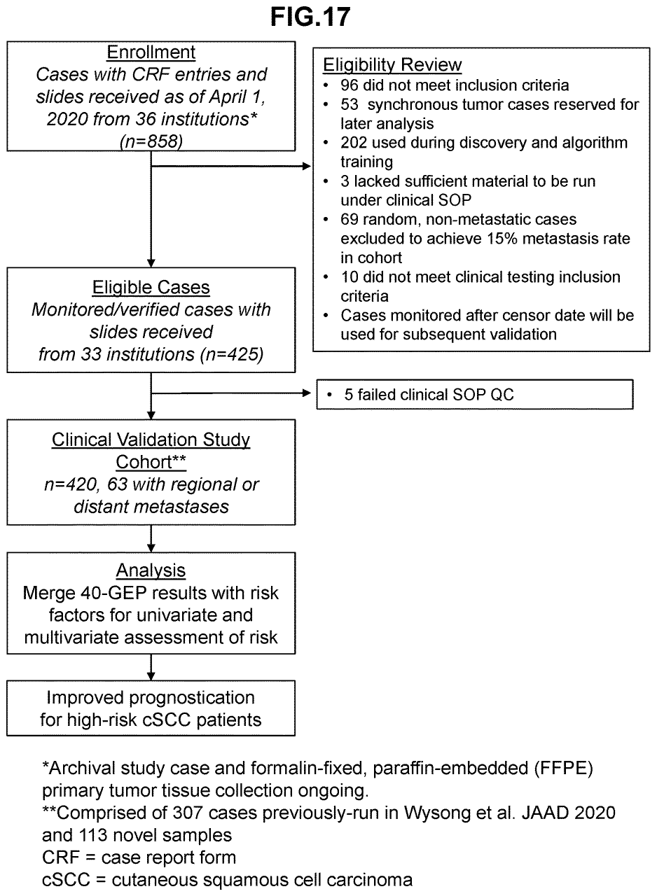

Using an ongoing, institutional review board-approved protocol, formalin-fixed paraffin-embedded (FFPE) samples from primary cSCC lesions and corresponding clinicopathologic and outcomes data were collected from 33 institutions from Sep. 3, 2016 to Apr. 1, 2020 (FIG. 17). All cases underwent review of biopsy and definitive surgery reports, and medical records. Tested tissue was independently reviewed by a board-certified dermatopathologist for tumor content and high-risk factors. Clinicopathologic factors were deemed positive/present if identified during any review step. All cases (307 previously-run and 113 new) were either high-risk by NCCN guidelines for localized cSCC or met Mohs Micrographic Surgery (MMS) appropriate use criteria (AUC). Methods of individual case risk factor assessment are noted in Table 16. The seven risk factors assessed include: tumor size and location, immune status, PNI, depth of invasion, differentiation, histological subtype, and lymphovascular invasion. For cases with metastases, all samples received and monitored during the time period were included. Random samples from non-metastatic cases were included to align with a ~15% overall metastasis rate, which corresponds with previously published metastasis rates in high-risk cSCC (FIG. 17).

TABLE 16
Risk factors captured and used for factor count by case

| | Factor |
|---|---|
| Risk Factors | Count |
| Tumor size and location* | 1 |
| ┐ Any size on the head, neck, genitalia, hands, | |
| feet or pretibial surface (Areas H or M), or | |
| ┘ ≥2 cm size (or ≥1 cm if keratoacanthoma type) | |
| on any other area of the body (Area L) | |
| Immunosuppressed** | 1 |
| Perineural involvement: Large (≥0.1 mm), named nerve | 1 |
| involvement, <0.1 mm in caliber, or unknown | |
| Depth (any one or combination of) | 1 |
| Invasion beyond subcutaneous fat | |
| Depth ≥ 2 mm | |
| Clark level ≥ IV | |
| Poorly differentiated tumor histology | 1 |
| Aggressive histologic subtypes*** | 1 |
| Lymphovascular invasion | 1 |
| TOTAL POSSIBLE COUNTS^{#} | 7 |

*Location definitions per National Comprehensive Cancer Network (NCCN) Guidelines: Area H, ‘mask areas’ of face (central face, eyelids, eyebrows, periorbital, nose, lips [cutaneous and vermillion], chin, mandible, preauricular and postauricular skin/sulci, temple, and ear), genitalia, hands, and feet; Area M, cheeks, forehead, scalp, neck, and pretibia; and Area L, trunk and extremities (excluding hands, nail units, pretibial, ankles, and feet).
**Types of immunosuppression included per protocol were from organ transplant, leukemia, lymphoma, HIV.
***Any of: Acantholytic, adenosquamous, desmoplastic, sclerosing, basosquamous, small cell, spindle cell, infiltrating, clear cell, lymphoepithelial, sarcomatoid, or metaplastic subtypes.
^{#}Note,
tumors with poorly defined borders, that were rapidly growing, with neurologic symptoms in tumor region, and/or at a site of prior radiation therapy or chronic inflammatory process were not captured by this current study but will be allowed for clinical testing as defined as high risk per NCCN.

### Gene Expression Analysis

All samples were assayed under clinical SOPs in a central College of American Pathologists-accredited laboratory with personnel blinded to patient outcomes. Briefly, FFPE primary cSCC tumor tissue was macrodissected, processed for real-time PCR, and assayed in triplicate. Duplicate sample runs were used to generate clinical 40-GEP Class scores.

### Statistics

Statistical analyses were performed in R (v3.6.3). Survival analyses were performed using Kaplan-Meier methods and log-rank test. Univariate and multivariate Cox regression analyses were performed using standard methods.

### Results

### Risk Assessment by Molecular Prognostication Alone

To validate the 40-GEP algorithm for use in the clinical setting, FFPE samples from 436 primary cSCC tumors were assessed using the 40-GEP algorithm disclosed herein and validated clinical SOPs. Six samples failed amplification at predetermined operating thresholds and 10 cases did not meet testing criteria of one high-risk factor, leaving 420 samples for final clinical validation (cohort characteristics, Table 17). The cohort included 63 cases with regional and/or distant metastases and 357 without an event within ≥3 years of follow-up. Median time-to-metastasis was 0.9 years (95th percentile: 2.7 years). The following clinicopathologic characteristics had significantly different rates for non-metastatic versus metastatic cases: male sex, location on the head and neck, tumor diameter, tumor thickness, poor differentiation, PNI, invasion beyond subcutaneous fat, and cases undergoing MMS (p<0.03; Table 17).

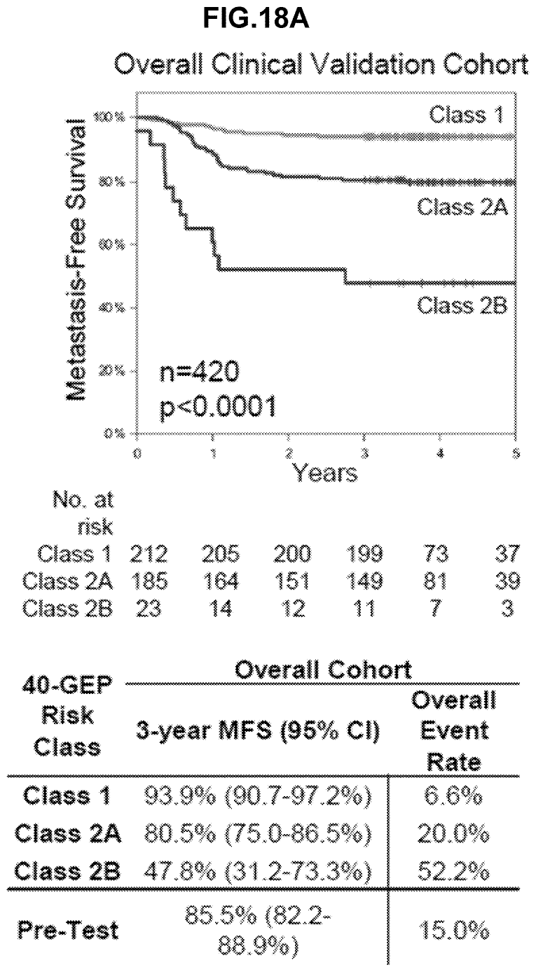

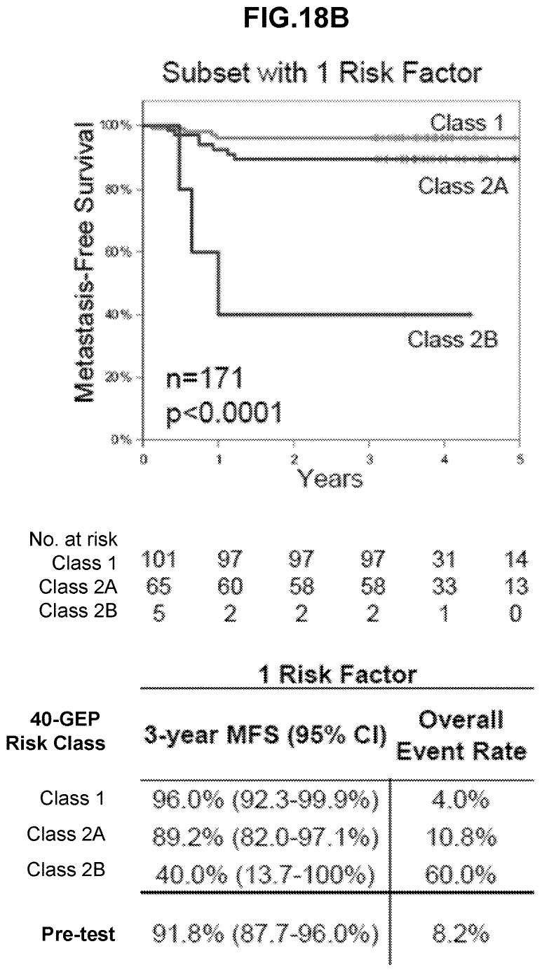

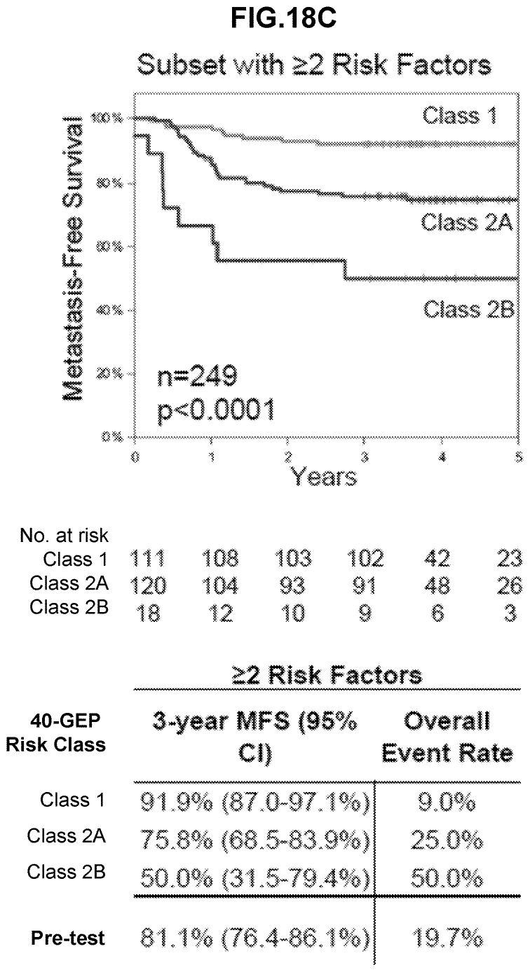

The 40-GEP test accurately stratified patients based on risk for regional or distant metastasis (FIG. 18A). Of the 420 cases included in the study, 212 were identified as Class 1 (low risk), 185 as Class 2A (moderate risk), and 23 as Class 2B (high risk), with metastasis rates of 6.6%, 20.0%, and 52.2%, respectively, and Kaplan-Meier 3-year MFS rates of 93.9%, 80.5% and 47.8% (log-rank, p<0.001, FIG. 18A).

TABLE 17
Demographics and clinical characteristics of the study cohort (n = 420)
Clinical Validation Cohort (n = 420)

| | All | | Non-Metastatic | | Regional/distant | | |
|---|---|---|---|---|---|---|---|
| Characteristics | (n = 420) | | (n = 357) | | Met (n = 63) | | p Value |
| Age: Median years (range) | 71 | (34-95) | 71 | (34-95) | 70 | (44-90) | ns |
| Male sex | 308 | (73.3%) | 253 | (70.9%) | 55 | (87.3%) | 0.007 |
| Caucasian | 417 | (99.3%) | 355 | (99.4%) | 62 | (98.4%) | ns |
| Immunosuppressed* | 103 | (24.5%) | 83 | (23.2%) | 20 | (31.7%) | ns |
| Located on H&N | 278 | (66.2%) | 224 | (62.7%) | 54 | (85.7%) | 0.0002 |
| Ear | 64 | (15.2%) | 53 | (14.8%) | 11 | (17.5%) | |
| Lip | 25 | (6.1%) | 17 | (4.8%) | 8 | (12.7%) | |
| Scalp | 56 | (13.3%) | 40 | (11.2%) | 16 | (25.4%) | |
| Tumor diameter: Mean | 2.01 | (±1.86) | 1.84 | (±1.67) | 3.11 | (±2.52) | <0.0001 |
| cm (StDev)** | | | | | | | |
| Tumor thickness: Mean | 4.34 | (±6.45) | 3.72 | (±6.63) | 7.71 | (±4.07) | <0.0001 |
| mm (StDev)*** | | | | | | | |
| Poorly differentiated | 8 | (13.8%) | 36 | (10.1%) | 22 | (34.9%) | <0.0001 |
| PNI present (≥0.1 mm) | 7 | (1.7%) | 5 | (1.4%) | 2 | (3.2%) | <0.0001 |
| present (<0.1 mm) | 22 | (5.2%) | 12 | (3.4%) | 10 | (15.9%) | |
| present (unknown) | 24 | (5.7%) | 17 | (4.8%) | 7 | (11.1%) | |
| not present | 367 | (87.4%) | 323 | (90.5%) | 44 | (69.8%) | |
| Invasion beyond | 51 | (12.1%) | 34 | (9.5%) | 17 | (27.0%) | <0.0001 |
| subcutaneous fat | | | | | | | |
| Definitive surgery MMS^{#} | 333 | (79.3%) | 291 | (81.5%) | 42 | (66.7%) | 0.023 |
| NCCN High risk | 407 | (96.9%) | 345 | (96.6%) | 62 | (98.4%) | ns |

Data analyzed using Chi-square test or Kruskal-Wallis F test as appropriate for variable type.
Abbreviations: H&N, head and neck; StDev, standard deviation; PNI, perineural invasion; MMS, Mohs micrographic surgery; NCCN, National Comprehensive Cancer Network.
*86 of 103 immunosuppressed patients were transplant patients. **Tumor diameter reported (n = 393). ***Tumor thickness reported (n = 123). ^{#}MMS or wide local excision (n = 415) with 2 cases not having additional surgery beyond biopsy, 3 with unknown definitive surgery.

Incorporation of the 40-GEP with Clinicopathologic Factor-Based Risk Assessment

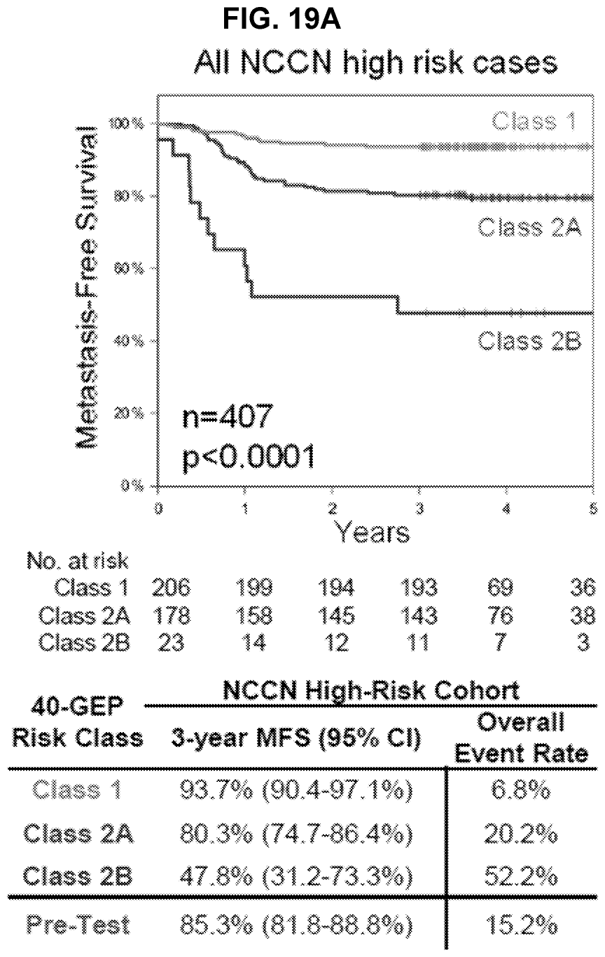

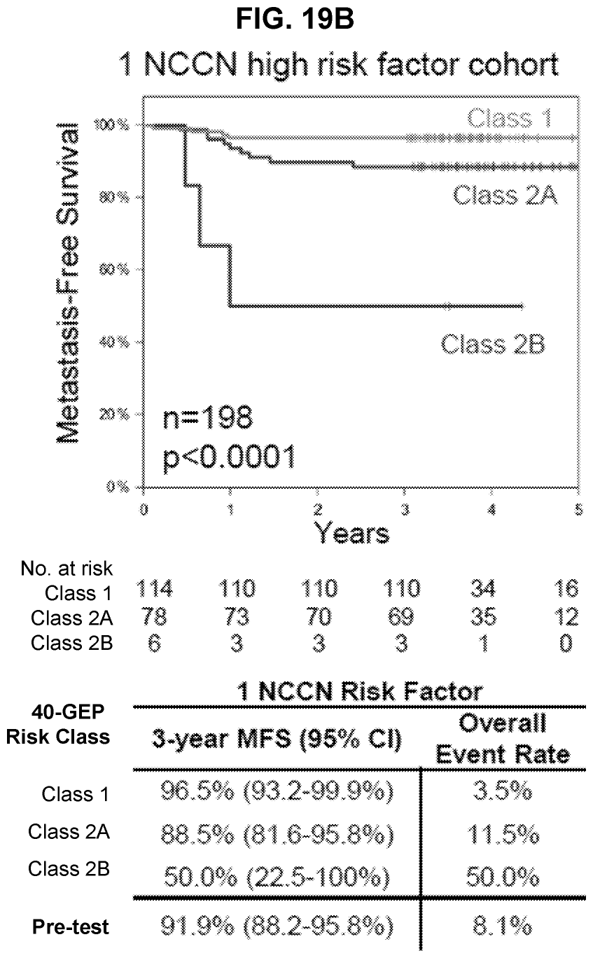

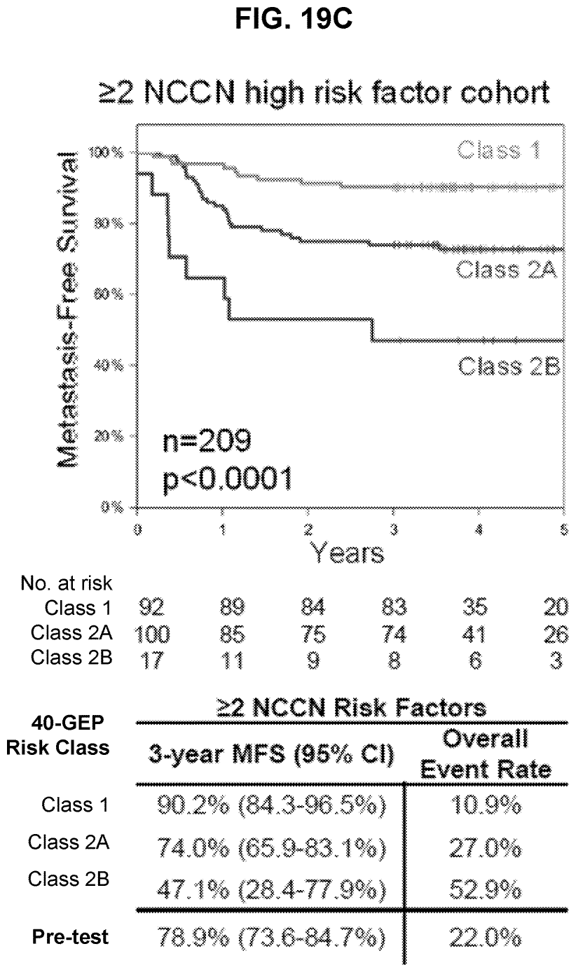

To determine the impact of molecular prognostication on existing risk assessment strategies, subgroups composed of molecular class and combinations of risk factors were interrogated by Kaplan-Meier and regression analysis. First, cases were assessed for total count of risk factors, as determined by NCCN risk criteria or Mohs AUC (as described in the methods, Table 16), and then binned into two groups: those with 1 risk factor (n=171) and those with >2 risk factors (n=249) (FIGS. 18B and 18C). The results demonstrate that there was a direct relationship between risk factor count and overall metastasis rates. The metastasis rate for those with 1 risk factor was 8.2% (versus 15.0% for the whole cohort) compared to 19.7% for cases with ≥2 risk factors. Incorporating the 40-GEP test results identified Class 1 subsets with metastasis rates of 4.0% and 9.0% for 1 and >2 risk factors, respectively (>50% lower than pre-40-GEP testing; FIGS. 18B and 18C). Combining Class 2A results with risk factors identified subsets with moderately higher metastasis rates relative to pre-40-GEP testing (10.8% and 25.0% for 1 and ≥2 factors, respectively). Regardless of risk factor count, Class 2B metastasis rates were ≥50% (more than double the rate for each subset prior to molecular prognostication). These findings were supported by statistically significant differences in 3-year MFS rates for the cohort and corresponding changes for each subset (FIGS. 18A, 18B, and 18C). Similar changes in metastasis rates were observed when cases were binned by NCCN risk factor count or BWH T stage, and assessed by 40-GEP results (Table 18). When only including cases classified as NCCN high risk (n=407, 62 metastatic cases), stratification of risk by the 40-GEP in line with that of the full cohort was observed (FIG. 19A-19C).

TABLE 18
Metastasis rates by 40-GEP and NCCN risk factor count or BWH T-stage

| 40-GEP | ≤1 NCCN Factors | | ≥2 NCCN Factors | |
|---|---|---|---|---|
| Result | n | Met rate | n | Met rate |
| Class 1 | 120 | 3.3% | 92 | 10.9% |
| Class 2A | 85 | 11.8% | 100 | 27.0% |
| Class 2B | 6 | 50.0% | 17 | 52.9% |
| Pre-Test | 211 | 8.1% | 209 | 22.0% |
| 40-GEP | T1/T2a BWH T-stage | | T2b/T3 BWH T-stage | |
| Result | n | Met rate | n | Met rate |
| Class 1 | 193 | 5.7% | 19 | 15.8% |
| Class 2A | 155 | 16.8% | 30 | 36.7% |
| Class 2B | 16 | 43.8% | 7 | 71.4% |
| Pre-Test | 364 | 12.1% | 56 | 33.9% |

Next, to understand how individual factors contribute to metastatic risk, factors with best-supported evidence for association with metastasis and molecular prognostication by the 40-GEP were assessed by Cox regression analyses (Table 19). Using univariate analysis, the risk of metastasis for Class 2A and 2B results was 3.22- and 11.61-fold greater, respectively, than that for Class 1 results (p<0.001). The presence of poor differentiation, PNI, and deep invasion (i.e., beyond the subcutaneous fat, depth >6 mm, or Clark level V) were significant risk factors for metastasis, with HRs of 3.93, 3.28, and 3.11, respectively (p<0.001). Tumor diameter was also predictive of metastatic risk with an HR of 1.15 per cm increase (p<0.001). Despite prior support for immunosuppression as a prognostic risk factor, this variable was not statistically significant in this cohort.

TABLE 19
Univariate and multivariate Cox regression analyses for risk of metastasis in
validation cases with common risk factors for poor outcomes in cSCC

| | | Univariate | | Multivariate Cox | |
|---|---|---|---|---|---|
| | | Cox Regression | | Regression* | |
| | | Hazard Ratio | p | Hazard Ratio | p |
| Risk Factor | n | (95% CI) | value | (95% CI) | value |

40-GEP Result

| | | | | | |
|---|---|---|---|---|---|
| Class 1 | 212 | 1.00 (—) | — | 1.00 (—) | — |
| Class 2A | 185 | 3.22 (1.74-5.95) | <0.001 | 2.33 (1.20-4.53) | 0.013 |
| Class 2B | 23 | 11.61 (5.36-25.15) | <0.001 | 6.86 (2.73-17.22) | <0.001 |

Clinicopathological Risk Factors

| | | | | | |
|---|---|---|---|---|---|
| Poor Differentiation | 58 | 3.93 (2.34-6.60) | <0.001 | 2.29 (1.21-4.33) | 0.011 |
| Perineural Invasion** | 53 | 3.28 (1.41-14.36) | <0.001 | 1.22 (0.58-2.59) | ns |
| Deep Invasion^{#} | 72 | 3.11 (1.86-5.20) | <0.001 | 2.05 (1.04-4.04) | 0.039 |
| Tumor Diameter^{##} | N/A | 1.15 (1.08-1.22) | <0.001 | 1.07 (0.97-1.17) | ns |
| Immunosuppressed | 103 | 1.46 (0.86-2.49) | ns | — | — |

*n = 393, 54 events, excluding cases without tumor diameter reported; **Perineural invasion was considered positive regardless of nerve caliber. ^{#}Deep invasion: beyond the subcutaneous fat, depth > 6 mm or Clark level V; ^{##}Tumor diameter: continuous variable per cm; ns: not statistically significant; N/A: not applicable.

When a multivariate model was generated using factors found to be significant in univariate analysis, only 40-GEP results, poor differentiation, and deep invasion were independent factors for metastatic risk (Table 19). In this multivariate analysis, similar HRs were observed for a Class 2A result, poor differentiation, and deep invasion (2.33, 2.29, and 2.05, respectively; p<0.05); whereas, a 40-GEP Class 2B result was found to have the greatest independent prognostic value (HR, 6.86; p<0.001). Based on MFS rates, as well as Cox regression analyses, incorporation of 40-GEP results with clinicopathologic risk factor-based assessment improved patient metastasis risk stratification.

### Discussion

This Example demonstrates that molecular prognostication, in conjunction with patient and tumor characteristics, increases accuracy and reproducibility of risk assessment for patients with cSCC. The 40-GEP test was further validated as a stand-alone clinical assay to identify cSCC tumors at low (Class 1), moderate (Class 2A), and high (Class 2B) risk for metastasis within 3 years of diagnosis, the time by which most metastatic events occur. This Example also further validates the algorithm for determining metastatic risk with improved accuracy metrics relative to currently available staging systems, validates the test under SOPs implemented for clinical testing, and demonstrates impactful incorporation with clinicopathologic risk factor-based assessment.

Current prognostication suffers from low accuracy, stemming from lack of standardization in reporting and subjectivity during histopathological assessment, and, importantly, failure to capture biological risk at the molecular level. For example, differentiation was removed from AJCC8 T staging as the definitions of well, moderate, and poor differentiation were deemed too inconsistent between centers, limiting its clinical application. While tumor depth/invasion is a well-accepted risk factor for metastasis, how this variable should be captured and what degree of invasion is needed to be considered high risk is debated. Additionally, the rarity of large nerve PNI20 (1.7% in this study) may limit its widespread utility for identifying high-risk patients. These caveats to clinical utility likely contribute to the poor PPV associated with traditional risk assessment, supporting the need for objective and consistent molecular tools to assess tumor biology.

Integration of molecular prognostication into risk assessment can mitigate limitations of assessment based on clinicopathologic factors alone. Findings from this Example demonstrate the 40-GEP complements clinicopathologic risk assessment. In a multivariate model with commonly-utilized high-risk factors, the 40-GEP provided independent prognostic value. Class 2B and Class 2A results were higher and equivalent indicators, respectively, of risk for metastasis relative to other significant factors (poor differentiation and deep invasion, Table 19), indicating the 40-GEP can further stratify patients. Both deep invasion and tumor diameter were significant risk factors in univariate, but tumor diameter was not significant in multivariate analysis, suggesting directionality of growth (i.e., invasive behavior rather than surface spread) is an important distinction. Perineural invasion was also only statistically significant in univariate analysis, consistent with prior studies showing PNI loses significance in analysis with other high-risk factors. In this high-risk cohort, immunosuppression did not reach statistical significance for association with metastasis, despite the fact that it has been strongly associated with risk for additional cSCC lesions and poor outcomes. The archival nature of samples and possible underreporting of high-risk factors are potential limitations of this study. However, the cohort represents a high-risk cSCC population (15% metastasis, 97% NCCN high-risk) and reflects current clinical pathology practices.

Incorporation of molecular prognostication with clinicopathologic factors has improved risk stratification in multiple cancer types. The 40-GEP test, along with clinicopathologic risk factor-based assessment, can identify a group of cSCC patients, in a high-risk cohort, with metastasis rates similar to the general cSCC population (Class 1 40-GEP with 1 risk factor). Even with ≥2 risk factors, the metastasis rate for patients with a Class 1 result was below 10%, (>50% lower than the rate for the whole cohort, pre-40-GEP). Patients identified by the 40-GEP as highest risk for metastasis (Class 2B) consistently had metastasis rates >50%, regardless of having 1 or >2 risk factors. Thus, inclusion of molecular prognostication improved risk stratification via a more objective and standardized risk assessment, and incorporating molecular tools into cSCC patient risk assessment, could lead to more personalized and risk-appropriate pathways to improve patient management and outcomes.

### Example 9: a 20-Gene Expression Profile Test Incorporating Risk Factors Predicts Comprehensive Risk of Disease Progression in High-Risk Cutaneous Squamous Carcinoma to Inform Management Decisions

A subset of patients diagnosed with cutaneous squamous cell carcinoma (cSCC) experience disease progression, resulting in a substantial number of at-risk patients, and highlights the clinical need for enhanced risk stratification to identify these patients. Staging and the National Comprehensive Cancer Network (NCCN) risk stratification are clinicopathological-based systems that do not include tumor molecular risk; thus, it can be clinically challenging to identify at-risk patients due to the heterogeneous presentation of cSCC. More accurate risk stratification is crucial for individualized and risk-aligned clinical management within guidelines to reduce over-/under-treatment. Here, a novel comprehensive gene expression profile (20-GEP) was validated to accurately stratify patient risk within the current NCCN framework, further enhancing the ability of physicians to optimize patient management.

### Methods

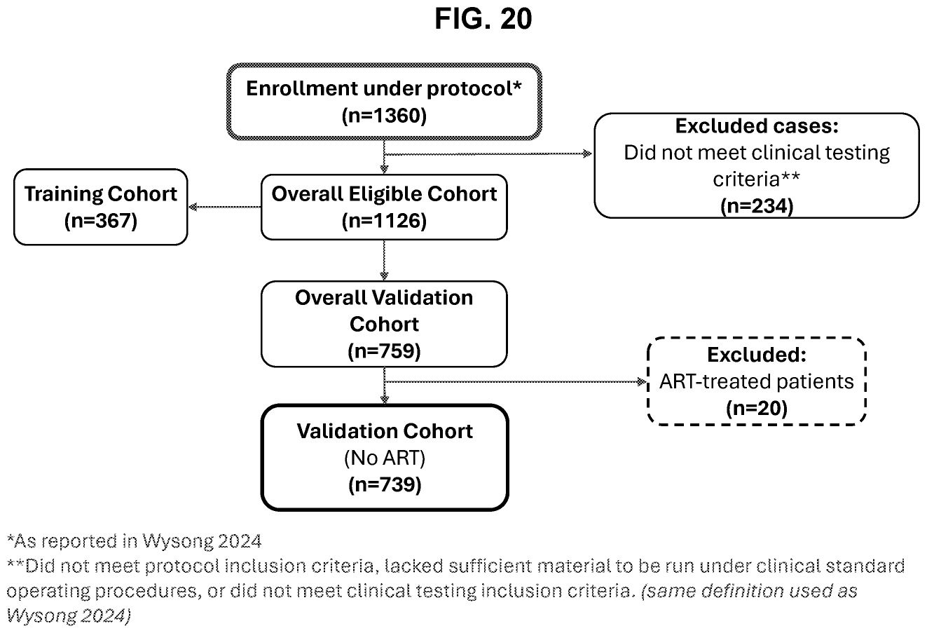

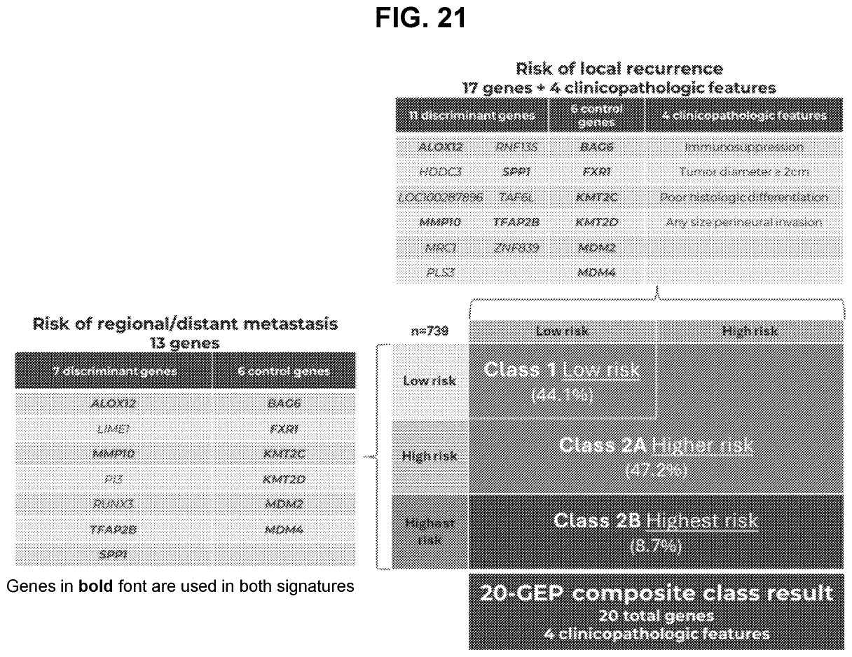

The 20-GEP test combines two independent algorithms consisting of 20 total genes and four clinicopathologic risk factors (immunosuppression, poor histologic differentiation, tumor diameter ≥2 cm, and perineural invasion) to predict risk of nonlocal metastasis (regional/distant) and local recurrence. The 20-GEP was trained and validated using previously published, multi-center, patient data (Wysong et al. Dermatol Ther 2024) (FIG. 21). The validation cohort comprised 739 patients with primary, localized, high-risk cSCC (having ≥1 high-risk factor) treated with definitive surgery and did not receive adjuvant radiation therapy (ART) (FIG. 20). Clinicopathologic data were used to classify patients into NCCN high-risk (HR) and very-high-risk (VHR) subsets. Survival was estimated using Kaplan-Meier survival analysis and compared between groups using the log-rank test. Event-free survival (EFS; local recurrence, regional/distant metastasis) and metastasis-free survival (MFS) are reported with 95% CIs. Negative predictive value (NPV) was calculated for Class 1 compared to Class 2A/B.

### Results

The 20-GEP classified patients as low (Class 1), higher (Class 2A), or highest risk (Class 2B) of disease progression. Of 739 tumors tested, the 20-GEP identified 326 (44.1%) Class 1, 349 (47.2%) Class 2A, and 64 (8.7%) Class 2B tumors (FIG. 21 and Table 20 below).

TABLE 20
20-GEP

| Gene symbol | Name | Specific biological functions | Category |
|---|---|---|---|
| ALOX12 | Arachidonate | Enzyme that oxidizes polyunsaturated | Immune |
| | 12-Lipoxygenase, | fatty acids to produce lipid mediators; | |
| | 12S Type | involved in platelet function and | |
| | | inflammation. | |
| HDDC3 | HD Domain | ppGpp hydrolase; regulates cellular | Metabolism |
| | Containing 3 | stress response and metabolism during | |
| | | starvation. | |
| LIME1 | Lck Interacting | Adaptor protein linking T/B-cell | Immune |
| | Transmembrane | receptor signaling to downstream Src | |
| | Adaptor 1 | family kinase pathways; mediates | |
| | | immune cell activation. | |
| LIPT2-AS1 | LIPT2 antisense | Limited functional data available; | Gene regulation |
| (previously | RNA 1 | likely a long non-coding (lnc) RNA. | |
| LOC100287896) | | | |
| MMP10 | Matrix | Zinc-dependent protease that degrades | Tissue |
| | Metallopeptidase | extracellular matrix components; | remodeling |
| | 10 | involved in tissue remodeling and | |
| | | metastasis. | |
| MRC1 | Mannose | Membrane receptor mediating | Immune |
| | Receptor C- | endocytosis of glycoproteins; | |
| | Type 1 | recognizes pathogens for phagocytosis. | |
| PI3 | Peptidase | Elastase-specific inhibitor with | Tissue |
| | Inhibitor 3 | antimicrobial activity; protects tissues | remodeling |
| | (Elafin) | from proteolytic damage; related to | |
| | | keratinization pathways. | |
| PLS3 | Plastin 3 | Actin-bundling protein; regulates | Migration |
| | | cytoskeletal organization and cell | |
| | | motility. | |
| RNF135 | Ring Finger | E3 ubiquitin ligase; activates antiviral | Immune |
| | Protein 135 | innate immune response via RIG-I | |
| | | signaling. | |
| RUNX3 | RUNX Family | Tumor suppressor transcription factor; | Gene regulation |
| | Transcription | regulates cell differentiation and | |
| | Factor 3 | apoptosis. | |
| SPP1 | Secreted | Extracellular matrix protein; mediates | Migration |
| | Phosphoprotein 1 | cell adhesion, migration, and immune | |
| | (Osteopontin) | signaling. | |
| TAF6L | TATA-Box | Component of transcription factor | Gene regulation |
| | Binding Protein | TFIID complex; involved in RNA | |
| | Associated Factor | polymerase II transcription initiation. | |
| | 6 Like | | |
| TFAP 2B | Transcription | Regulates gene expression, especially | Gene regulation |
| | Factor AP-2 Beta | during development; implicated in cell | |
| | | growth and differentiation. | |
| ZNF839 | Zinc Finger | Putative transcriptional regulator; | Gene regulation |
| | Protein 839 | predicted to enable zinc ion binding | |
| | | activity. | |

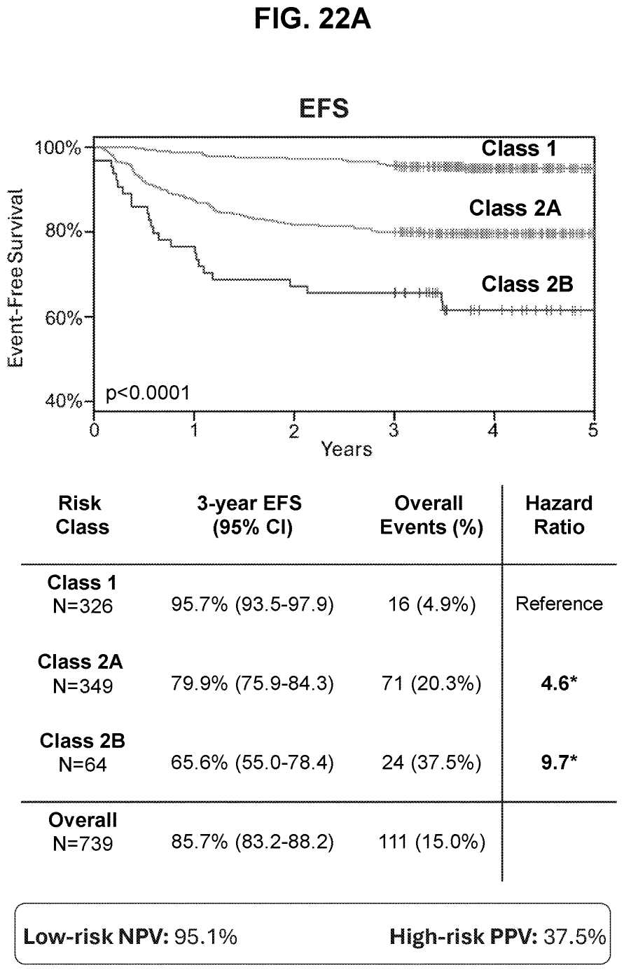

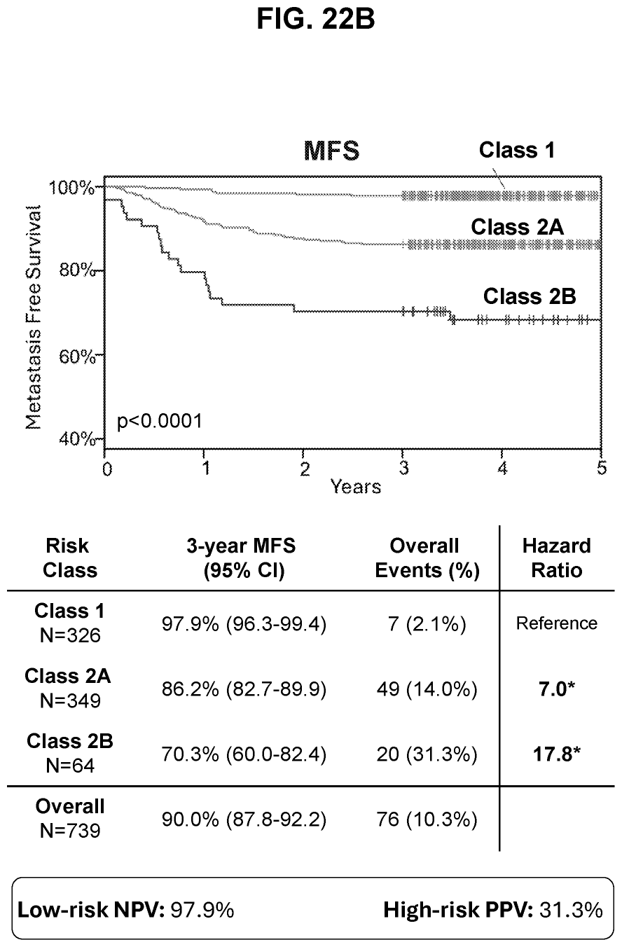

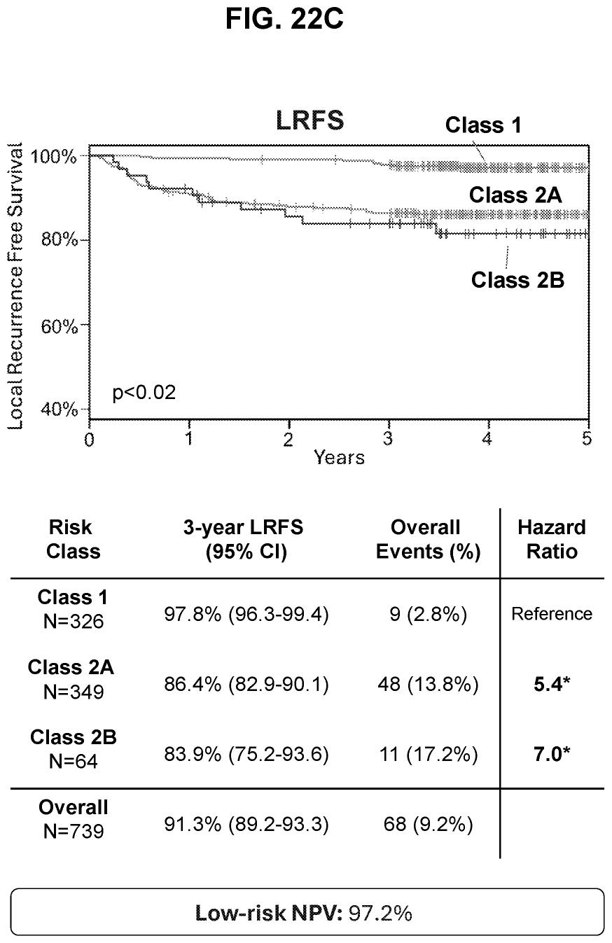

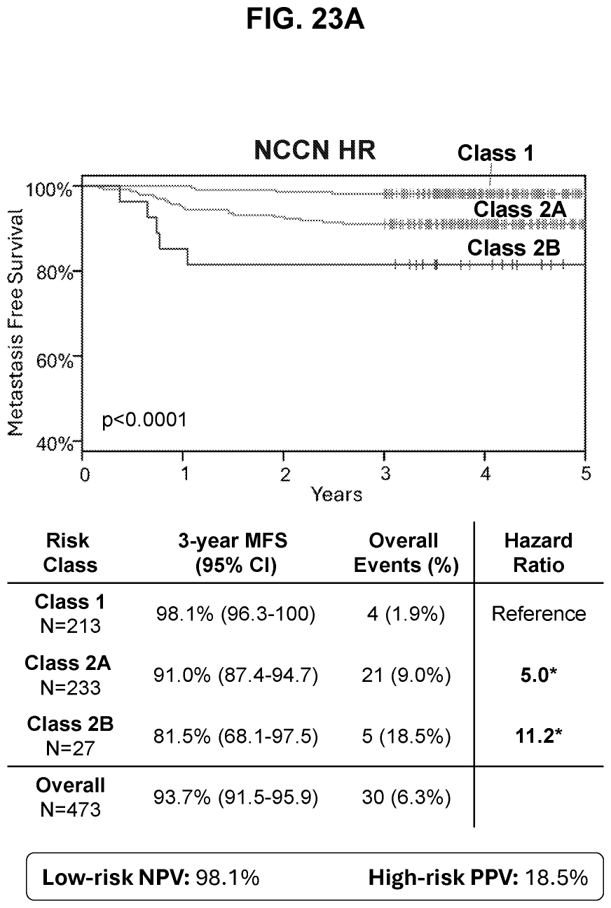

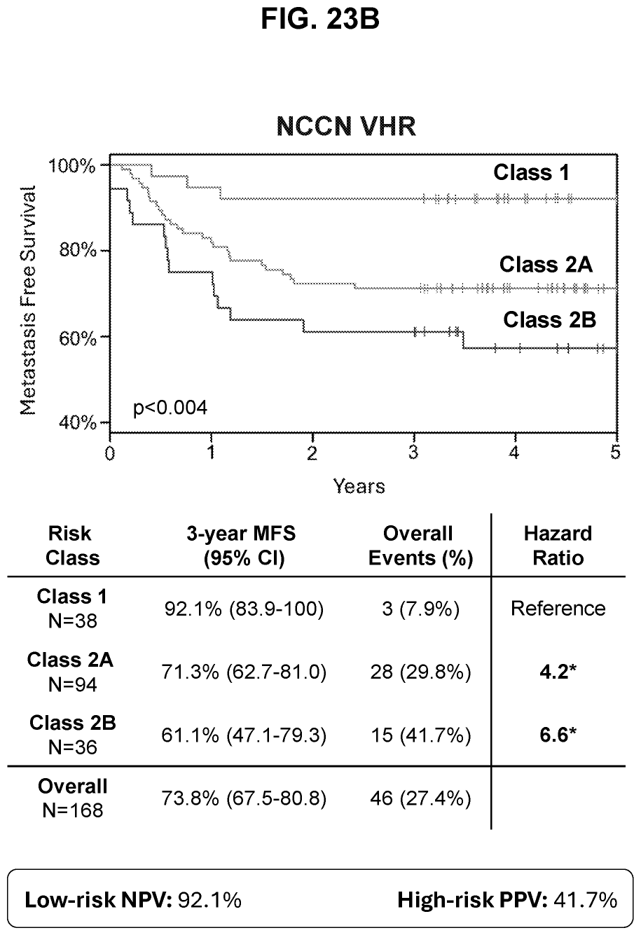

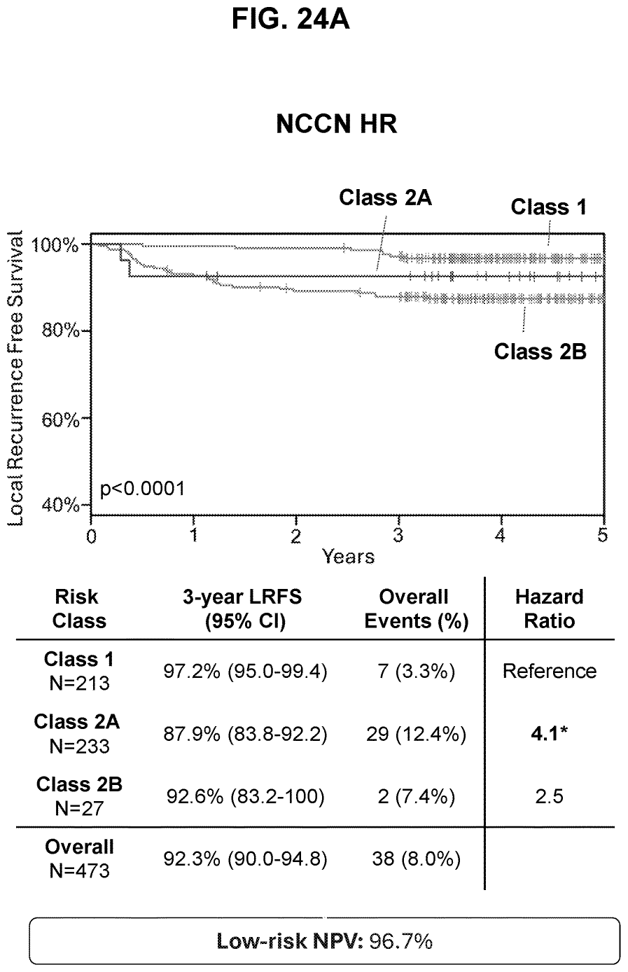

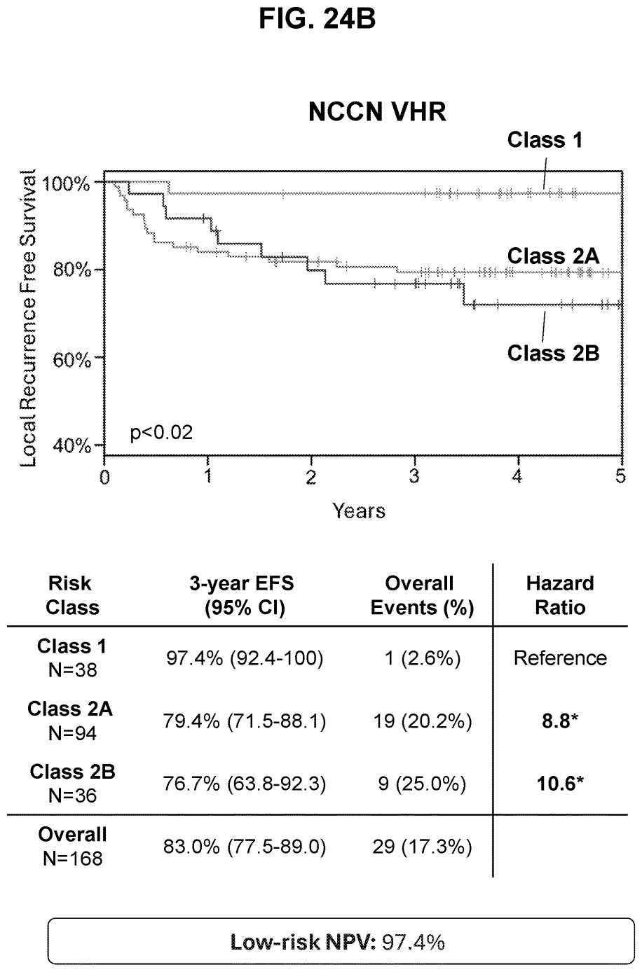

The 20-GEP significantly stratified patient risk of poor outcomes (p<0.001) with an NPV of 95.1% (FIG. 22A). Three-year EFS for Class 1 was 95.7% (93.5-97.9) compared with 79.9% (75.9-84.3) for Class 2A, and 65.6% (55.0-78.4) for Class 2B (FIG. 22A). The 20-GEP also significantly stratified risk of metastasis (log-rank, p<0.001) with an NPV of 97.9% (FIG. 22B). Three-year MFS was 97.9% (96.3-99.4), 86.2% (82.7-89.9), and 70.3% (60.0-82.4) for Class 1, Class 2A, and Class 2B, respectively (FIG. 22B). Similarly, the 20-GEP significantly differentiated metastatic risk within NCCN HR (log-rank, n=473; p<0.001) and VHR (log-rank, n=168; p<0.01) patient groups, with a high 3-year MFS in patients with Class 1 results (HR, 98.1%; VHR, 92.1%) and low MFS in patients with Class 2B results (HR, 81.5%; VHR, 61.1%) (FIG. 23). The 20-GEP also significantly stratified risk of local recurrence (p=0.02) with an NPV of 97.2% (FIG. 22C). Three-year LRFS was 97.8% (96.3-99.4), 86.4% (82.9-90.1), and 83.9% (75.2-93.6) for Class 1, Class 2A, and Class 2B, respectively (FIG. 22C). Similarly, the 20-GEP significantly differentiated local recurrence risk within NCCN HR (n=473; p<0.001) and VHR (n=168; p=0.02) patient groups, with a high 3-year LRFS in patients with Class 1 results (HR, 97.2%; VHR, 97.4%) and low LRFS in patients with Class 2B results (HR, 92.6%; VHR, 76.7%) (FIG. 24).

The 20-GEP significantly stratified risk of poor outcomes with a high NPV, thus enabling clinicians to make risk-aligned management decisions for their patients.

### Example 10: Association of the 40 Gene Expression Profile with Risk of Metastatic Disease Progression of Cutaneous Squamous Cell Carcinoma and Specification of Benefit of Adjuvant Radiation Therapy (ART)

Of nearly 2 million cases of cutaneous squamous cell carcinoma (cSCC) diagnosed each year, approximately 74,000 (3.7%-5.2%4) will progress to nodal or distant metastasis. For patients deemed to be at high risk of recurrence, current management guidelines typically recommend definitive surgical resection where possible, followed by consideration of adjuvant radiation therapy (ART), possibly in combination with systemic therapies. The decision to prescribe ART is challenging given the large number and extent of recommended clinicopathologic factors that inform risk assessment and indications for ART. These decisions are further complicated because these clinicopathologic factors have not been consistently and independently demonstrated to predict benefit from ART.

Adjuvant radiation therapy (ART) for cutaneous squamous cell carcinoma is recommended based on a number of wide-ranging clinicopathologic features, which encompass a broad array of patients. The 40-gene expression profile (GEP) test classifies cutaneous squamous cell carcinoma tumors into low (class1), higher (class2A), or highest (class2B) risk of nodal and/or distant metastasis. This study demonstrates that: (1) local recurrence is associated with metastatic disease progression; and (2) the 40-GEP predicts an increase (improvement) in metastasis-free survival after receiving ART.

### Methods

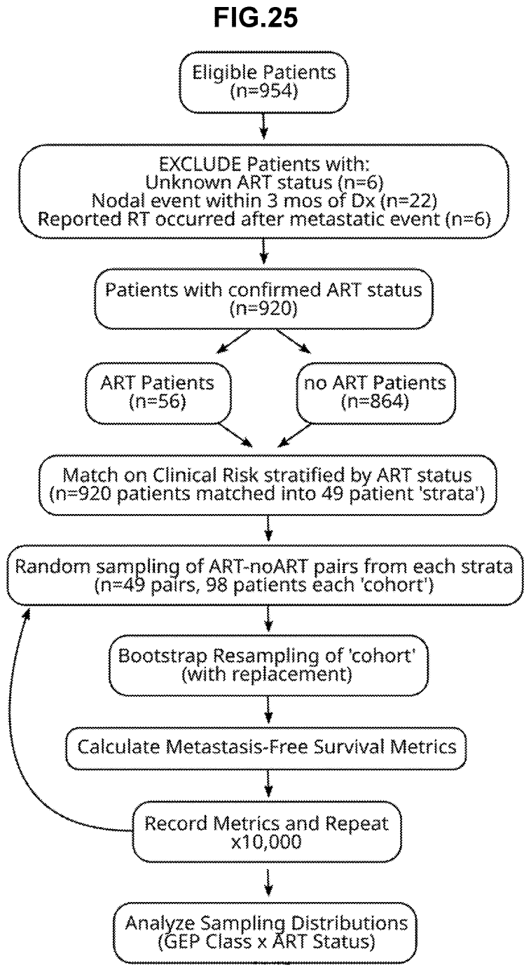

This study was conducted under an institutional review board approval including waiver of patient consent. Archival formalin-fixed, paraffin embedded cSCC tumor tissue and associated clinicopathologic data were obtained for patients diagnosed with cSCC. Tissue from 1 single primary tumor for each patient could be enrolled in the study, and in patients with multiple cSCCs, clinicians were asked to submit the tumor believed to have the greatest risk of disease progression. To be accepted into the study, cases were required to have availability of the original paraffin-embedded tissue block from the primary tumor and a minimum of 3 years of documented follow-up or report of a nonlocal metastatic event (regional or distant). Selection of samples was without consideration or stipulation regarding treatments, and 40-GEP test results were unknown to the sites. Inclusion/exclusion criteria; of note, cases with a prior history of cSCC, cutaneous basal cell carcinoma, or melanoma in situ were accepted if these prior malignancies were considered cured. Only patients who would qualify for 40-GEP testing on the basis of having at least 1 qualifying risk feature were included. Qualifying features were those listed in the National Comprehensive Cancer Network (NCCN) risk assessment categories of “High” or “Very High” risk, plus 1 additional qualifying feature of tumor diameter ≥2 cm (1.7% of this cohort). Eligibility for this present analysis is shown in the flow diagram in FIG. 25, and demographics of the cohort are presented in Table 21. The 40-GEP risk status of the included patients was unknown by clinicians at the time of diagnosis and treatment decisions.

TABLE 21
Patient and Tumor demographics for Example 10.

| | | | | |
|---|---|---|---|---|
| | | Non-ART- | ART- | |
| | All | treated | treated | |
| Risk factor | (n = 920) | (n = 864) | (n = 56) | P value* |

40-GEP class, n (%)

| | | | | | | | |
|---|---|---|---|---|---|---|---|
| Class 1 | 507 | (55.1%) | 496 | (57.4%) | 11 | (19.6%) | <.001 |
| Class 2A | 370 | (40.2%) | 335 | (38.8%) | 35 | (62.5%) | |
| Class 2B | 43 | (4.7%) | 33 | (3.8%) | 10 | (17.9%) | |

Metastatic event (nodal and/or distant), n (%)

| | | | | | | | |
|---|---|---|---|---|---|---|---|
| Yes | 113 | (12.3%) | 91 | (10.5%) | 22 | (39.3%) | <.001 |
| No | 807 | (87.7%) | 773 | (89.5%) | 34 | (60.7%) | |

Follow-up time, median (IQR; range)

| | | | | | | | |
|---|---|---|---|---|---|---|---|
| Total follow-up or time | 4.2 | (§2.0; | 4.23 | (§1.9; | 3.4 | (§4.1; | .01 |
| to event (y) | | 0.12 to | | 0.12 to | | 0.47 to | |
| | | 15.53) | | 15.53) | | 13.5) | |

Risk matching

| | | | | | | | |
|---|---|---|---|---|---|---|---|
| Age (y), median (range) | 72 | (26 to ≥90) | 70 | (26 to ≥90) | 72 | (41 to ≥90) | .89 |
| Location, n (%) | | | | | | | |
| Head and neck | 598 | (65.0%) | 553 | (64.0%) | 45 | (80.4%) | .04 |
| Anogenital, acral, pretibial | 132 | (14.4%) | 127 | (14.7%) | 5 | (8.9%) | |
| Trunk, extremities | 190 | (20.7%) | 184 | (21.3%) | 6 | (10.7%) | |
| Immunocompromised, n (%) | 233 | (25.3%) | 222 | (25.7%) | 11 | (19.6%) | .31 |
| Poorly differentiated, n (%) | 147 | (16.0%) | 117 | (13.5%) | 30 | (53.6%) | <.001 |
| Tumor diameter ≥2 cm, n (%) | 362 | (39.4%) | 319 | (36.9%) | 43 | (76.8%) | <.001 |
| Tumor thickness ≥6 mm, n (%) | 74 | (8.0%) | 53 | (6.1%) | 21 | (37.5%) | <.001 |
| Invasion beyond subcutaneous | 136 | (14.8%) | 98 | (11.3%) | 38 | (67.9%) | <.001 |
| fat, n (%) | | | | | | | |
| Perineural invasion,^{y} n (%) | 41 | (4.5%) | 26 | (3.0%) | 15 | (26.8%) | <.001 |
| Surgery type, n (%) | | | | | | | |
| Mohs | 606 | (65.9%) | 588 | (68.1%) | 18 | (32.1%) | <.001 |
| Wide local excision | 278 | (30.2%) | 242 | (28.0%) | 36 | (64.3%) | |
| Otherz | 36 | (3.9%) | 34 | (3.9%) | 2 | (3.6%) | |
| Other characteristics, n (%) | | | | | | | |
| Male sex | 675 | (73.4%) | 630 | (72.9%) | 45 | (80.4%) | .22 |
| Race | | | | | | | |
| Caucasian | 825 | (89.7%) | 778 | (90%) | 47 | (83.9%) | .004 |
| Hispanic or Latino | 54 | (5.9%) | 45 | (5.2%) | 9 | (15.1%) | |
| African American | 6 | (0.7%) | 6 | (0.7%) | 0 | (0%) | |
| Asian | 2 | (0.2%) | 2 | (0.2%) | 0 | (0%) | |
| Not specified | 33 | (3.6%) | 33 | (3.8%) | 0 | (0%) | |
| Lymphovascular invasion | 17 | (1.9%) | 11 | (1.3%) | 6 | (10.7%) | <.001 |

Staging and risk assessment, n (%)

| BWH | | | | | | | |
|---|---|---|---|---|---|---|---|
| T1 | 437 | (47.5%) | 435 | (50.4%) | 2 | (3.6%) | <.001 |
| T2a | 339 | (36.6%) | 326 | (37.7%) | 13 | (23.2%) | |
| T2b | 105 | (11.4%) | 82 | (9.5%) | 23 | (41.1%) | |
| T3 | 39 | (4.2%) | 21 | (2.4%) | 18 | (32.1%) | |
| AJCC8 | | | | | | | |
| T1 | 491 | (53.4%) | 486 | (56.3%) | 5 | (8.9%) | <.001 |
| T2 | 214 | (23.3%) | 210 | (24.3%) | 4 | (7.1%) | |
| T3 | 185 | (20.1%) | 153 | (17.7%) | 32 | (57.1%) | |
| T4 | 30 | (3.3%) | 15 | (1.7%) | 15 | (26.8%) | |
| NCCN risk | | | | | | | |
| Low^{x} | 15 | (1.6%) | 15 | (1.7%) | 0 | (0.0%) | <.001 |
| High | 568 | (61.7%) | 562 | (65.1%) | 6 | (10.7%) | |
| Very high | 337 | (36.6%) | 287 | (33.2%) | 50 | (89.3%) | |

Abbreviations: AJCC8 = American Joint Committee on Cancer Eighth Edition; ART = adjuvant radiation therapy; BWH = Brigham and Women's Hospital; GEP = gene expression profile; NCCN = National Comprehensive Cancer Network.
*P value reported for Person x2 or Wilcoxon F test, as appropriate.
^{y}Presence of perineural invasion.
^{z}Other surgery types: biopsy and electrocauterization.
^{x}Tumor size 2 cm, n = 6; infiltrating histology, n = 7; tumor thickness 2 to 3 mm, n = 2.

All analyzed patients had definitive surgery and a subset had ART. ART-treated patients were enrolled by 29 of the 59 contributing sites, with each site contributing 2% to 14% of the total number of ART-treated patients in this study (mean, 2.15 patients/site). Treatment volume (available for 54 of the 56 ART-treated patients) indicated that in addition to the primary site, the regional basin was treated in 17% of patients with class 2A cSCC and 20% of patients with class 2B cSCC. From responses of 916 of 920 patients originally screened for this study, only 1.7% received adjuvant systemic therapy; given its low prevalence, it was not feasible to control for this feature in addition to ART. Among those patients who received ART, the percentages of patients also receiving adjuvant systemic therapy were 18% with class 1 cSCC, <10% with class 2A cSCC, and 20% with class 2B cSCC.

Due to the inherent biases that would have guided ART treatment decisions at certain centers, ART-treated and ART-untreated patients were matched according to clinicopathologic risk factors in order to control the analyses. Based on propensity score matching using a general linear model (MatchIt_4.5.3 package in R v4.3.0, using “optimal full” matching for a 1:1 ratio), ART-treated and ART-untreated patients were matched on age, immunosuppression status, tumor location, tumor diameter, tumor thickness, invasion of fat, perineural invasion, and type of definitive surgical treatment received. Propensity score matching resulted in a successful match for all 920 patients into 49 matched strata, in which 1 or more ART-treated patients were matched to 1 or more ART-untreated patients. To use all possible matched patient pairs without excluding any patients, ×10,000 iterations of random pairs of ART-treated and ART-untreated patients from each matched stratum were sampled (n=98 patients, 49 pairs), and then bootstrap resampling (with replacement, n=98) was used to allow generalization of the results to the global sample set (FIG. 20). Each iterated cohort was analyzed as described below. Note that the number of iterations invoked when using random resampling methods is a function of result stability, such that the greater the number of iterations the less likely that the results may be different depending on the random seed set at the start; this is different than a “modeling” effort for which overfitting is a concern.

Samples were obtained from 920 patients who were matched on clinical risk factors and stratified by ART status, to create 49 matched patient strata. Randomly sampled pairs of matched ART and non-ART patients comprising 10,000 resampled cohorts were each analyzed for 5-year metastasis-free survival (nodal and/or distant events; MFS) and predicted time to metastatic event (using a standard ‘life expectancy’ algorithm).

### Statistical Analyses

Each sample for each iteration formed a cohort. Patients within each sample represented a range of risk levels, matched between ART-treated and ART-untreated patients. Each sample cohort was analyzed using survival analysis (survival_3.5-3 package in R v4.3.0), and using the statistical method of “life expectancy” (substituting time to metastatic event in place of death), estimations for each sampled cohort were calculated for regional or distant metastatic events and recorded for each bootstrapping iteration, for each 40-GEP class×ART treatment status group (k=6 groups). The primary comparisons were between ART-treated and ART-untreated patients within a given GEP class. The distribution of metastatic event types from this analysis was 66.4% regional only, 29.2% regional and distant, and 4.4% distant only.

The Kolmogorov-Smirnov 2-sample test was used to determine statistical differences in sampling distributions of estimated time to metastatic event between ART-treated and ART-untreated patients. However, because the number of observations in each of the tested distributions reflected the number of bootstrap iterations and not the sample number, the critical value (Dcrit) for significance was adjusted to appropriately reflect the n of each sample group (n=98/k=16.33 per group), in:

$${D}_{crit} = c ( α ) \times sqrt ( [ {n}_{1} + {n}_{2} / {n}_{1} \times {n}_{2} ] )$$

for alpha (α) values of 0.05, 0.01, and 0.001. An observed D value (D_{obs}) greater than Derit indicated a significant difference between the distributions. For this data set, the D_{crit} values were 0.48, 0.57, and 0.68, respectively, for the a values of 0.05, 0.01, and 0.001. Significance was treated as a 1-sided test, in which significance would result if the ART-treated group showed a benefit of a longer time to predict metastatic events. It is very important to note that the time to predict a metastatic event for a given resampled cohort reflects the point at which >50% of the cohort is projected to experience an event, with longer event times reflecting a mathematical ceiling on that projection, rather than indicating that a given group will experience actual events at that time point. In order to address the frequency with which an ART-treated group consistently showed better outcomes within each 40-GEP class for each sampled cohort, the estimated time to a metastatic event for the ART-untreated group was subtracted from that of the ART-treated group for each sample iteration. Therefore a positive difference indicated a longer time to a metastatic event for the ART-treated group.

Extensive control analyses were performed to test the robustness of the results as well as to check for any procedural biases or spurious correlations. One important control was to randomize the GEP result for each sampled cohort to ensure that the low frequency of the class 2B designation could not procedurally generate the observed results. Other control analyses tested whether any of the individual matched risk factors alone could identify an ART benefit as well as 40-GEP, or which in matching might have led to a spurious result falsely attributed to 40-GEP. For the former, the entire analysis was repeated excluding each risk factor from the matching and stratifying that factor with ART treatment status. For the latter, each matching factor was excluded 1 at a time to determine whether the 40-GEP determinations held. Finally, alternative risk and staging schemas were tested to determine whether any could identify an ART-benefiting group of patients as well as 40-GEP.

### Results

### Patterns of Disease Relapse and Temporal Sequence of Progression

In this cohort of 920 patients, 113 of 920 experienced a nonlocal recurrence (regional or distant, 12.3%) and 96 of 920 experienced a local recurrence (10.4%). Of the 96 patients reporting a local recurrence, all had received definitive surgical treatment and 54 (56.3%) had progressed to metastasis (nodal or distant). Of these 54 patients who developed metastasis, 98% developed nodal disease and 64.8% also developed distant metastases. This sequence of disease-associated events supports a mechanism of progression from local recurrence to regional and distant metastatic events.

Among patients who had both local recurrence and metastasis, 88.9% reported the local recurrence event before or in conjunction with the metastatic event, and 41 (75.9%) reported the local event as occurring before the metastatic event. The median time between local recurrence and metastasis was 3 months (IQR, 1.2-8 months; Wilcoxon test, P<001; H0: time delta, 0), but 21 (51.2%) experienced a regional or distant metastasis at >3 months after local recurrence. Similarly, 47.8% (54/113) of metastases were associated with a local recurrence, and in the presence of local recurrence, metastasis occurred in an accelerated timeframe as compared with metastasis in the absence of local recurrence (median of 9.3 months from diagnosis to metastatic event versus 13 months; Wilcoxon test, P=0.06).

### Analysis of 40-GEP Testing in Relation to Differences in Metastatic Risk Depending on ART Treatment

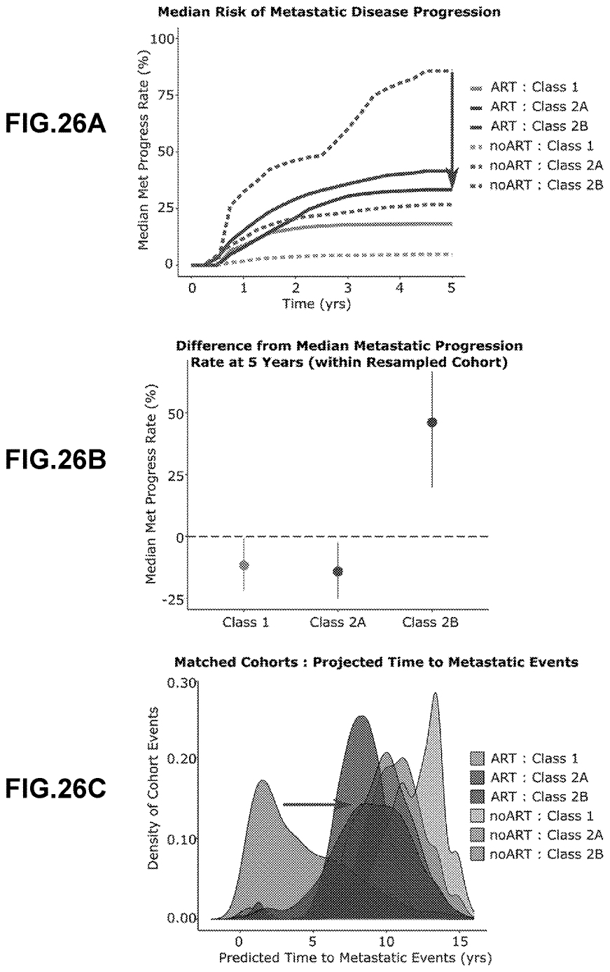

Patients with verified ART-treated or -untreated status were risk-matched on multiple clinical features, analyzed for risk of metastatic disease progression (using survival methods) and projected time to metastatic event, and stratified by 40-GEP results and ART status (see Methods; ART-untreated: 496 class 1, 335 class 2A, and 33 class 2B; ART-treated: 11 class 1 [nodal region irradiated in 1/11], 35 class 2A [nodal region irradiated in 6/35], and 10 class 2B [nodal region irradiated in 2/10]). Median disease progression rates for the resampled cohorts demonstrated a 50% decrease, on average, specifically for class 2B ART-treated patient cohorts compared with class 2B untreated patient cohorts at 5 years after diagnosis (FIG. 26A). Improvement was also observed in the within-cohort differences in 5-year metastatic progression rates only for the patients with class 2B cSCC (ART-untreated-ART-treated, 46.2% median difference; FIG. 26B). ART decreased the disease progression rate of patients with class 2B cSCC to a rate similar to that of patients with class 2A.

The estimated time to metastatic event predictions was generated for each sampled cohort. Note that the time to metastasis is based on cohort probability trajectories as used in traditional life expectancy calculations (see Methods). The observed data for a given sampled cohort (accumulation of events over time) are used to project the point in time where at least half of the cohort would have experienced an event based on the observed trajectory, such that longer times reflect the ceiling of that projection (mathematically). The distributions of results from the 10,000 resampled cohorts are shown in FIG. 26C and reveal a substantial difference in projected times to metastatic events between ART-treated and -untreated patients with class 2B cSCC. For ART-untreated patients, there is a peak rate of metastasis around 2 years, whereas for ART-treated class 2B patient cohorts there is a nearly 5-fold increase in the mathematically projected time to metastatic events. It is critical to understand that the peak timing observed for ART-treated patients with class 2B cSCC is near those observed for patients with class 1 and 2A cSCC independent of ART treatment and thus likely reflects a ceiling in the mathematical projection, not a prediction for event occurrence at that time.

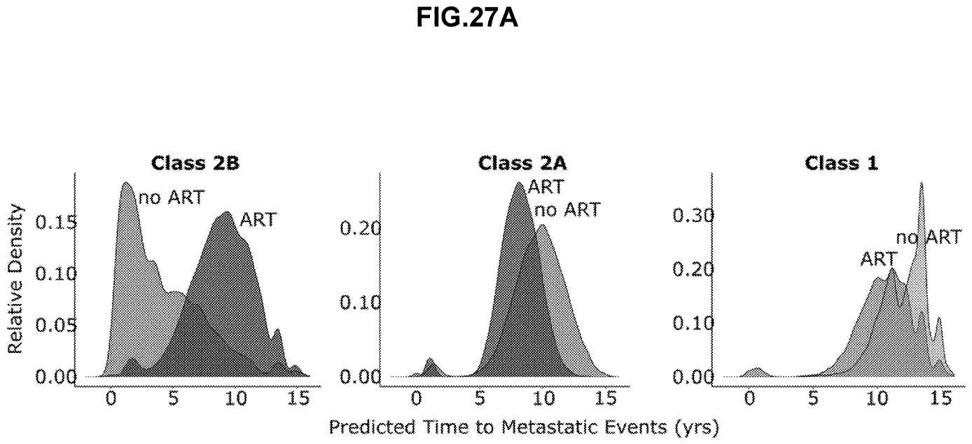

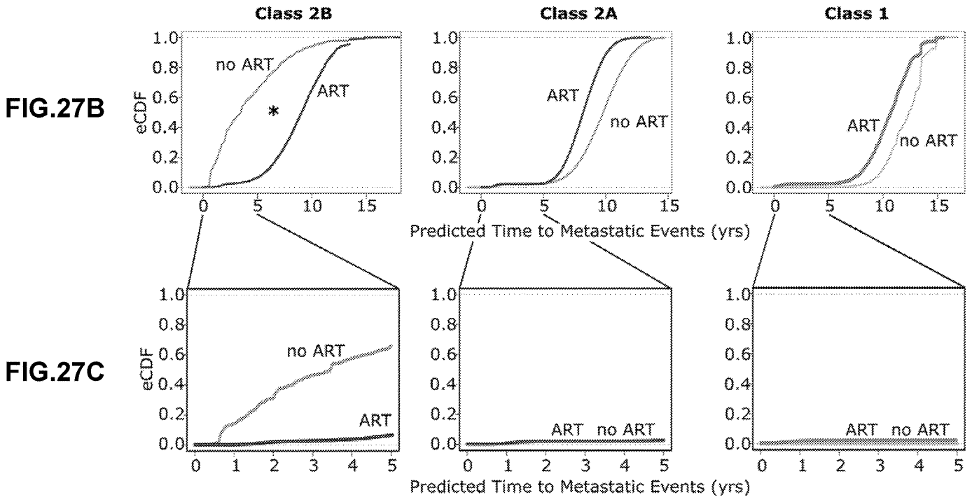

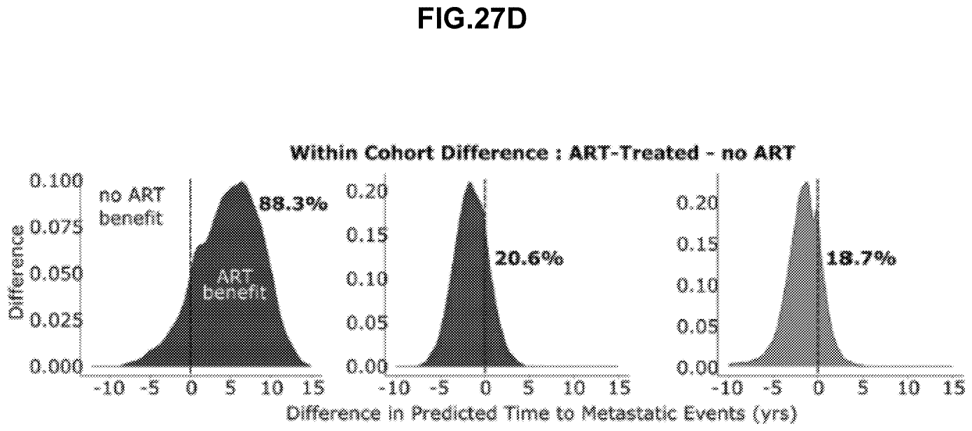

The Kolmogorov-Smirnov test was used to determine whether the distributions of time to metastatic event were significantly different between ART-treated and ART-untreated patients for each 40-GEP class result. ART difference in the rate of accumulated cohort events (P<0.01; class 2B D_{obs}=0.61; FIG. 27A, 27B), whereas differences between ART-treated and ART-untreated in class 2A and class 1 were not statistically significant (P>0.05; class 2A and class 1 D_{obs}=0.38 and 0.31, respectively; FIG. 27A, 27B). The cumulative probability distributions for each 40-GEP group of ART-treated and ART-untreated patients (a simple transformation of the time to event distributions as accumulating over time, reflecting the velocity of distribution shape) demonstrated the underlying functions associated with disease progression in each patient group (FIG. 27B, 27C). ART-treated and ART-untreated class 1 and class 2A patient cohorts accumulated metastatic events according to a sigmoidal function, in which events began to accumulate at a low rate and then increased monotonically over a 5- to 6-year period until the prediction became asymptotic. In sharp contrast, ART-untreated class 2B patient cohorts showed an abrupt increasing exponential accumulation of events, suggesting a different underlying process of disease progression that was accelerated relative to patients with class 1 and 2A cSCC. Interestingly, the disease progression of patients with class 2B cSCC treated with ART reverted to a function and time course that mimicked those of patients with class 2A cSCC, reflecting a decreased velocity in the accumulation of metastatic events over time as compared with ART-untreated patients with class 2B cSCC.

Of further interest is how often the ART-treated class 2B group might show deceleration of projected disease progression relative to the ART-untreated patients with class 2B cSCC. Therefore, for each of the 10,000 resampled iterations, the predicted time to metastatic events between the ART-treated class 2B group was subtracted from that of the ART-untreated class 2B group (FIG. 27D). When estimated in this manner, the ART-treated class 2B subset demonstrated a longer time to disease progression than the ART-untreated class 2B subset in 88.3% of the resampled cohorts. In contrast, class 1 and 2A patient subsets showed this benefit from ART in only 18.7% and 20.6% of resampled cohorts, respectively (FIG. 27D).

### Control Analyses

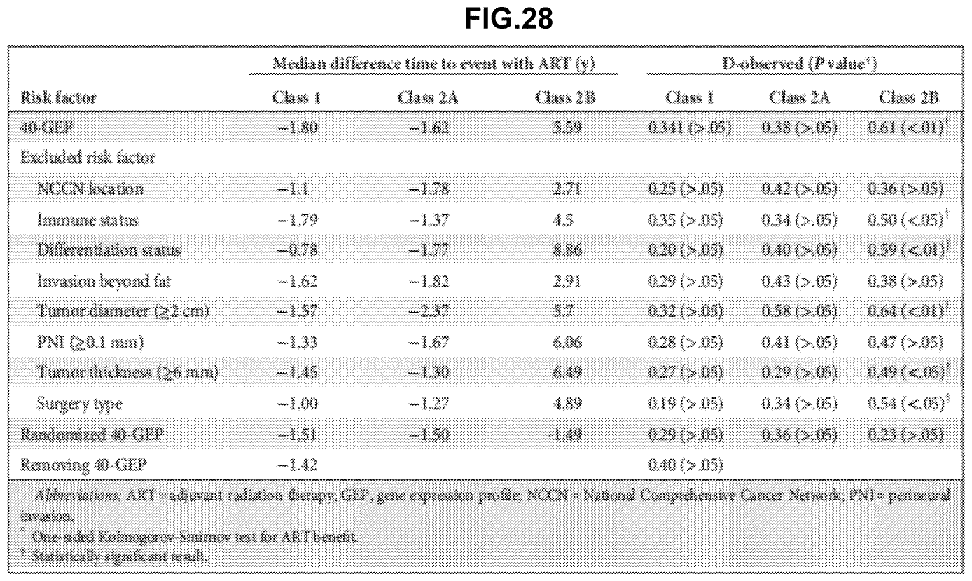

First, to determine whether the observed results could have been driven by the matching to any single clinicopathologic risk factor, each factor was systematically excluded from the matching scenario. In this control analysis, the same overall result (that a class 2B result identifies patients who will benefit from ART in terms of metastatic progression) was observed regardless of which factor was not used in matching, including type of definitive surgery used (FIG. 28, “Excluded risk factor”). Although not every result was statistically significant, each revealed a benefit for ART-treated class 2B patient cohorts as a positive difference in median time to metastatic event, which was not observed for class 2A or class 1. Second, as the proportion of 40-GEP class results was not the same within the cohort, with class 2B being observed less frequently than class 1 or class 2A (FIG. 28), it was tested whether these proportion differences could have influenced the findings. Therefore, 40-GEP results were randomly reassigned to ART-treated and ART-untreated patients after each resampling procedure. The results clearly showed that the proportional differences of 40-GEP class results did not drive a spurious finding and that the randomization of the test result negated the findings (FIG. 28, “Randomized 40-GEP”). Third, and perhaps most important, removing the 40-GEP results from the analysis and stratifying by ART status alone also did not reveal a metastatic prevention benefit from ART in patient cohorts matched by clinical factors (FIG. 28, “Removing 40-GEP”).

### Experimental Analyses

Additional analyses tested whether any of the clinicopathologic risk factors used for matching, when combined with ART status, could themselves be used to identify patients who would benefit from ART treatment (Table 22; note that the stratifying feature was excluded from matching for this test). No risk factor combined with ART status was able to better identify a patient group that would potentially benefit from prevention of metastatic progression through ART treatment. Finally, a last set of comparisons was to use the NCCN risk category (high or very high risk) or stage (Brigham and Women's Hospital [BWH] T1/2a and T2b/3, or American Joint Committee on Cancer T1/2 and T3/4, as binary risk levels) in place of the 40-GEP result to determine whether a group of patients who would have prevention of metastatic progression could be identified solely based on these risk assessment criteria; no patient cohort groups could be identified (Table 22, bottom).

TABLE 22
Experimental analyses results.

| | | |
|---|---|---|
| Prediction | Median difference | |
| variable | time to event | D-observed |
| Level | with ART (y) | (P value*) |

40-GEP

| | | |
|---|---|---|
| Class 1 | −1.80 | 0.34 (>.05) |
| Class 2A | −1.62 | 0.38 (>.05) |
| Class 2B | +5.59 | 0.61 (<.01)^{y} |

Risk factor
Location

| | | |
|---|---|---|
| L | −2.98 | 0.45 (>.05) |
| M | −3.41 | 0.47 (>.05) |
| H | −1.37 | 0.36 (>.05) |

Immune status

| | | |
|---|---|---|
| Compromised | −0.78 | 0.177 (>.05) |
| Competent | −2.09 | 0.519 (>.05) |

Differentiation status

| | | |
|---|---|---|
| Well or moderate | −0.62 | 0.16 (>.05) |
| Poor | −0.98 | 0.29 (>.05) |

Invasion beyond fat

| | | |
|---|---|---|
| Yes | −1.23 | 0.33 (>.05) |
| No | −0.75 | 0.18 (>.05) |

Tumor diameter

| | | |
|---|---|---|
| ≥2 cm | −1.51 | 0.40 (>.05) |
| <2 cm | −2.01 | 0.43 (>.05) |

PNI

| | | |
|---|---|---|
| ≥0.1 mm | −1.4 | 0.31 (>.05) |
| <0.1 mm | −2.3 | 0.60 (>.05) |

Tumor thickness

| | | |
|---|---|---|
| ≥6 cm | −1.76 | 0.37 (>.05) |
| <6 cm | −1.10 | 0.29 (>.05) |

Surgery type

| | | |
|---|---|---|
| Mohs | −11.8 | 0.85 (>.05) |
| WLE | −0.02 | 0.09 (>.05) |
| Other | −1.07 | 0.61 (>.05) |

Risk assessment method
NCCN risk category

| | | |
|---|---|---|
| High | −1.49 | 0.26 (>.05) |
| Very high | −0.85 | 0.29 (>.05) |

BWH T-stage

| | | |
|---|---|---|
| Low (T1/T2a) | −0.77 | 0.18 (>.05) |
| High (T2b/T3) | −0.89 | 0.26 (>.05) |

AJCC8 T-stage

| | | |
|---|---|---|
| Low (T1/T2) | −1.80 | 0.32 (>.05) |
| High (T3/T4) | −0.40 | 0.18 (>.05) |

Abbreviations: AJCC8 = American Joint Committee on Cancer Eighth Edition; ART = adjuvant radiation therapy; BWH = Brigham and Women's Hospital; GEP, gene expression profile; NCCN = National Comprehensive Cancer Network; PNI = perineural invasion; WLE = wide local excision; L = Trunk, Extremities; M = Anogenital, Acral, Pretibial; H = Head, Neck.
*One-sided Kolmogorov-Smirnov test for ART benefit.
yStatistically significant result.

The distribution of 40-GEP results across patients with BWH T3 cSCC was 21%, 64%, and 15% for class 1, 2A, and 2B, and 33%, 53% and 13% for patients with T2b cSCC, respectively. The odds ratio of a T3 tumor receiving a class 2B was only 1.18 times that of a T2b tumor. The distribution of patients with class 2B cSCC according to 40-GEP across BWH stages was 28%, 26%, 33%, and 14% respectively for T1 to T3, with a patient with T3 tumor being twice as likely to receive a class 2B than class 2A result and a T2b being 2.7 times as likely to receive a class 2B versus a class 2A result. Together, these data suggest that there is not a strong relationship between class 2B 40-GEP results and BWH T3. Similarly, the distribution of 40-GEP results for patients with tumors ≥6 cm in diameter was 22%, 59%, and 19%, again failing to show a strong relationship between class 2B and large tumor diameter. Overall, when compared with all other methods of risk assessment, only patients with class 2B cSCC according to 40-GEP were independently identified as benefiting from reduced metastatic risk after ART treatment.

Of 96 patients experiencing local recurrence, 56.3% experienced metastasis; of those experiencing both, 88.9% experienced local recurrence before (75.9%) or concurrently (13.0%) with metastasis. After matching for clinicopathologic risk factor, median 5-year disease progression rates for resampled cohorts demonstrated approximately 50% improvement for class 2B ART-treated compared with ART-untreated cohorts. ART-treated class 2B cohorts had a 5-fold delay in predicted time to metastatic event and deceleration of disease progression compared with ART-untreated cohorts (Kolmogorov-Smirnov test, P<01); this was not observed for patients with class 1 or 2A cSCC (P>0.05 for each). No risk factor or staging system combined with ART status identified groups that would benefit from ART as well as 40-GEP.

The 40-GEP test identifies patients both at highest risk of nodal or distant metastasis as well as those patients who are most likely to benefit from ART. Additionally, as the 40-GEP test identifies low-risk patients who are less likely to benefit from ART, and given a low risk of metastasis can consider deferring treatment. These findings provide greater specificity than current clinicopathologic guidelines on which to base decisions for ART in patients.

### Example 11. Predicting Adjuvant Radiation Therapy Benefit in Cutaneous Squamous Cell Carcinoma with the 40-Gene Expression Profile

### Study Protocol & Patient Eligibility

Institutional review board approval was obtained for the research described. Tumors were eligible for inclusion if they met the following criteria: pathologically confirmed primary, invasive cSCC diagnosed between 1 Jan. 2011 and 1 Jan. 2022 for whom formalin-fixed, paraffin-embedded primary or recurrent tumor tissue was available for 40-GEP testing, associated de-identified tumor and patient clinicopathological information, treatment and outcomes data (local recurrence, regional or distant metastasis and death) were available, a minimum of 2 years of documented clinical follow-up or until time of regional or distant metastasis, or death and at least one of the following risk factors: tumor diameter ≥2 cm, poor or moderate differentiated histopathology, >6 mm depth of invasion or invasion into or beyond subcutaneous fat, small or large caliber PNI, LVI, or desmoplastic subtype. Tumors were excluded from the study if: the patient was <18 years of age and the tumor was recurrent. Tumors with a multigene failure 40-GEP result were excluded from analysis (n=23 tumors; FIG. 24). For the purposes of the current study and in alignment with the original findings, ART was defined as treatment prior to any recurrent event, including local, regional/nodal or distant metastasis.

### Clinicopathologic Risk Factor Matching in Resampled Patient Cohorts

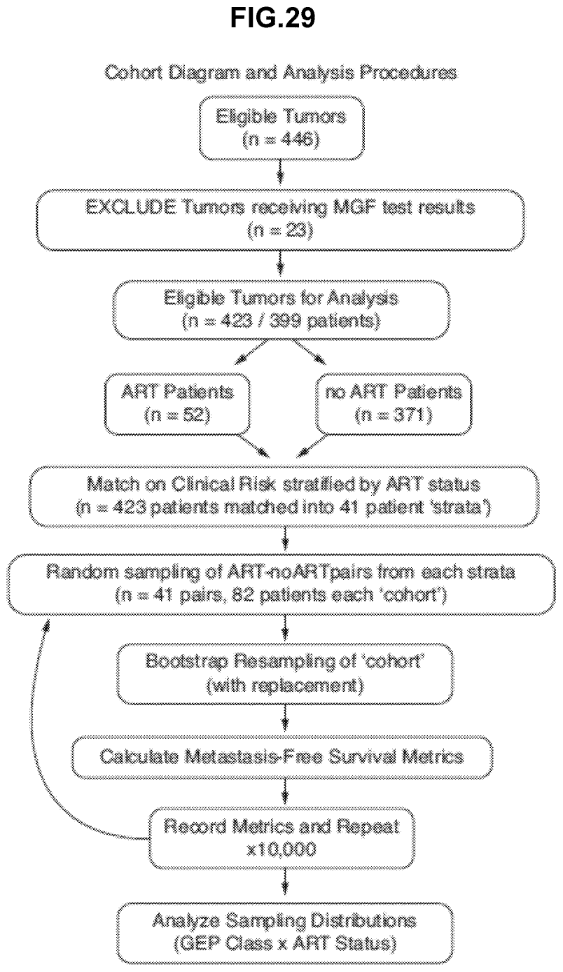

Primary methodology and the statistical analysis plan have been described previously (Arron et al. Int J Radiat Oncol Biol Phys. Vol. 120, No. 3, pp. 760-71, 2024). Briefly, ART-treated and untreated tumors were matched according to clinicopathologic risk factors typically used for formal risk assessment and definitive surgical treatment based on propensity score matching using a general linear model (MatchIt_4.5.3 package in R v4.3.0, using “optimal full” matching for a 1:1 ratio). Due to the inherent biases that could have guided ART treatment decisions, ART-treated and untreated tumors were matched on age, immune status, tumor location (head or neck, special site such as pretibial, anogenital, or acral, or trunk/limbs), tumor diameter (<2 cm, 2 to <4 cm, 4 to <6 cm or >6 cm), tumor thickness (<6 or ≥6 mm), invasion beyond subcutaneous fat, any PNI, and type of definitive surgical treatment received (Mohs, wide-local excision [WLE] or other). Propensity score matching resulted in a successful match for all 423 tumors in to 41 matched strata, in which one or more ART-treated tumors were matched to one or more ART untreated tumors. To utilize all possible matched patient pairs without excluding any qualifying tumors, ×10,000 iterations of random pairs of ART treated and ART untreated tumors from each matched strata were sampled (n=82 tumors, 41 pairs for each sampled cohort), and then bootstrap resampling (with replacement, n=82) was used to allow generalization of the results to the global sample set (FIG. 29).

### Statistical Analyses

Each sample cohort was analyzed using survival analysis and “life expectancy” estimations (modified for time to metastatic events in place of death; survival_3.5-3 package in R v4.3.0). Survival analysis yields a survival table, much like the life tables used in actuarial analyses. The observed rate of events over time can be used to mathematically project the amount of time until a defined cohort is dominated by events (i.e., the time at which the sample cohort has experienced >50% projected metastatic events, in this case). Metastasis-free survival and time to metastatic predominance were calculated for regional or distant metastatic events and recorded for each bootstrapping iteration, for each 40-GEP Class xART treatment status group (k=6 groups). The primary comparisons were between ART-treated and untreated patients within a given 40-GEP class. Note that metastasis-free survival is inverted and reported/plotted here as disease progression (95% metastasis-free survival for a given group at time X becomes 5% disease progression at time X). The Kolmogoro-v-Smirnov two-sample test was used to determine statistical differences in sampling distributions of projected time to metastatic events between ART-treated and ART-untreated patients (with the D-critical test statistic adjusted for the average number of tumors per group and was not based on the number of sampling iterations). It is important to note that the time to metastatic events is a projection regarding when a given resampled cohort will exceed a 50% event rate. This means that the longer event times projected reflect the mathematical ceiling on the projection, rather than indicating that a given cohort experienced events at that timepoint. A previous study did not report a benefit of ART in Class 2A tumors, therefore we tested the hypothesis that inclusion of the regional lymphatics (the “lymph node” or “nodal basin”) in addition to the tumor bed conferred a modest benefit to Class 2A tumors by removing all tumor bed+nodal basin ART treated tumors (n=17) and repeating the analysis. All procedures, including matching, were repeated on the modified dataset after dropping the aforementioned samples.

### Results

### Independent Cohort Description

All tumors included for analysis were diagnosed as primary cSCC (n=423) from 399 unique patients. The overall nonlocal metastasis rate for the cohort under study was 9.7% (with a 95% CI of 6.9-12.5%; 9.2% for regional/nodal metastases and 2.4% for distant metastases) and the overall local recurrence rate was 4.0% (95% CI: 2.4-5.9%). Most patients received Mohs as their definitive surgical treatment (74.7%), and 48.7% had a tumor located on the head or neck area (Table 23). 29.1% of patients were immunocompromised in this cohort. The cohort included 52 ART-treated tumors and 371 untreated tumors (FIG. 24) with a median follow-up of 3.0 years and 4.15 years, respectively. The distribution of 40-GEP results was 20 (Class 1), 24 (Class 2A), and 8 (Class 2B) for ART treated tumors, and 208, 144, and 19 for untreated tumors, respectively (Table 23). No difference in radiation dosages were observed between the 40-GEP risk classes that could account for differences in ART benefit (median±IQR, Class 1:5650±1000 cGy, Class 2A: 5900±1000 cGy, Class 2B: 6000±1000 cGy; Wilcoxon tests, p=0.98, 0.85, 0.87). Only 1.9 and 3.8% of ART-treated and 0 and 1.3% of untreated patients received chemotherapy or immunotherapy, respectively, and so these adjuvant therapies were not controlled for in the current analysis due to low prevalence.

TABLE 23
Cohort demographics-Example 11.

| | | |
|---|---|---|
| ART treated | No ART | Total |
| (n = 52) | ( n = 371) | (n = 423) |

40-GEP result

| | | | | |
|---|---|---|---|---|
| Class 1 | 20 | 208 | 228 | (53.9%) |
| Class 2A | 24 | 144 | 168 | (39.7%) |
| Class 2B | 8 | 19 | 27 | (6.4%) |

Max follow-up

| | | | | | | |
|---|---|---|---|---|---|---|
| Years | 3.02 | (0.38-8.4) | 4.15 | (0.19-10.9) | 4.13 | (0.19-10.9) |
| (median, range) | | | | | | |

Age at Dx

| | | | | | | |
|---|---|---|---|---|---|---|
| Years | 74.5 | (43-90+) | 76 | (30-90+) | 76 | (30-90+) |
| (median, range) | | | | | | |

Sex at birth

| | | | | |
|---|---|---|---|---|
| Female | 10 | 119 | 129 | (30.5%) |
| Male | 42 | 252 | 294 | (69.5%) |

Immune status

| | | | | |
|---|---|---|---|---|
| Immuno- | 37 | 263 | 300 | (70.9%) |
| competent | | | | |
| Immuno- | 15 | 108 | 123 | (28.1%) |
| suppressed | | | | |

Tumor location

| | | | | |
|---|---|---|---|---|
| Head/neck | 44 | 162 | 206 | (48.7%) |
| Trunk/ | 7 | 165 | 172 | (40.7%) |
| extremities | | | | |
| Special site ª | 1 | 44 | 45 | (10.6%) |

Tumor diameter ^{b}

| | | | | | | |
|---|---|---|---|---|---|---|
| largest dim | 3 | (0.3-15) | 2.2 | (0.3-18) | 2.3 | (0.3-18) |
| (cm) | | | | | | |

Histodifferentiation

| | | | | |
|---|---|---|---|---|
| Not Poor | 30 | 281 | 311 | (73.5%) |
| Poor | 22 | 90 | 112 | (26.5%) |

Invasion of fat

| | | | | |
|---|---|---|---|---|
| False | 16 | 291 | 307 | (72.6%) |
| True | 36 | 80 | 116 | (27.4%) |

Perineural invasion

| | | | | |
|---|---|---|---|---|
| False | 15 | 232 | 338 | (79.9%) |
| True | 37 | 48 | 85 | (20.1%) |

Lymphovascular invasion

| | | | | |
|---|---|---|---|---|
| False | 44 | 361 | 405 | (95.7%) |
| True | 8 | 10 | 18 | (4.3%) |

Primary treatment

| | | | | |
|---|---|---|---|---|
| Mohs | 37 | 279 | 316 | (74.7%) |
| WLE | 15 | 86 | 101 | (23.9%) |
| Other | 0 | 6 | 6 | (1.4%) |

BWH stage

| | | | | |
|---|---|---|---|---|
| T1 | 0 | 12 | 12 | (2.8%) |
| T2a | 5 | 283 | 288 | (68.1%) |
| T2b | 43 | 72 | 115 | (27.2%) |
| T3 | 4 | 4 | 8 | (1.9%) |

AJCCv8 stage

| | | | | |
|---|---|---|---|---|
| T1 | 2 | 67 | 69 | (16.3%) |
| T2 | 2 | 197 | 199 | (47.0%) |
| T3 | 48 | 107 | 155 | (36.4%) |

NCCN risk group

| | | | | |
|---|---|---|---|---|
| Low | 0 | 39 | 39 | (9.2%) |
| High | 0 | 164 | 164 | (38.8%) |
| Very high | 52 | 168 | 220 | (52.0%) |

^{a} Acral , pretibial , anogenital.
^{b} Missing = Three tumors.
40-GEP test results, patient and tumor characteristics and staging breakdowns, stratified by ART treatment status and including total breakdown for each variable. Categorical variables include distribution of levels observed and continuous variables report medians and ranges.
40-GEP: 40-gene expression profile; AJCCv8: American Joint Commission on Cancer, version 8; ART: Adjuvant radiation therapy; BWH: Brigham & Women's Hospital; dim: Dimension; Dx: Diagnosis; Histo: Histopathological; Lymphovas: Lymphovascular; NCCN: National Comprehensive Cancer Network; WLE: Wide-local excision.

The 40-GEP Test Identifies Tumors Most Likely to Benefit from ART

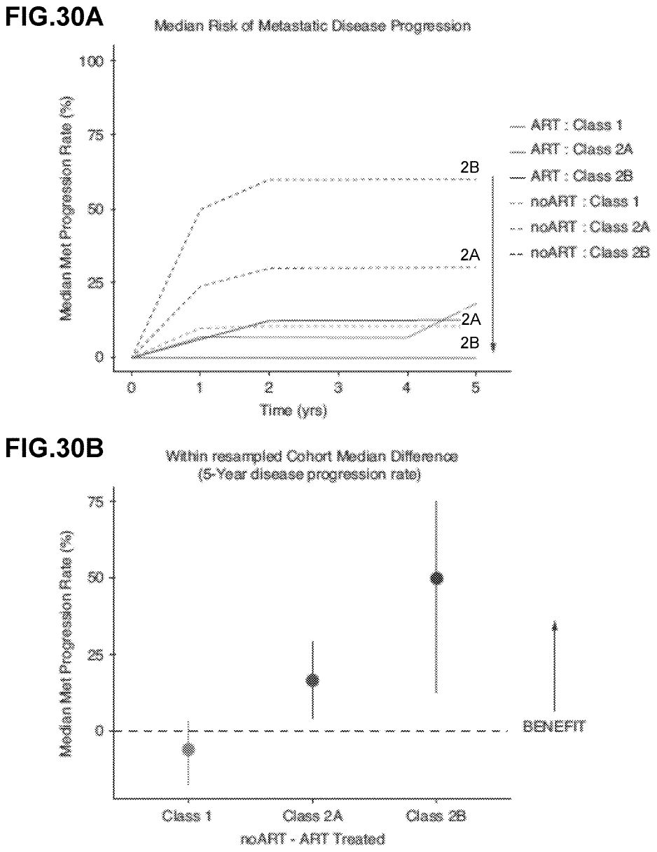

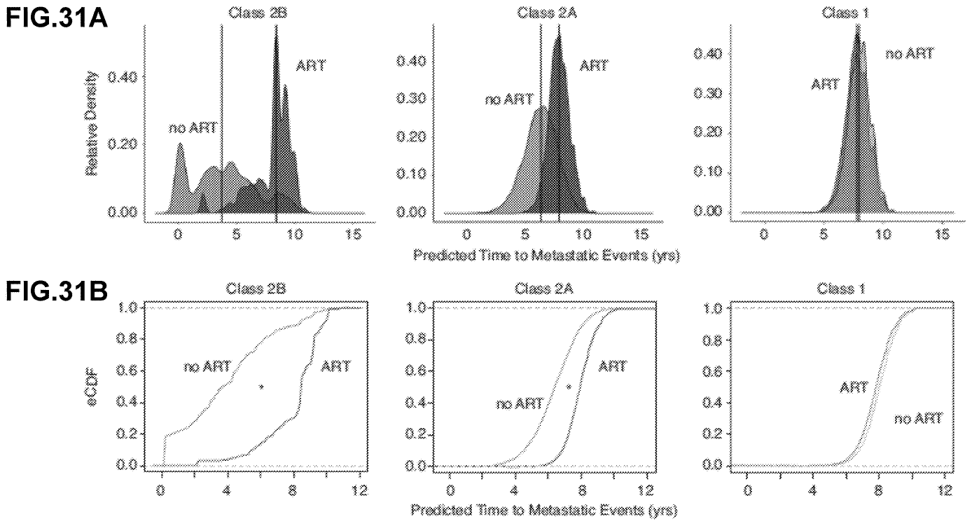

The median disease progression rates at 5 years post-diagnosis observed across all resampled cohorts was 60% for Class 2B untreated tumors and 0% for Class 2B ART-treated tumors (a >50% decrease) (FIG. 30A). Improvement was also observed between Class 2B ART-treated and untreated tumors within resampled cohorts at 5 years post diagnosis (ART-untreated-ART-treated, 50.0% median difference, FIG. 30B). A more modest benefit was observed for Class 2A ART-treated tumors (median disease progression: 30.5% untreated versus 12.5% ART-treated, FIG. 25A; within cohort difference: 16.6%, FIG. 30B). For Class 2B ART-untreated tumors, the median time to cohort metastases is 3.7 years, while for Class 2B, ART-treated tumor cohorts there is a 2.4-fold increase to 8.4 years. For Class 2A tumors there was a more modest shift from 6.4 years to 7.9 years (a 1.25-fold increase). The full distributions for each group are shown in FIG. 31A.

The time-to-metastases distributions of all Class 2B ART-treated versus untreated tumor cohorts was significantly different (p<0.01, Dobs=0.65). A statistically significant but more modest difference was observed between ART-treated and untreated Class 2A tumor cohorts (p<0.05, Dobs=0.53) and no significant difference for Class 1 tumor cohorts (p>0.05, Dobs=0.11; FIG. 31B). The cumulative probability distributions for each 40-GEP group of ART-treated and untreated patients show the underlying temporal functions of disease progression in each tumor group (FIG. 31B). The shift in the curve to the right for Class 2A ART-treated tumors relative to Class 2A untreated tumors indicates a delay in disease progression for ART-treated tumors. Class 2B untreated tumor cohorts showed a fast acceleration of disease progression, while Class 2B ART-treated tumor cohorts showed a significant delay in disease progression, similar to the time courses observed for Class 1 and 2A tumor cohorts (FIG. 31B).

### Control Analysis for Tumor Bed+Nodal Basin-Treated Tumors

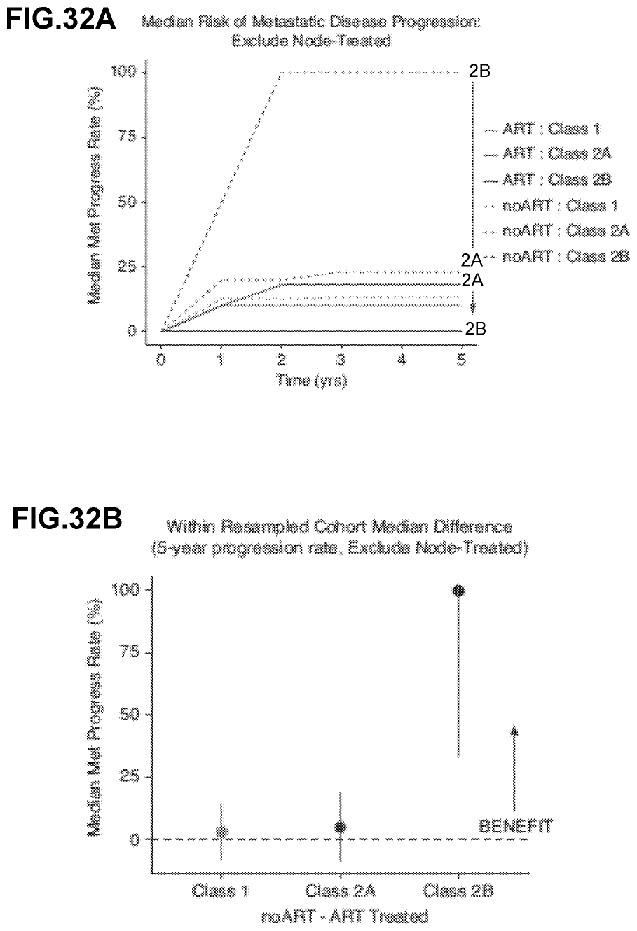

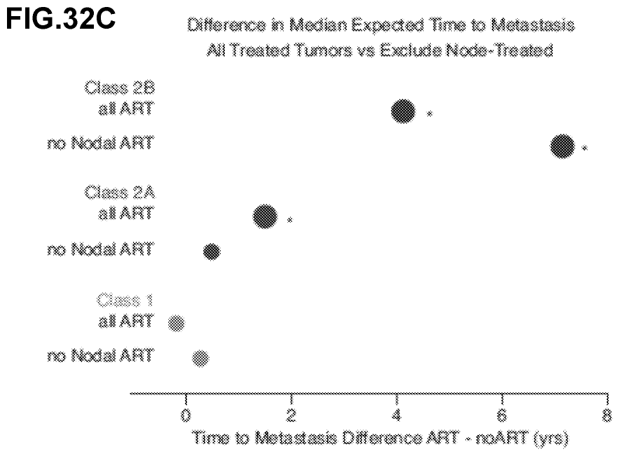

The number of tumors that received ART treatment at both the tumor bed and regional nodal basin was 35% (7/20) of Class 1 tumors, 37.5% (9/24) of Class 2A tumors, and 12.5% (1/8) of Class 2B tumors. The radiation dosages reported for tumor site plus nodal ART treatment were not different than for tumor site only treatment (median±IQR, Tumor+Node: 6000±600 cGy, Tumor Site only: 5500±1000 cGy; Wilcoxon test, p=0.25). Propensity score matching successfully matched all tumors to 28 strata. After controlling for nodal ART, a benefit was no longer observed between Class 2A ART-treated and untreated tumors in the median 5-year disease progression rates and within cohort differences (FIG. 32A-32B). When only the tumor bed was treated with ART, the 40-GEP Class 2A difference between ART treated and untreated tumors decreased from a benefit of 1.5-0.47 years, and no longer reflected a statistically significant benefit (p>0.05, Dobs=0.18; FIG. 32C). A significant difference was maintained for Class 2B tumors (p<0.001, Dobs=0.78; FIG. 32C; note that only a single patient was removed for this analysis), and there was no significant change for Class 1 regardless of ART treatment location (p>0.05, Dobs=0.11; FIG. 27C).

### Discussion

The data presented herein confirms the previously published utility of the 40-GEP test in differentiating high-risk cSCC tumors most likely to benefit from ART (Class 2B) and those less likely to benefit (Class 1) (Arron et al. Int J Radiat Oncol Biol Phys. Vol. 120, No. 3, pp. 760-71, 2024). Given the consistency of the results from two independent cohorts, the 40-GEP test can aid in ART decision-making for patients with high-risk cSCC tumors. A comparison of this study to the previous study from Arron and colleagues (Arron et al. Int J Radiat Oncol Biol Phys. Vol. 120, No. 3, pp. 760-71, 2024) demonstrates similarities in the observed results. Both studies controlled for treatment bias by clinicopathologically matching ART-treated and untreated patients. Both studies show that Class 2B ART-treated tumors have on average approximately a 50% decrease in metastatic disease progression compared with untreated tumors and a significant deceleration of disease progression. In addition, both studies report that there is no difference in progression rates when comparing Class 1 ART-treated and untreated tumors. Interestingly, Class 2A tumors showed a modest benefit in the current cohort that was not observed previously. In the current cohort under study, the Class 2A benefit could be attributed to the inclusion of patients treated with ART at both the primary tumor site and the nodal bed (versus primary site alone). This difference was not observed in Class 1 tumors which had a similar proportion of tumor site+node ART-treated patients and still showed no significant benefit. Confirmation of the possible benefit based on ART site selection for Class 2A patients requires additional investigation. The absence of a modest benefit for Class 2A tumors when treating the tumor site alone suggests that the metastatic risk of the tumor is unlikely to be the primary driver of identifying tumors that may benefit from ART.

Alternatively, it may be the specific gene expression signature captured by a 40-GEP Class 2B result that not only signals a tumor at advanced metastatic risk, but also captures a tumor that is more susceptible to radiation than a Class 1 or 2A tumor. Although ART can benefit a subset of patients with cSCC, it also has associated increased morbidity, including but not limited to, inflammation, pruritis, pain, ulcers, hair loss, nausea, lymphedema, and, rarely, secondary malignancy. In addition to ART-associated complications, ART can cost the healthcare system up to $1.4 billion annually for Medicare patients. One study showed that incorporating the 40-GEP into ART decision-making (specifically, deferring ART for patients with GEP Class 1 results) could save up to $784 million annually in direct costs (Somani et al. J Clin Aesthet Dermatol. 17:41-44, 2024). Therefore, integrating the 40-GEP into ART decision-making could mitigate ART-associated side effects in patients unlikely to benefit from ART (Class 1), identify patients who may benefit from ART (Class 2B), and result in substantial cost savings.

The data presented here demonstrate that the 40-GEP test can provide utility in this decision-making. A Class 2B result indicates both substantial risk for metastases and an increased likelihood to benefit from ART. A Class 1 result indicates both a low risk of metastasis and decreased likelihood to benefit from ART, and in the absence of a particularly worrisome clinical factor (such as LVI), can defer treatment until circumstances suggest intervention be reconsidered (such as a local recurrence). Finally, a Class 2A result indicates moderate risk that, in the presence of concerning risk factors, may guide a clinician to consider ART even though the likelihood of benefit may be lower for that patient.

The results from this study provide a validation of the performance of the previously reported benefit of ART for Class 2B tumors and a lack of benefit for Class 1 tumors, which in addition to the low rate of disease progression suggests these patients may consider deferring treatment. Given the risk of morbidities and cost of ART, the 40-GEP test can aid clinical decision-making for high-risk cSCC tumors where ART is being considered.

### Example 12. Prognostic and Adjuvant Radiation Therapy Benefit Prediction with the Integrated 40-Gene Expression Profile (i40-GEP) for High-Risk Cutaneous Squamous Cell Carcinoma

Clinicopathologic (CP) features are used for risk assessment in patients with high-risk cutaneous squamous cell carcinoma (cSCC), but this can result in under-treatment as well as over-treatment, and is the focus of continued improvements. Integration with tumor biology via a proven gene expression profile (GEP) can significantly improve risk stratification. Thus, a refined risk stratification of the 40-GEP through incorporation of clinicopathologic features was generated. The integrated 40-GEP (i40-GEP) adds five CP factors (immunosuppression, tumor location, poor histologic differentiation, tumor diameter, and perineural invasion), and reports results as Class 1A (Lowest Risk), Class 1B (Low Risk), Class 2A (High Risk), and Class 2B (Highest Risk).

The i40-GEP significantly stratified 3-year MFS (Class 1A=97.3%; Class 1B=94.7%; Class 2A=77.2%; and Class 2B=67.1%; p<0.001). Univariate analysis showed Class 2B carries the greatest metastatic risk (HR: 15.3) followed by Class 2A (HR=9.4). Perineural invasion (HR=3.9), lymphovascular invasion (HR-6.6), tumor diameter >4 cm (HR=3.6), high-risk location (HR=2.4), poor differentiation (HR=4.8), and depth >6 mm (HR=4.5) were also significant predictors of metastasis. ART-treated Class 2B patients had a ~50% reduction in metastatic rates compared to untreated matched Class 2B patients. This i40-GEP accurately stratifies the risk of metastasis and local recurrence to inform clinical decision-making and identify ideal candidates for ART.

### Methods

### Patient Cohort

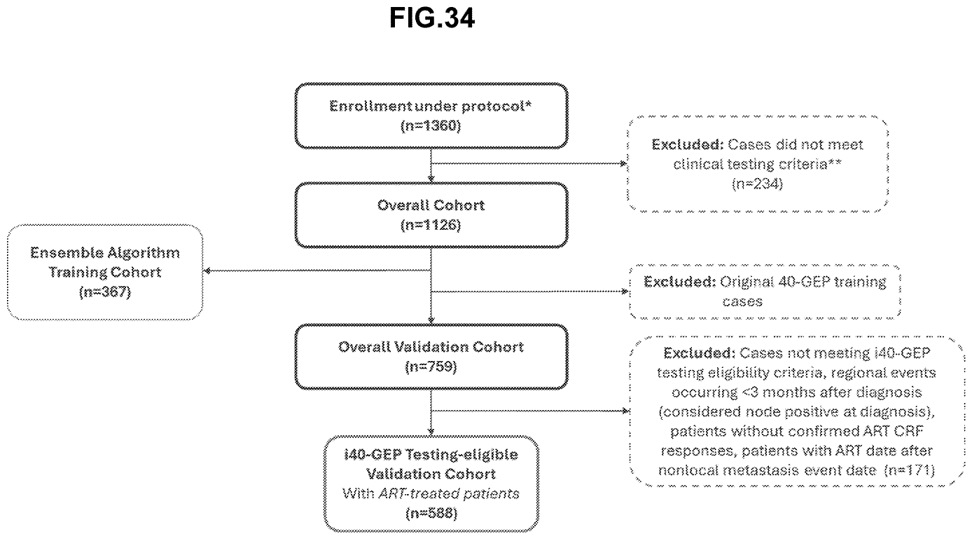

All patient samples used in development and validation of the algorithm for the i40-GEP (FIG. 34) were enrolled from a previously described, multi-center retrospective study [5-7] for patients with invasive cSCC [14]. Complete monitoring was performed for 100% of study participants. Independent histological review was performed by a board-certified dermatopathologist to confirm the diagnosis of invasive cSCC and to record pathologic features in addition to those provided on the pathology report on record. All patients in the validation cohort had a minimum of 3 years of follow-up or a local recurrence or metastasis event.

Development of Extended Predictions from the 40-GEP

To integrate CP factors with the 40-GEP [5] and refine outcome predictions, subsets of discriminative genes, specific to local recurrence and specific to metastasis, were identified using random forest models (for metastases) and multivariable Cox regressions (for local recurrences). Seven genes were identified for prediction of metastasis, and 11 genes for temporally aggressive local recurrences occurring within the first year post-diagnosis of cSCC. A predictive model of metastasis used Cox regression and gene predictors with generalizability assessed with the test set. The test set performance was then used to establish prediction cut points with the criterion of optimizing a lowest-risk Class 1A and highest-risk Class 2B, which were then validated on the hold-out data set. An elastic net model with 4×10-fold cross-validation was used to predict temporally aggressive local recurrences, and model performance was significantly improved when the 11 gene predictors were combined with the CP factors; immunocompromised status, tumor diameter <2 or ≥2 cm, histological differentiation (poor/undifferentiated or not poor/differentiated), and any reported perineural invasion (PNI). CP factors were chosen by multivariable Cox regression on local recurrence and availability at time of biopsy, as significant or non-significantly trending coefficients. The elastic net model with 11 genes and 4 CP factors was tested, and a cut point to discriminate between low versus high risk of local recurrence was identified and validated on the hold-out data set.

The combined model outputs were used to categorize risk class using a decision-tree, with a Class 1A, indicating lowest risk of both local recurrence and metastasis, and a Class 2B, indicating highest risk reported by the 40-GEP and extended metastasis predictions. The remaining moderate risk cases as indicated by GEP were then refined as ‘low risk’ and ‘high risk’ using a combination of tumor location (is or is not located on the head or neck) and tumor diameter (binned as unknown or <2 cm, 2 cm to <4 cm or ≥4 cm) as proxies for deep invasion along with histological differentiation in an elastic net model prediction for metastasis using 5×10-fold cross-validation. The test-set predictions were used to determine optimal cut point accuracy, resulting in the final low- and high-risk classes of Class 1B and Class 2A, respectively. The final extended 40-GEP test integrating CP factors (i40-GEP) yielded 4 risk classes: Class 1A (Lowest Risk), Class 1B (Low Risk), Class 2A (High Risk) and Class 2B (Highest Risk), each anchored to a given utility.

### ART Benefit Analysis

Methods to determine benefit of ART for Class 2A and 2B patients were performed as previously described [8-10]. Briefly, patients with a regional metastasis event date <3 months after diagnosis (considered node-positive at diagnosis for this analysis) and any patients who received radiation therapy after the date of diagnosis of regional/distant metastasis (not considered adjuvant) were removed (FIG. 34). Five cases were missing a date for ART treatment and were presumed adjuvant. Statistical analyses were performed using a bootstrapped cohort-sampling framework. For each iteration, matched cohorts of ART-treated and untreated patients (Table 24) were generated and stratified by i40-GEP Class×ART treatment status in 4 groups overall. Analyses used survival and actuarial life expectancy methods, substituting time to metastatic progression (regional and/or distant) for death (survival_3.5 3 package, R v4.3.0).

TABLE 24
Univariable analysis of i40-GEP and NCCN

| | | |
|---|---|---|
| Factor | HR (95% CI) | p-value |

140-GEP results

| | | |
|---|---|---|
| Class 1A | Reference | — |
| Class 1B | 2.3 (0.82-6.2) | 0.111 |
| Class 2A | 9.2 (4.0-21.2) | <0.001 |
| Class 2B | 15.2 (6.6-35.1) | <0.001 |

NCCN Risk Groups*

| | | |
|---|---|---|
| NCCN high-risk | Reference | — |
| NCCN very-high-risk | 4.5 (2.7-7.3) | <0.001 |

Survival tables were used to project the time at which a cohort exceeded 50% projected metastatic progression, and this estimate was recorded for each bootstrap iteration. Primary comparisons assessed differences between ART-treated and untreated patients within each i40-GEP Class. Sampling distributions of projected time to metastatic progression were compared using the Kolmogorov-Smirnov two-sample test, with the critical Dcrit statistic (adjusted to reflect effective cohort size rather than the number of bootstrap iterations) and interpreted as a one-sided test favoring ART benefit, as previously reported [8, 9].

### Validation and Statistical Analyses

Metastasis-free survival (MFS; including regional and distant metastasis) and local recurrence-free survival (LRFS) were estimated using Kaplan-Meier (KM) analysis and compared using the log-rank test. Univariable and multivariable Cox regression analyses were performed. The multivariable Cox modeling was performed controlling for the overlapping risk factors between the i40-GEP and individual clinicopathologic factors assessed in the Cox model by implementing a variance inflation factor to control for potential interactions. Model likelihood ratios and C-index, obtained from the univariable Cox models, Akaike Information Criterion (AIC), and Area Under the Curve (AUC) were used to compare the models of i40-GEP and NCCN risk groups for their performance in predicting metastatic events. Positive (PPV) and negative (NPV) predictive values were calculated to assess the accuracy of metastasis risk prediction for the i40-GEP in combination with NCCN risk category. Calculation for i40-GEP used Class 1A versus Class 2B as these results relate to clear management decision points of defer treatment or escalate to ART.

Statistical analysis, predictive modeling, and feature selection were all performed using R Statistical Software (v4.5.0) running custom written programs and freely available R packages (survival v3.8-3, MASS v7.3-65).

### Results

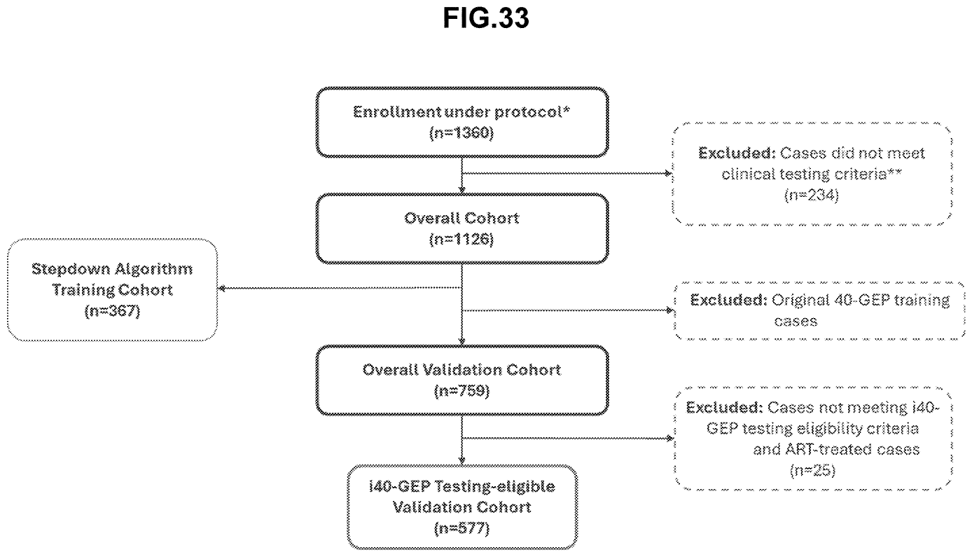

Training of prognostic signatures were optimized for risk of local recurrence and metastasis (regional [i.e., lymph node] or distant) (FIG. 33, and Table 25). The validation cohort included 577 patients that were 71.8% male with a median age of 72 years. The cohort was 70.2% NCCN high risk and 27.9% very high risk (Table 1 and FIG. 33). The i40-GEP risk classification results were reported as Class 1A (Lowest Risk), Class 1B (Low Risk), Class 2A (High Risk), and Class 2B (Highest Risk).

TABLE 25
Ensemble algorithm training cohort demographics.

| | Model training cohort | |
|---|---|---|
| Factor | n = 367 | |
| Age in years¹, median (range) | 71 | (35-90+) |
| Male sex, n (%) | 266 | (72.5%) |
| Median follow-up, years | 4.4 | |
| (patients with no event) | | |
| Anatomic location, n (%) | | |
| Trunk and extremities (non-pretibial) | 98 | (26.7%) |
| Special site² | 52 | (14.2%) |
| Head and neck | 217 | (59.1%) |
| Immunosuppression, n (%) | 79 | (21.5%) |
| Tumor diameter³ | | |
| Mean (SD), cm | 1.8 | (1.4) |
| ≥2 cm, n (%) | 126 | (34.3%) |
| Poor histologic differentiation | 40 | (10.9%) |
| Perineural invasion, n (%)⁴ | 26 | (7.1%) |
| Lymphovascular invasion, n (%) | 5 | (1.4%) |
| Mohs micrographic surgery, n (%) | 241 | (65.7%) |
| Negative surgical margins, n (%) | 367 | (100%) |
| Nodal/distant metastasis, n (%) | 43 | (11.7%) |
| Local recurrence, n (%) | 25 | (6.8%) |

TABLE 26
Patient characteristics for the i40-GEP validation cohort

| | 140-GEP validation cohort | |
|---|---|---|
| Factor | n = 577 | |
| Age in years¹, median (range) | 72 | (32-90+) |
| Male sex, n (%) | 414 | (71.8%) |
| Follow-up in years, median (range) | 4.3 | (0.5-15.5) |
| Anatomic location², n (%) | | |
| Trunk/extremities (non-pretibial) | 121 | (21.0%) |
| Special site³ | 34 | (5.9%) |
| Head and neck | 421 | (73.0%) |
| Immunosuppression, n (%) | 146 | (25.3%) |
| Tumor diameter, n (%) | | |
| <2 cm/unknown | 344 | (59.6%) |
| 2-4 cm | 206 | (35.7%) |
| >4 cm | 27 | (4.7%) |
| Poor histologic differentiation | 63 | (10.9%) |
| Perineural invasion, n (%) | 25 | (4.3%) |
| Lymphovascular invasion, n (%) | 8 | (1.4%) |
| Mohs micrographic surgery, n (%) | 409 | (71.1%) |
| Negative surgical margins, n (%) | 577 | (100%) |
| 140-GEP result, n (%) | | |
| Class 1A (Lowest Risk) | 256 | (44.4%) |
| Class 1B (Low Risk) | 131 | (22.7%)) |
| Class 2A (High Risk) | 114 | (19.8%) |
| Class 2B (Highest Risk) | 76 | (13.2%) |
| NCCN risk groups, n (%) | | |
| Low risk4 | 11 | (1.9%) |
| High risk | 405 | (70.2%) |
| Very high risk | 161 | (27.9%) |
| Regional/distant metastasis, n (%) | 66 | (11.4%) |
| Local recurrence, n (%) | 58 | (10.1%) |

¹Patient ages over 90 years are reported as 90+ for protecting identity.
²n = 1 patient unclassifiable due to missing information.
³Special site includes acral, anogenital, and pretibial.
⁴NCCN low-risk patients were included if they had documented invasiveness of Clarks' level IV (reticular dermis, n=10) or perineural invasion <0.1 mm/unknown size (n = 1).
Abbreviations: BWH, Brigham & Women's Hospital T-staging; NCCN, National Comprehensive Cancer Network Guidelines risk stratification.

Validation of the i40-GEP for Disease Progression

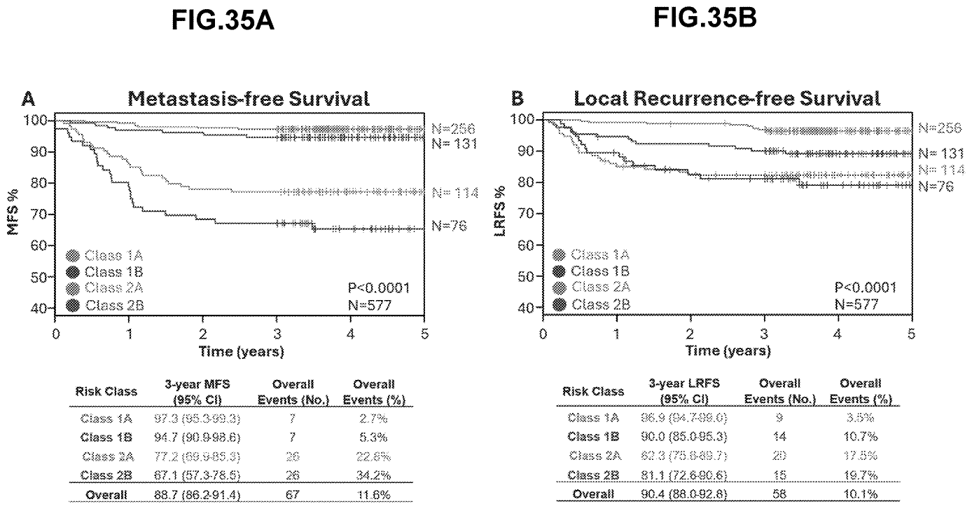

The i40-GEP identified 256 Class 1A (44.4%), 131 Class 1B (22.7%), 114 Class 2A (19.8%), and 76 Class 2B (13.2%) tumors in the validation cohort of 577 patients with cSCC (Table 25). The i40-GEP significantly stratified 3-year MFS (Class 1A=97.3%, 95% CI, 95.3-99.3%; Class 1B=94.7%, 95% CI, 90.9-98.6; Class 2A=77.2%, 95% CI, 69.9-85.3%; and Class 2B=67.1%, 95% CI, 57.3-78.5%; p<0.001) in the overall cohort (3 yr MFS=88.7%, 95% CI, 86.2-91.4%;) (FIG. 35A). Local recurrence-free survival (LRFS) was 90.4% (88.0-92.8%) for the overall cohort at 3 years and was significantly stratified by the i40-GEP (p<0.001). Three-year LRFS was 96.9% (94.7-99.0%) for Class 1A, compared with 90.0% (85.0-95.3) for Class 1B, 82.3% (75.6-89.7) for Class 2A, and 81.1% (72.6-90.6%) for Class 2B (FIG. 35B).

The i40-GEP Stratifies Risk in NCCN Patient Risk Groups

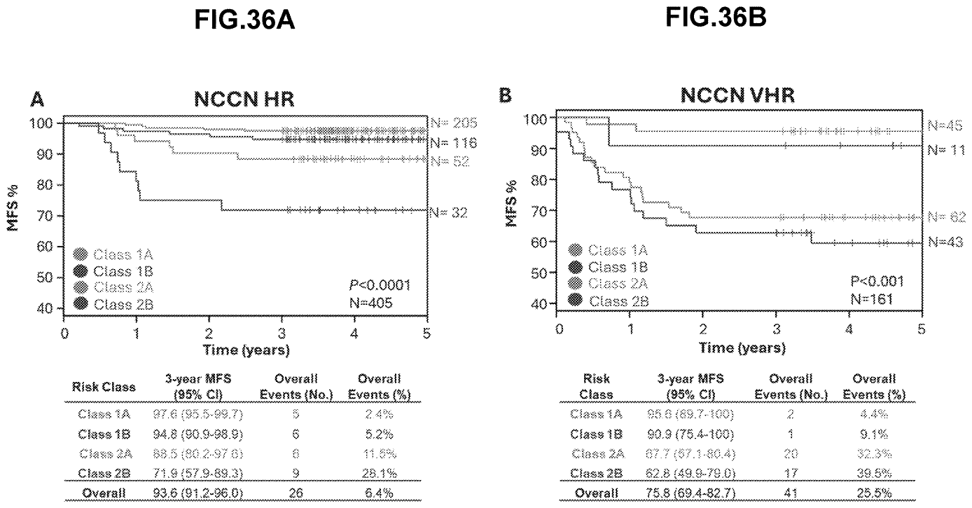

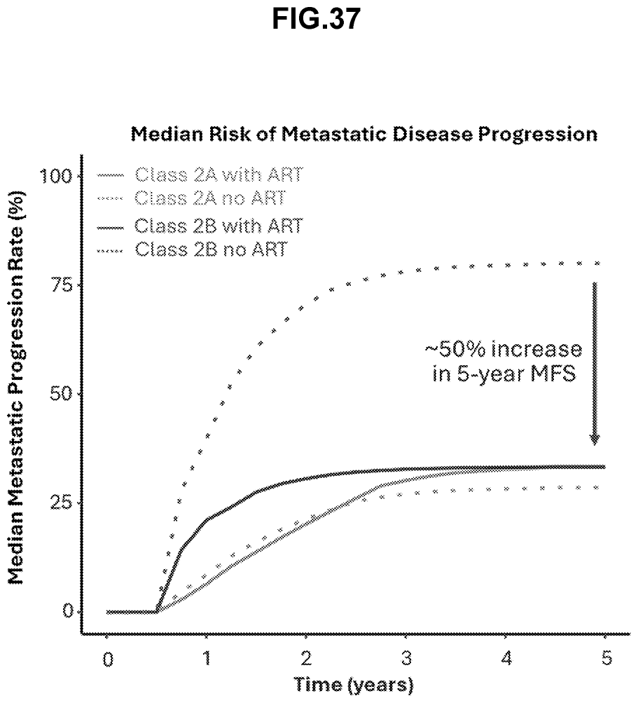

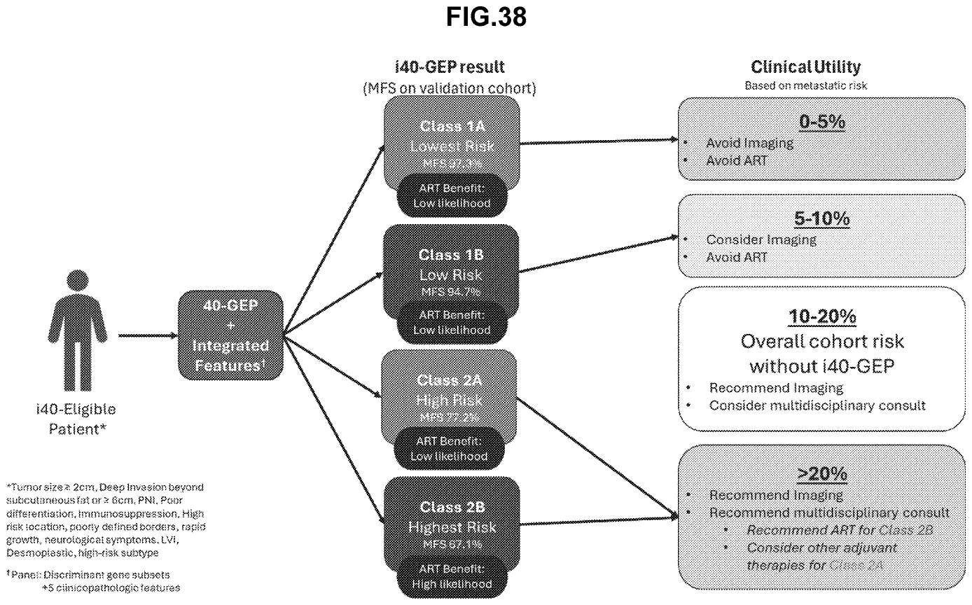

The i40-GEP significantly differentiated metastatic risk even within NCCN high-risk (HR) (n=405; p<0.001) and very-high-risk (VHR) (n=161; p<0.001) patient groups (FIGS. 36A and 36B). Notably, 3-year MFS was consistently high in patients with Class 1A i40-GEP results in both NCCN HR (97.6%) NCCN VHR (95.6%) subgroups. The <5% risk across NCCN risk groups represents robust and stable risk prediction to better support clinical decisions. A lower MFS was seen in Class 1B patients (NCCN HR, 94.8%; VHR, 90.9%) and Class 2A patients (NCCN HR, 88.5%; VHR, 67.7%). The lowest MFS was seen in patients with Class 2B results (NCCN HR, 71.9%; VHR, 62.8%) (FIGS. 36A and 36B).

The i40-GEP Refines Risk Prediction Beyond Individual Clinicopathologic Risk Factors and NCCN Risk Groups

Univariate analysis with clinicopathologic (CP) features (‘CP feature not present’ as the reference) and i40-GEP (Class 1A as reference) in the validation cohort (n=577) showed that Class 2A (HzRt=9.4, p<0.001) and Class 2B (HzRt=15.3, p<0.001) are significantly associated with metastasis and have a larger hazard ratio (HzRt) than any CP features. Clinicopathologic features significantly associated with metastasis (p<0.05) include the presence of any PNI (HzRt=3.9), LVI (HR=6.6), tumor diameter >4 cm (HzRt-3.6), high-risk location (HzRt=2.4), immunosuppression (HzRt=2.1), poor differentiation (HzRt=4.8), tumor depth >6 mm (HR=4.5) and tumor depth beyond subcutaneous fat (HzRt-2.7) (Table 26). Univariate analysis with i40-GEP (Class 1A as reference) and NCCN risk groups (NCCN HR as reference) demonstrated that within the same cohort (n=566; n=11 NCCN low-risk removed from analysis, there were no events), Class 2A (HzRt-9.2, p<0.001) and Class 2B (HzRt=15.2, p<0.001) have a hazard ratio 2× and 3.3× larger than the hazard ratio outcome of NCCN VHR (HzRt=4.5, p<0.001) (Table 24).

TABLE 27
Univariable analysis of i40-GEP and
individual clinicopathologic risk factors

| | | |
|---|---|---|
| Factor | HR (95% CI) | p-value |

140-GEP results

| Class 1A | Reference | | — |
|---|---|---|---|
| Class 1B | 2.3 | (0.82-6.2) | 0.114 |
| Class 2A | 9.4 | (4.1-21.7) | <0.001 |
| Class 2B | 15.3 | (6.6-35.3) | <0.001 |

Clinicopathologic Features

| | | | |
|---|---|---|---|
| PNI (any) | 3.9 | (1.9-7.9) | <0.001 |
| PNI (large caliber) | 3.0 | (1.2-7.5) | 0.018 |
| LVI | 6.6 | (2.4-18.3) | <0.001 |
| Diameter (≥2-4 cm) | 1.2 | (0.7-1.9) | 0.563 |
| Diameter (>4 cm) | 3.6 | (1.8-7.3) | <0.001 |
| Location (high-risk) | 2.4 | (1.1-5.2) | 0.029 |
| Immunosuppressed | 2.1 | (1.3-3.4) | 0.003 |
| Poor differentiation | 4.8 | (2.9-7.8) | <0.001 |
| Depth (2-6 mm) | 1.2 | (0.5-3.1) | 0.640 |
| Depth (>6 mm) | 4.5 | (2.5-8.0) | <0.001 |
| Depth beyond fat | 2.7 | (1.6-4.8) | <0.001 |
| High-risk subtype | 1.1 | (0.1-7.6) | 0.960 |

Multivariable analysis (Table 28) further assessed the impact of i40-GEP in context with CPs and showed that Class 2A (HzRt=6.1) and Class 2B (HzRt=9.9) i40-GEP test results were significant predictors of metastasis and had a greater hazard ratio than any clinicopathologic factors included. Clinicopathologic features that were significant predictors of metastasis included high-risk tumor location (HzRt=2.7), immunosuppression (HzRt=2.4), invasion depth >6 mm (HzRt-2.2), and poor differentiation (HzRt-2.7). Multivariable analysis also showed that i40-GEP Class results 1B (HzRt=2.7), 2A (HzRt=3.4) and Class 2B (HzRt-5.7) were significant predictors of local recurrence with higher hazard ratios than high-risk tumor location (HR=2.4), and immunosuppression (HR=2.2), which were also significantly associated with local recurrence. Importantly, the NCCN high-risk subtype was not significantly associated with outcome prediction in either multivariable model for metastasis or local recurrence.

TABLE 28
Multivariable analysis for predicting risk non-local metastasis using
i40-GEP, controlling for individual clinicopathologic factors

| | | |
|---|---|---|
| Factor | HR (95% CI) | p-value |

140-GEP results

| | | |
|---|---|---|
| Class 1A | Reference | — |
| Class 1B | 2.0 (0.7-5.7) | 0.166 |
| Class 2A | 6.1 (2.4-16) | <0.001 |
| Class 2B | 9.9 (4.0-24.5) | <0.001 |

Clinicopathologic Features

| | | |
|---|---|---|
| PNI (non-large caliber ) | 1.9 (0.8-4.1) | 0.124 |
| PNI (large caliber) | 0.6 (0.2-1.9) | 0.422 |
| LVI | 2.3 (0.7-7.0) | 0.152 |
| Tumor Diameter (≥2-4 cm) | 0.7 (0.4-1.3) | 0.224 |
| Tumor Diameter (>4 cm) | 1.9 (0.7-4.8) | 0.176 |
| Location (high-risk) | 2.7 (1.1-6.3) | 0.027 |
| Immunosuppressed | 2.4 (1.4-4.2) | 0.002 |
| Poor differentiation | 2.7 (1.5-5.0) | <0.001 |
| Tumor Thickness (≥2-6 mm) | 1.5 (0.6-4.0) | 0.412 |
| Tumor Thickness (>6 mm) | 2.2 (1.1-4.3) | 0.031 |
| Depth beyond fat | 1.3 (0.7-2.6) | 0.383 |
| NCCN High-risk subtype | 0.3 (0.0-2.1) | 0.203 |

The i40-GEP Provides Greater Predictive Accuracy/Discriminative Capacity Over NCCN Risk Groups

To ensure that i40-GEP adds value to existing stratification, performance analyses and accuracy metrics were used to compare NCCN metastatic risk stratification to stratification by i40-GEP. The i40-GEP outperformed NCCN in C-index (i40-GEP=0.77, NCCN=0.68), AUC (i40-GEP=0.78, NCCN=0.69), and AIC (i40-GEP=774, NCCN=804) (Table 29). Further, the likelihood ratio (LR) of the i40-GEP model was 69.8 whereas the LR of the NCCN model was lower at 36.6. Finally, the NPV and PPV for Class 1A compared to Class 2B were 97.3% and 34.2% respectively, which were greater than the accuracy metrics for NCCN (NPV=93.6%, PPV=25.5%).

TABLE 29
Head-to-head predictive model
comparisons of i40-GEP and NCCN

| Accuracy | | |
|---|---|---|
| Metric | Metastatic risk | |
| Model | 140-GEP | NCCN |
| C-Index* | 0.77 | 0.68 |
| AUC* | 0.78 | 0.69 |
| AIC* | 774 | 804 |
| Likelihood ratio* | 69.8 | 36.6 |
| NPV, % | 97.3% | 93.6% |
| PPV, % | 34.2% | 25.5% |

Validation of i40-GEP Results in Identifying Patients Most Likely to Benefit from ART

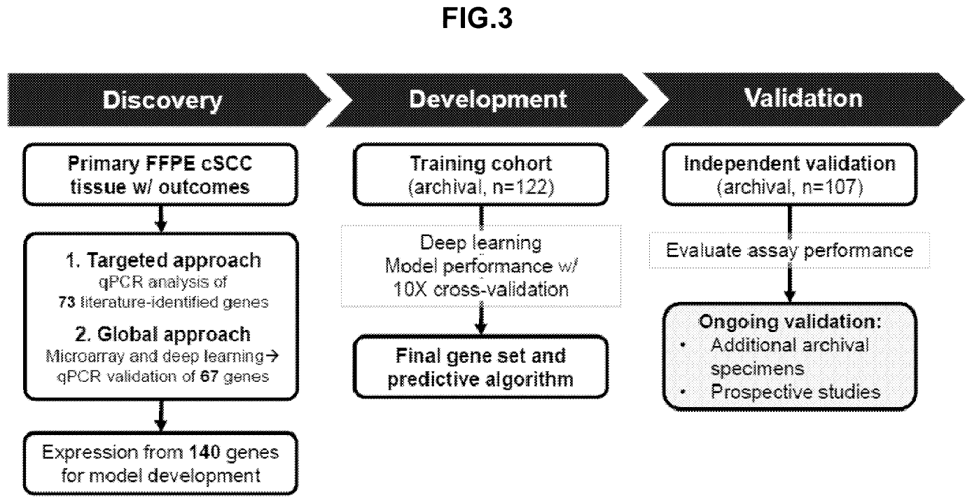

Clinicians navigate treatment pathways, including consideration for ART, using risk-aligned decision-making. Building upon our foundational work that found a Class 2B 40-GEP (Highest Risk) result associated with survival benefit from ART [8-10], thus we assessed the Class 2A (High Risk) and Class 2B (Highest Risk) patients for a benefit from ART. A validation cohort including ART-treated patients was used for analysis with n=25 ART-treated and n=563 untreated patients that were available for propensity score matching on clinicopathologic risk factors that are typically used in risk assessment as previously described (FIG. 34) [8,9]. After matching, there were n=25 i40-GEP×ART-matched treatment groups for resampled cohort analysis. Median disease progression rates for Class 2B ART-treated patients showed a robust 50% decrease in metastatic rate at 5 years compared with untreated Class 2B patients (FIG. 3), indicating a high likelihood to benefit from ART for patients with i40-GEP Class 2B results. No difference in 5-year progression rates was observed based on ART treatment status in patients with a Class 2A result, indicating these patients have a low likelihood of benefit from ART.

### Discussion

The i40-GEP builds on the prognostic information provided by the 40-GEP test to further refine risk stratification in patients with cSCC. The i40-GEP utilized a larger, more diverse training set, resulting in a more refined outcome predictor for both regional/distant metastasis and temporally aggressive local recurrence. Results presented here demonstrated that the i40-GEP significantly stratified risk of metastasis and local recurrence among patients with cSCC and more specifically among patients classified as NCCN HR and VHR. Further, the i40-GEP maintained robust prediction of ART benefit among patients with a Class 2B result.

Traditional staging systems rely on clinicopathologic factors to attempt to identify patients at risk of disease progression [4,17, 19-23]. Relying on individual CP factors alone or in risk assessment systems, such as NCCN, provides limited personalized risk information to guide management decisions. Many clinicians report they do not stage cSCC patients, or only stage those with the highest risk tumors [16,24]. New risk stratification systems relying solely on CP factors continue to be developed [25,26], and investigational online tools, including riSCC nomogram and Erasmus Medical Center nomogram, have recently attempted to improve the overall risk prediction accuracy by inclusion of various CP variables, but they are not clinically available nor independently validated [15, 25-30].

The established 40-GEP provided prognostic information to enhance traditional risk stratification systems, representing an important step forward in patient care [5]. The i40-GEP test provides additional capabilities to further refine risk stratification for patients with cSCC. Multivariable analysis confirmed i40-GEP Class results had the highest hazard ratios when compared to CP factors. A Class 2B result had 3× and 4× increased metastatic risk relative to poor differentiation and LVI, respectively. Direct comparisons of the i40-GEP and NCCN further affirmed the superior performance of the i40-GEP.

Clinicians generally utilize a risk threshold <10% to consider deferring treatment for their patients with cSCC [15,16]. Overall, the i40-GEP identified nearly half of the validation cohort (44.4%) as Class 1A (a <5% metastatic risk), which supports shifting management considerations of patients with this test result to treatment deferment. Impactfully, the i40-GEP classified 30% of patients in the NCCN VHR group as Class 1A (<5% risk). This group is typically recommended for ART, and could now consider deferring ART. The i40-GEP Class 1B result helps refine the intermediate risk signatures, identifying a population with metastatic risk <10%, and who can consider de-escalated management (FIG. 4).

The i40-GEP Class 2A patients had a low MFS (77.2%). Based on a >20% metastasis risk threshold, clinicians would generally consider these patients for ART [15,16]. The results presented here indicate that patients with a Class 2A result did not show a significant ART benefit, but clinicians may consider other adjuvant treatments and multidisciplinary care for this subset of patients. This is especially critical for the 38.5% of the NCCN VHR subset who would traditionally be considered for ART, but with a Class 2A result they are unlikely to benefit and may additionally experience side effects from treatment. Further, the i40-GEP classified 13.2% of patients as Class 2B and they exhibited poor MFS (67.1%), a risk prompting recommendation for ART. The i40-GEP validates a Class 2B result to identify patients with a high likelihood of benefit from ART. A robust 50% decrease in progression rates was observed in Class 2B patients who underwent ART compared to those with a Class 2B result who did not undergo ART. Thus, the i40-GEP identified a substantial proportion of patients with the highest risk of disease progression and most likely to benefit from intensified disease management, including ART and multi-disciplinary care. Moreover, there are no risk assessment or staging systems, and no other clinically validated and commercially available ART response biomarkers that have demonstrated the capability to predict response to ART other than the 40-GEP.

These results demonstrate that integrating 40-GEP with clinicopathologic factors in the i40-GEP increased prognostic value compared with traditional risk assessment systems for cSCC. The i40-GEP provides a comprehensive platform with a single output for risk of local recurrence, metastasis, and likelihood of ART benefit; and, to guide treatment and management decisions for the rapidly expanding portfolio of immunotherapy options and ongoing trials [31-37], imaging, and follow-up in a personalized manner. The i40-GEP utilizes clinicopathologic features largely available at biopsy and shows greater predictive capacity beyond clinicopathologic risk features alone to refine patient risk. This information availability translates to the clinic to promote decisive patient care for earlier disease management and improves patient care. Coordination and communication between multidisciplinary care team specialties can reduce the time to starting treatment and the total number of pre-op visits [38]. Moreover, the augmented prognostic information provided by the i40-GEP test further enhances risk-aligned decision-making within the established NCCN treatment pathways.

### CONCLUSIONS

The i40-GEP provides robust and comprehensive prediction of ART benefit, likelihood of regional/distant metastasis across NCCN risk groups, and local recurrence risk. The i40-GEP risk classifications are associated with utilities for risk-aligned management of cSCC, informing clinical decision-making for patient follow-up, surveillance imaging, and ART. The evidence presented demonstrates that the i40-GEP provides significant prognostic value for risk stratification in cSCC. Using the i40-GEP independently or within NCCN risk groups to identify patients unlikely to progress and those at-risk of progression enables more precise alignment of management strategies with individual risk profiles, working within treatment pathways to support confident decision making for clinicians and improved outcomes for cSCC patients at elevated risk.
1. Olsen et al., International patterns and trends in the incidence of melanoma and cutaneous squamous cell carcinoma, 1989-2020. Br J Dermatol. 2024 Mar. 15; 190(4):492-500. doi: 10.1093/bjd/ljad425 PubMed PMID: 37890023.
2 Eggermont C J, Eggermont A M M. Shifting landscape in skin cancer incidence: the rising tide of cutaneous squamous cell carcinoma and potential implications for prevention. Br J Dermatol. 2024 Mar. 15; 190(4):460-1. doi: 10.1093/bjd/ljad480 PubMed PMID: 38099564.
3 Tokez et al., Cumulative incidence and disease-specific survival of metastatic cutaneous squamous cell carcinoma: a nationwide cancer registry study. J Am Acad Dermatol. 2021 Oct. 12; S0190-9622(21)02644-X. doi: 10.1016/j.jaad.2021.09.067 PubMed PMID: 34653569.
4. Ruiz et al., Performance of the American Joint Committee on Cancer Staging Manual, 8th Edition vs the Brigham and Women's Hospital Tumor Classification System for Cutaneous Squamous Cell Carcinoma. JAMA Dermatol. 2019 Jul. 1; 155(7):819. doi: 10.1001/jamadermatol.2019.0032
5. Wysong et al., Validation of a 40-gene expression profile test to predict metastatic risk in localized high-risk cutaneous squamous cell carcinoma. J Am Acad Dermatol. 2021 February; 84(2):361-9. doi: 10.1016/j.jaad.2020.04.088
6. Ibrahim et al., Enhanced metastatic risk assessment in cutaneous squamous cell carcinoma with the 40-gene expression profile test. Future Oncol. 2022 March; 18(7):833-47. doi: 10.2217/fon-2021-1277 PubMed PMID: 34821148.
7. Wysong et al., Integrating the 40-Gene Expression Profile (40-GEP) Test Improves Metastatic Risk-Stratification Within Clinically Relevant Subgroups of High-Risk Cutaneous Squamous Cell Carcinoma (cSCC) Patients. Dermatol Ther (Heidelb). 2024 March; 14(3):593-612.
8. Ruiz et al., Predicting adjuvant radiation therapy benefit in cutaneous squamous cell carcinoma with the 40-gene expression profile. Future Oncol. 2024 Sep. 4; 20(35):2737. doi: 10.1080/14796694.2024.2390820 PubMed PMID: 39229801.
9. Arron et al., Association of a 40-Gene Expression Profile With Risk of Metastatic Disease Progression of Cutaneous Squamous Cell Carcinoma and Specification of Benefit of Adjuvant Radiation Therapy. International Journal of Radiation Oncology, Biology, Physics. 2024 Nov. 1; 120(3):760-71. doi: 10.1016/j.ijrobp.2024.05.022 PubMed PMID: 38810706.
10. Moody et al., Inconsistent Associations Between Risk Factor Profiles and Adjuvant Radiation Therapy (ART) Treatment in Patients with Cutaneous Squamous Cell Carcinoma and Utility of the 40-Gene Expression Profile to Refine ART Guidance. Dermatol Ther (Heidelb). 2024 Mar. 23; 14(4):861-73. doi: 10.1007/s13555-024-01125-z PubMed PMID: 38521873; PubMed Central PMCID: PMC11052963.
11. Saleeby et al., A Prospective, Multi-Center Clinical Utility Study Demonstrates That the 40-Gene Expression Profile (40-GEP) Test Impacts Clinical Management for Medicare-Eligible Patients with High-Risk Cutaneous Squamous Cell Carcinoma (cSCC). J of Skin. 2022 Nov. 16; 6(6):482-96. doi: 10.25251/skin.6.6.5
12. Jambusaria-Pahlajani et al., Evaluation of AJCC Tumor Staging for Cutaneous Squamous Cell Carcinoma and a Proposed Alternative Tumor Staging System. JAMA Dermatol. 2013 Apr. 1; 149(4):402. doi: 10.1001/jamadermatol.2013.2456
13. Amin et al., editors. AJCC Cancer Staging Manual, Eighth Edition (2017). New York, NY: Springer International Publishing; 2017.
14. NCCN Clinical Practice Guidelines in Oncology (NCCN Guidelines®) for Squamous Cell Skin Cancer V.1.2026. @ National Comprehensive Cancer Network, Inc. 2026. [Internet]. Available from: https://www.nccn.org/professionals/physician_gls/pdf/squamous.pdf
15. Rentroia-Pacheco B et al., Personalised decision making to predict absolute metastatic risk in cutaneous squamous cell carcinoma: development and validation of a clinicopathological model. eClinicalMedicine. 2023 September; 63:102150. doi: 10.1016/j.eclinm.2023.102150
16. Shah et al., Actionable Risk Thresholds for Adjuvant Radiation Therapy and Surveillance Imaging in High-Risk Cutaneous Squamous Cell Carcinoma. J of Skin. 2025 Jul. 23; 9(4):4. doi: 10.25251/83v2 hr07
17 Ratner D et al., The 40-Gene Expression Profile Test Identifies Patients with National Comprehensive Cancer Network High-Risk Cutaneous Squamous Cell Carcinoma at High Risk of Poor Outcomes to Inform Management Decisions. J of Skin. 2025 Jul. 23; 9(4):4. doi: 10.25251/mvr2rn83
18 Wright et al., ranger: A Fast Implementation of Random Forests for High Dimensional Data in C++ and R. Journal of Statistical Software. 2017 Mar. 31; 77:1-17. doi: 10.18637/jss.v077.i01
19. Girardi et al., Validation of Current Staging Systems in HNCSCC: A Multinational Cohort Study. Head Neck. 2025 Jul. 25. doi: 10.1002/hed.28259 PubMed PMID: 40709408.
20. Voller et al., Performance of Staging Systems for Non-head and Neck Cutaneous Squamous Cell Carcinoma. Am J Clin Dermatol. 2025 Sep. 25. doi: 10.1007/s40257-025-00987-z PubMed PMID: 40996598.
21. Stevens et al., Validation of the 2022 National Comprehensive Cancer Network Risk Stratification for Cutaneous Squamous Cell Carcinoma. JAMA Dermatol. 2023; 159(7):728-35. doi: 10.1001/jamadermatol.2023.1353
22. Sax et al., Limitations of the commercially available gene expression test in predicting cutaneous squamous cell carcinoma metastasis and clinical outcomes. J Am Acad Dermatol. 2024 May 16; S0190-9622(24)00746-1. doi: 10.1016/j.jaad.2024.05.014 PubMed PMID: 38762012.
23. Goldberg M S et al., Re: Limitations of the commercially available gene expression test in predicting cutaneous squamous cell carcinoma metastasis and clinical outcomes. Journal of the American Academy of Dermatology. 2025 July; 93(1):155-6. doi: 10.1016/j.jaad.2025.02.092
24 Patel et al., Cutaneous squamous cell carcinoma staging may influence management in users: A survey study. Cancer Med. 2022 January; 11(1):94-103. doi: 10.1002/cam4.4426 PubMed PMID: 34796670; PubMed Central PMCID: PMC8704160.
25. Jambusaria-Pahlajani et al., riSCC: A personalized risk model for the development of poor outcomes in cutaneous squamous cell carcinoma. J Am Acad Dermatol. 2025 July; 93(1):73-81. doi: 10.1016/j.jaad.2025.02.076 PubMed PMID: 40024391.
26 Steijlen et al., Enhanced metastatic risk stratification for Cutaneous Squamous Cell Carcinoma by combining clinical guidelines with the Erasmus MC model: Results from Two Nationwide Nested Case-Control Studies. J Am Acad Dermatol. 2025 May 16; S0190-9622(25)02129-2. doi: 10.1016/j.jaad.2025.05.1390 PubMed PMID: 40383274.
27 Leibovit-Reiben et al., Development of Gene-Expression Panel Predictive of Local Recurrence, Metastasis, and Overall Survival in Intermediate to High-risk Cutaneous Squamous Cell Carcinoma: A Cohort Study. J Am Acad Dermatol. 2025 Nov. 3; S0190-9622(25)03136-6. doi: 10.1016/j.jaad.2025.10.136 PubMed PMID: 41192515.
28. Wang et al., Transcriptomic analysis of cutaneous squamous cell carcinoma reveals a multigene prognostic signature associated with metastasis. Journal of the American Academy of Dermatology. 2023 Dec. 1; 89(6):1159-66. doi: 10.1016/j.jaad.2023.08.012 PubMed PMID: 37586461.
29 Rentroia-Pacheco et al., Abstract 4869: Efficient study design for the discovery of a gene expression signature predicting metastasis in cutaneous squamous cell carcinoma. Cancer Research. 2024 Mar. 22; 84(6_Supplement):4869. doi: 10.1158/1538-7445.AM2024-4869
30. Bapna et al., Comparative Analysis of Staging and Metastatic Risk Stratification Systems for Cutaneous Squamous Cell Carcinoma. J Invest Dermatol. 2025 Nov. 14; S0022-202X(25)03545-6. doi: 10.1016/j.jid.2025.11.001 PubMed PMID: 41242653.
31. Guy et al., Anti-EGFR plus anti-PD1 in advanced and refractory cutaneous squamous cell carcinoma: a cohort study. Oncologist. 2026 Mar. 13; oyag080. doi: 10.1093/oncolo/oyag080 PubMed PMID: 41830427.
32. Giacomantonio et al., Intralesional Interleukin-2 Therapy for Treatment of Cutaneous Squamous Cell Carcinoma. JAMA Dermatol. 2026 Mar. 18. doi: 10.1001/jamadermatol.2026.0181 PubMed PMID: 41848721.
33. Gross et al., Neoadjuvant Cemiplimab for Stage II to IV Cutaneous Squamous-Cell Carcinoma. N Engl J Med. 2022 Oct. 27; 387(17):1557-68. doi: 10.1056/NEJMoa2209813 PubMed PMID: 36094839; PubMed Central PMCID: PMC9844515.
34. Gross et al., Neoadjuvant cemiplimab and surgery for stage II-IV cutaneous squamous-cell carcinoma: follow-up and survival outcomes of a single-arm, multicentre, phase 2 study. Lancet Oncol. 2023 November; 24(11):1196-205. doi: 10.1016/S1470-2045(23)00459-X PubMed PMID: 37875144.
35. Rischin et al., Adjuvant Cemiplimab or Placebo in High-Risk Cutaneous Squamous-Cell Carcinoma. N Engl J Med. 2025 May 31; NEJMoa2502449. doi: 10.1056/NEJMoa2502449
36. McLean et al., Cemiplimab in the treatment of cutaneous squamous cell carcinoma. Immunotherapy. 2026 Jan. 29; 0(0):1-14. doi: 10.1080/1750743X.2026.2619658 PubMed PMID: 41607313.
37. Adkins et al., Neoadjuvant and Adjuvant Amivantamab Plus Pembrolizumab in Resectable, Locally Advanced HPV-Unrelated Head & Neck Squamous Cell Cancer: Cohort 6 of the Phase 1b/2 OrigAMI-4 Study. International Journal of Radiation Oncology, Biology, Physics. 2026 May 1; 125(1):e60-1. doi: 10.1016/j.ijrobp.2026.02.119
38. Ran et al., Integrated multidisciplinary care reduces time to treatment in patients with advanced cutaneous squamous cell carcinoma: A retrospective cohort study. Journal of the American Academy of Dermatology. 2026 Apr. 2. doi: 10.1016/j.jaad.2026.03.096

All references cited in this application are expressly incorporated by reference herein.

What is claimed is:

1: A method for treating a patient with a cutaneous squamous cell carcinoma (cSCC) tumor, the method comprising:
(a) obtaining a first risk classification in a cSCC tumor sample from the patient, wherein the first risk classification is obtained by:
determining the expression levels in a first gene set comprising: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839; and comparing the expression levels of the first gene set to a predictive training set to generate the first risk classification;
wherein the first risk classification provides an indication as to whether the cSCC tumor has a low risk (Class 1), a moderate risk (Class 2A), or a high risk (Class 2B), and
wherein when the cSCC tumor is identified as having a low risk (Class 1) or a moderate risk (Class 2A) the method further comprises:
(b) obtaining a second risk classification in the cSCC tumor sample from the patient, wherein the second risk classification is obtained by:
determining the expression levels in a second gene set comprising: ALOX12, LIME1, MMP10, PI3, RUNX3, TFAP2B, SPP1, HDDC3, LOC100287896, TAF6L, MRC1, RNF135, PLS3, and ZNF839; and comparing the expression levels of the second gene set to a predictive training set to generate the second risk classification;
wherein the second risk classification provides an indication as to whether the cSCC tumor has a low risk (Class 1), a moderate risk (Class 2A), or a high risk (Class 2B), and;
(c) administering to the patient an aggressive treatment when the cSCC tumor is identified as having a moderate risk (Class 2A) or a high risk (Class 2B) based on the second risk classification.

2: The method of claim 1, wherein the first risk classification and/or the second risk classification indicates risk of local recurrence and/or risk of metastasis.

3: The method of claim 1 further comprising generating the second risk classification based on the combination with at least one risk factor.

4: The method of claim 3, wherein the at least one risk factor is selected from immune status (competent vs suppressed), histological differentiation (well/moderate vs poor), tumor diameter (less than 2 cm or greater than or equal to 2 cm), perineural invasion (PNI), tumor location, immunosuppression, depth of invasion, differentiation, histological subtype, and lymphovascular invasion.

5: The method of claim 3, wherein the at least one risk factor is immune status (competent vs suppressed), histological differentiation (well/moderate vs poor) tumor diameter (less than 2 cm or greater than 2 cm), and perineural invasion (PNI).

6: The method of claim 1 further comprising determining that the subject benefit from adjuvant radiotherapy (ART) when the patient has a cSCC tumor with a high risk of local recurrence and/or risk of metastasis.

7: The method of claim 1 further comprising administering the aggressive treatment selected from one or more of:
a) performing a resection of the cSCC tumor;
b) clinical follow-up of four to twelve times per year for about 3 years;
c) performing baseline and annual nodal imaging at least twice a year for about 2 years;
d) performing a nodal biopsy or a neck dissection;
e) administering an adjuvant treatment; and
f) enrolling in a clinical trial.

8: The method of claim 7, wherein the adjuvant treatment is one or more of chemoradiation, chemotherapy, regional limb therapy, surgery, systemic therapy, or immunotherapy.

9: The method of claim 1, wherein the expression level of each gene in the first gene set and/or the second gene set is determined by reverse transcribing the isolated mRNA and measuring a level of fluorescence for each gene in the gene set by a nucleic acid sequence detection system following RT-PCR.

10: The method of claim 1, wherein the cSCC tumor sample is obtained from a formalin-fixed, paraffin embedded sample.

11: The method of claim 1, wherein the first gene set and/or the second gene set further comprise at least one control gene, and wherein the at least one control gene is selected from the group consisting of BAG6, KMT2D/MLL2, MDM2, FXR1, KMT2C, MDM4, VIM, and NF1B, optionally wherein the control genes are MDM2, KMT2D, BAG6, FXR1, MDM4, and KMT2C.

12: The method of claim 1, wherein the expression level of:
ACSBG1 is decreased, ALOX12 is decreased, APOBEC3G is increased, ATP6V0E2 is increased, BBC3 is increased, BHLHB9 is decreased, CEP76 is decreased, DUXAP8 is increased, GTPBP2 is decreased, HDDC3 is increased, ID2 is decreased, LCE2B is decreased, LIME1 (ZGPAT) is increased, LOC100287896 is increased, LOC101927502 is increased, MMP10 is decreased, MRC1 is decreased, MSANTD4 is decreased, NFASC is decreased, NFIC is decreased, PDPN is increased, PI3 is decreased, PLS3 is decreased, RCHY1 is increased, RNF135 is increased, RPP38 is decreased, RUNX3 is increased, SLC1A3 is increased, SPP1 is increased, TAF6L is increased, TFAP2B is decreased, ZNF48 is increased, ZNF496 is increased, and ZNF839 is increased, when comparing a recurrent tumor to a non-recurrent sample.

13: The method of claim 1, wherein the expression level of:
ALOX12 is decreased, HDDC3 is increased, LIME1 (ZGPAT) is increased, LOC100287896 is increased, MMP10 is decreased, MRC1 is decreased, PI3 is decreased, PLS3 is decreased, RNF135 is increased, RUNX3 is increased, SPP1 is increased, TAF6L is increased, TFAP2B is decreased, and ZNF839 is increased, when comparing a recurrent tumor to a non-recurrent sample.

14. A method for determining benefit from adjuvant radiotherapy in a subject having a cutaneous squamous cell carcinoma (cSCC) tumor, the method comprising:
a) determining the expression level of at least 10 genes in a gene set; wherein the at least 10 genes in the gene set are selected from: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839;
b) identifying a risk of metastasis of the cSCC tumor and providing an indication as to whether the cSCC tumor has a low risk (Class 1), a moderate risk (Class 2A), or a high risk (Class 2B) of metastasis; and
c) determining that the subject benefit from adjuvant radiotherapy when the determination is made that the subject has a cSCC tumor with a high risk (Class 2B) of metastasis, wherein the risk is determined in combination with at least one risk factor selected from depth of tumor invasion and tumor differentiation.

15. The method of claim 14 further comprising administering adjuvant radiotherapy to the subject when the cSCC tumor is identified as a moderate risk (Class 2A) or a high risk (Class 2B) of metastasis, wherein the moderate risk is determined in combination with at least one risk factor depth of tumor invasion, and/or tumor differentiation.

16: The method of claim 14 further comprising identifying that the cSCC tumor has a moderate risk (Class 2A) or a high risk (Class 2B) of metastasis based on the expression level of at least 10 genes in the gene set in combination with at least one risk factor.

17: The method of claim 16, wherein the at least one risk factor is selected from tumor size, tumor location, immune status, perineural invasion (PNI), depth of tumor invasion, tumor differentiation, histological subtype, and lymphovascular invasion.

18. The method of claim 14, wherein the at least 10 genes in the gene set are: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839.

19. The method of claim 14, wherein the at least 10 genes in the gene set are: ALOX12, LIME1, MMP10, PI3, RUNX3, TFAP2B, SPP1, HDDC3, LOC100287896, TAF6L, MRC1, RNF135, PLS3, and ZNF839.

20: The method of claim 14, wherein the gene set further comprises at least one control gene, and wherein the at least one control gene is selected from the group consisting of BAG6, KMT2D/MLL2, MDM2, FXR1, KMT2C, MDM4, VIM, and NF1B.

21: The method of claim 14, wherein the expression level of each gene in the gene set is determined by reverse transcribing the isolated mRNA and measuring a level of fluorescence for each gene in the gene set by a nucleic acid sequence detection system following RT-PCR.

22: The method of claim 14, wherein the cSCC tumor sample is obtained from a formalin-fixed, paraffin embedded sample.

23: The method of claim 15 further comprising administering a treatment selected from one or more of:
a) performing a resection of the cSCC tumor;
b) clinical follow-up of four to twelve times per year for about 3 years;
c) performing baseline and annual nodal imaging at least twice a year for about 2 years;
d) performing a nodal biopsy or a neck dissection;
e) administering an additional adjuvant treatment; and
f) enrolling in a clinical trial.

24: The method of claim 23, wherein the additional adjuvant treatment is one or more of radiation, chemoradiation, chemotherapy, regional limb therapy, surgery, systemic therapy, or immunotherapy.

25: The method of claim 5, wherein the expression level of:
ACSBG1 is decreased, ALOX12 is decreased, APOBEC3G is increased, ATP6V0E2 is increased, BBC3 is increased, BHLHB9 is decreased, CEP76 is decreased, DUXAP8 is increased, GTPBP2 is decreased, HDDC3 is increased, ID2 is decreased, LCE2B is decreased, LIME1 (ZGPAT) is increased, LOC100287896 is increased, LOC101927502 is increased, MMP10 is decreased, MRC1 is decreased, MSANTD4 is decreased, NFASC is decreased, NFIC is decreased, PDPN is increased, PI3 is decreased, PLS3 is decreased, RCHY1 is increased, RNF135 is increased, RPP38 is decreased, RUNX3 is increased, SLC1A3 is increased, SPP1 is increased, TAF6L is increased, TFAP2B is decreased, ZNF48 is increased, ZNF496 is increased, and ZNF839 is increased when comparing a recurrent tumor to a non-recurrent sample.

26: A method for treating a patient with a cutaneous squamous cell carcinoma (cSCC) tumor, the method comprising:
(a) obtaining a first risk classification in a cSCC tumor sample from the patient, wherein the first risk classification is obtained by:
determining the expression levels in a first gene set comprising: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839; and comparing the expression levels of the first gene set to a predictive training set to generate the first risk classification;
wherein the first risk classification provides an indication as to whether the cSCC tumor has a low risk (Class 1), a moderate risk (Class 2A), or a high risk (Class 2B), and
wherein when the cSCC tumor is identified as having a low risk (Class 1) or a moderate risk (Class 2A) the method further comprises:
(b) obtaining a second risk classification in the cSCC tumor sample from the patient, wherein the second risk classification is obtained by:
determining the expression levels in a second gene set comprising: ALOX12, LIME1, MMP10, PI3, RUNX3, TFAP2B, SPP1, HDDC3, LOC100287896, TAF6L, MRC1, RNF135, PLS3, and ZNF839; and comparing the expression levels of the second gene set to a predictive training set to generate the second risk classification;
wherein the second risk classification provides an indication as to whether the cSCC tumor has a lowest risk (Class 1A), a low risk (Class 1B), a high risk (Class 2A), or the highest risk (Class 2B), and;
(c) administering to the patient an aggressive treatment when the cSCC tumor is identified as having the high risk (Class 2A) or the highest risk (Class 2B) based on the second risk classification.

27: The method of claim 26, wherein the first risk classification and/or the second risk classification indicates risk of local recurrence and/or risk of metastasis.

28: The method of claim 26, further comprising generating the second risk classification based on the combination with at least one risk factor.

29: The method of claim 28, wherein the at least one risk factor is selected from immune status (competent vs suppressed), histological differentiation (well/moderate vs poor) tumor diameter (unknown or <2 cm or 2 cm to less than 4 cm or greater than or equal to 4 cm), perineural invasion (PNI), tumor location, immunosuppression, depth of invasion, differentiation, histological subtype, and lymphovascular invasion.

30: The method of claim 28, wherein the at least one risk factor is histological differentiation (well/moderate vs poor), tumor diameter (unknown or <2 cm or 2 cm to less than 4 cm or greater than or equal to 4 cm), and tumor location.

31: The method of claim 26 further comprising determining that the subject benefit from adjuvant radiotherapy when the patient has a cSCC tumor with a high risk of local recurrence and/or risk of metastasis.

32: The method of claim 26 further comprising administering the aggressive treatment, wherein the treatment is selected from one or more of:
a) performing a resection of the cSCC tumor;
b) clinical follow-up of four to twelve times per year for about 3 years;
c) performing baseline and annual nodal imaging at least twice a year for about 2 years;
d) performing a nodal biopsy or a neck dissection;
e) administering an adjuvant treatment; and
f) enrolling in a clinical trial.

33: The method of claim 32, wherein the adjuvant treatment is one or more of radiation, chemoradiation, chemotherapy, regional limb therapy, surgery, systemic therapy, or immunotherapy.

34: The method of claim 26, wherein the expression level of each gene in the first gene set and/or the second gene set is determined by reverse transcribing the isolated mRNA and measuring a level of fluorescence for each gene in the gene set by a nucleic acid sequence detection system following RT-PCR.

35: The method of claim 26, wherein the cSCC tumor sample is obtained from a formalin-fixed, paraffin embedded sample.

36: The method of claim 26, wherein the first gene set and/or the second gene set further comprise at least one control gene, and wherein the at least one control gene is selected from the group consisting of BAG6, KMT2D/MLL2, MDM2, FXR1, KMT2C, MDM4, VIM, and NF1B, optionally wherein the control genes are MDM2, KMT2D, BAG6, FXR1, MDM4, and KMT2C.

37: The method of claim 26, wherein the expression level of:
ACSBG1 is decreased, ALOX12 is decreased, APOBEC3G is increased, ATP6V0E2 is increased, BBC3 is increased, BHLHB9 is decreased, CEP76 is decreased, DUXAP8 is increased, GTPBP2 is decreased, HDDC3 is increased, ID2 is decreased, LCE2B is decreased, LIME1 (ZGPAT) is increased, LOC100287896 is increased, LOC101927502 is increased, MMP10 is decreased, MRC1 is decreased, MSANTD4 is decreased, NFASC is decreased, NFIC is decreased, PDPN is increased, PI3 is decreased, PLS3 is decreased, RCHY1 is increased, RNF135 is increased, RPP38 is decreased, RUNX3 is increased, SLC1A3 is increased, SPP1 is increased, TAF6L is increased, TFAP2B is decreased, ZNF48 is increased, ZNF496 is increased, and ZNF839 is increased, when comparing a recurrent tumor to a non-recurrent sample.

38: The method of claim 26, wherein the expression level of:
ALOX12 is decreased, HDDC3 is increased, LIME1 (ZGPAT) is increased, LOC100287896 is increased, MMP10 is decreased, MRC1 is decreased, PI3 is decreased, PLS3 is decreased, RNF135 is increased, RUNX3 is increased, SPP1 is increased, TAF6L is increased, TFAP2B is decreased, and ZNF839 is increased, when comparing a recurrent tumor to a non-recurrent sample.

39. A method for determining benefit from adjuvant radiotherapy in a subject having a cutaneous squamous cell carcinoma (cSCC) tumor, the method comprising:
a) determining the expression level of at least 10 genes in a gene set; wherein the at least 10 genes in the gene set are selected from: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839;
b) identifying a risk of metastasis of the cSCC tumor and providing an indication as to whether the cSCC tumor has a lowest risk (Class 1A), a low risk (Class 1B). a high risk (Class 2A), or the highest risk (Class 2B) of metastasis; and
c) determining that the subject will benefit from adjuvant radiotherapy when the determination is made that the subject has a cSCC tumor with the highest risk (Class 2B) of metastasis.

40. The method of claim 39 further comprising administering adjuvant radiotherapy to the subject when the cSCC tumor is identified as having the highest risk (Class 2B) of metastasis.

41: The method of claim 39 further comprising identifying that the cSCC tumor has the highest risk (Class 2B) of metastasis based on the expression level of at least 10 genes in the gene set in combination with at least one risk factor.

42: The method of claim 41, wherein the at least one risk factor is selected from tumor size, tumor location, immune status, perineural invasion (PNI), depth of tumor invasion, tumor differentiation, histological subtype, and lymphovascular invasion.

43. The method of claim 39, wherein the at least 10 genes in the gene set are: ACSBG1, ALOX12, APOBEC3G, ATP6V0E2, BBC3, BHLHB9, CEP76, DUXAP8, GTPBP2, HDDC3, ID2, LCE2B, LIME1 (ZGPAT), LOC100287896, LOC101927502, MMP10, MRC1, MSANTD4, NFASC, NFIC, PDPN, PI3, PLS3, RCHY1, RNF135, RPP38, RUNX3, SLC1A3, SPP1, TAF6L, TFAP2B, ZNF48, ZNF496, and ZNF839.

44. The method of claim 39, wherein the wherein the at least 10 genes in the gene set are: ALOX12, LIME1, MMP10, PI3, RUNX3, TFAP2B, SPP1, HDDC3, LOC100287896, TAF6L, MRC1, RNF135, PLS3, and ZNF839.

45: The method of claim 39, wherein the gene set further comprises at least one control gene, wherein the at least one control gene is selected from the group consisting of BAG6, KMT2D/MLL2, MDM2, FXR1, KMT2C, MDM4, VIM, and NF1B.

46: The method of claim 39, wherein the expression level of each gene in the gene set is determined by reverse transcribing the isolated mRNA and measuring a level of fluorescence for each gene in the gene set by a nucleic acid sequence detection system following RT-PCR.

47: The method of claim 39, wherein the cSCC tumor sample is obtained from a formalin-fixed, paraffin embedded sample.

48: The method of claim 40 further comprising administering a treatment selected from one or more of:
a) performing a resection of the cSCC tumor;
b) clinical follow-up of four to twelve times per year for about 3 years;
c) performing baseline and annual nodal imaging at least twice a year for about 2 years;
d) performing a nodal biopsy or a neck dissection;
e) administering an additional adjuvant treatment; and
f) enrolling in a clinical trial.

49: The method of claim 48, wherein the additional adjuvant treatment is one or more of radiation, chemoradiation, chemotherapy, regional limb therapy, surgery, systemic therapy, or immunotherapy.

50: The method of claim 30, wherein the expression level of:
ACSBG1 is decreased, ALOX12 is decreased, APOBEC3G is increased, ATP6V0E2 is increased, BBC3 is increased, BHLHB9 is decreased, CEP76 is decreased, DUXAP8 is increased, GTPBP2 is decreased, HDDC3 is increased, ID2 is decreased, LCE2B is decreased, LIME1 (ZGPAT) is increased, LOC100287896 is increased, LOC101927502 is increased, MMP10 is decreased, MRC1 is decreased, MSANTD4 is decreased, NFASC is decreased, NFIC is decreased, PDPN is increased, PI3 is decreased, PLS3 is decreased, RCHY1 is increased, RNF135 is increased, RPP38 is decreased, RUNX3 is increased, SLC1A3 is increased, SPP1 is increased, TAF6L is increased, TFAP2B is decreased, ZNF48 is increased, ZNF496 is increased, and ZNF839 is increased when comparing a recurrent tumor to a non-recurrent sample.
